(19) United States
(12) Patent Application Publication
(10) Pub. No.: US 2025/0092036 A1
(43) Pub. Date: Mar. 20, 2025

(54) COMPOUNDS AND COMPOSITIONS FOR MODULATING THE ACTIVITY OF ERK

(71) Applicant: Novartis AG, Basel (CH)

(72) Inventors: Erich W. BAUM, South Hamilton, MA (US); Thomas A. DINEEN, JR., Somerville, MA (US); Jonathan Edward HEMPEL, Arlington, MA (US); Matthew James HESSE, Oakland, CA (US); Robert KOENIG, Braintree, MA (US); Matthew James LAMARCHE, Reading, MA (US); Jay Francis LARROW, Wakefield, MA (US); Ruowei MO, New Hope, PA (US); Henrik MOEBITZ, Arlesheim (CH); Rukundo NTAGANDA, Weymouth, MA (US); Keith Bruce PFISTER, San Ramon, CA (US); Martin SENDZIK, Belmont, MA (US); Colin Keith SKEPPER, Alameda, CA (US); Joseph WZOREK, JR., Watertown, MA (US); Frédéric ZECRI, Belmont, MA (US)

(21) Appl. No.: 18/800,559

(22) Filed: Aug. 12, 2024

Related U.S. Application Data
(60) Provisional application No. 63/519,379, filed on Aug. 14, 2023.

(51) Int. Cl. C07D 471/04; A61K 31/454; A61K 31/4545; A61P 35/00; C07D 401/06; C07D 405/14; C07D 491/052

(52) U.S. Cl. CPC C07D 471/04; A61K 31/454; A61K 31/4545; A61P 35/00; C07D 401/06; C07D 405/14; C07D 491/052

(57) ABSTRACT

The invention relates to compounds which modulate the activity of ERK. The present invention also relates to processes for the preparation of said compounds, pharmaceutical compositions comprising said compounds, and use of said compounds in the treatment of conditions, diseases and disorders mediated by ERK.

### FIELD

The invention relates to compounds which modulate the activity of ERK (extracellular signal-regulated kinase). The present invention also relates to processes for the preparation of said compounds, pharmaceutical compositions comprising said compounds, and use of said compounds in the treatment of conditions, diseases and disorders mediated by ERK.

### BACKGROUND

The MAPK (mitogen-activated protein kinase) pathway is a key signaling cascade that drives cell proliferation, differentiation, and survival. Dysregulation of this pathway underlies many instances of tumorigenesis. Aberrant signaling or inappropriate activation of the MAPK pathway has been shown in multiple tumor types and can occur through several distinct mechanisms, including activating mutations in RAS (rat sarcoma virus) and BRAF (B-Raf proto-oncogene, serine/theonine kinase). The MAPK pathway is frequently mutated in human cancer with KRAS (Kirsten rat sarcoma virus) and BRAF mutations being among the most frequent (approximately 30%). RAS mutations, particularly gain of function mutations, have been detected in 9-30% of all cancers, with KRAS mutations having the highest prevalence (86%).

The extracellular signal-regulated kinases are one class of signaling kinases that are involved in conveying extracellular signals into cells and subcellular organelles. ERK1 and ERK2 are involved in regulating a wide range of activities and dysregulation of the ERK1/2 cascade is known to cause a variety of pathologies including neurodegenerative diseases, developmental diseases, diabetes and cancer. The role of ERK1/2 in cancer is of special interest because activating mutations upstream of ERK1/2 in its signaling cascade are believed to be responsible for more than half of all cancers. Moreover, excessive ERK1/2 activity was also found in cancers where the upstream components were not mutated, suggesting that ERK1/2 signaling plays a role in carcinogenesis even in cancers without mutational activations. The ERK pathway has also been shown to control tumor cell migration and invasion, and thus may be associated with metastasis.

The prognosis for patients suffering from certain cancers remains poor. Resistance to treatment occurs frequently and not all patients respond to available treatments. For example, the median survival for patients suffering from advanced colorectal cancer with BRAF mutation is less than 12 months. In normal cell signaling, the MAPK pathway is held under tight regulation by negative feedback at multiple levels. In BRAF V600-mutant melanomas, for example, the negative regulation upstream of BRAF is lost, leading to an increased dependence of these cells on negative regulation at the level of ERK.

While inhibition of the MAPK pathway can negatively affect melanoma cell growth, hyperactivation of ERK can also be detrimental to cell survival. Hyperactivation of ERK via this mechanism leads to an increase in MAPK output, cell cycle arrest, intolerable levels of cell stress, and cell death. In vivo, MAPK hyperactivation leads to profound and durable tumor regressions in BRAF-mutant melanoma cell lines and patient derived xenografts.

It is important to develop new therapies for patients suffering from cancer to achieve better clinical outcomes. Treatment options which are better tolerated and/or provide durable anti-tumor responses are also desired.

### SUMMARY

The compounds of the present invention are small molecular protein-protein interaction disruptors that are capable of blocking the negative regulation of ERK as a novel therapeutic approach for the treatment of cancers, for example, MAPK dysregulated cancers, BRAF and/or RAS mutant cancers, melanoma, lung cancer, colorectal cancer, pancreatic cancer and thyroid cancer.

Thus, according to a first aspect of the invention, there is hereby provided a compound of formula (I):

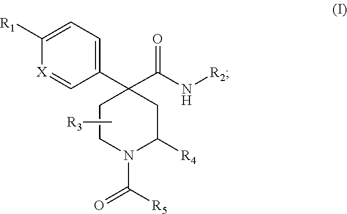

wherein:
X is selected from N and CR₆, wherein R₆ is selected from hydrogen and halo;
R₁ is selected from hydrogen and halo;
R₂ is selected from —X₁—R₂ₐ and R₂ₐ;
X₁ is selected from C₁-C₄alkylene and C₂-C₄haloalkylene;
R₂ₐ is selected from i) hydrogen, and ii) a ring substituted with 0 to 3 substituents R_{2b}, wherein the ring is selected from a) 3-6 membered saturated or partially unsaturated carbocyclic ring, b) 5-6 membered heteroaryl, c) phenyl, and d) 4 to 6 membered heterocyclyl (e.g. fully saturated heterocyclyl) containing 1 or 2 heteroatoms independently selected from oxygen and nitrogen;
each R_{2b} is independently selected from halo, hydroxyl, C₁-C₃alkyl, C₃-C₄cycloalkyl, C₁-C₃haloalkyl, C₁-C₃hydroxyalkyl, O—C₁-C₃alkyl, C₁-C₃alkylene-O—C₁-C₃alkyl, cyano, —CO₂R₁₁, —CO₂N(R₁₁)₂, —X₂—CO₂R₁₁ and —X₂—CO₂N(R₁₁)₂;
X₂ is selected from C₁-C₅alkylene and C₃-C₆cycloalkylene;
each R₁₁ is independently selected from hydrogen, C₁-C₅alkyl, C₃-C₅cycloalkyl, C₁-C₅haloalkyl and C₃-C₅cyclohaloalkyl, or two R₁₁ groups together with the nitrogen atom to which they are mutually attached join to form a 4 to 6 membered heterocyclic ring containing 1 heteroatom which is nitrogen;
R₃ is selected from hydrogen, C₁-C₃alkyl and C₁-C₃haloalkyl, and R₄ is hydrogen, or R₃ and R₄ together with the piperidinyl ring of formula (I) to which R₃ and R₄ are attached join to form a 7 or 8 membered bridged or fused heterocyclic ring;
R₅ is selected from:

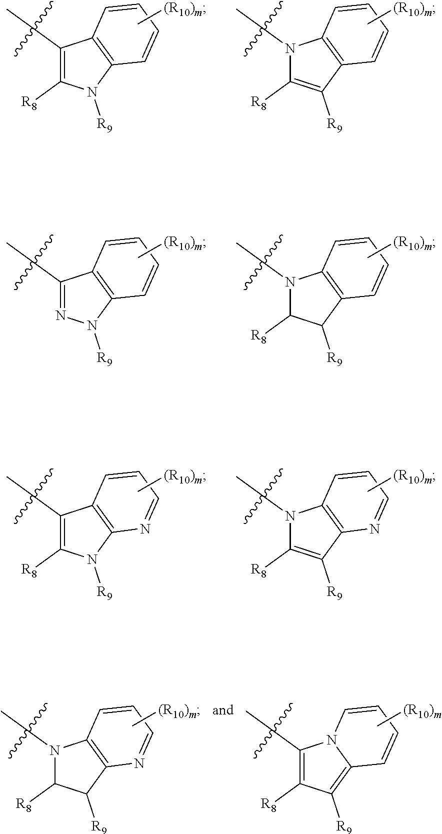

wherein:
R₃ is selected from hydrogen, halo, C₁-C₆alkyl, C₃-C₄cycloalkyl, C₁-C₆haloalkyl, C₁-C₆alkylene-O—C₁-C₄alkyl, C₁-C₆haloalkylene-O—C₁-C₄alkyl, C₁-C₅hydroxyalkyl, C₁-C₆haloalkylene-O—C₁-C₄haloalkyl, C₁-C₆alkylene-O—C₁-C₄haloalkyl, C(═O)H and cyano, and R₉ is selected from —X₃—R₉ₐ and R₉ₐ;
or R₃ and R₉ together with the carbon atoms to which R₃ and R₉ are attached form a ring substituted with 0 to 3 R_{9b} groups, wherein the ring is a) 5-6 membered saturated or partially unsaturated carbocyclic ring, or b) 5-6 membered heterocyclyl comprising 1 heteroatom which is O;
X₃ is selected from C₁-C₂alkylene and C₃-C₅cycloalkylene;
R₉ₐ is selected from i) hydrogen and ii) a ring substituted with 0 to 3 R_{9b} groups, wherein the ring is a) phenyl, b) 5-6 membered heteroaryl, c) C₃-C₇cycloalkyl, d) C₇-C₉spiroalkyl, e) 4 to 7 membered heterocyclyl comprising 1 or 2 heteroatoms which is/are each O, or f) 7 to 9 membered spiroheterocyclyl comprising 1 or 2 heteroatoms which is/are each O;
each R_{9b} is independently selected from halo, hydroxy, C₁-C₄alkyl, C₁-C₄haloalkyl, O—C₁-C₄alkyl, O—C₁-C₄haloalkyl, C₁-C₄alkylene-O—C₁-C₄alkyl, C₁-C₄haloalkylene-O—C₁-C₄alkyl, C₁-C₄alkylene-O—C₁-C₄haloalkyl, C₁-C₄haloalkylene-O—C₁-C₄haloalkyl, C₃-C₇cycloalkyl substituted with 0 to 2 R_{9c} groups, C₃-C₇cyclohaloalkyl substituted with 0 to 2 R_{9c} groups, O—C₃-C₇cycloalkyl substituted with 0 to 2 R_{9c} groups, O—C₃-C₇cyclohaloalkyl substituted with 0 to 2 R_{9c} groups, 3-7 membered heterocyclyl comprising 1 heteroatom which is O substituted with 0 to 2 R_{9c} groups, O-3-7 membered heterocyclyl comprising 1 heteroatom which is O substituted with 0 to 2 R_{9c} groups, phenyl substituted with 0 to 2 R_{9c} groups, pyridinyl substituted with 0 to 2 R_{9c} groups, O—C₁-C₃alkylene-C₃-C₇cycloalkyl substituted with 0 to 2 R_{9c} groups, O—C₁-C₃alkylene-C₃-C₇cyclohaloalkyl substituted with 0 to 2 R_{9c} groups and O—C₁-C₃alkylene-3-7 membered heterocyclyl comprising 1 heteroatom which is O substituted with 0 to 2 R_{9c} groups;
or wherein two R_{9b} groups in combination with the carbon atom to which they are mutually attached form a ring substituted with 0 to 2 R_{9c} groups, wherein the ring is i) C₃-C₆cycloalkyl, or ii) 4-6 membered heterocyclyl containing one heteroatom which is oxygen;
each R_{9c} is independently selected from halo (e.g. fluoro), CH₃ and OCH₃;
each R₁₀ is halo; and
m is an integer from 0 to 2;
or a pharmaceutically acceptable salt thereof.

According to a second aspect of the invention, there is hereby provided a compound according to any one of the Examples, or a pharmaceutically acceptable salt thereof.

According to a third aspect of the invention, there is hereby provided a pharmaceutical composition comprising the compound or pharmaceutically acceptable salt thereof according to the first or second aspect of the invention, and one or more pharmaceutically acceptable carriers.

According to a fourth aspect of the invention, there is hereby provided a combination comprising the compound or pharmaceutically acceptable salt thereof according to the first or second aspect of the invention, and one or more additional therapeutically active agents.

According to a fifth aspect of the invention, there is hereby provided a method of modulating ERK activity in a subject, the method comprising administering to the subject a therapeutically effective amount of the compound or pharmaceutically acceptable salt thereof according to the first or the second aspect of the invention, or the pharmaceutical composition according to the third aspect of the invention.

According to a sixth aspect of the invention, there is hereby provided a method of treating a patient having a disease associated with aberrant activity of the MAP kinase pathway comprising administering to said patient a therapeutically effective amount of the compound or pharmaceutically acceptable salt thereof according to the first or the second aspect of the invention, or the pharmaceutical composition according to the third aspect of the invention.

According to a seventh aspect of the invention, there is hereby provided a compound or pharmaceutically acceptable salt thereof according to the first or the second aspect of the invention for use as a medicament.

According to an eighth aspect of the invention, there is hereby provided a compound or pharmaceutically acceptable salt thereof according to the first or the second aspect of the invention for use in the treatment of cancer.

According to a ninth aspect of the invention, there is hereby provided use of a compound or pharmaceutically acceptable salt thereof according to the first or the second aspect of the invention in the manufacture of a medicament for the treatment of cancer.

### BRIEF DESCRIPTION OF THE DRAWINGS

FIGS. 1A-1B show (FIG. 1A) change in tumor volume (mm³) and (FIG. 1B) body weight change (%) following administration of a compound of Formula I (specifically, Example 13) in WM793 BRAFV600E tumor xenograft in nude mice.

FIGS. 2A-2B show (FIG. 2A) change in tumor volume (mm³) and (FIG. 2B) body weight change (%) following administration of a compound of Formula I (specifically, Example 4 or Example 5) in WM793 BRAFV600E tumor xenograft in nude rats.

### DETAILED DESCRIPTION

The invention therefore, in a first aspect, provides a compound of formula (I):

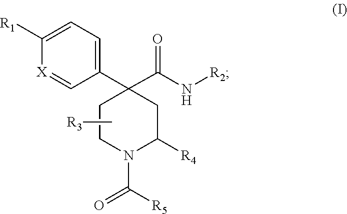

wherein:
X is selected from N and CR₆, wherein R₆ is selected from hydrogen and halo;
R₁ is selected from hydrogen and halo;
R₂ is selected from —X₁—R₂ₐ and R₂ₐ;
X₁ is selected from C₁-C₄alkylene and C₂-C₄haloalkylene;
R₂ₐ is selected from i) hydrogen, and ii) a ring substituted with 0 to 3 substituents R_{2b}, wherein the ring is selected from a) 3-6 membered saturated or partially unsaturated carbocyclic ring, b) 5-6 membered heteroaryl, c) phenyl, and d) 4 to 6 membered heterocyclyl (e.g. fully saturated heterocyclyl) containing 1 or 2 heteroatoms independently selected from oxygen and nitrogen;
each R_{2b} is independently selected from halo, hydroxyl, C₁-C₃alkyl, C₃-C₄cycloalkyl, C₁-C₃haloalkyl, C₁-C₃hydroxyalkyl, O—C₁-C₃alkyl, C₁-C₃alkylene-O—C₁-C₃alkyl, cyano, —CO₂R₁₁, —CO₂N(R₁₁)₂, —X₂—CO₂R₁₁ and —X₂—CO₂N(R₁₁)₂;
X₂ is selected from C₁-C₅alkylene and C₃-C₆cycloalkylene;
each R₁ is independently selected from hydrogen, C₁-C₅alkyl, C₃-C₅cycloalkyl, C₁-C₅haloalkyl and C₃-C₅cyclohaloalkyl, or two R₁ groups together with the nitrogen atom to which they are mutually attached join to form a 4 to 6 membered heterocyclic ring containing 1 heteroatom which is nitrogen;
R₃ is selected from hydrogen, C₁-C₃alkyl and C₁-C₃haloalkyl, and R₄ is hydrogen, or R₃ and R₄ together with the piperidinyl ring of formula (I) to which R₃ and R₄ are attached join to form a 7 or 8 membered bridged or fused heterocyclic ring;
R₅ is selected from:

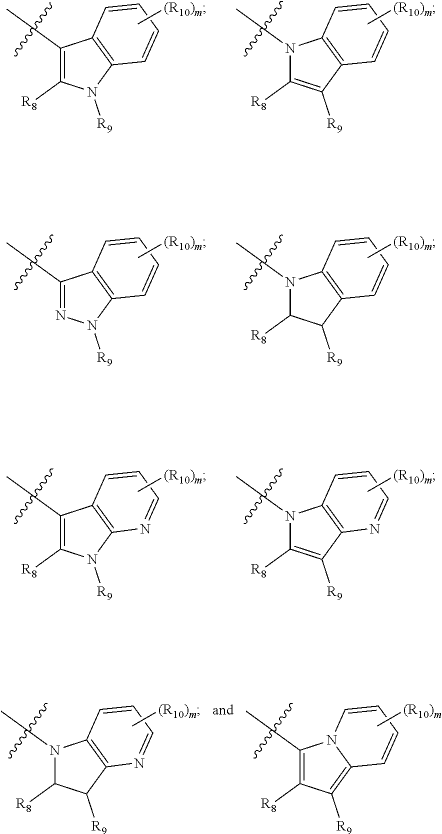

wherein:
R₃ is selected from hydrogen, halo, C₁-C₆alkyl, C₃-C₄cycloalkyl, C₁-C₆haloalkyl, C₁-C₆alkylene-O—C₁-C₄alkyl, C₁-C₆haloalkylene-O—C₁-C₄alkyl, C₁-C₆hydroxyalkyl, C₁-C₆haloalkylene-O—C₁-C₄haloalkyl, C₁-C₆alkylene-O—C₁-C₄haloalkyl, C(═O)H and cyano, and R₉ is selected from —X₃—R₉ₐ and R₉ₐ;
or R₃ and R₉ together with the carbon atoms to which R₃ and R₉ are attached form a ring substituted with 0 to 3 R_{9b} groups, wherein the ring is a) 5-6 membered saturated or partially unsaturated carbocyclic ring, or b) 5-6 membered heterocyclyl comprising 1 heteroatom which is O;
X₃ is selected from C₁-C₂alkylene and C₃-C₅cycloalkylene;
R₉ₐ is selected from i) hydrogen and ii) a ring substituted with 0 to 3 R_{9b} groups, wherein the ring is a) phenyl, b) 5-6 membered heteroaryl, c) C₃-C₇cycloalkyl, d) C₇-C₉spiroalkyl, e) 4 to 7 membered heterocyclyl comprising 1 or 2 heteroatoms which is/are each O, or f) 7 to 9 membered spiroheterocyclyl comprising 1 or 2 heteroatoms which is/are each O;
each R_{9b} is independently selected from halo, hydroxy, C₁-C₄alkyl, C₁-C₄haloalkyl, O—C₁-C₄alkyl, O—C₁-C₄haloalkyl, C₁-C₄alkylene-O—C₁-C₄alkyl, C₁-C₄haloalkylene-O—C₁-C₄alkyl, C₁-C₄alkylene-O—C₁-C₄haloalkyl, C₁-C₄haloalkylene-O—C₁-C₄haloalkyl, C₃-C₇cycloalkyl substituted with 0 to 2 R_{9c} groups, C₃-C₇cyclohaloalkyl substituted with 0 to 2 R₉, groups, O—C₃-C₇cycloalkyl substituted with 0 to 2 R_{9c} groups, O—C₃-C₇cyclohaloalkyl substituted with 0 to 2 R_{9c} groups, 3-7 membered heterocyclyl comprising 1 heteroatom which is O substituted with 0 to 2 R_{9c} groups, O-3-7 membered heterocyclyl comprising 1 heteroatom which is O substituted with 0 to 2 R_{9c} groups, phenyl substituted with 0 to 2 R_{9c} groups, pyridinyl substituted with 0 to 2 R_{9c} groups, O—C₁-C₃alkylene-C₃-C₇cycloalkyl substituted with 0 to 2 R_{9c} groups, O—C₁-C₃alkylene-C₃-C₇cyclohaloalkyl substituted with 0 to 2 R_{9c} groups and O—C₁-C₃alkylene-3-7 membered heterocyclyl comprising 1 heteroatom which is O substituted with 0 to 2 R_{9c} groups;
or wherein two R_{9b} groups in combination with the carbon atom to which they are mutually attached form a ring substituted with 0 to 2 R_{9c} groups, wherein the ring is i) C₃-C₆cycloalkyl, or ii) 4-6 membered heterocyclyl containing one heteroatom which is oxygen;
each R_{9c} is independently selected from halo (e.g. fluoro), CH₃ and OCH₃;
each R₁₀ is halo; and
m is an integer from 0 to 2;
or a pharmaceutically acceptable salt thereof.

In an embodiment, the compound is of formula (Ia):

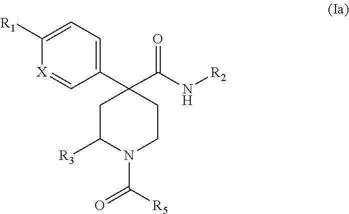

in which:
X is selected from N and CR₆; wherein R₆ is selected from hydrogen and halo;
R₁ is selected from hydrogen and halo;
R₂ is selected from —X₁—R₂ₐ and R₂ₐ;
X₁ is selected from C₁-C₂alkylene and C₂haloalkylene;
R₂ₐ is selected from i) hydrogen and ii) a ring substituted with 0 to 3 substituents R_{2b}; wherein the ring is selected from a) 3-6 membered saturated or partially unsaturated carbocyclic ring, b) 5-6 membered heteroaryl, c) phenyl, and d) 4 to 6 membered heterocyclyl (e.g. fully saturated heterocyclyl) containing 1 or 2 heteroatoms independently selected from oxygen and nitrogen;
each R_{2b} is independently selected from halo, hydroxyl, C₁-C₃alkyl, C₁-C₃haloalkyl, C₁-C₃hydroxyalkyl, O—C₁-C₃alkyl, C₁-C₃alkylene-O—C₁-C₃alkyl, cyano, —CO₂R₁₁, —CO₂N(R₁₁)₂, —X₂—CO₂R₁₁ and —X₂—CO₂N(R₁₁)₂;
X₂ is selected from C₁-C₅alkylene and C₃-C₆cycloalkylene;
each R₁₁ is independently selected from hydrogen, C₁-C₅alkyl and C₃-C₆cycloalkyl;
R₃ is selected from hydrogen and methyl;
R₅ is selected from:

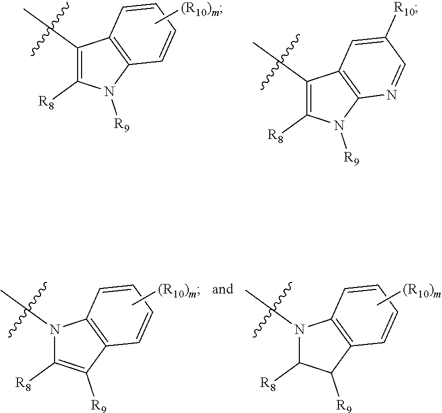

wherein:
R₃ is selected from hydrogen, methyl, ethyl, CHF₂, CH₂OCH₂CH₃, CH₂CH₂OCH₃, C(═O)H and cyano, and R₉ is selected from X₃—R₉ₐ and R₉ₐ;
or R₃ and R₉ together with the carbon atoms to which R₃ and R₉ are attached form a ring substituted with 0 to 1 R_{9b} groups, wherein the ring is a 5-6 membered heterocyclyl comprising 1 heteroatom which is O;
X₃ is C₁-C₂alkylene;
R₉ₐ is selected from i) hydrogen, ii) a ring substituted with 0 to 3 R_{9b} groups, wherein the ring is a) C₄-C₆cycloalkyl, b) 6 membered heterocyclyl comprising 1 heteroatom which is O or c) 7 to 9 membered spiroheterocyclyl comprising 1 or 2 heteroatoms which is/are each O;
each R_{9b} is independently selected from halo, C₁-C₃alkyl, O—C₁-C₃alkyl, O-4-6 membered heterocyclyl comprising 1 heteroatom which is O substituted with 0 to 2 R_{9c} groups, pyridinyl substituted with 0 to 2 R_{9c} groups and O—C₁-C₃alkylene-4-6 membered heterocyclyl comprising 1 heteroatom which is O substituted with 0 to 2 R_{9c} groups;
or wherein two R_{9b} groups in combination with the carbon atom to which they are mutually attached form a ring substituted with 0 to 2 R_{9c} groups, wherein the ring is i) C₃-C₆cycloalkyl, or ii) 4-6 membered heterocyclyl containing one heteroatom which is oxygen;
each R_{9c} is independently selected from halo (e.g. fluoro), CH₃ and OCH₃;
each R₁₀ is halo; and
m is an integer from 0 to 2;
or a pharmaceutically acceptable salt thereof.

In an embodiment, X is CH or CF.

In an embodiment, X is CH.

In an embodiment, R₂ is R₂ₐ.

In an embodiment, R₂ₐ is a ring substituted with 0 to 3 substituents R_{2b}; wherein the ring is selected from a) C₄-C₆ cycloalkyl, and b) 4 to 6 membered heterocyclyl (e.g. fully saturated heterocyclyl) containing 1 or 2 heteroatoms independently selected from oxygen and nitrogen.

In a preferred embodiment, R₂ is R₂ₐ, and R₂ₐ is a ring substituted with 0 to 3 substituents R_{2b}; wherein the ring is selected from a) C₄-C₆ cycloalkyl, and b) 4 to 6 membered heterocyclyl (e.g. fully saturated heterocyclyl) containing 1 or 2 heteroatoms independently selected from oxygen and nitrogen. In a particularly preferred embodiment, R₂ₐ is a ring substituted with 1 to 3 substituents R_{2b}; wherein the ring is selected from a) C₅-C₆ cycloalkyl, and b) 4 to 6 membered heterocyclyl (e.g. fully saturated heterocyclyl) containing 1 or 2 heteroatoms independently selected from oxygen and nitrogen.

In an alternative embodiment, R₂ is —X₁—R₂ₐ, X₁ is selected from C₁-C₄alkylene and C₂-C₄haloalkylene and R₂ₐ is H.

In an embodiment, each R_{2b} is independently selected from halo, C₁-C₃alkyl and CO₂H.

In an embodiment, each R_{2b} is independently selected from fluoro, chloro, methyl and CO₂H.

In an embodiment, R₃ is H.

In an embodiment R₅ is selected from:

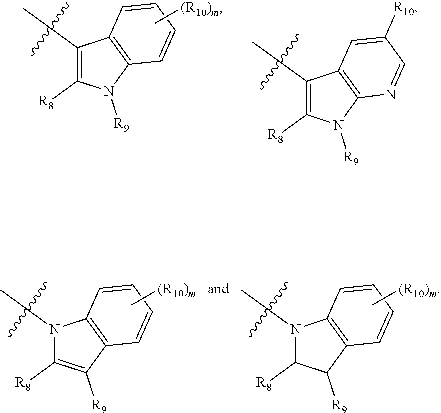

In an embodiment, m is 1 or 2.

In an embodiment, R₅ is selected from:

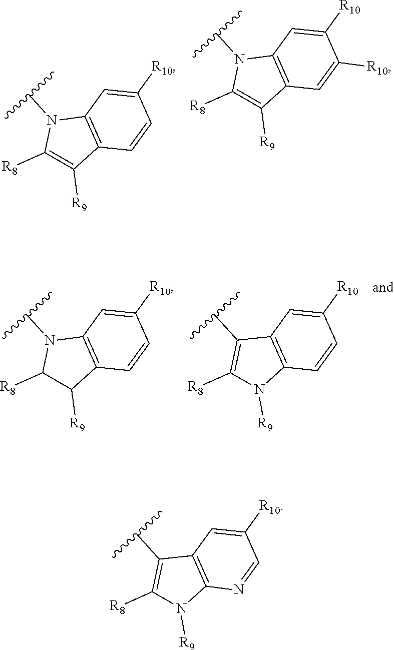

In an embodiment, each R₁₀ is independently selected from fluoro, chloro and bromo.

In an embodiment, R₂ₐ is C₅-C₆cycloalkyl substituted with 1 to 3 substituents R_{2b}. In an embodiment, R₂ₐ is C₆cycloalkyl substituted with 1 to 3 substituents R_{2b}. In an embodiment, one R_{2b} is CO₂H, and the other 0 to 2 R_{2b} groups are each independently selected from methyl, fluoro and chloro.

In an embodiment, R₃ is selected from hydrogen, C₁-C₃alkyl and C₁-C₃haloalkyl, and R₄ is hydrogen.

In an embodiment, R₉ₐ is selected from the group consisting of

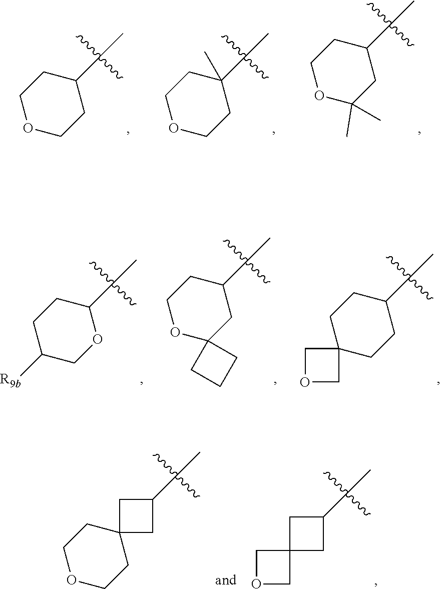

Where R₂ is

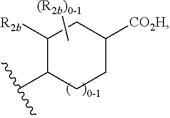

and there are two R_{2b} groups present, the R_{2b} group with an undefined position can be situated on any ring carbon atom, including (but not limited to) the ring carbon atom bonded to the R_{2b} group having a defined position or the ring carbon atom bonded to the CO₂H group

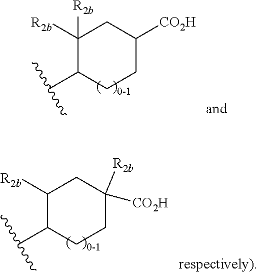

In an embodiment, the compound is of formula (Ib):

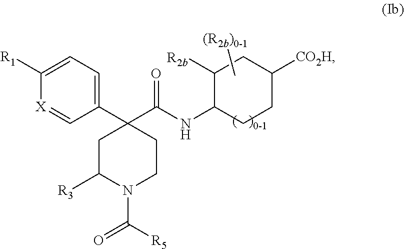

wherein X, R₁, R₃, R₅ and each R_{2b} are as defined above (e.g. as defined in the broadest embodiment of the first aspect of the invention). In an embodiment, each R_{2b} is independently selected from methyl, fluoro and chloro.

In an embodiment, R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b}. In an embodiment, R₂ₐ is 4 to 6 membered heterocyclyl (e.g. fully saturated heterocyclyl) containing 1 heteroatom which is nitrogen substituted with 0 to 2 substituents R_{2b}. In an embodiment, R₂ₐ is 4 to 6 membered heterocyclyl (e.g. fully saturated heterocyclyl) containing 1 heteroatom which is nitrogen substituted with 1 or 2 substituents R_{2b}, and wherein each R_{2b} substituent is independently selected from methyl and halo (e.g. fluoro).

In an embodiment, R₃ is selected from hydrogen, methyl, ethyl, CHF₂, CH₂CH₂OCH₃, C(═O)H and cyano.

In an embodiment, R₉ is X₃—R₉ₐ. In an embodiment, X₃ is CH₂.

In an embodiment, R₉ₐ is a ring substituted with 0 to 2 R_{9b} groups, wherein the ring is a) C₅-C₆cycloalkyl or b) 6 membered heterocyclyl comprising 1 heteroatom which is O. In an embodiment, R₉ₐ is a ring substituted with 0 or 1 R_{9b} groups, wherein the ring is a) C₆cycloalkyl or b) 6 membered heterocyclyl comprising 1 heteroatom which is O.

In an embodiment, each R_{9b} is independently selected from O—C₁-C₃alkyl and O-6 membered heterocyclyl comprising 1 heteroatom which is O.

In an embodiment, R₃ and R₉ together with the carbon atoms to which R₃ and R₉ are attached form a ring substituted with 0 to 1 R_{9b} groups, wherein the ring is a 5-6 membered heterocyclyl comprising 1 heteroatom which is O.

In an embodiment, the compound is of formula (Ic) or (Id):

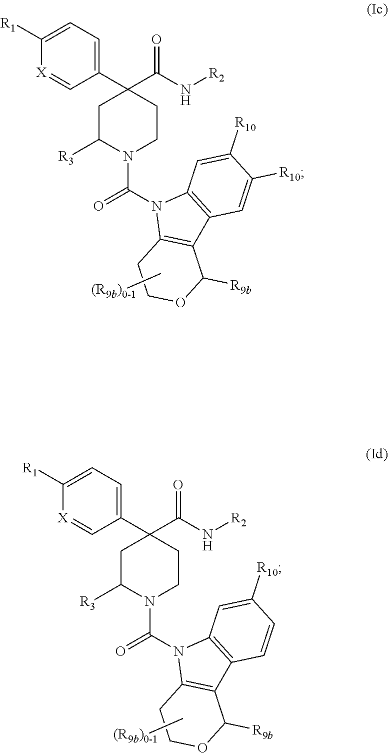

wherein X, R₁, R₂, R₃, R_{9b} and each R₁₀ independently are as defined above (e.g. as defined in the broadest embodiment of the first aspect of the invention).

In an embodiment, the compound is of formula (Ic-1) or (Id-1):

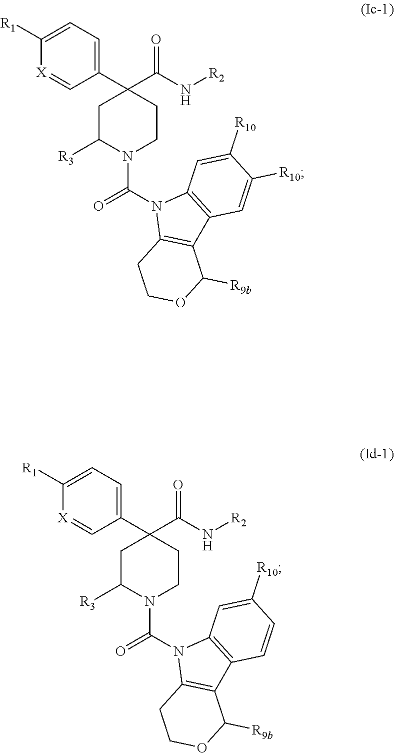

wherein X, R₁, R₂, R₃, R_{9b} and each R₁₀ independently are as defined above (e.g. as defined in the broadest embodiment of the first aspect of the invention). In an embodiment, R_{9b} is selected from O-6 membered heterocyclyl comprising 1 heteroatom which is O, and phenyl.

In an embodiment, the compound is of formula (Ic-2) or (Id-2):

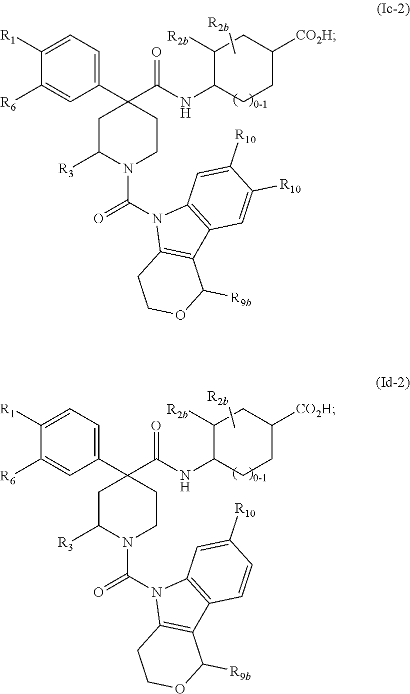

wherein R₁, each R_{2b} independently, R₃, R₆, R_{9b} and each R₁₀ independently are as defined above (e.g. as defined in the broadest embodiment of the first aspect of the invention). In an embodiment, R_{9b} is selected from O-6 membered heterocyclyl comprising 1 heteroatom which is O, and phenyl.

In an embodiment, the compound is of formula (II):

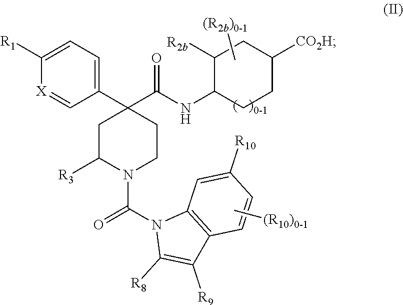

wherein X, R₁, R₃, each R_{2b} independently, and each R₁₀ independently are as defined above (e.g. as defined in the broadest embodiment of the first aspect of the invention),
R₈ is selected from hydrogen, methyl, ethyl, CHF₂, CH₂CH₂OCH₃, C(═O)H and cyano;
R₉ is X₃—R₉ₐ;
X₃ is CH₂;
R₉ₐ is selected from i) hydrogen and ii) a ring substituted with 0 to 2 (e.g. 0 to 1) R_{9b} groups, wherein the ring is a) C₅-C₆cycloalkyl or b) 6 membered heterocyclyl comprising 1 heteroatom which is O; and each R_{9b} is as defined above (e.g. as defined in the broadest embodiment of the first aspect of the invention), (e.g. each R_{9b} is independently selected from O—C₁-C₃alkyl and 6 membered heterocyclyl comprising 1 heteroatom which is O).

In an embodiment, the compound is of formula (III):

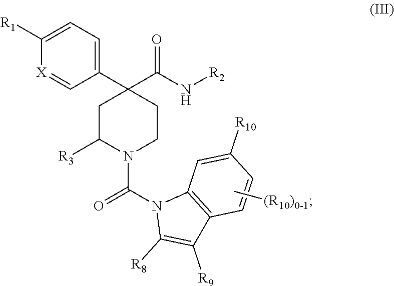

wherein X, R₁, R₃, and each R₁₀ independently are as defined above (e.g. as defined in the broadest embodiment of the first aspect of the invention),
R₂ is R₂ₐ;
R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b};
each R_{2b} substituent is independently selected from methyl and halo (e.g. fluoro);
R₃ is selected from hydrogen, methyl, ethyl, CHF₂, CH₂CH₂OCH₃, C(═O)H and cyano;
R₉ is X₃—R₉ₐ;
X₃ is CH₂; and
R₉ₐ is selected from i) hydrogen and ii) a ring substituted with 0 to 2 (e.g. 0 to 1) R_{9b} groups, wherein the ring is a) C₅-C₆cycloalkyl or b) 6 membered heterocyclyl comprising 1 heteroatom which is O; and each R_{9b} is as defined above (e.g. as defined in the broadest embodiment of the first aspect of the invention), (e.g. each R_{9b} is independently selected from O—C₁-C₃alkyl and 6 membered heterocyclyl comprising 1 heteroatom which is O).

In an embodiment, the compound is of formula (IV):

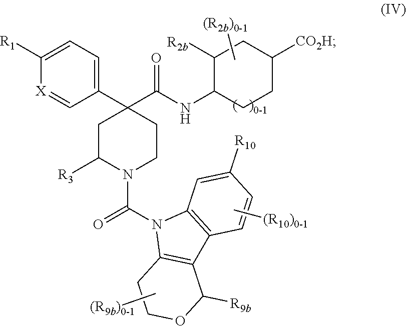

wherein X, R₁, R₃, each R_{2b} independently, each R_{9b} independently and each R₁₀ independently are as defined above (e.g. as defined in the broadest embodiment of the first aspect of the invention).

In an embodiment, the compound is of formula (V):

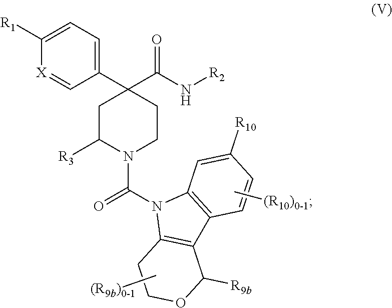

wherein X, R₁, R₃, each R_{9b} independently and each R₁₀ independently are as defined above (e.g. as defined in the broadest embodiment of the first aspect of the invention), R₂ is R₂ₐ;
R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b}; and
each R_{2b} substituent is independently selected from methyl and halo (e.g. fluoro).

In an embodiment, the compound is of formula (VI):

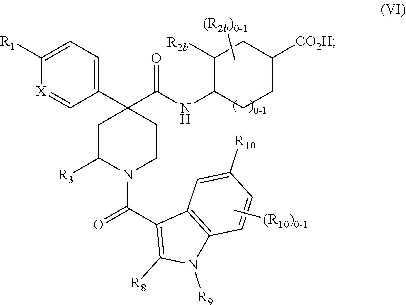

wherein:
X, R₁, R₃, each R_{2b} independently, and each R₁₀ independently are as defined above (e.g. as defined in the broadest embodiment of the first aspect of the invention),
R₃ is selected from hydrogen, methyl, ethyl, CHF₂, CH₂CH₂OCH₃, C(═O)H and cyano;
R₉ is X₃—R₉ₐ;
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and each R_{9b} is as defined above (e.g. as defined in the broadest embodiment of the first aspect of the invention), (e.g. each R_{9b} is independently selected from O—C₁-C₃alkyl and 6 membered heterocyclyl comprising 1 heteroatom which is O).

In an embodiment, the compound is of formula (VII):

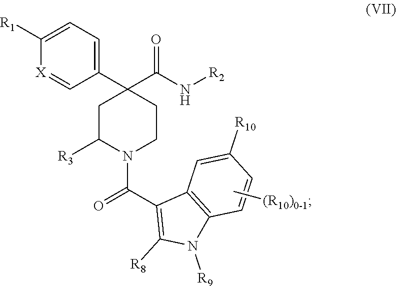

wherein:
X, R₁, R₃, and each R₁₀ independently are as defined above (e.g. as defined in the broadest embodiment of the first aspect of the invention),
R₂ is R₂ₐ;
R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b};
each R_{2b} substituent is independently selected from methyl and halo (e.g. fluoro);
R₃ is selected from hydrogen, methyl, ethyl, CHF₂, CH₂CH₂OCH₃, C(═O)H and cyano;
R₉ is X₃—R₉ₐ;
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and each R_{9b} is as defined above (e.g. as defined in the broadest embodiment of the first aspect of the invention), (e.g. each R_{9b} is independently selected from O—C₁-C₃alkyl and 6 membered heterocyclyl comprising 1 heteroatom which is O).

In an embodiment, the compound is of formula (VIII):

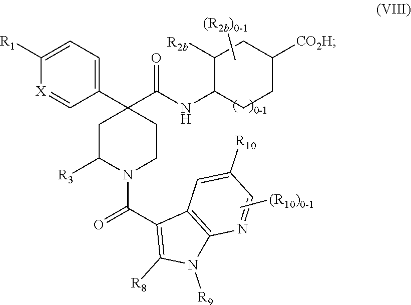

wherein:
X, R₁, R₃, each R_{2b} independently, and each R₁₀ independently are as defined above (e.g. as defined in the broadest embodiment of the first aspect of the invention),
R₃ is selected from hydrogen, methyl, ethyl, CHF₂, CH₂CH₂OCH₃, C(═O)H and cyano;
R₉ is X₃—R₉ₐ;
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and each R_{9b} is as defined above (e.g. as defined in the broadest embodiment of the first aspect of the invention), (e.g. each R_{9b} is independently selected from O—C₁-C₃alkyl and 6 membered heterocyclyl comprising 1 heteroatom which is O).

In an embodiment, the compound is of formula (IX):

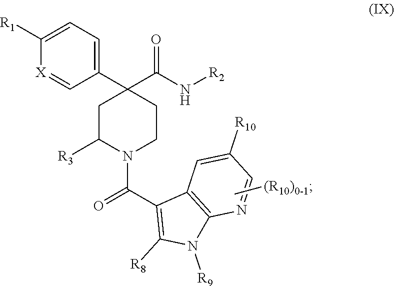

wherein:
X, R₁, R₃, and each R₁₀ independently are as defined above (e.g. as defined in the broadest embodiment of the first aspect of the invention),
R₂ is R₂ₐ;
R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b};
each R_{2b} substituent is independently selected from methyl and halo (e.g. fluoro);
R₃ is selected from hydrogen, methyl, ethyl, CHF₂, CH₂CH₂OCH₃, C(═O)H and cyano;
R₉ is X₃—R₉ₐ;
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and
each R_{9b} is as defined above (e.g. as defined in the broadest embodiment of the first aspect of the invention), (e.g. each R_{9b} is independently selected from O—C₁-C₃alkyl and 6 membered heterocyclyl comprising 1 heteroatom which is O).

In an embodiment, the compound is of formula (X):

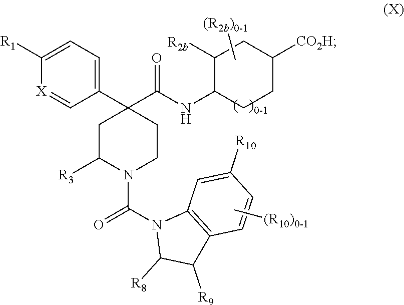

wherein:
X, R₁, R₃, each R_{2b} independently, and each R₁₀ independently are as defined above (e.g. as defined in the broadest embodiment of the first aspect of the invention),
R₃ is selected from hydrogen, methyl, ethyl, CHF₂, CH₂CH₂OCH₃, C(═O)H and cyano;
R₉ is R₉ₐ or X₃—R₉ₐ (particularly R₉ₐ);
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and each R_{9b} is as defined above (e.g. as defined in the broadest embodiment of the first aspect of the invention), (e.g. each R_{9b} is independently selected from O—C₁-C₃alkyl and 6 membered heterocyclyl comprising 1 heteroatom which is O).

In an embodiment, the compound is of formula (XI):

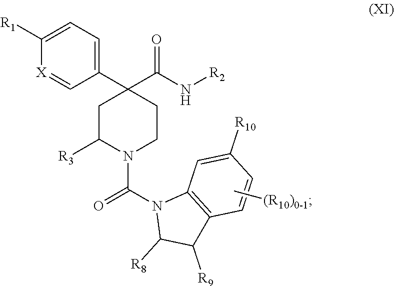

wherein:
X, R₁, R₃, and each R₁₀ independently are as defined above (e.g. as defined in the broadest embodiment of the first aspect of the invention),
R₂ is R₂ₐ;
R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b};
each R_{2b} substituent is independently selected from methyl and halo (e.g. fluoro);
R₃ is selected from hydrogen, methyl, ethyl, CHF₂, CH₂CH₂OCH₃, C(═O)H and cyano;
R₉ is R₉ₐ or X₃—R₉ₐ (particularly R₉ₐ);
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and each R_{9b} is as defined above (e.g. as defined in the broadest embodiment of the first aspect of the invention), (e.g. each R_{9b} is independently selected from O—C₁-C₃alkyl and 6 membered heterocyclyl comprising 1 heteroatom which is O).

According to a second aspect of the invention, there is hereby provided a compound selected from:

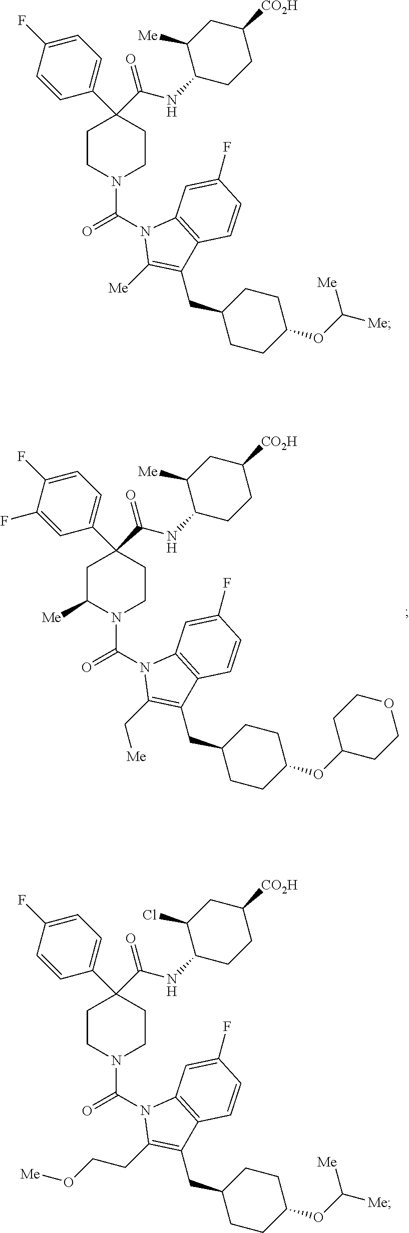 [SMILES: Cc1c(C[C@H]2CC[C@H](OC(C)C)CC2)c2ccc(F)cc2n1C(=O)N1CCC(C(=O)N[C@H]2CC[C@H](C(=O)O)C[C@@H]2C)(c2ccc(F)cc2)CC1 ; CCc1c(C[C@H]2CC[C@H](OC3CCOCC3)CC2)c2ccc(F)cc2n1C(=O)N1CC[C@@](C(=O)N[C@H]2CC[C@H](C(=O)O)C[C@@H]2C)(c2ccc(F)c(F)c2)C[C@@H]1C ; COCCc1c(C[C@H]2CC[C@H](OC(C)C)CC2)c2ccc(F)cc2n1C(=O)N1CCC(C(=O)N[C@H]2CC[C@H](C(=O)O)C[C@@H]2Cl)(c2ccc(F)cc2)CC1]

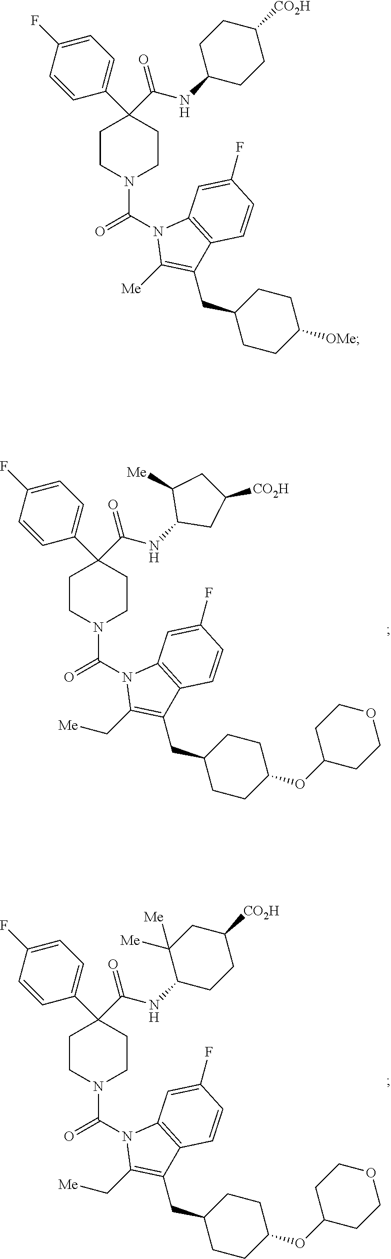 [SMILES: CO[C@H]1CC[C@H](Cc2c(C)n(C(=O)N3CCC(C(=O)N[C@H]4CC[C@H](C(=O)O)CC4)(c4ccc(F)cc4)CC3)c3cc(F)ccc23)CC1 ; CCc1c(C[C@H]2CC[C@H](OC3CCOCC3)CC2)c2ccc(F)cc2n1C(=O)N1CCC(C(=O)N[C@H]2C[C@H](C(=O)O)C[C@@H]2C)(c2ccc(F)cc2)CC1 ; CCc1c(C[C@H]2CC[C@H](OC3CCOCC3)CC2)c2ccc(F)cc2n1C(=O)N1CCC(C(=O)N[C@H]2CC[C@H](C(=O)O)CC2(C)C)(c2ccc(F)cc2)CC1]

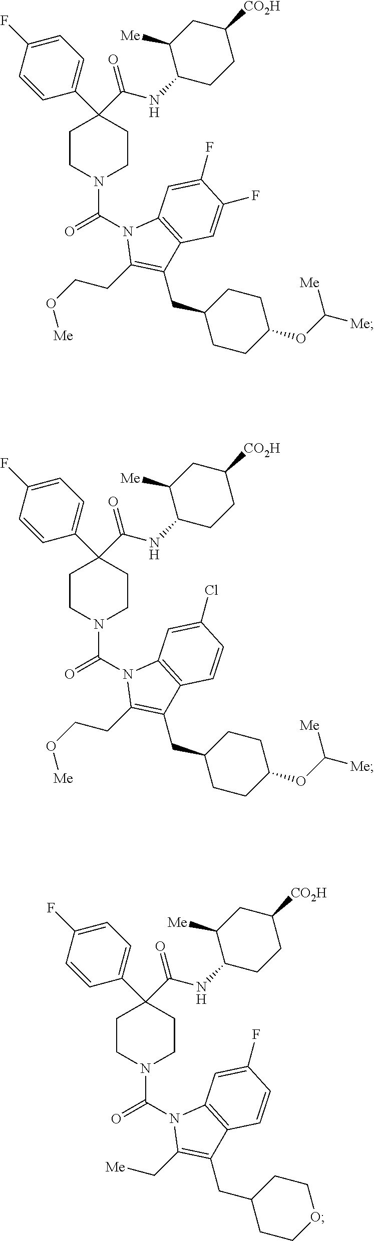 [SMILES: COCCc1c(C[C@H]2CC[C@H](OC(C)C)CC2)c2cc(F)c(F)cc2n1C(=O)N1CCC(C(=O)N[C@H]2CC[C@H](C(=O)O)C[C@@H]2C)(c2ccc(F)cc2)CC1 ; COCCc1c(C[C@H]2CC[C@H](OC(C)C)CC2)c2ccc(Cl)cc2n1C(=O)N1CCC(C(=O)N[C@H]2CC[C@H](C(=O)O)C[C@@H]2C)(c2ccc(F)cc2)CC1 ; CCc1c(CC2CCOCC2)c2ccc(F)cc2n1C(=O)N1CCC(C(=O)N[C@H]2CC[C@H](C(=O)O)C[C@@H]2C)(c2ccc(F)cc2)CC1]

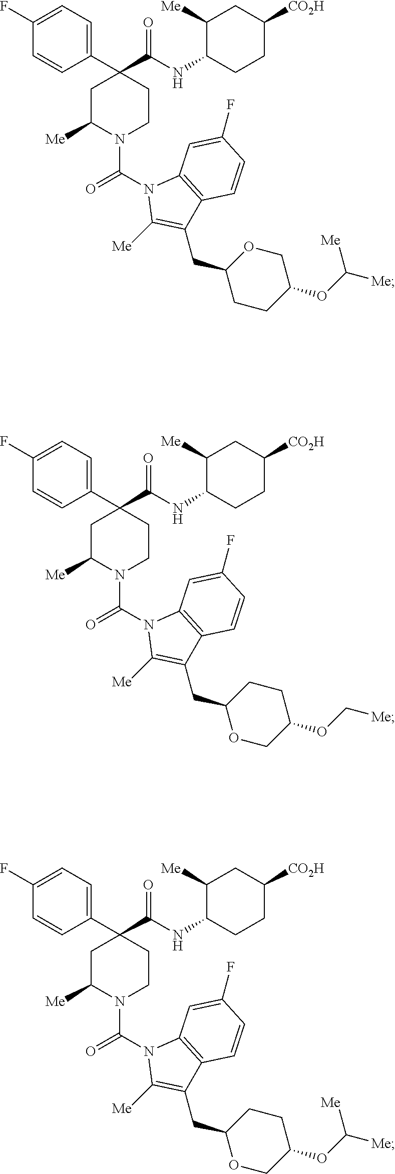 [SMILES: Cc1c(C[C@@H]2CC[C@@H](OC(C)C)CO2)c2ccc(F)cc2n1C(=O)N1CC[C@@](C(=O)N[C@H]2CC[C@H](C(=O)O)C[C@@H]2C)(c2ccc(F)cc2)C[C@@H]1C ; CCO[C@H]1CC[C@H](Cc2c(C)n(C(=O)N3CC[C@@](C(=O)N[C@H]4CC[C@H](C(=O)O)C[C@@H]4C)(c4ccc(F)cc4)C[C@@H]3C)c3cc(F)ccc23)OC1 ; Cc1c(C[C@H]2CC[C@H](OC(C)C)CO2)c2ccc(F)cc2n1C(=O)N1CC[C@@](C(=O)N[C@H]2CC[C@H](C(=O)O)C[C@@H]2C)(c2ccc(F)cc2)C[C@@H]1C]

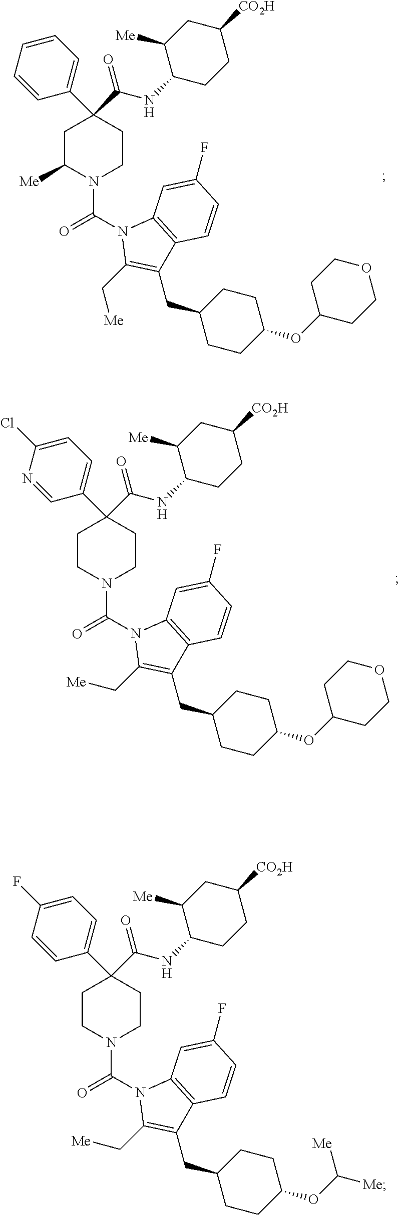 [SMILES: CCc1c(C[C@H]2CC[C@H](OC3CCOCC3)CC2)c2ccc(F)cc2n1C(=O)N1CC[C@@](C(=O)N[C@H]2CC[C@H](C(=O)O)C[C@@H]2C)(c2ccccc2)C[C@@H]1C ; CCc1c(C[C@H]2CC[C@H](OC3CCOCC3)CC2)c2ccc(F)cc2n1C(=O)N1CCC(C(=O)N[C@H]2CC[C@H](C(=O)O)C[C@@H]2C)(c2ccc(Cl)nc2)CC1 ; CCc1c(C[C@H]2CC[C@H](OC(C)C)CC2)c2ccc(F)cc2n1C(=O)N1CCC(C(=O)N[C@H]2CC[C@H](C(=O)O)C[C@@H]2C)(c2ccc(F)cc2)CC1]

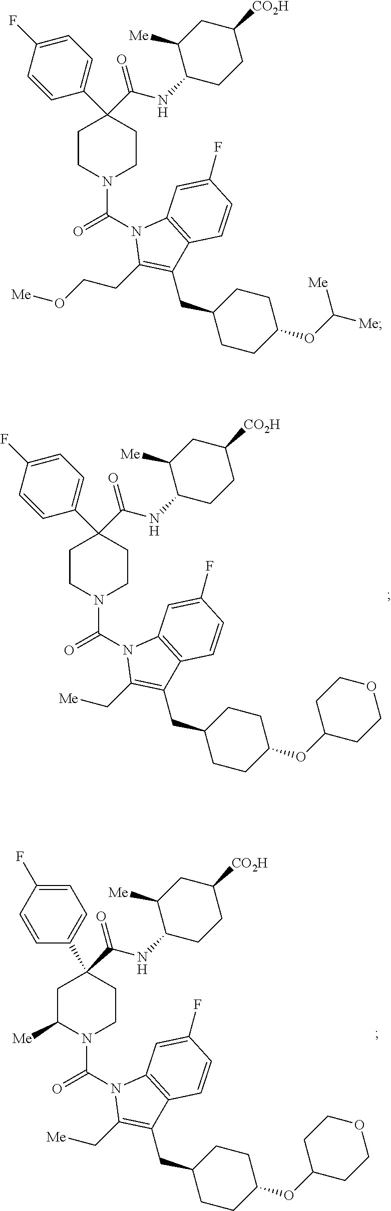 [SMILES: COCCc1c(C[C@H]2CC[C@H](OC(C)C)CC2)c2ccc(F)cc2n1C(=O)N1CCC(C(=O)N[C@H]2CC[C@H](C(=O)O)C[C@@H]2C)(c2ccc(F)cc2)CC1 ; CCc1c(C[C@H]2CC[C@H](OC3CCOCC3)CC2)c2ccc(F)cc2n1C(=O)N1CCC(C(=O)N[C@H]2CC[C@H](C(=O)O)C[C@@H]2C)(c2ccc(F)cc2)CC1 ; CCc1c(C[C@H]2CC[C@H](OC3CCOCC3)CC2)c2ccc(F)cc2n1C(=O)N1CC[C@@](C(=O)N[C@H]2CC[C@H](C(=O)O)C[C@@H]2C)(c2ccc(F)cc2)C[C@@H]1C]

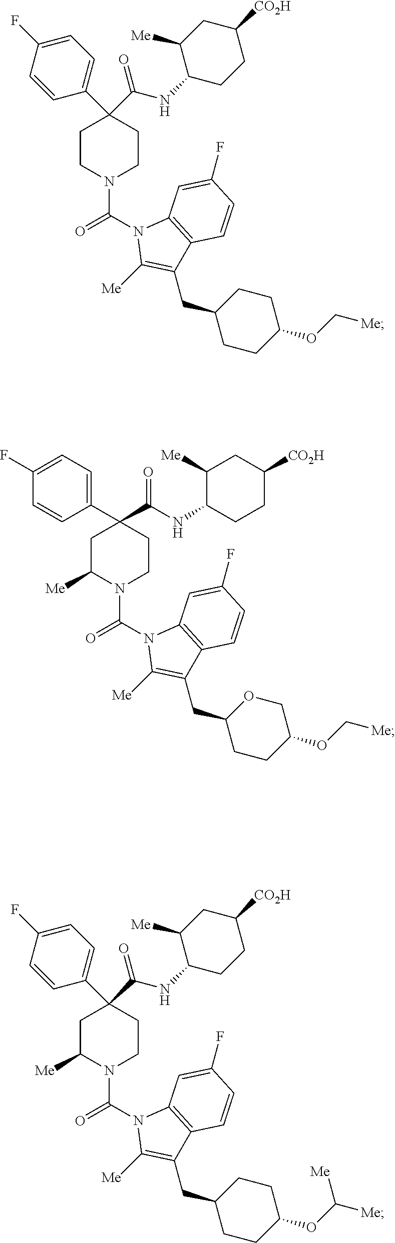 [SMILES: CCO[C@H]1CC[C@H](Cc2c(C)n(C(=O)N3CCC(C(=O)N[C@H]4CC[C@H](C(=O)O)C[C@@H]4C)(c4ccc(F)cc4)CC3)c3cc(F)ccc23)CC1 ; CCO[C@@H]1CC[C@@H](Cc2c(C)n(C(=O)N3CC[C@@](C(=O)N[C@H]4CC[C@H](C(=O)O)C[C@@H]4C)(c4ccc(F)cc4)C[C@@H]3C)c3cc(F)ccc23)OC1 ; Cc1c(C[C@H]2CC[C@H](OC(C)C)CC2)c2ccc(F)cc2n1C(=O)N1CC[C@@](C(=O)N[C@H]2CC[C@H](C(=O)O)C[C@@H]2C)(c2ccc(F)cc2)C[C@@H]1C]

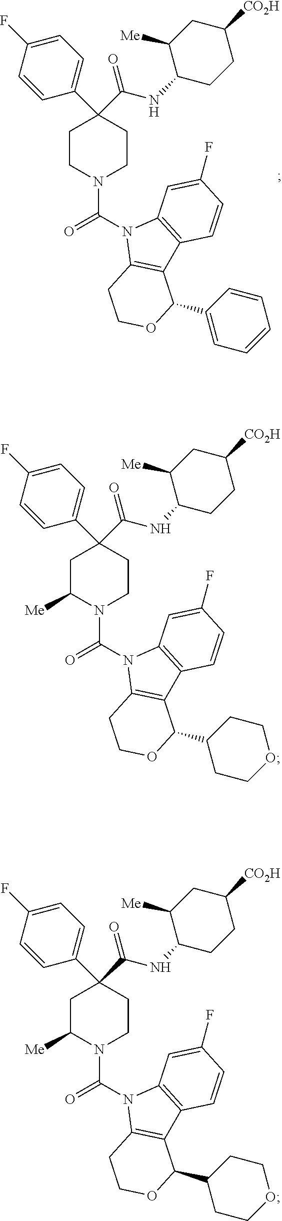 [SMILES: C[C@H]1C[C@@H](C(=O)O)CC[C@@H]1NC(=O)C1(c2ccc(F)cc2)CCN(C(=O)n2c3c(c4ccc(F)cc42)[C@H](c2ccccc2)OCC3)CC1 ; C[C@H]1C[C@@H](C(=O)O)CC[C@@H]1NC(=O)C1(c2ccc(F)cc2)CCN(C(=O)n2c3c(c4ccc(F)cc42)[C@H](C2CCOCC2)OCC3)[C@@H](C)C1 ; C[C@H]1C[C@@H](C(=O)O)CC[C@@H]1NC(=O)[C@@]1(c2ccc(F)cc2)CCN(C(=O)n2c3c(c4ccc(F)cc42)[C@@H](C2CCOCC2)OCC3)[C@@H](C)C1]

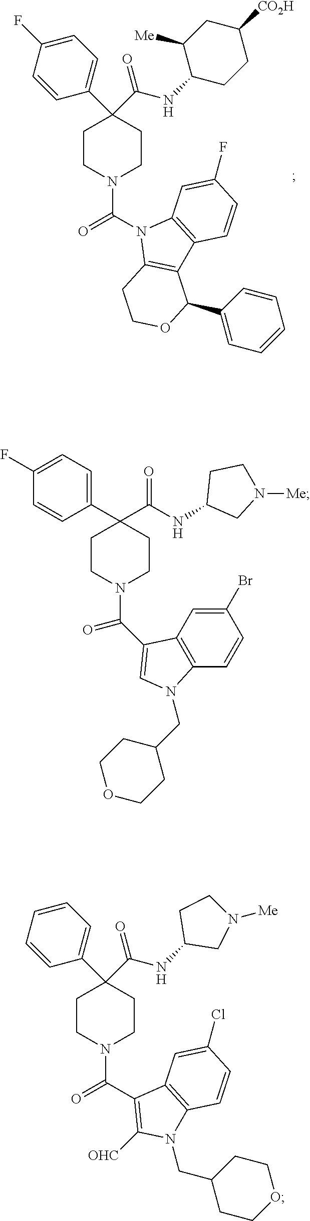 [SMILES: C[C@H]1C[C@@H](C(=O)O)CC[C@@H]1NC(=O)C1(c2ccc(F)cc2)CCN(C(=O)n2c3c(c4ccc(F)cc42)[C@@H](c2ccccc2)OCC3)CC1 ; CN1CC[C@@H](NC(=O)C2(c3ccc(F)cc3)CCN(C(=O)c3cn(CC4CCOCC4)c4ccc(Br)cc34)CC2)C1 ; CN1CC[C@@H](NC(=O)C2(c3ccccc3)CCN(C(=O)c3c(C=O)n(CC4CCOCC4)c4ccc(Cl)cc34)CC2)C1]

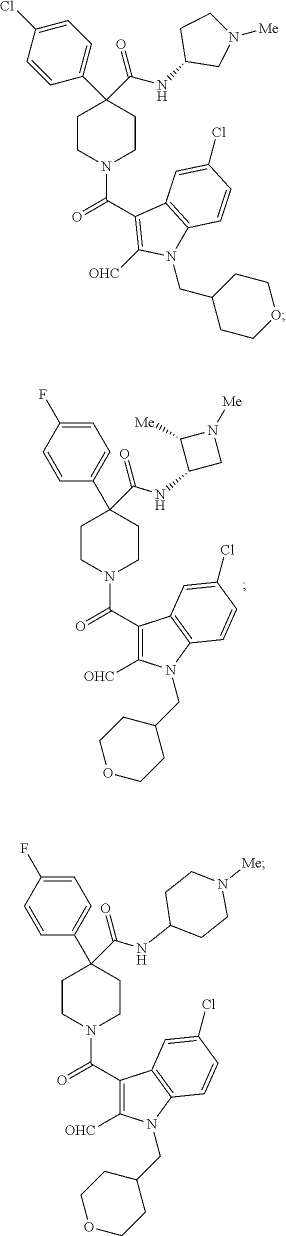 [SMILES: CN1CC[C@@H](NC(=O)C2(c3ccc(Cl)cc3)CCN(C(=O)c3c(C=O)n(CC4CCOCC4)c4ccc(Cl)cc34)CC2)C1 ; C[C@H]1[C@@H](NC(=O)C2(c3ccc(F)cc3)CCN(C(=O)c3c(C=O)n(CC4CCOCC4)c4ccc(Cl)cc34)CC2)CN1C ; CN1CCC(NC(=O)C2(c3ccc(F)cc3)CCN(C(=O)c3c(C=O)n(CC4CCOCC4)c4ccc(Cl)cc34)CC2)CC1]

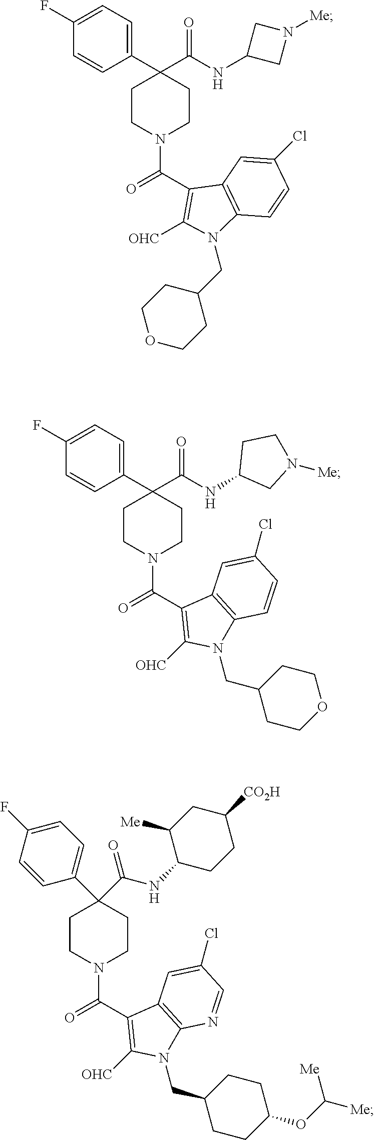 [SMILES: CN1CC(NC(=O)C2(c3ccc(F)cc3)CCN(C(=O)c3c(C=O)n(CC4CCOCC4)c4ccc(Cl)cc34)CC2)C1 ; CN1CC[C@@H](NC(=O)C2(c3ccc(F)cc3)CCN(C(=O)c3c(C=O)n(CC4CCOCC4)c4ccc(Cl)cc34)CC2)C1 ; CC(C)O[C@H]1CC[C@H](Cn2c(C=O)c(C(=O)N3CCC(C(=O)N[C@H]4CC[C@H](C(=O)O)C[C@@H]4C)(c4ccc(F)cc4)CC3)c3cc(Cl)cnc32)CC1]

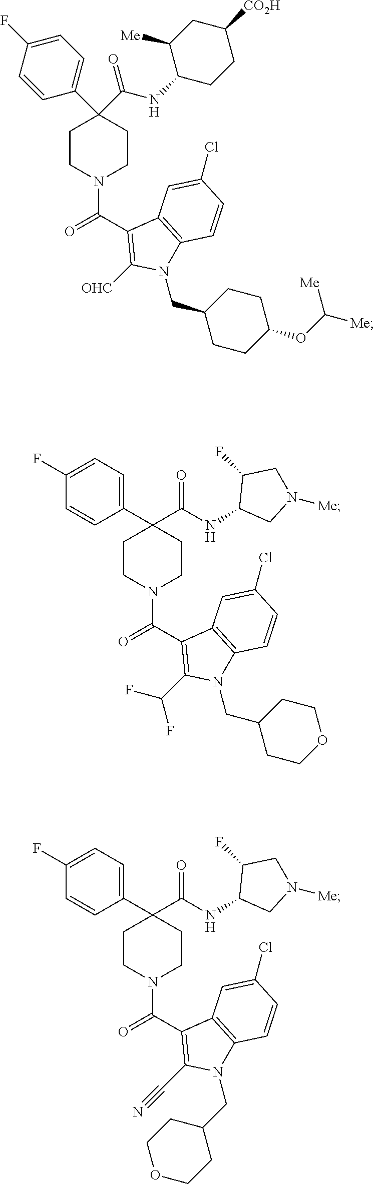 [SMILES: CC(C)O[C@H]1CC[C@H](Cn2c(C=O)c(C(=O)N3CCC(C(=O)N[C@H]4CC[C@H](C(=O)O)C[C@@H]4C)(c4ccc(F)cc4)CC3)c3cc(Cl)ccc32)CC1 ; CN1C[C@@H](F)[C@@H](NC(=O)C2(c3ccc(F)cc3)CCN(C(=O)c3c(C(F)F)n(CC4CCOCC4)c4ccc(Cl)cc34)CC2)C1 ; CN1C[C@@H](F)[C@@H](NC(=O)C2(c3ccc(F)cc3)CCN(C(=O)c3c(C#N)n(CC4CCOCC4)c4ccc(Cl)cc34)CC2)C1]

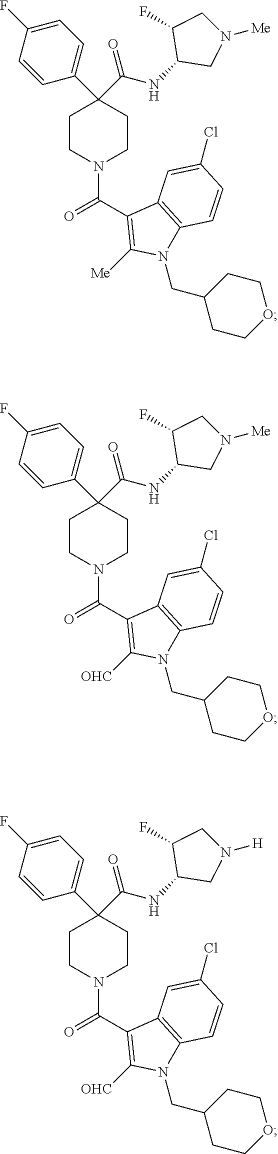 [SMILES: Cc1c(C(=O)N2CCC(C(=O)N[C@H]3CN(C)C[C@H]3F)(c3ccc(F)cc3)CC2)c2cc(Cl)ccc2n1CC1CCOCC1 ; CN1C[C@@H](F)[C@@H](NC(=O)C2(c3ccc(F)cc3)CCN(C(=O)c3c(C=O)n(CC4CCOCC4)c4ccc(Cl)cc34)CC2)C1 ; O=Cc1c(C(=O)N2CCC(C(=O)N[C@H]3CNC[C@H]3F)(c3ccc(F)cc3)CC2)c2cc(Cl)ccc2n1CC1CCOCC1]

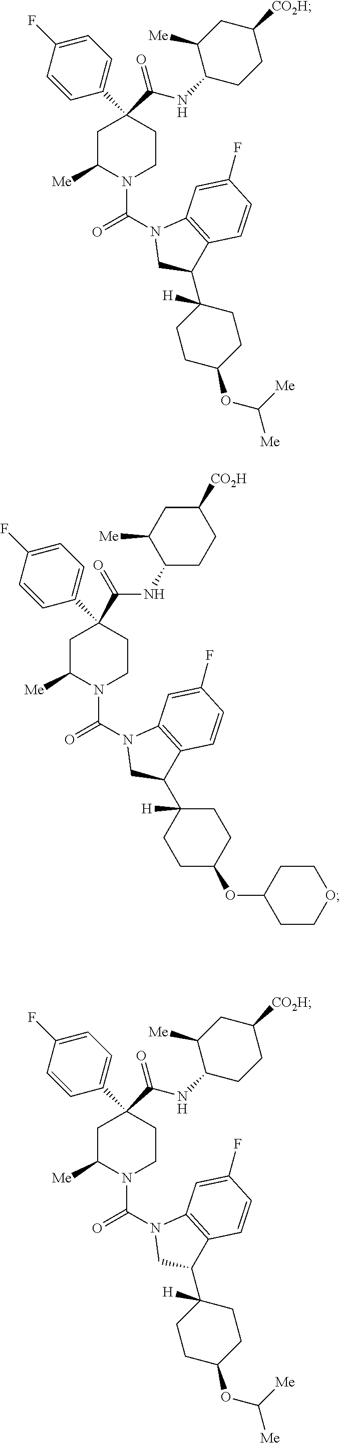 [SMILES: CC(C)O[C@H]1CC[C@H]([C@H]2CN(C(=O)N3CC[C@@](C(=O)N[C@H]4CC[C@H](C(=O)O)C[C@@H]4C)(c4ccc(F)cc4)C[C@@H]3C)c3cc(F)ccc32)CC1 ; C[C@H]1C[C@@H](C(=O)O)CC[C@@H]1NC(=O)[C@@]1(c2ccc(F)cc2)CCN(C(=O)N2C[C@H]([C@H]3CC[C@H](OC4CCOCC4)CC3)c3ccc(F)cc32)[C@@H](C)C1 ; CC(C)O[C@H]1CC[C@H]([C@@H]2CN(C(=O)N3CC[C@@](C(=O)N[C@H]4CC[C@H](C(=O)O)C[C@@H]4C)(c4ccc(F)cc4)C[C@@H]3C)c3cc(F)ccc32)CC1]

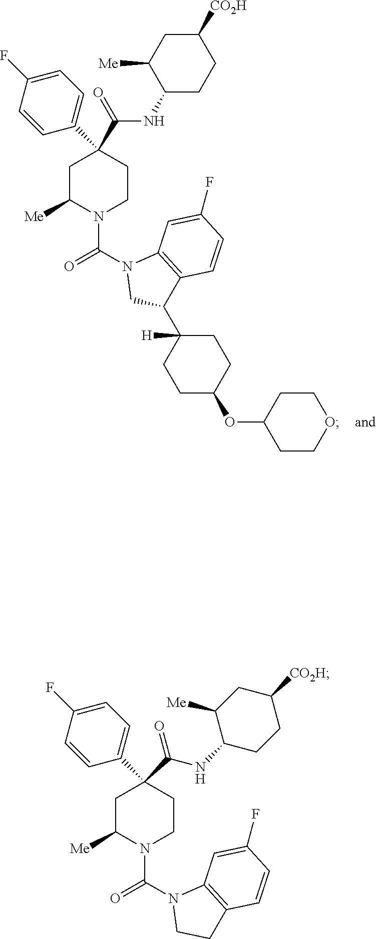 [SMILES: C[C@H]1C[C@@H](C(=O)O)CC[C@@H]1NC(=O)[C@@]1(c2ccc(F)cc2)CCN(C(=O)N2C[C@@H]([C@H]3CC[C@H](OC4CCOCC4)CC3)c3ccc(F)cc32)[C@@H](C)C1 ; C[C@H]1C[C@@H](C(=O)O)CC[C@@H]1NC(=O)[C@@]1(c2ccc(F)cc2)CCN(C(=O)N2CCc3ccc(F)cc32)[C@@H](C)C1]

or a pharmaceutically acceptable salt thereof.

According to a third aspect of the invention, there is hereby provided a pharmaceutical composition comprising the compound or pharmaceutically acceptable salt thereof according to the first or second aspect of the invention, and one or more pharmaceutically acceptable carriers.

According to a fourth aspect of the invention, there is hereby provided a combination comprising the compound or pharmaceutically acceptable salt thereof according to the first or second aspect of the invention, and one or more additional therapeutically active agents.

According to a fifth aspect of the invention, there is hereby provided a method of modulating ERK activity in a subject, the method comprising administering to the subject a therapeutically effective amount of the compound or pharmaceutically acceptable salt thereof according to the first or second aspect of the invention, or the pharmaceutical composition according to the third aspect of the invention.

According to a sixth aspect of the invention, there is hereby provided a method of treating a patient having a disease associated with aberrant activity of the MAP kinase pathway comprising administering to said patient a therapeutically effective amount of the compound or pharmaceutically acceptable salt thereof according to the first or second aspect of the invention, or the pharmaceutical composition according to the third aspect of the invention.

In an embodiment, the disease associated with aberrant activity of the MAP kinase pathway is cancer.

In an embodiment, the cancer is selected from melanoma, lung cancer, colorectal cancer (CRC), pancreatic cancer and thyroid cancer.

In an embodiment, the cancer contains a BRAF and/or a RAS mutation.

According to a seventh aspect of the invention, there is hereby provided the compound or pharmaceutically acceptable salt thereof according to the first or second aspect of the invention for use as a medicament.

According to an eighth aspect of the invention, there is hereby provided the compound or pharmaceutically acceptable salt thereof according to the first or second aspect of the invention for use in the treatment of cancer.

In an embodiment, the cancer is selected from melanoma, lung cancer, colorectal cancer (CRC), pancreatic cancer and thyroid cancer.

In an embodiment, the cancer contains a BRAF and/or a RAS mutation.

According to a ninth aspect of the invention, there is hereby provided a use of a compound or pharmaceutically acceptable salt thereof according to the first or second aspect of the invention in the manufacture of a medicament for the treatment of cancer.

In an embodiment, the cancer is selected from melanoma, lung cancer, colorectal cancer (CRC), pancreatic cancer and thyroid cancer.

In an embodiment, the cancer contains a BRAF and/or a RAS mutation.

In an embodiment, the compound is administered parenterally. In another embodiment, the compound is administered intramuscularly, intravenously, subcutaneously, orally, pulmonary, intrathecally, topically or intranasally. In yet another embodiment, the compound is administered systemically.

In an embodiment, the patient is a mammal, for example a primate, for example a human.

The invention therefore provides the following numbered embodiments. It will be recognized that features specified in each embodiment may be combined with other specified features to provide further embodiments of the present invention.

Embodiment 1. A compound of formula (I):

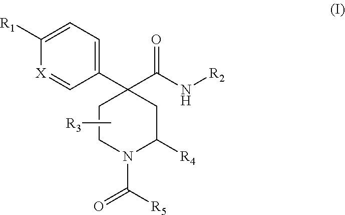

wherein:
X is selected from N and CR₆, wherein R₆ is selected from hydrogen and halo;
R₁ is selected from hydrogen and halo;
R₂ is selected from —X₁—R₂ₐ and R₂ₐ;
X₁ is selected from C₁-C₄alkylene and C₂-C₄haloalkylene;
R₂ₐ is selected from i) hydrogen, and ii) a ring substituted with 0 to 3 substituents R_{2b}, wherein the ring is selected from a) 3-6 membered saturated or partially unsaturated carbocyclic ring, b) 5-6 membered heteroaryl, c) phenyl, and d) 4 to 6 membered heterocyclyl (e.g. fully saturated heterocyclyl) containing 1 or 2 heteroatoms independently selected from oxygen and nitrogen;
each R_{2b} is independently selected from halo, hydroxyl, C₁-C₃alkyl, C₃-C₄cycloalkyl, C₁-C₃haloalkyl, C₁-C₃hydroxyalkyl, O—C₁-C₃alkyl, C₁-C₃alkylene-O—C₁-C₃alkyl, cyano, —CO₂R₁₁, —CO₂N(R₁₁)₂, —X₂—CO₂R₁₁ and —X₂—CO₂N(R₁₁)₂;
X₂ is selected from C₁-C₅alkylene and C₃-C₆cycloalkylene;
each R₁₁ is independently selected from hydrogen, C₁-C₅alkyl, C₃-C₅cycloalkyl, C₁-C₅haloalkyl and C₃-C₅cyclohaloalkyl, or two R₁ groups together with the nitrogen atom to which they are mutually attached join to form a 4 to 6 membered heterocyclic ring containing 1 heteroatom which is nitrogen;
R₃ is selected from hydrogen, C₁-C₃alkyl and C₁-C₃haloalkyl, and R₄ is hydrogen, or R₃ and R₄ together with the piperidinyl ring of formula (I) to which R₃ and R₄ are attached join to form a 7 or 8 membered bridged or fused heterocyclic ring;
R₅ is selected from:

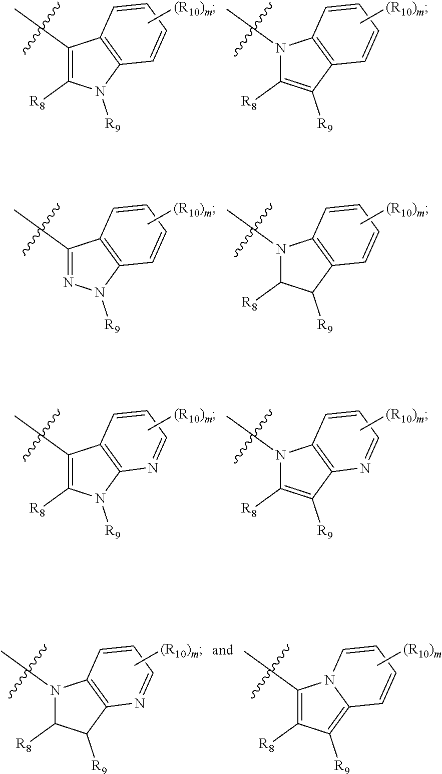

wherein:
R₈ is selected from hydrogen, halo, C₁-C₆alkyl, C₃-C₄cycloalkyl, C₁-C₅haloalkyl, C₁-C₆alkylene-O—C₁-C₄alkyl, C₁-C₅haloalkylene-O—C₁-C₄alkyl, C₁-C₅hydroxyalkyl, C₁-C₅haloalkylene-O—C₁-C₄haloalkyl, C₁-C₆alkylene-O—C₁-C₄haloalkyl, C(═O)H and cyano, and R₉ is selected from —X₃—R₉ₐ and R₉ₐ;
or R₈ and R₉ together with the carbon atoms to which R₃ and R₉ are attached form a ring substituted with 0 to 3 R_{9b} groups, wherein the ring is a) 5-6 membered saturated or partially unsaturated carbocyclic ring, or b) 5-6 membered heterocyclyl comprising 1 heteroatom which is O;
X₃ is selected from C₁-C₂alkylene and C₃-C₅cycloalkylene;
R₉ₐ is selected from i) hydrogen and ii) a ring substituted with 0 to 3 R_{9b} groups, wherein the ring is a) phenyl, b) 5-6 membered heteroaryl, c) C₃-C₇cycloalkyl, d) C₇-C₉spiroalkyl, e) 4 to 7 membered heterocyclyl comprising 1 or 2 heteroatoms which is/are each O, or f) 7 to 9 membered spiroheterocyclyl comprising 1 or 2 heteroatoms which is/are each O;
each R_{9b} is independently selected from halo, hydroxy, C₁-C₄alkyl, C₁-C₄haloalkyl, O—C₁-C₄alkyl, O—C₁-C₄haloalkyl, C₁-C₄alkylene-O—C₁-C₄alkyl, C₁-C₄haloalkylene-O—C₁-C₄alkyl, C₁-C₄alkylene-O—C₁-C₄haloalkyl, C₁-C₄haloalkylene-O—C₁-C₄haloalkyl, C₃-C₇cycloalkyl substituted with 0 to 2 R_{9c} groups, C₃-C₇cyclohaloalkyl substituted with 0 to 2 R₉, groups, O—C₃-C₇cycloalkyl substituted with 0 to 2 R_{9c} groups, O—C₃-C₇cyclohaloalkyl substituted with 0 to 2 R_{9c} groups, 3-7 membered heterocyclyl comprising 1 heteroatom which is O substituted with 0 to 2 R_{9c} groups, O-3-7 membered heterocyclyl comprising 1 heteroatom which is O substituted with 0 to 2 R_{9c} groups, phenyl substituted with 0 to 2 R_{9c} groups, pyridinyl substituted with 0 to 2 R_{9c} groups, O—C₁-C₃alkylene-C₃-C₇cycloalkyl substituted with 0 to 2 R_{9c} groups, O—C₁-C₃alkylene-C₃-C₇cyclohaloalkyl substituted with 0 to 2 R_{9c} groups and O—C₁-C₃alkylene-3-7 membered heterocyclyl comprising 1 heteroatom which is O substituted with 0 to 2 R_{9c} groups;
or wherein two R_{9b} groups in combination with the carbon atom to which they are mutually attached form a ring substituted with 0 to 2 R_{9c} groups, wherein the ring is i) C₃-C₆cycloalkyl, or ii) 4-6 membered heterocyclyl containing one heteroatom which is oxygen;
each R_{9c} is independently selected from halo (e.g. fluoro), CH₃ and OCH₃;
each R₁₀ is halo; and
m is an integer from 0 to 2;
or a pharmaceutically acceptable salt thereof.

Embodiment 2. The compound or pharmaceutically acceptable salt thereof of Embodiment 1, wherein the compound is of formula (Ia):

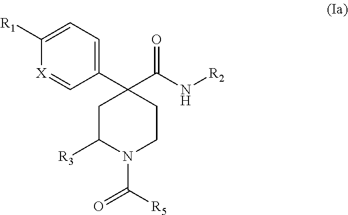

in which:
X is selected from N and CR₆; wherein R₆ is selected from hydrogen and halo;
R₁ is selected from hydrogen and halo;
R₂ is selected from —X₁—R₂ₐ and R₂ₐ;
X₁ is selected from C₁-C₂alkylene and C₂haloalkylene;
R₂ₐ is selected from i) hydrogen and ii) a ring substituted with 0 to 3 substituents R_{2b}; wherein the ring is selected from a) 3-6 membered saturated or partially unsaturated carbocyclic ring, b) 5-6 membered heteroaryl, c) phenyl, and d) 4 to 6 membered heterocyclyl (e.g. fully saturated heterocyclyl) containing 1 or 2 heteroatoms independently selected from oxygen and nitrogen;
each R_{2b} is independently selected from halo, hydroxyl, C₁-C₃alkyl, C₁-C₃haloalkyl, C₁-C₃hydroxyalkyl, O—C₁-C₃alkyl, C₁-C₃alkylene-O—C₁-C₃alkyl, cyano, —CO₂R₁₁, —CO₂N(R₁₁)₂, —X₂—CO₂R₁₁ and —X₂—CO₂N(R₁₁)₂;
X₂ is selected from C₁-C₅alkylene and C₃-C₆cycloalkylene;
each R₁ is independently selected from hydrogen, C₁-C₅alkyl and C₃-C₆cycloalkyl;
R₃ is selected from hydrogen and methyl;
R₅ is selected from:

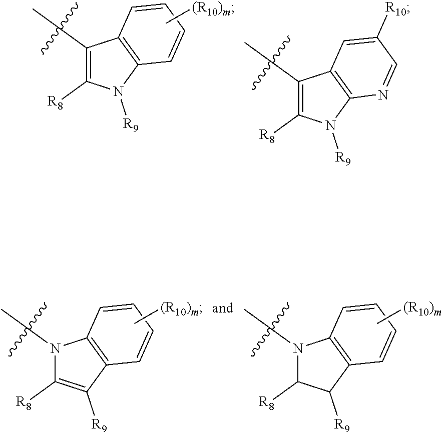

wherein:
R₈ is selected from hydrogen, methyl, ethyl, CHF₂, CH₂OCH₂CH₃, CH₂CH₂OCH₃, C(═O)H and cyano, and R₉ is selected from X₃—R₉ₐ and R₉ₐ;
or R₈ and R₉ together with the carbon atoms to which R₈ and R₉ are attached form a ring substituted with 0 to 1 R_{9b} groups, wherein the ring is a 5-6 membered heterocyclyl comprising 1 heteroatom which is O;
X₃ is C₁-C₂alkylene;
R₉ₐ is selected from i) hydrogen, ii) a ring substituted with 0 to 3 R_{9b} groups, wherein the ring is a) C₄-C₆cycloalkyl, b) 6 membered heterocyclyl comprising 1 heteroatom which is O or c) 7 to 9 membered spiroheterocyclyl comprising 1 or 2 heteroatoms which is/are each 0;
each R_{9b} is independently selected from halo, C₁-C₃alkyl, O—C₁-C₃alkyl, O-4-6 membered heterocyclyl comprising 1 heteroatom which is O substituted with 0 to 2 R_{9c} groups, pyridinyl substituted with 0 to 2 R_{9c} groups and O—C₁-C₃alkylene-4-6 membered heterocyclyl comprising 1 heteroatom which is O substituted with 0 to 2 R_{9c} groups;
or wherein two R_{9b} groups in combination with the carbon atom to which they are mutually attached form a ring substituted with 0 to 2 R_{9c} groups, wherein the ring is i) C₃-C₆cycloalkyl, or ii) 4-6 membered heterocyclyl containing one heteroatom which is oxygen;
each R_{9c} is independently selected from halo (e.g. fluoro), CH₃ and OCH₃;
each R₁₀ is halo; and
m is an integer from 0 to 2;
or a pharmaceutically acceptable salt thereof.

Embodiment 3. The compound or pharmaceutically acceptable salt thereof according to Embodiment 1 or Embodiment 2, wherein X is CH or CF.

Embodiment 4. The compound or pharmaceutically acceptable salt thereof according to Embodiment 3, wherein X is CH.

Embodiment 5. The compound or pharmaceutically acceptable salt thereof according to any one of the preceding Embodiments, wherein R₂ is R₂ₐ.

Embodiment 6. The compound or pharmaceutically acceptable salt thereof according to any one of the preceding Embodiments, wherein R₂ₐ is a ring substituted with 0 to 3 substituents R_{2b}; wherein the ring is selected from a) C₄-C₆ cycloalkyl, and b) 4 to 6 membered heterocyclyl (e.g. fully saturated heterocyclyl) containing 1 or 2 heteroatoms independently selected from oxygen and nitrogen.

Embodiment 7. The compound or pharmaceutically acceptable salt thereof according to Embodiment 6, wherein R₂ₐ is a ring substituted with 1 to 3 substituents R_{2b}; wherein the ring is selected from a) C₅-C₆ cycloalkyl, and b) 4 to 6 membered heterocyclyl (e.g. fully saturated heterocyclyl) containing 1 or 2 heteroatoms independently selected from oxygen and nitrogen.

Embodiment 8. The compound or pharmaceutically acceptable salt thereof according to any one of the preceding Embodiments, wherein each R_{2b} is independently selected from halo, C₁-C₃alkyl and CO₂H.

Embodiment 9. The compound or pharmaceutically acceptable salt thereof according to any one of the preceding Embodiments, wherein each R_{2b} is independently selected from fluoro, chloro, methyl and CO₂H.

Embodiment 10. The compound or pharmaceutically acceptable salt thereof according to any one of the preceding Embodiments, wherein R₃ is H.

Embodiment 11. The compound or pharmaceutically acceptable salt thereof according to any one of the preceding Embodiments, wherein R₅ is selected from:

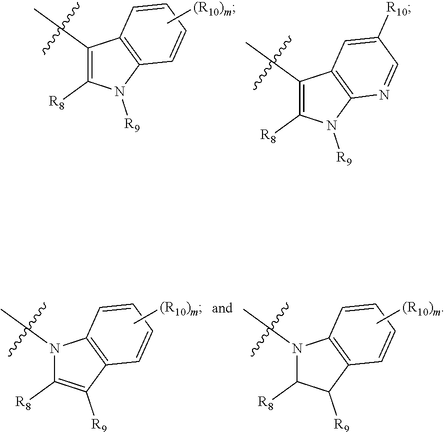

Embodiment 12. The compound or pharmaceutically acceptable salt thereof according to Embodiment 11, wherein m is 1 or 2.

Embodiment 13. The compound or pharmaceutically acceptable salt thereof according to any one of the preceding Embodiments, wherein R₅ is selected from:

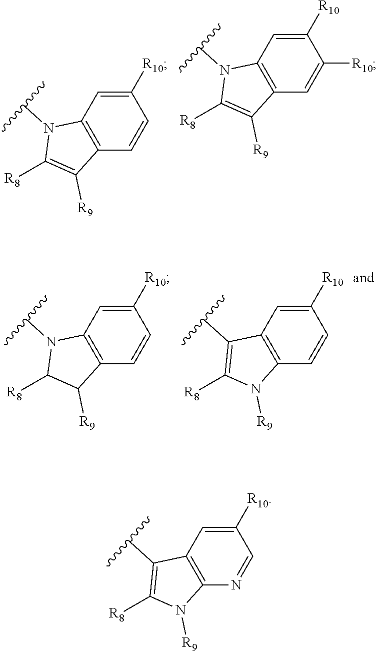

Embodiment 14. The compound or pharmaceutically acceptable salt thereof according to any one of the preceding Embodiments, wherein each R₁₀ is independently selected from fluoro, chloro and bromo.

Embodiment 15. The compound or pharmaceutically acceptable salt thereof according to any one of the preceding Embodiments, wherein R₂ₐ is C₅-C₆cycloalkyl substituted with 1 to 3 substituents R_{2b}.

Embodiment 16. The compound or pharmaceutically acceptable salt thereof according to any one of the preceding Embodiments, wherein R₂ₐ is C₆cycloalkyl substituted with 1 to 3 substituents R_{2b}.

Embodiment 17. The compound or pharmaceutically acceptable salt thereof according to Embodiment 15 or Embodiment 16, wherein one R_{2b} is CO₂H, and the other 0 to 2 R_{2b} groups are each independently selected from methyl, fluoro and chloro.

Embodiment 18. The compound or pharmaceutically acceptable salt thereof according to any one of Embodiments 1 to 16, wherein the compound is of formula (Ib):

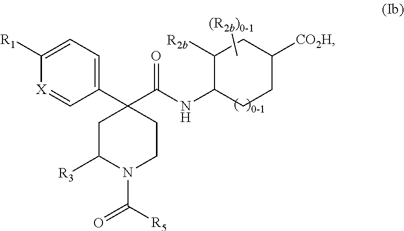

wherein X, R₁, R₃, R₅ and each R_{2b} are as defined in any one of Embodiments 1 to 16.

Embodiment 19. The compound or pharmaceutically acceptable salt thereof according to Embodiment 18, wherein each R_{2b} is independently selected from methyl, fluoro and chloro.

Embodiment 20. The compound or pharmaceutically acceptable salt thereof according to any one of Embodiments 1 to 14, wherein R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b}.

Embodiment 21. The compound or pharmaceutically acceptable salt thereof according to Embodiment 20, wherein R₂ₐ is 4 to 6 membered heterocyclyl (e.g. fully saturated heterocyclyl) containing 1 heteroatom which is nitrogen substituted with 0 to 2 substituents R_{2b}.

Embodiment 22. The compound or pharmaceutically acceptable salt thereof according to Embodiment 20 or Embodiment 21, wherein R₂ₐ is 4 to 6 membered heterocyclyl (e.g. fully saturated heterocyclyl) containing 1 heteroatom which is nitrogen substituted with 1 or 2 substituents R_{2b}, and wherein each R_{2b} substituent is independently selected from methyl and halo (e.g. fluoro).

Embodiment 23. The compound or pharmaceutically acceptable salt thereof according to any one of the preceding Embodiments, wherein R₃ is selected from hydrogen, methyl, ethyl, CHF₂, CH₂CH₂OCH₃, C(═O)H and cyano.

Embodiment 24. The compound or pharmaceutically acceptable salt thereof according to any one of the preceding Embodiments, wherein R₉ is X₃—R₉ₐ.

Embodiment 25. The compound or pharmaceutically acceptable salt thereof according to Embodiment 24, wherein X₃ is CH₂.

Embodiment 26. The compound or pharmaceutically acceptable salt thereof according to any one of the preceding Embodiments, wherein R₉ₐ is a ring substituted with 0 to 2 R_{9b} groups, wherein the ring is a) C₅-C₆cycloalkyl or b) 6 membered heterocyclyl comprising 1 heteroatom which is O.

Embodiment 27. The compound or pharmaceutically acceptable salt thereof according to Embodiment 26, wherein R₉ₐ is a ring substituted with 0 or 1 R_{9b} groups, wherein the ring is a) C₆cycloalkyl or b) 6 membered heterocyclyl comprising 1 heteroatom which is O.

Embodiment 28. The compound or pharmaceutically acceptable salt thereof according to any one of the preceding Embodiments, wherein each R_{9b} is independently selected from O—C₁-C₃alkyl and O-6 membered heterocyclyl comprising 1 heteroatom which is O.

Embodiment 29. The compound or pharmaceutically acceptable salt thereof according to any one of Embodiments 1 to 22, wherein R₃ and R₉ together with the carbon atoms to which R₃ and R₉ are attached form a ring substituted with 0 to 1 R_{9b} groups, wherein the ring is a 5-6 membered heterocyclyl comprising 1 heteroatom which is O.

Embodiment 30. The compound or pharmaceutically acceptable salt thereof according to Embodiment 29, wherein R₃ and R₉ together with the carbon atoms to which R₃ and R₉ are attached form a ring substituted with an R_{9b} group, wherein the ring is a 6 membered heterocyclyl comprising 1 heteroatom which is O, and wherein R_{9b} is selected from O-4-6 membered heterocyclyl comprising 1 heteroatom which is O substituted with 0 to 2 (e.g. 0) R_{9c} groups, and phenyl substituted with 0 to 2 (e.g. 0) R_{9c} groups.

Embodiment 31. The compound or pharmaceutically acceptable salt thereof according to Embodiment 29 or Embodiment 30, of formula (Ic) or (Id):

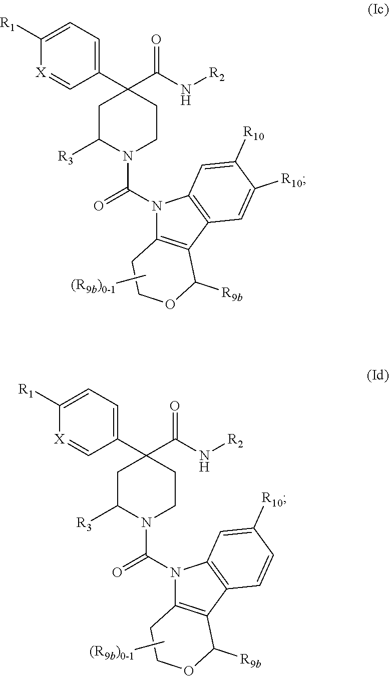

wherein X, R₁, R₂, R₃, R_{9b} and each R₁₀ independently are as defined in Embodiment 29 or Embodiment 30.

Embodiment 32. The compound or pharmaceutically acceptable salt thereof according to Embodiment 31, of formula (Ic-1) or (Id-1):

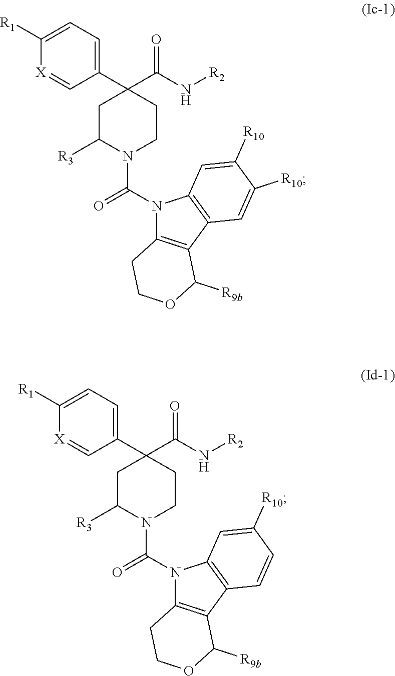

wherein X, R₁, R₂, R₃, R_{9b} and each R₁₀ independently are as defined in Embodiment 31.

Embodiment 33. The compound or pharmaceutically acceptable salt thereof according to Embodiment 32, of formula (Ic-2) or (Id-2):

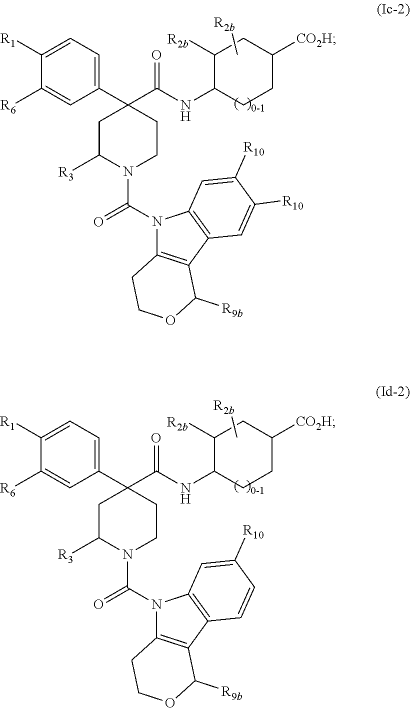

wherein R₁, each R_{2b} independently, R₃, R₆, R_{9b} and each R₁₀ independently are as defined in Embodiment 32.

Embodiment 34. The compound or pharmaceutically acceptable salt thereof according to Embodiment 32 or Embodiment 33, wherein R_{9b} is selected from O-6 membered heterocyclyl comprising 1 heteroatom which is O, and phenyl.

Embodiment 35. The compound or pharmaceutically acceptable salt thereof according to any one of Embodiments 1 to 14, wherein the compound is of formula (II):

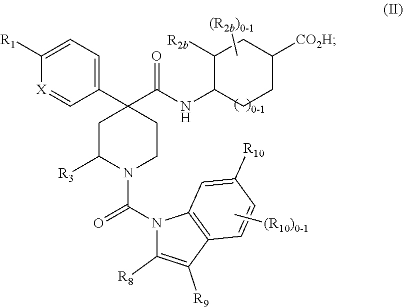

wherein X, R₁, R₃, each R_{2b} independently, and each R₁₀ independently are as defined in any one of Embodiments 1 to 14;
R₈ is selected from hydrogen, methyl, ethyl, CHF₂, CH₂CH₂OCH₃, C(═O)H and cyano;
R₉ is X₃—R₉ₐ;
X₃ is CH₂;
R₉ₐ is selected from i) hydrogen and ii) a ring substituted with 0 to 2 (e.g. 0 to 1) R_{9b} groups, wherein the ring is a) C₅-C₆cycloalkyl or b) 6 membered heterocyclyl comprising 1 heteroatom which is O; and each R_{9b} is as defined in any one of Embodiments 1 to 16 (e.g. each R_{9b} is independently selected from O—C₁-C₃alkyl and 6 membered heterocyclyl comprising 1 heteroatom which is O).

Embodiment 36. The compound or pharmaceutically acceptable salt thereof according to any one of Embodiments 1 to 14, wherein the compound is of formula (III):

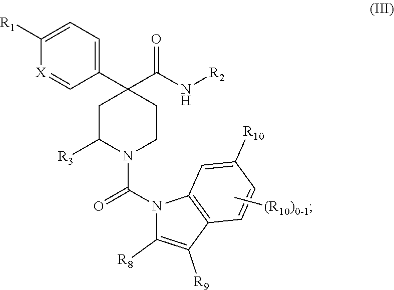

wherein X, R₁, R₃, and each R₁₀ independently are as defined in any one of Embodiments 1 to 14;
R₂ is R₂ₐ;
R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b};
each R_{2b} substituent is independently selected from methyl and halo (e.g. fluoro);
R₃ is selected from hydrogen, methyl, ethyl, CHF₂, CH₂CH₂OCH₃, C(═O)H and cyano;
R₉ is X₃—R₉ₐ;
X₃ is CH₂; and
R₉ₐ is selected from i) hydrogen and ii) a ring substituted with 0 to 2 (e.g. 0 to 1) R_{9b} groups, wherein the ring is a) C₅-C₆cycloalkyl or b) 6 membered heterocyclyl comprising 1 heteroatom which is O; and each R_{9b} is as defined in any one of Embodiments 1 to 16 (e.g. each R_{9b} is independently selected from O—C₁-C₃alkyl and 6 membered heterocyclyl comprising 1 heteroatom which is O).

Embodiment 37. The compound or pharmaceutically acceptable salt thereof according to any one of Embodiments 1 to 14, wherein the compound is of formula (IV):

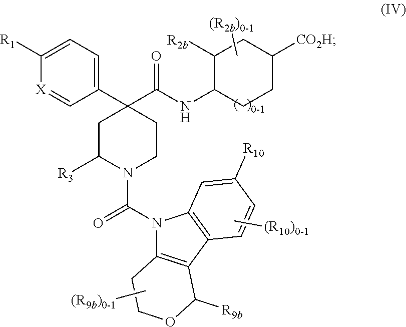

wherein X, R₁, R₃, each R_{2b} independently, each R_{9b} independently and each R₁₀ independently are as defined in any one of Embodiments 1 to 14.

Embodiment 38. The compound or pharmaceutically acceptable salt thereof according to any one of Embodiments 1 to 14, wherein the compound is of formula (V):

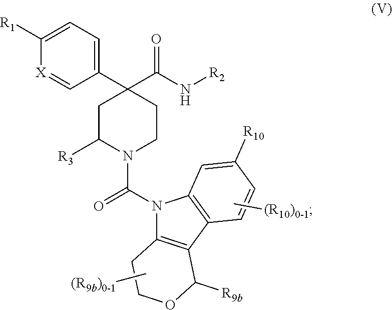

wherein X, R₁, R₃, each R_{9b} independently and each R₁₀ independently are as defined in any one of Embodiments 1 to 14;
R₂ is R₂ₐ;
R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b}; and
each R_{2b} substituent is independently selected from methyl and halo (e.g. fluoro).

Embodiment 39. The compound or pharmaceutically acceptable salt thereof according to any one of Embodiments 1 to 14, wherein the compound is of formula (VI):

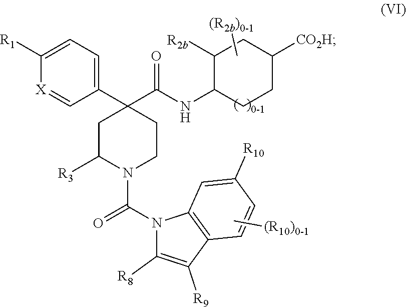

wherein:
X, R₁, R₃, each R_{2b} independently, and each R₁₀ independently are as defined in any one of Embodiments 1 to 14;
R₃ is selected from hydrogen, methyl, ethyl, CHF₂, CH₂CH₂OCH₃, C(═O)H and cyano;
R₉ is X₃—R₉ₐ;
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and
each R_{9b} is as defined in any one of Embodiments 1 to 16 (e.g. each R_{9b} is independently selected from O—C₁-C₃alkyl and 6 membered heterocyclyl comprising 1 heteroatom which is O).

Embodiment 40. The compound or pharmaceutically acceptable salt thereof according to any one of Embodiments 1 to 14, wherein the compound is of formula (VII):

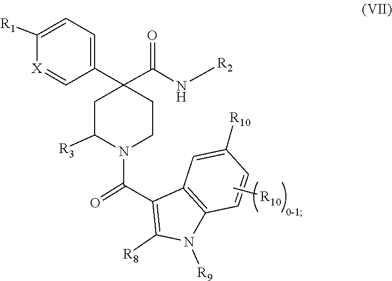

wherein:
X, R₁, R₃, and each R₁₀ independently are as defined in any one of Embodiments 1 to 14;
R₂ is R₂ₐ;
R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b};
each R_{2b} substituent is independently selected from methyl and halo (e.g. fluoro);
R₃ is selected from hydrogen, methyl, ethyl, CHF₂, CH₂CH₂OCH₃, C(═O)H and cyano;
R₉ is X₃—R₉ₐ;
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and
each R_{9b} is as defined in any one of Embodiments 1 to 16 (e.g. each R_{9b} is independently selected from O—C₁-C₃alkyl and 6 membered heterocyclyl comprising 1 heteroatom which is O).

Embodiment 41. The compound or pharmaceutically acceptable salt thereof according to any one of Embodiments 1 to 14, wherein the compound is of formula (VIII):

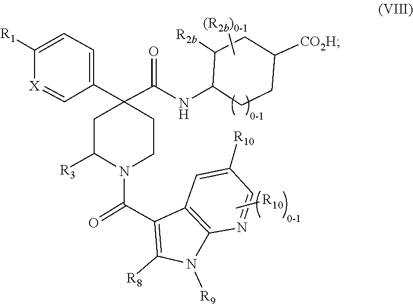

wherein:
X, R₁, R₃, each R_{2b} independently, and each R₁₀ independently are as defined in any one of Embodiments 1 to 14;
R₈ is selected from hydrogen, methyl, ethyl, CHF₂, CH₂CH₂OCH₃, C(═O)H and cyano;
R₉ is X₃—R₉ₐ;
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and each R_{9b} is as defined in any one of Embodiments 1 to 16 (e.g. each R_{9b} is independently selected from O—C₁-C₃alkyl and 6 membered heterocyclyl comprising 1 heteroatom which is O).

Embodiment 42. The compound or pharmaceutically acceptable salt thereof according to any one of Embodiments 1 to 14, wherein the compound is of formula (IX):

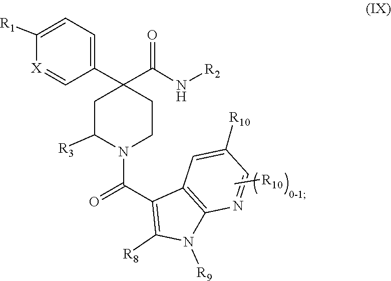

wherein:
X, R₁, R₃, and each R₁₀ independently are as defined in any one of Embodiments 1 to 14;
R₂ is R₂ₐ;
R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b};
each R_{2b} substituent is independently selected from methyl and halo (e.g. fluoro);
R₃ is selected from hydrogen, methyl, ethyl, CHF₂, CH₂CH₂OCH₃, C(═O)H and cyano;
R₉ is X₃—R₉ₐ;
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and each R_{9b} is as defined in any one of Embodiments 1 to 16 (e.g. each R_{9b} is independently selected from O—C₁-C₃alkyl and 6 membered heterocyclyl comprising 1 heteroatom which is O).

Embodiment 43. The compound or pharmaceutically acceptable salt thereof according to any one of Embodiments 1 to 14, wherein the compound is of formula (X):

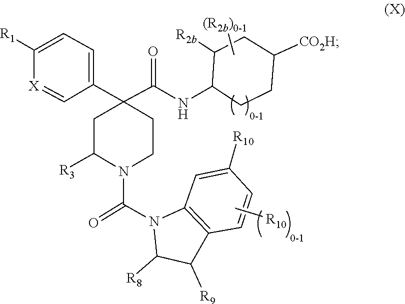

wherein:
X, R₁, R₃, each R_{2b} independently, and each R₁₀ independently are as defined in any one of Embodiments 1 to 14;
R₃ is selected from hydrogen, methyl, ethyl, CHF₂, CH₂CH₂OCH₃, C(═O)H and cyano;
R₉ is R₉ₐ or X₃—R₉ₐ (particularly R₉ₐ);
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and each R_{9b} is as defined in any one of Embodiments 1 to 16 (e.g. each R_{9b} is independently selected from O—C₁-C₃alkyl and 6 membered heterocyclyl comprising 1 heteroatom which is O).

Embodiment 44. The compound or pharmaceutically acceptable salt thereof according to any one of Embodiments 1 to 14, wherein the compound is of formula (XI):

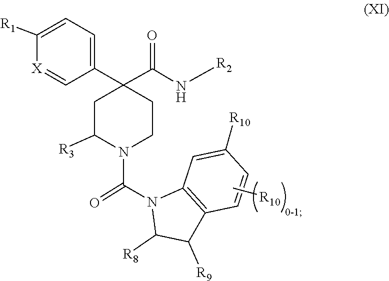

wherein:
X, R₁, R₃, and each R₁₀ independently are as defined in any one of Embodiments 1 to 14;
R₂ is R₂ₐ;
R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b};
each R_{2b} substituent is independently selected from methyl and halo (e.g. fluoro);
R₃ is selected from hydrogen, methyl, ethyl, CHF₂, CH₂CH₂OCH₃, C(═O)H and cyano;
R₉ is R₉ₐ or X₃—R₉ₐ (particularly R₉ₐ);
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and each R_{9b} is as defined in any one of Embodiments 1 to 16 (e.g. each R_{9b} is independently selected from O—C₁-C₃alkyl and 6 membered heterocyclyl comprising 1 heteroatom which is O).

Embodiment 45. A compound selected from:

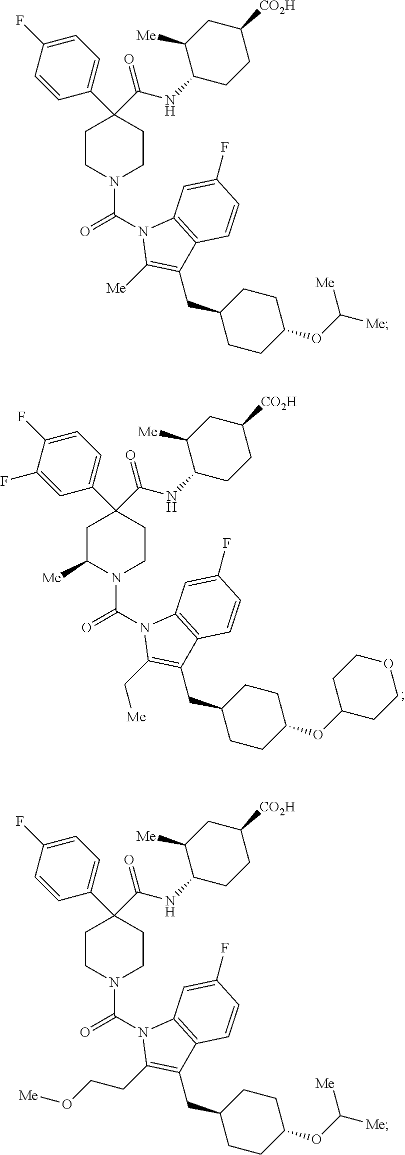 [SMILES: Cc1c(C[C@H]2CC[C@H](OC(C)C)CC2)c2ccc(F)cc2n1C(=O)N1CCC(C(=O)N[C@H]2CC[C@H](C(=O)O)C[C@@H]2C)(c2ccc(F)cc2)CC1 ; CCc1c(C[C@H]2CC[C@H](OC3CCOCC3)CC2)c2ccc(F)cc2n1C(=O)N1CCC(C(=O)N[C@H]2CC[C@H](C(=O)O)C[C@@H]2C)(c2ccc(F)c(F)c2)C[C@@H]1C ; ";" ; COCCc1c(C[C@H]2CC[C@H](OC(C)C)CC2)c2ccc(F)cc2n1C(=O)N1CCC(C(=O)N[C@H]2CC[C@H](C(=O)O)C[C@@H]2C)(c2ccc(F)cc2)CC1]

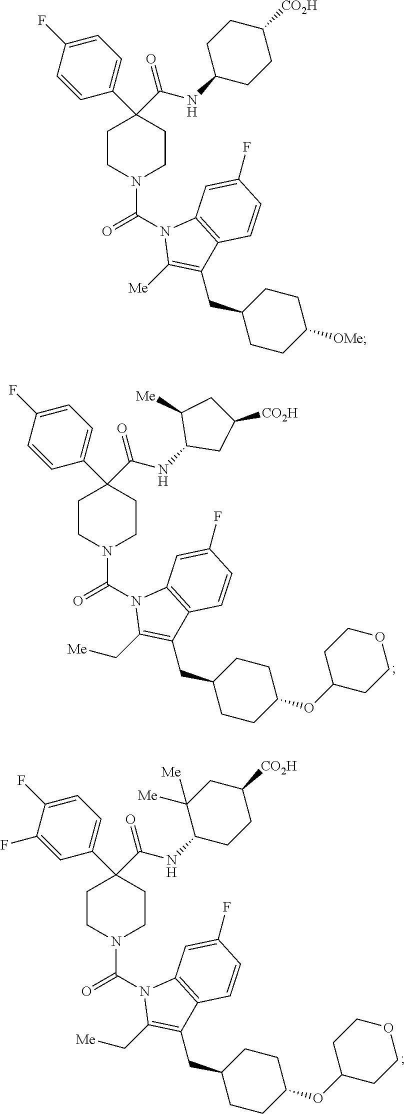

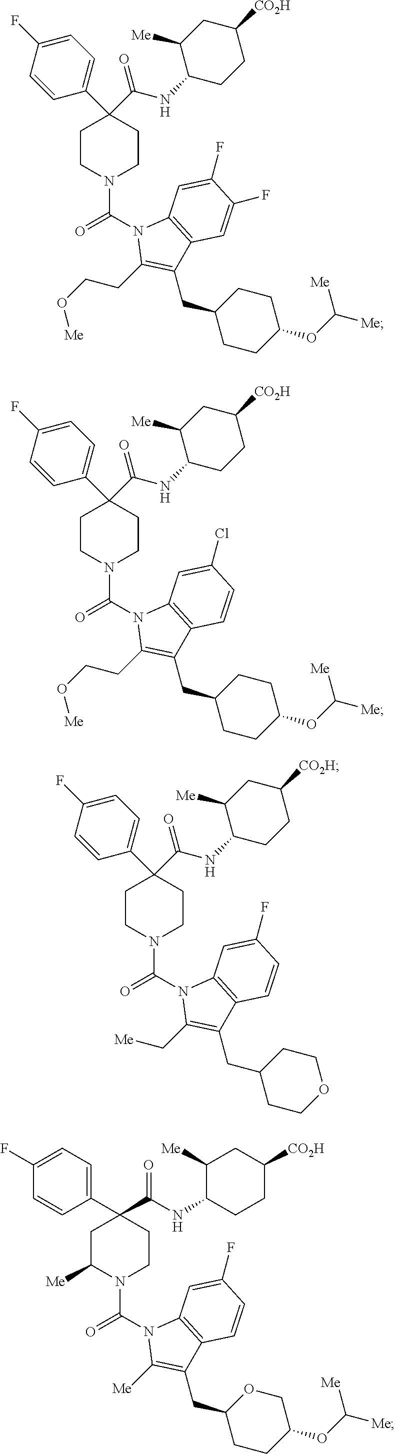

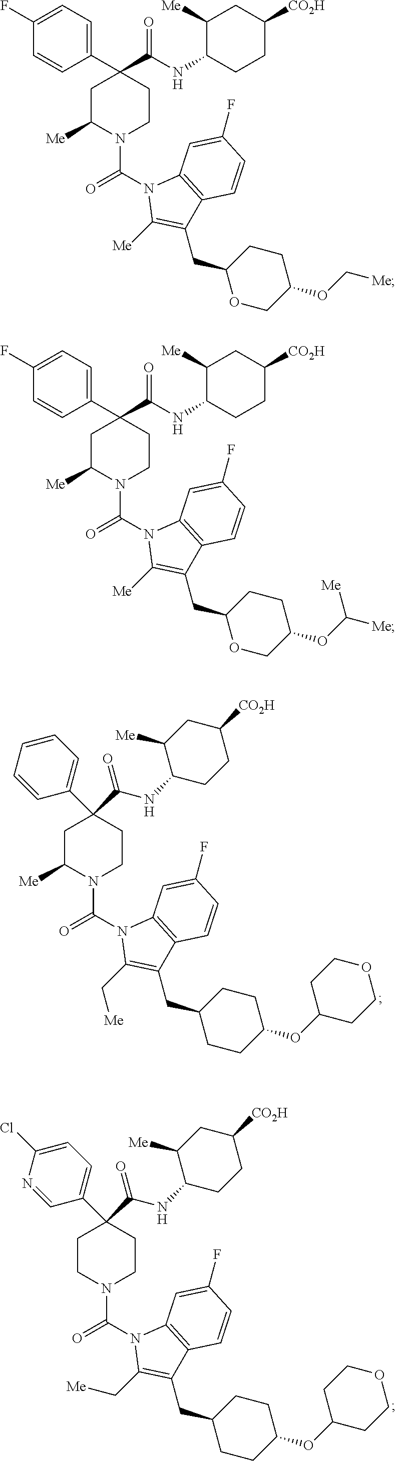

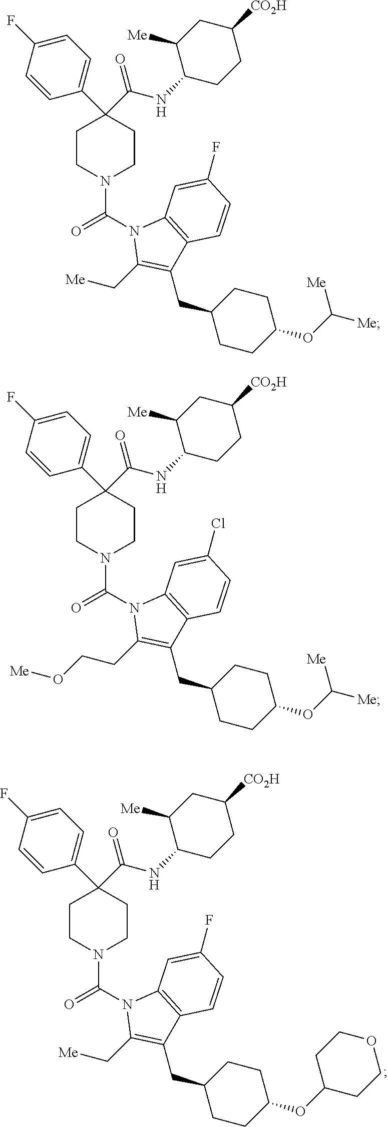

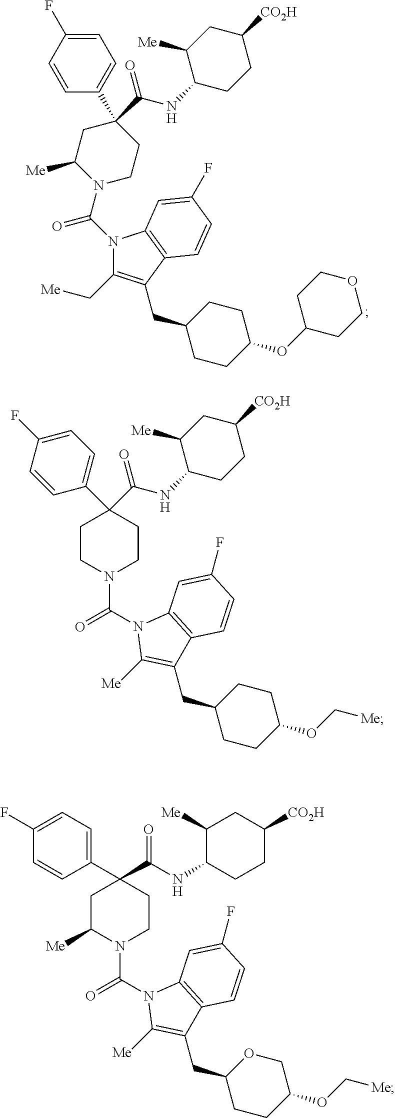 [SMILES: CCc1c(C[C@H]2CC[C@H](OC3CCOCC3)CC2)c2ccc(F)cc2n1C(=O)N1CC[C@@](C(=O)N[C@H]2CC[C@H](C(=O)O)C[C@@H]2C)(c2ccc(F)cc2)C[C@@H]1C ; ";" ; CCO[C@H]1CC[C@H](Cc2c(C)n(C(=O)N3CCC(C(=O)N[C@H]4CC[C@H](C(=O)O)C[C@@H]4C)(c4ccc(F)cc4)CC3)c3cc(F)ccc23)CC1 ; CCO[C@@H]1CC[C@@H](Cc2c(C)n(C(=O)N3CC[C@@](C(=O)N[C@H]4CC[C@H](C(=O)O)C[C@@H]4C)(c4ccc(F)cc4)C[C@@H]3C)c3cc(F)ccc23)OC1]

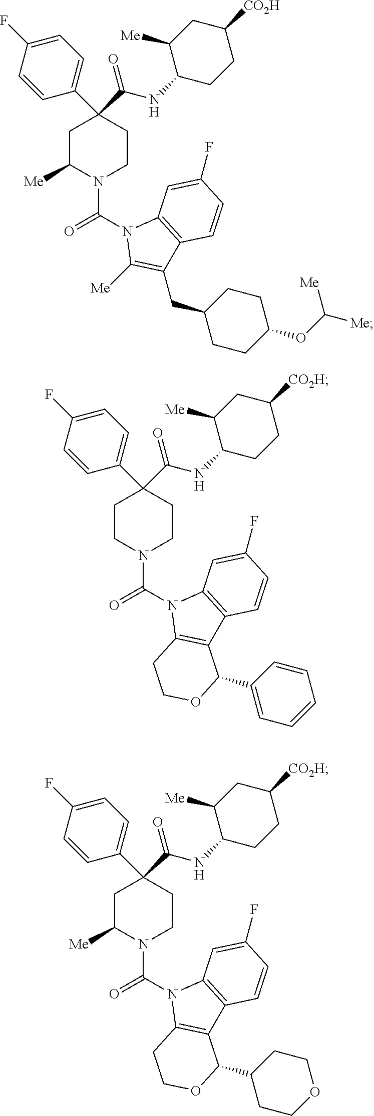

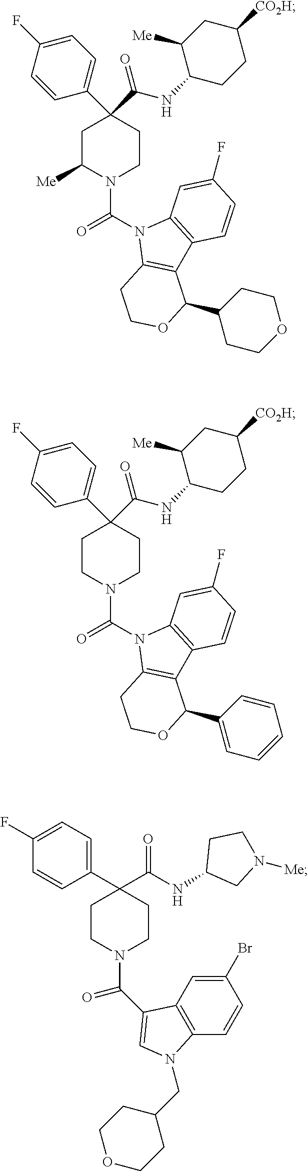 [SMILES: C[C@H]1C[C@@H](C(=O)O)CC[C@@H]1NC(=O)[C@@]1(c2ccc(F)cc2)CCN(C(=O)n2c3c(c4ccc(F)cc42)[C@@H](C2CCOCC2)OCC3)[C@@H](C)C1 ; C[C@H]1C[C@@H](C(=O)O)CC[C@@H]1NC(=O)C1(c2ccc(F)cc2)CCN(C(=O)n2c3c(c4ccc(F)cc42)[C@@H](c2ccccc2)OCC3)CC1 ; CN1CC[C@@H](NC(=O)C2(c3ccc(F)cc3)CCN(C(=O)c3cn(CC4CCOCC4)c4ccc(Br)cc34)CC2)C1]

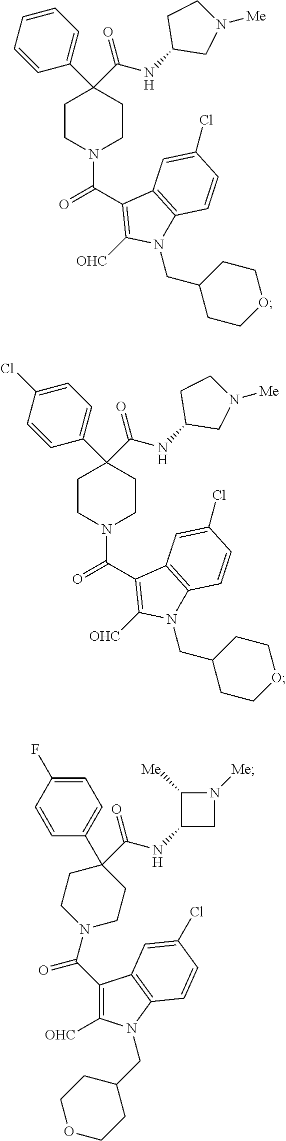 [SMILES: CN1CC[C@@H](NC(=O)C2(c3ccccc3)CCN(C(=O)c3c(C=O)n(CC4CCOCC4)c4ccc(Cl)cc34)CC2)C1 ; CN1CC[C@@H](NC(=O)C2(c3ccc(Cl)cc3)CCN(C(=O)c3c(C=O)n(CC4CCOCC4)c4ccc(Cl)cc34)CC2)C1 ; C[C@H]1[C@@H](NC(=O)C2(c3ccc(F)cc3)CCN(C(=O)c3c(C=O)n(CC4CCOCC4)c4ccc(Cl)cc34)CC2)CN1C]

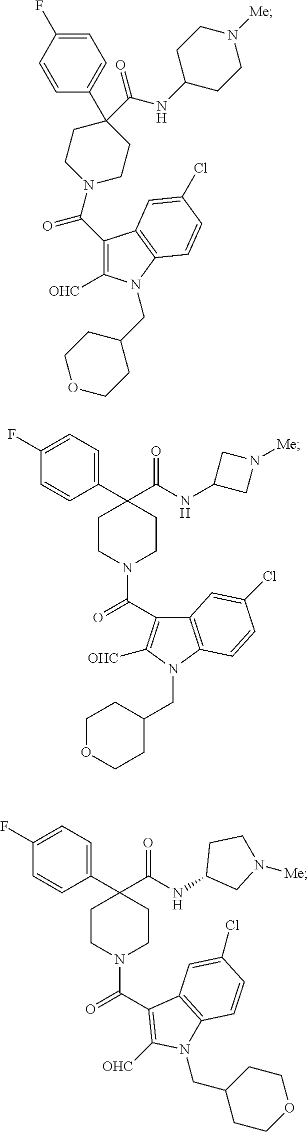 [SMILES: CN1CCC(NC(=O)C2(c3ccc(F)cc3)CCN(C(=O)c3c(C=O)n(CC4CCOCC4)c4ccc(Cl)cc34)CC2)CC1 ; CN1CC(NC(=O)C2(c3ccc(F)cc3)CCN(C(=O)c3c(C=O)n(CC4CCOCC4)c4ccc(Cl)cc34)CC2)C1 ; CN1CC[C@@H](NC(=O)C2(c3ccc(F)cc3)CCN(C(=O)c3c(C=O)n(CC4CCOCC4)c4ccc(Cl)cc34)CC2)C1]

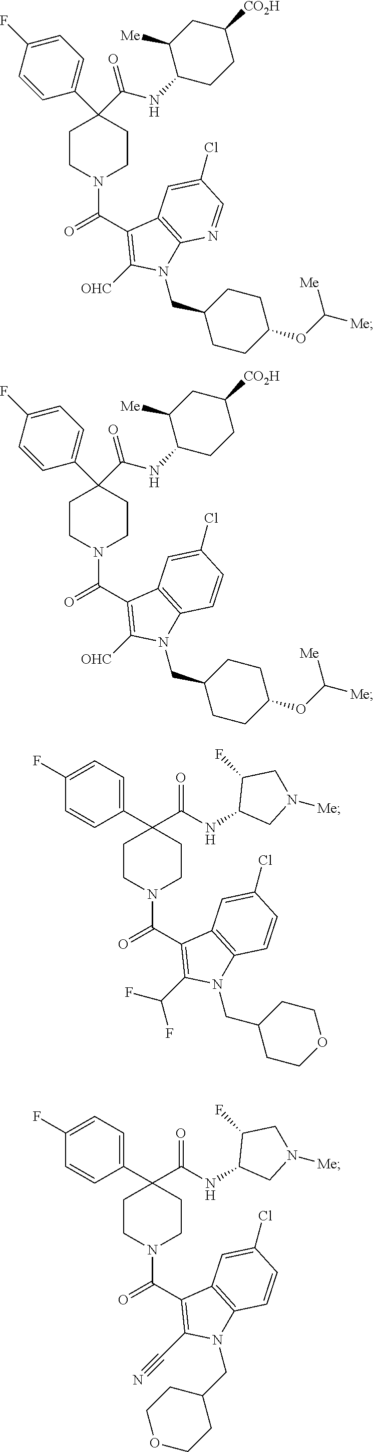

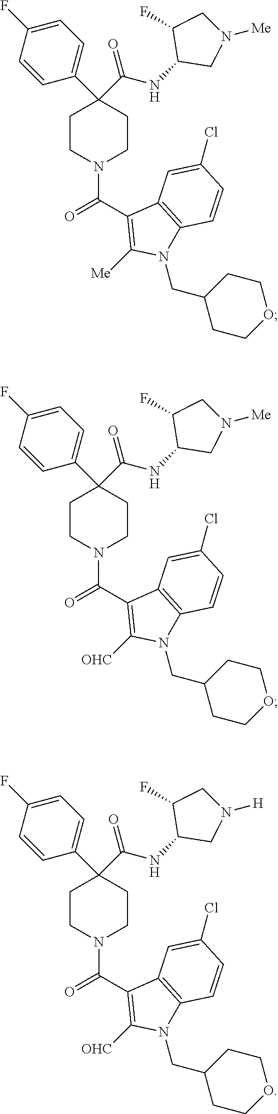 [SMILES: Cc1c(C(=O)N2CCC(C(=O)N[C@H]3CN(C)C[C@H]3F)(c3ccc(F)cc3)CC2)c2cc(Cl)ccc2n1CC1CCOCC1 ; CN1C[C@@H](F)[C@@H](NC(=O)C2(c3ccc(F)cc3)CCN(C(=O)c3c(C=O)n(CC4CCOCC4)c4ccc(Cl)cc34)CC2)C1 ; O=Cc1c(C(=O)N2CCC(C(=O)N[C@H]3CNC[C@H]3F)(c3ccc(F)cc3)CC2)c2cc(Cl)ccc2n1CC1CCOCC1]

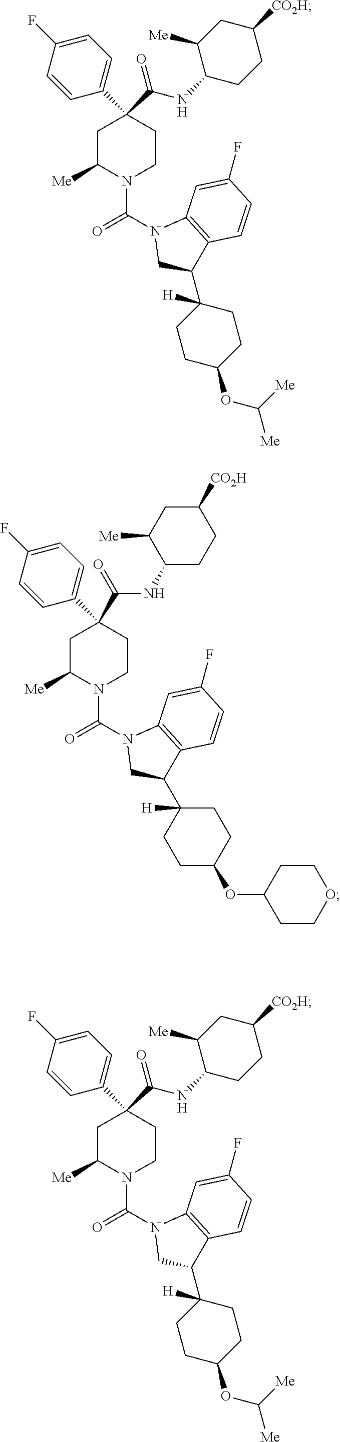 [SMILES: CC(C)O[C@H]1CC[C@H]([C@H]2CN(C(=O)N3CC[C@@](C(=O)N[C@H]4CC[C@H](C(=O)O)C[C@@H]4C)(c4ccc(F)cc4)C[C@@H]3C)c3cc(F)ccc32)CC1 ; C[C@H]1C[C@@H](C(=O)O)CC[C@@H]1NC(=O)[C@@]1(c2ccc(F)cc2)CCN(C(=O)N2C[C@H]([C@H]3CC[C@H](OC4CCOCC4)CC3)c3ccc(F)cc32)[C@@H](C)C1 ; CC(C)O[C@H]1CC[C@H]([C@@H]2CN(C(=O)N3CC[C@@](C(=O)N[C@H]4CC[C@H](C(=O)O)C[C@@H]4C)(c4ccc(F)cc4)C[C@@H]3C)c3cc(F)ccc32)CC1]

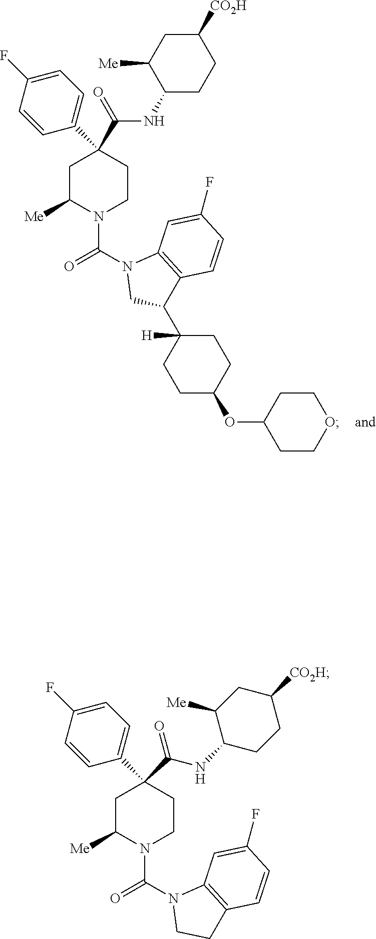 [SMILES: C[C@H]1C[C@@H](C(=O)O)CC[C@@H]1NC(=O)[C@@]1(c2ccc(F)cc2)CCN(C(=O)N2C[C@@H]([C@H]3CC[C@H](OC4CCOCC4)CC3)c3ccc(F)cc32)[C@@H](C)C1 ; C[C@H]1C[C@@H](C(=O)O)CC[C@@H]1NC(=O)[C@@]1(c2ccc(F)cc2)CCN(C(=O)N2CCc3ccc(F)cc32)[C@@H](C)C1]

or a pharmaceutically acceptable salt thereof.

Embodiment 46. A pharmaceutical composition comprising the compound or pharmaceutically acceptable salt thereof according to any one of the preceding Embodiments, and one or more pharmaceutically acceptable carriers.

Embodiment 47. A combination comprising the compound or pharmaceutically acceptable salt thereof according to any one of Embodiments 1 to 45, and one or more additional therapeutically active agents.

Embodiment 48. A method of modulating ERK activity in a subject, the method comprising administering to the subject a therapeutically effective amount of the compound or pharmaceutically acceptable salt thereof according to any one of Embodiments 1 to 45, or the pharmaceutical composition according to Embodiment 46.

Embodiment 49. A method of treating a patient having a disease associated with aberrant activity of the MAP kinase pathway comprising administering to said patient a therapeutically effective amount of the compound or pharmaceutically acceptable salt thereof according to any one of Embodiments 1 to 45, or the pharmaceutical composition according to Embodiment 46.

Embodiment 50. The method according to Embodiment 49, wherein the disease associated with aberrant activity of the MAP kinase pathway is cancer.

Embodiment 51. The method according to Embodiment 50, wherein the cancer is selected from melanoma, lung cancer, colorectal cancer (CRC), pancreatic cancer and thyroid cancer.

Embodiment 52. The method according to Embodiment 50 or Embodiment 51, wherein the cancer contains a BRAF and/or a RAS mutation.

Embodiment 53. A compound or pharmaceutically acceptable salt thereof according to any one of Embodiments 1 to 46 for use as a medicament.

Embodiment 54. A compound or pharmaceutically acceptable salt thereof according to any one of Embodiments 1 to 46 for use in the treatment of cancer.

Embodiment 55. The compound or pharmaceutically acceptable salt thereof for use according to Embodiment 54, wherein the cancer is selected from melanoma, lung cancer, colorectal cancer (CRC), pancreatic cancer and thyroid cancer.

Embodiment 56. The compound or pharmaceutically acceptable salt thereof for use according to Embodiment 54 or Embodiment 55, wherein the cancer contains a BRAF and/or a RAS mutation.

Embodiment 57. Use of a compound or pharmaceutically acceptable salt thereof according to any one of Embodiments 1 to 46 in the manufacture of a medicament for the treatment of cancer.

Embodiment 58. The use according to Embodiment 57, wherein the cancer is selected from melanoma, lung cancer, colorectal cancer (CRC), pancreatic cancer and thyroid cancer.

Embodiment 59. The use according to Embodiment 57 or Embodiment 58, wherein the cancer contains a BRAF and/or a RAS mutation.

### Definitions

For the purpose of interpreting this specification, the following definitions will apply unless specified otherwise and when appropriate, terms used in the singular will also include the plural and vice versa. It must be noted that as used herein and in the appended claims, the singular forms “a”, “an” and “the” include the plural unless the context clearly dictates otherwise. Thus, for example, reference to “the compound” includes reference to one or more compounds, and so forth.

As used herein, the term “substituent” refers to a radical group which replaces a hydrogen atom in a given molecule.

As used herein, the term “alkyl” refers to a straight or branched hydrocarbon chain radical consisting solely of carbon and hydrogen atoms, containing no unsaturation, and which is attached to the rest of the molecule by a single bond. For instance, C₁-C₄alkyl contains from 1 to 4 carbon atoms. Examples of C₁-C₄alkyl include, but are not limited to, methyl (Me), ethyl (Et), n-propyl, 1-methylethyl (iso-propyl), n-butyl and t-butyl.

As used herein, the term “halogen”, “halo”, “hal”, etc. refers to fluorine, chlorine, bromine or iodine.

Halogen-substituted groups and moieties, such as alkyl substituted by halogen (haloalkyl) can be mono-, poly- or per-halogenated.

As used herein, the term “haloalkyl” refers to an alkyl radical as defined herein, wherein one or more of the hydrogen atoms of said alkyl has been replaced with a halogen atom. For instance, C₁-C₄haloalkyl contains from 1 to 4 carbon atoms (and 1 or more halogen atoms).

As used herein, the term “hydroxyalkyl” refers to an alkyl radical as defined herein, wherein one or more of the hydrogen atoms of said alkyl has been replaced with an —OH group. For instance, C₁-C₄hydroxyalkyl contains from 1 to 4 carbon atoms (and 1 or more OH groups).

As used herein, the term “alkylene” refers to a straight-chain or branched divalent radical of an alkyl group. For instance, “C₁-C₄alkylene” contains from 1 to 4 carbon atoms e.g., —CH₂—, —CH₂CH₂—, —CH₂CH₂CH₂—, —CH(CH₃)₂—, —CH₂CH(CH₃)CH₂—.

Likewise, as used herein, the term “haloalkylene” refers to a straight-chain or branched divalent radical of a haloalkyl group.

As used herein, the term “cycloalkyl” refers to a saturated carbocyclic ring radical. C₃-C₆cycloalkyl for instance, is any such ring radical containing 3 to 6 carbon atoms, and is particularly monocyclic i.e. cyclobutyl, cyclopentyl and cyclohexyl. However, the cycloalkyl (e.g. C₃-C₆cycloalkyl) can also be a fused (e.g.

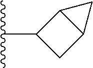 [SMILES: *C1CC2CC12]

a 5 membered fused ring) or bridged (e.g.

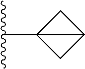 [SMILES: *C12CC1C2]

a 4 membered bridged ring) bicyclic ring system.

As used herein, the term “cyclohaloalkyl” refers to a cycloalkyl radical as defined herein, wherein one or more of the hydrogen atoms of said cycloalkyl has been replaced with a halogen atom. Particularly said one or more halogen atom(s) are each fluorine atom(s), in which case the “cyclohaloalkyl” is a “cyclofluoroalkyl”. As with cycloalkyls, a halocycloalkyl can be a fused or bridged bicyclic ring system.

As used herein, the term “heterocyclyl”, “heterocycle”, “heterocyclic”, “heterocyclic ring” etc. refers to a heterocyclic radical that is saturated or partially unsaturated but not aromatic, and can be a monocyclic or a polycyclic ring, including a fused or bridged bicyclic ring system. Particularly, however, the heterocyclyl is a monocyclic ring. A heterocyclyl contains at least one non-carbon atom as a ring member, typically N, O or S unless otherwise specified, the remaining ring atoms therefore being carbon. Where a (n unsubstituted) heterocyclyl contains S as a heteroatom, the S can be in the form of S, SO or SO₂ (in other words, the oxygen atoms bonded to the sulphur do not constitute substitutions). For example, the term “4-6 membered heterocyclyl comprising 1 heteroatom selected from the group consisting of O, N and S” refers to a ring radical containing 4 to 6 ring atoms comprising 1 heteroatom (either O, N, or S [the latter including S, SO and SO₂]), with the remaining ring atoms being carbon. An example of a 7 membered bridged heterocyclic ring is

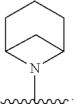 [SMILES: *N1C2CCCC1C2]

An example of a 7 membered fused heterocyclic ring is

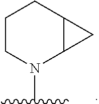 [SMILES: *N1CCCC2CC21]

As used herein, the term “spirocycloalkyl” refers to a ring system comprising a first carbocyclic ring comprising from 3 to 6 ring carbon atoms, wherein two of the substituents on a carbon ring atom in said first carbocyclic ring join together to form a second carbocyclic ring comprising from 3 to 6 ring carbon atoms. Particularly, the spirocycloalkyl is saturated. The term 6-8 membered spirocycloalkyl, as used herein means that the total number of carbon ring atoms in the first carbocyclic ring and the second carbocyclic ring is from 6 to 8. As will be appreciated by the skilled person a “spirocycloalkylene” is a di-radical equivalent to a “spirocycloalkyl”.

As used herein, the term “spiroheterocyclyl”, refers to ring system comprising a first carbocyclic or heterocyclic ring comprising from 3 to 6 ring atoms wherein two of the substituents on a carbon ring atom in said first carbocyclic or heterocyclic ring join together to form a second carbocyclic or heterocyclic ring comprising from 3 to 6 ring atoms, with the proviso that at least one of the first and second rings is a heterocyclic ring comprising one or more heteroatoms selected from the group consisting of O, N and S (the latter can be in the form of S, SO or SO₂), particularly selected from the group consisting of O and N. Particularly, the spiroheterocyclyl is saturated. The term 7-9 membered spiroheterocyclyl, as used herein means that the total number of ring atoms in the first carbocyclic or heterocyclic ring and the second carbocyclic or heterocyclic ring is from 7 to 9.

For instance, the spiroheterocyclyl

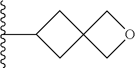 [SMILES: *C1CC2(COC2)C1]

is a 7 membered spiroheterocyclyl, as there are 7 ring atoms present. As will be appreciated by the skilled person, a “spiroheterocyclyl” is a mono-radical, whereas a “spiroheterocyclylene” is a di-radical (analogous to alkyl and alkylene).

The term “heteroaryl” refers as used herein to a monocyclic aromatic ring radical which, unless otherwise stated, comprises 1, 2, 3 or 4 heteroatoms individually selected from nitrogen, oxygen and sulfur (in the form of S, SO or SO₂) in the ring radical. Typical monocyclic heteroaryl groups include 2- or 3-thienyl, 2- or 3-furyl, 2- or 3-pyrrolyl, 2-, 4-, or 5-imidazolyl, 1-, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-(1,2,4-triazolyl), 4- or 5-(1,2, 3-triazolyl), 2-, 3-, or 4-pyridyl, 3- or 4-pyridazinyl, 2-pyrazinyl, and 2-, 4-, or 5-pyrimidinyl. Particularly the heteroaryl contains 1-3 heteroatoms individually selected from nitrogen, oxygen and sulfur.

As used herein, the terms “3-6 membered saturated or partially unsaturated carbocyclic ring” and “5-6 membered saturated or partially unsaturated carbocyclic ring” refer to a radical monocyclic ring that is saturated or partially unsaturated but not aromatic, and has no non-carbon atoms as a ring member. The terms thus include cycloalkyls such as cyclopentane, cyclo(mono)alkenes such as cyclopentene, as well as cyclodienes such as 1,3-cyclohexadiene. The former term includes 3, 4, 5 and 6 membered rings, whereas the latter is limited to 5 and 6 membered rings.

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the present invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto. When both a basic group and an acid group are present in the same molecule, the compounds of the present invention may also form internal salts, e.g., zwitterionic molecules. Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the present invention provides compounds of the present invention in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

In another aspect, the present invention provides compounds according to any one of embodiments 1 to 45, in sodium, potassium, ammonium, calcium, magnesium, iron, silver, zinc, copper, isopropylamine, benzathine, cholinate, diethanolamine, diethylamine, lysine, meglumine, piperazine or tromethamine salt form.

Any formula given herein is also intended to represent unlabelled forms as well as isotopically labelled forms of the compounds. Isotopically labelled compounds have structures depicted by the formulae given herein except that one or more atoms are replaced by an atom having a selected atomic mass or mass number. Isotopes that can be incorporated into compounds of the invention include, for example, isotopes of hydrogen.

Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present invention. The concentration of deuterium, may be defined by the isotopic enrichment factor. The term “isotopic enrichment factor” as used herein means the ratio between the isotopic abundance and the natural abundance of a specified isotope. If a substituent in a compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the invention include isotopes of hydrogen, carbon, nitrogen, oxygen, phosphorous, fluorine, and chlorine, such as ³H, ¹¹C, ¹³C, ¹⁴C, ¹⁵N, ¹⁸F, ³¹P, ³²P, ³⁵S, ³⁶Cl, ¹²³I, ¹²⁴I, ¹²⁵I respectively. Accordingly it should be understood that the invention includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagent in place of the non-labeled reagent previously employed.

### Pharmaceutical Composition

As used herein, the term “pharmaceutical composition” refers to a compound of the invention, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22^{nd} Ed. Pharmaceutical Press, 2013, pp. 1049-1070). The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease (i) mediated by ERK, or (ii) associated with ERK activity, or (iii) characterized by activity (normal or abnormal) of ERK; or (2) reduce or inhibit the activity of ERK; or (3) reduce or inhibit the expression of ERK. In another non-limiting embodiment, the term “a therapeutically effective amount” refers to the amount of the compound of the present invention that, when administered to a cell, or a tissue, or a non-cellular biological material, or a medium, is effective in at least partially reducing or inhibiting the activity of ERK; or at least partially reducing or inhibiting the expression of ERK.

As used herein, the term “subject” refers to primates (e.g., humans, male or female), dogs, rabbits, guinea pigs, pigs, rats and mice. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder.

As used herein, the term “a”, “an”, “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the present invention or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds of the present invention or racemic intermediates can also be resolved by chiral chromatography, e.g., high pressure liquid chromatography (HPLC) using a chiral adsorbent.

### Methods of Synthesizing the Compounds of the Invention

All methods described herein can be performed in any suitable order unless otherwise indicated herein or otherwise clearly contradicted by context. The use of any and all examples, or exemplary language (e.g. “such as”) provided herein is intended merely to better illustrate the invention and does not pose a limitation on the scope of the invention otherwise claimed.

The compounds of the present application can be prepared by those skilled in the art of organic synthesis using commercially available starting materials, compounds known in the literature, or from readily prepared intermediates, by employing standard synthetic methods and procedures either known to those skilled in the art, or which will be apparent to the skilled chemist in light of the teachings herein.

The compounds of Formula (I) may be prepared by methods as set forth in the following synthetic reaction schemes. In the schemes described below, it is well understood that protecting groups for sensitive or reactive groups are employed where necessary in accordance with general principles of chemistry. Protecting groups are manipulated according to standard methods of organic synthesis as described for example in Protective Groups in Organic Synthesis, 3rd edition, John Wiley & Sons: New York, 1999 or Protecting Groups, 3rd edition, Thieme, Stuttgart, 2004. Protective groups are removed at a convenient stage of the compound synthesis using methods that are readily apparent to those skilled in the art.

Those skilled in the art will recognize if a stereocentre exists in the compounds disclosed herein. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

Compounds of the present disclosure can be synthesized by following the steps outlined in the reaction schemes. Starting materials are either commercially available or made by known procedures in the reported literature or as illustrated.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure material. Compounds of the invention and intermediates can also be converted into each other according to methods generally known to those skilled in the art.

In another aspect, the present invention provides a pharmaceutical composition comprising a compound of the present invention, or a pharmaceutically acceptable salt thereof, and a pharmaceutically acceptable carrier. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration. Topical administration may also pertain to inhalation or intranasal application. The pharmaceutical compositions of the present invention can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

### Methods of Use of the Invention

The compounds of formula (I), in free form or in pharmaceutically acceptable salt form, exhibit valuable pharmacological properties, for example ERK modulating properties, for example as indicated in in vitro tests as provided in the next sections, and are therefore indicated for therapy or for use as research chemicals, e.g. as tool compounds.

Compounds of the present invention may be useful in the treatment of cancer, for example a cancer selected from melanoma, lung cancer, colorectal cancer (CRC), pancreatic cancer and thyroid cancer.

Thus, as a further aspect, the present invention provides the use of a compound of the present invention as a medicament. In a further embodiment, the therapy is selected from a disease which may be treated by modulation of ERK. In another embodiment, the disease is cancer, for example a cancer selected from melanoma, lung cancer, colorectal cancer (CRC), pancreatic cancer and thyroid cancer.

In another aspect, the invention provides a method of treating a patient having a disease associated with aberrant activity of the MAP kinase pathway comprising administering to said patient a therapeutically effective amount of the compound or pharmaceutically acceptable salt thereof according to the invention. In another embodiment, the disease is cancer, for example a cancer selected from melanoma, lung cancer, colorectal cancer (CRC), pancreatic cancer and thyroid cancer.

The pharmaceutical composition or combination of the present invention may, for example, be in unit dosage of about 1-1000 mg of active ingredient(s) for a subject of about 50-70 kg.

The compound of the present invention may be administered either simultaneously with, or before or after, one or more other therapeutic agent. The compound of the present invention may be administered separately, by the same or different route of administration, or together in the same pharmaceutical composition as the other agents. A therapeutic agent is, for example, a chemical compound, peptide, antibody, antibody fragment or nucleic acid, which is therapeutically active or enhances the therapeutic activity when administered to a patient in combination with a compound of the present invention.

In one embodiment, the invention provides a product comprising a compound of the present invention and at least one other therapeutic agent as a combined preparation for simultaneous, separate or sequential use in therapy. In one embodiment, the therapy is the treatment of a disease or condition mediated by ERK. Products provided as a combined preparation include a composition comprising the compound of the present invention and the other therapeutic agent(s) together in the same pharmaceutical composition, or the compound of the present invention and the other therapeutic agent(s) in separate form, e.g. in the form of a kit.

In one embodiment, the invention provides a pharmaceutical composition comprising a compound of the present invention and another therapeutic agent(s). Optionally, the pharmaceutical composition may comprise a pharmaceutically acceptable carrier, as described above.

In one embodiment, the invention provides a kit comprising two or more separate pharmaceutical compositions, at least one of which contains a compound of the present invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the present invention and the other therapeutic agent may be manufactured and/or formulated by the same or different manufacturers. Moreover, the compound of the present invention and the other therapeutic may be brought together into a combination therapy: (i) prior to release of the combination product to physicians (e.g. in the case of a kit comprising the compound of the present invention and the other therapeutic agent); (ii) by the physician themselves (or under the guidance of the physician) shortly before administration; (iii) in the patient themselves, e.g. during sequential administration of the compound of the present invention and the other therapeutic agent.

Accordingly, the invention provides the use of a compound of the present invention for treating a disease or condition mediated by ERK, wherein the medicament is prepared for administration with another therapeutic agent. The invention also provides the use of another therapeutic agent for treating a disease or condition mediated by ERK, wherein the medicament is administered with a compound of the present invention.

The invention also provides a compound of the present invention for use in a method of treating a disease or condition mediated by ERK, wherein the compound of the present invention is prepared for administration with another therapeutic agent. The invention also provides another therapeutic agent for use in a method of treating a disease or condition mediated by ERK, wherein the other therapeutic agent is prepared for administration with a compound of the present invention. The invention also provides a compound of the present invention for use in a method of treating a disease or condition mediated by ERK, wherein the compound of the present invention is administered with another therapeutic agent. The invention also provides another therapeutic agent for use in a method of treating a disease or condition mediated by ERK, wherein the other therapeutic agent is administered with a compound of the present invention.

The invention also provides the use of a compound of the present invention for treating a disease or condition mediated by ERK, wherein the patient has previously (e.g. within 24 hours) been treated with another therapeutic agent. The invention also provides the use of another therapeutic agent for treating a disease or condition mediated by ERK, wherein the patient has previously (e.g. within 24 hours) been treated with compound of the present invention.

### General Reaction Scheme

Compounds of the invention can be prepared by proceeding as in the following general Reaction Schemes:

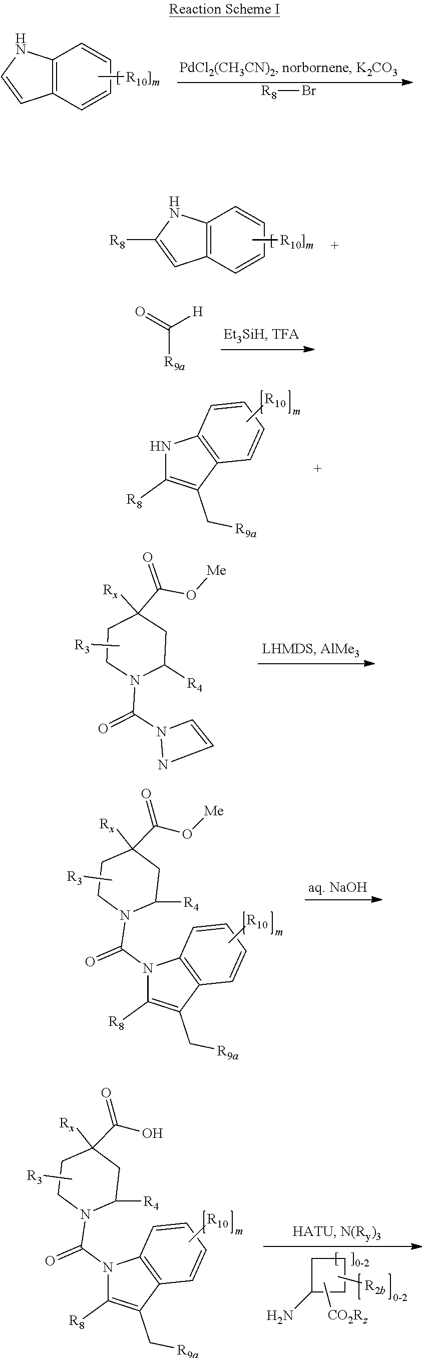

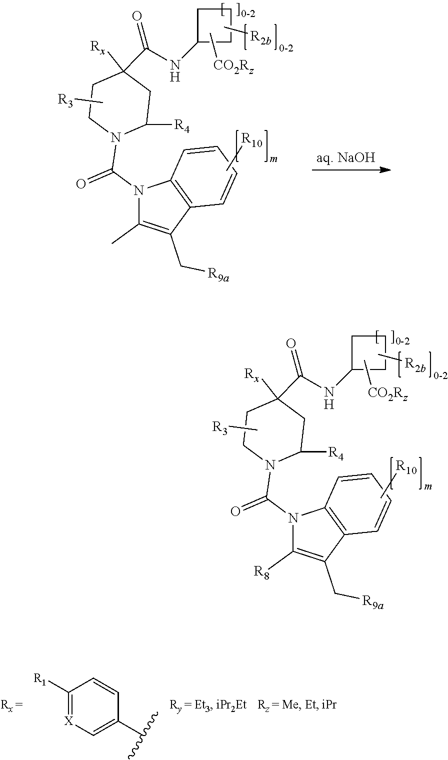

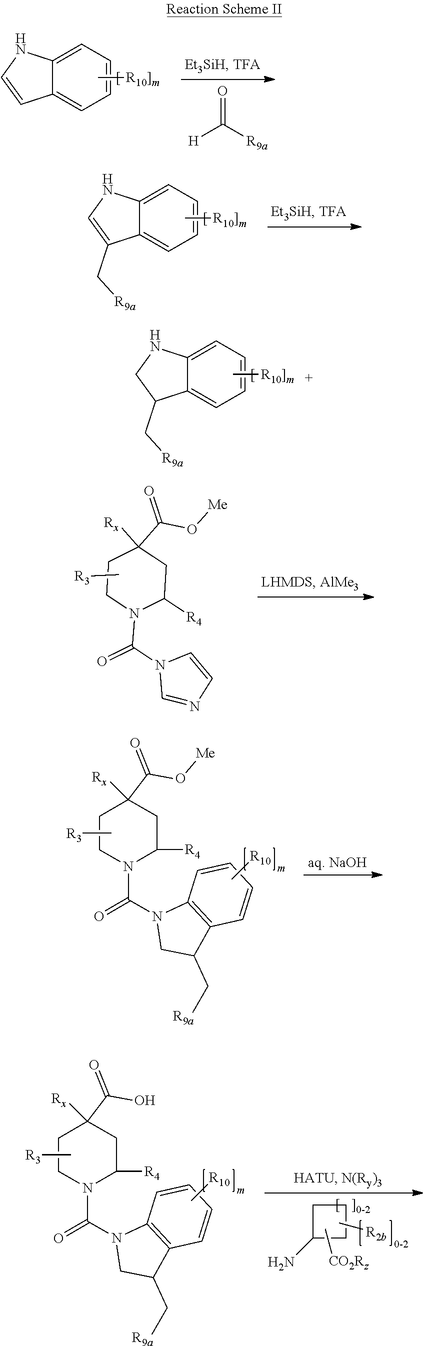

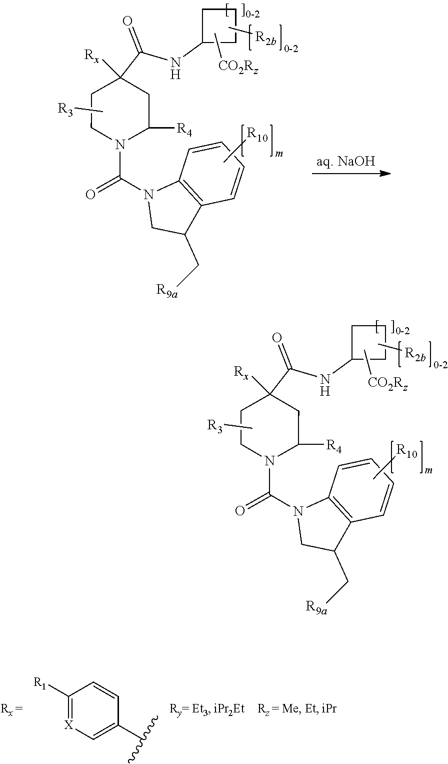

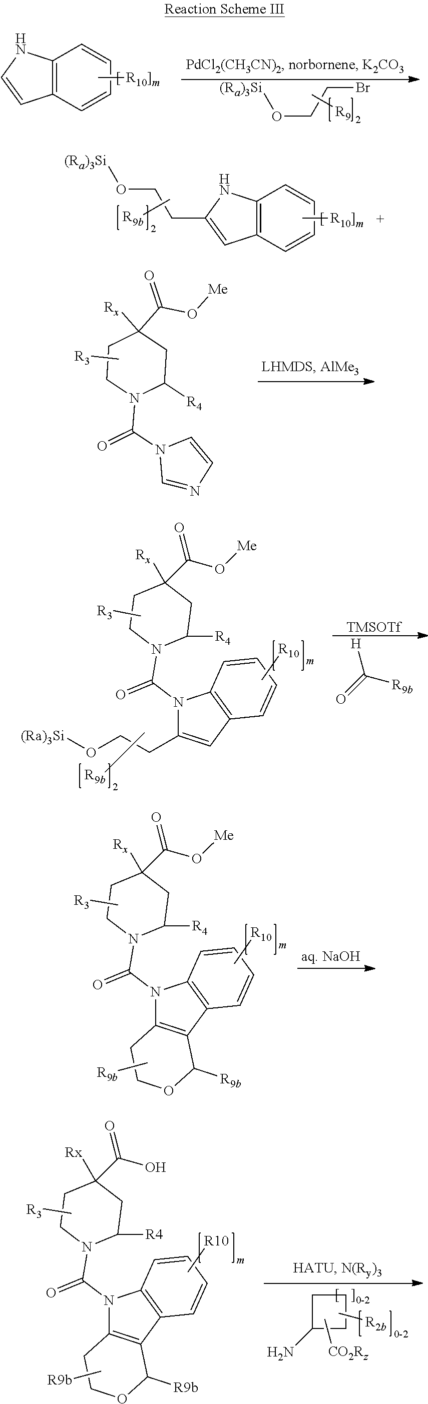

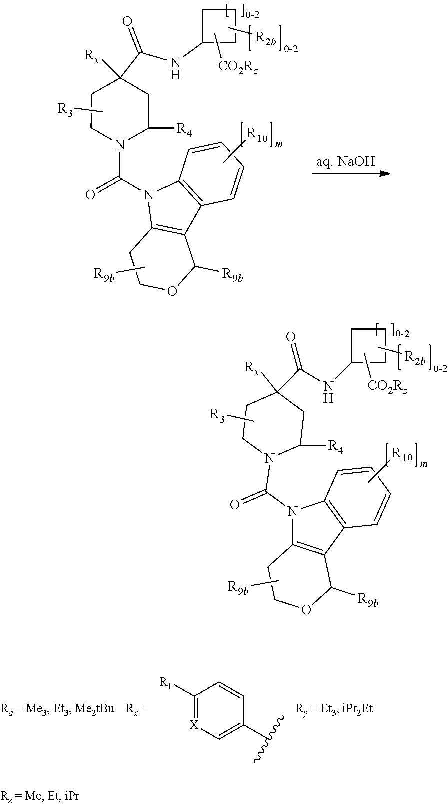

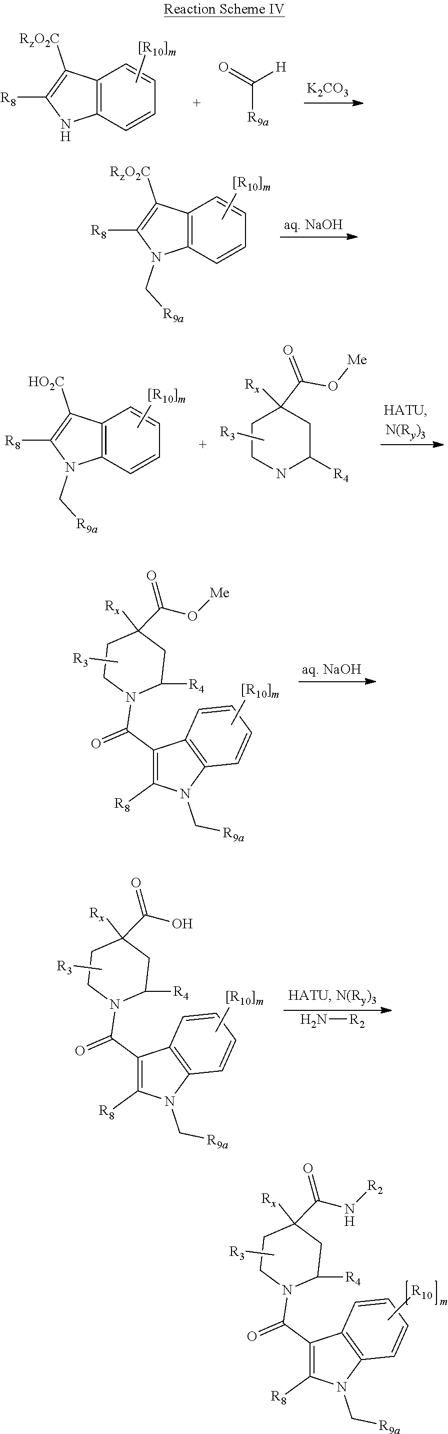

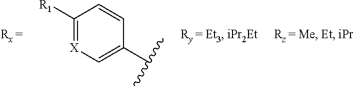

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Compounds of the present disclosure may be prepared by methods known in the art of organic synthesis. In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (1999) Protective Groups in Organic Synthesis, 3rd edition, John Wiley & Sons). These groups are removed at a convenient stage of the compound synthesis using methods that are readily apparent to those skilled in the art.

### Analytical Methods, Materials, and Instrumentation

Unless otherwise noted, reagents and solvents were used as received from commercial suppliers. Proton nuclear magnetic resonance (¹H NMR) spectra were acquired on Bruker AVANCE 400 MHz, 500 MHz or 600 MHz NMR spectrometers using ICON-NMR, under TopSpin program control unless otherwise noted. Spectra were measured at 298 K, unless indicated otherwise, and were referenced relative to the solvent resonance. Tetramethylsilane (TMS) was used as an internal standard. Chemical shifts are reported in ppm relative to dimethyl sulfoxide (δ 2.50), methanol (δ 3.31), chloroform (δ 7.26) or other solvent as indicated in NMR spectral data. A small amount of the dry sample (2 to 5 mg) is dissolved in an appropriate deuterated solvent (1 mL). The chemical names were generated using ChemBioDraw Ultra v19 from CambridgeSoft.

Mass spectra were acquired on LC-MS, SFC-MS, or GC-MS systems using electrospray, chemical and electron impact ionization methods from a range of instruments of the following configurations: Waters Acquity UPLC/SQD system, using a photodiode array detector and a single quadrupole mass detector; Agilent 1200 systems with G 6110 series mass detector; Agilent 1290 Infinity II with DAD (photodiode array detector) and single quadrupole mass detector with ESI and APCI ionization (multi-mode); Waters AcQuity UPLC with PDA (photodiode array detector), ELSD and single quadrupole mass detector with ESI ionization; Waters AutoPurification System with PDA (photodiode array detector) and single quadrupole mass detector with ESI ionization; [M+H]⁺ refers to protonated molecular ion of the chemical species; [M−H]⁻ refers to molecular ion of the chemical species with loss of one proton; [M+Na]⁺ refers to molecular ion of the chemical species with addition of one sodium ion; [M-Boc+H]⁺ refers to protonated molecular ion of the chemical species without a Boc protecting group; [M-tBu+2H]⁺ refers to protonated molecular ion of the chemical species without a tert-butyl group.

### Abbreviations

Some abbreviations used in the examples are as follows: 1,1-bis(diphenylphosphino)-ferrocenedichloropalladium (II) (PdCl₂(dppf)); 1,1-carbonyldiimidazole (CDI); 1-hydroxy-7-azabenzotriazole (HOAt); 2-(1H-7-azabenzotriazol-1-yl)-1,1,3,3-tetramethyluronium hexafluorophosphate (HATU); 2,2′-bis-diphenylphosphanyl-[1,1′]binaphthalenyl (BINAP); 4-dimethylaminopyridine (DMAP); 3-morpholinopropane-1-sulfonic acid (MOPS); acetic acid (AcOH); acetic anhydride (Ac₂O); acetonitrile (CH₃CN); aqueous (aq.); atmosphere (atm.); back pressure regulator (BPR); broad (br); benzotriazole-1-yl-oxy-tris-(dimethylamino)-phosphonium hexafluorophosphate (BOP); doublet (d); dichloromethane (DCM); diethyl ether (Et₂O); diisopropyl azodicarboxylate (DIAD); dimethyl sulfoxide (DMSO); diphenylphosphoryl azide (DPPA); di-tert-butyl dicarbonate (Boc₂O); equivalent(s) (equiv.); ethanol (EtOH); ethyl acetate (EtOAc); fetal bovine serum (FBS); Förster resonance energy transfer (FRET); gram(s) (g); high performance liquid chromatography (HPLC); high-resolution mass spectrum (HRMS); homogeneous time-resolved FRET (HTRF); hour(s) (h); hydrochloric acid (HCl); inner diameter (I.D.); isopropanol (iPrOH); isopropylamine (iPr₂NH); liquid chromatography coupled with mass spectrometry (LCMS); liter(s) (L); lithium aluminium hydride (LAH); lithium bis(trimethylsilyl)amide (LHMDS); lithium diisopropylamide (LDA); lithium hydroxide (LiOH); luminescence (LUM); magnesium sulfate (MgSO₄); mass spectrum (MS); meta-chloroperoxybenzoic acid (mCPBA); metabolism (MT); methanol (MeOH); methyl iodide (Mel); methyl tert-butyl ether (MBTE); microwave (MW); microliter(s) (μL); micrometer(s) (μm); micromole(s) (μmol); milliliter(s) (mL); millimeter(s) (mm); millimole(s) (mmol); minute(s) (min); mole(s) (mol); multiplet (m); N-(3-dimethylaminopropyl)-N′-ethylcarbodiimide hydrochloride (EDC); n-butyllithium (n-BuLi); N-chlorosuccinimide (NCS); N-hydroxy succinimide (NHS); N,N-diisopropyl-ethylamine (DIPEA); N,N-dimethylformamide (DMF); isopropanol (iPrOH); pentet (p); potassium hydroxide (KOH); potassium tert-butoxide (KOtBu); palladium on carbon (Pd/C); para-toluene sulfonic acid (PTSA); para-toluenesulfonyl chloride (TsCl); phosphate buffered saline (PBS); quartet (q); retention time (Rₜ); Roswell Park Memorial Institute medium (RPMI); room temperature (RT); saturated (sat.); singlet (s); sodium bicarbonate (NaHCO₃); sodium borohydride (NaBH₄); sodium carbonate (Na₂CO₃); sodium hydride (NaH); sodium hydroxide (NaOH); sodium sulfate (Na₂SO₃); sodium thiosulate (Na₂S₂O₃); supercritical fluid chromatography (SFC); tert-butoxycarbonyl (Boc); tert-butyldimethylsilyl chloride (TBSCI); tetrahydrofuran (THF); toluenesulfonylmethyl isocyanide (TosMIC); triethylamine (NEt₃); triethylsilane (Et₃SiH); trifluoroacetic acid (TFA); trimethylaluminum (AlMe₃); trimethylsilyl trifluoromethanesulfonate (TMSOTf); triplet (t); tris(2-carboxyethyl)phosphine (TCEP); tri-tert-butylphosphonium tetrafluoroborate (TTBP-HBF₄); thionyl chloride (SOCl₂); trimethylsilyl chloride (TMSCI); weight (wt.).

### Intermediate 1

### 2-ethyl-6-fluoro-1H-indole

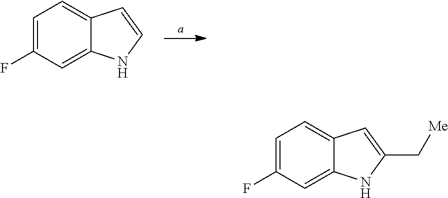 [SMILES: Fc1ccc2cc[nH]c2c1 ; CCc1cc2ccc(F)cc2[nH]1]

Step a: To 6-fluoro-1H-indole (62.5 g, 462 mmol) in dimethylacetamide (2.0 L) and H₂O (200 mL) at RT, was added bicyclo[2.2.1]hept-2-ene (87.1 g, 925 mmol) followed by K₂CO₃ (128 g, 927 mmol). The reaction mixture was sparged with N₂ while stirring for 15 min. Bromoethane (150 g, 1.38 mol) was added followed by bis(acetonitrile)palladium chloride (14.4 g, 55.0 mmol). A reflux condenser was fitted to the flask, and the head space was sparged with N₂ for an additional 45 min. The outlet was removed and the reaction mixture was stirred for an additional 14 h at 70° C., upon which time it was cooled to RT, diluted with MTBE (1.4 L) and filtered. The resulting mixture was poured into H₂O (650 mL), and the organic layer was partitioned. The aq. layer was extracted with MTBE (650 mL×2), and combined organic extracts were washed with H₂O (500 mL×2), dried over Na₂SO₄, and concentrated under reduced pressure to yield the crude product. The crude product was purified via silica gel chromatography (EtOAc:heptane, 1:10). Desired fractions were combined and concentrated under reduced pressure. This material was further purified via recrystallization from hexane (200 mL) to yield 2-ethyl-6-fluoro-1H-indole (95.0 g) as an off-white solid. ¹H NMR (400 MHz, DMSO-d₆) δ 10.98 (s, 1H), 7.36 (dd, J=8.4, 5.2 Hz, 1H), 7.03 (dd, J=10.0, 2.4 Hz, 1H), 6.79-6.74 (m, 1H), 6.12 (dd, J=2.0, 0.8 Hz, 1H), 2.71 (m, J=7.6 Hz, 2H), 1.25 (t, J=7.6 Hz, 3H). MS m/z 164.2 [M+H]⁺.

The following compounds of table 1 were synthesized using the above procedure or modifications to the above procedure using the corresponding functionalized indole and alkyl bromide.

TABLE 1

| Intermediate ID | Structure | Analytical data |
|---|---|---|
| 1a | 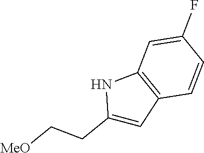 [SMILES: COCCc1cc2ccc(F)cc2[nH]1] | ¹H NMR (300 MHz, DMSO-d₆) δ 10.96 (s, 1H), 7.43 − 7.29 (m, 1H), 7.11 − 7.00 (m, 1H), 6.82 − 6.70 (m, 1H), 6.16 (s, 1H), 3.61 (t, J = 6.6 Hz, 2H), 3.26 (s, 3H), 2.91 (t, J = 6.6 Hz, 2H). |
| 1b | 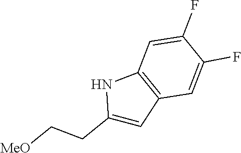 [SMILES: COCCc1cc2cc(F)c(F)cc2[nH]1] | ¹H NMR (300 MHz, DMSO-d₆) δ 11.08 (s, 1H), 7.32 (ddd, J = 28.6, 11.3, 7.5 Hz, 2H), 6.18 (d, J = 1.6 Hz, 1H), 3.62 (t, J = 6.6 Hz, 2H), 3.27 (s, 3H), 2.93 (t, J = 6.6 Hz, 2H). |
| 1c | 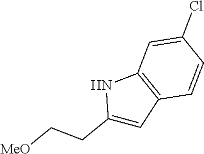 [SMILES: COCCc1cc2ccc(Cl)cc2[nH]1] | ¹H NMR (400 MHz, DMSO-d₆) δ 2.93 (t, J = 6.80 Hz, 2H), 3.27 (s, 3H), 3.63 (t, J = 6.80 Hz, 2H), 6.20 (s, 1H), 6.93 (dd, J = 8.33, 1.75 Hz, 1H), 7.31 (d, J = 1.32 Hz, 1H), 7.40 (d, J = 8.33 Hz, 1H), 10.99 − 11.11 (m, 1H). |

### Intermediate 2

### ethyl (1S,3S,4S)-4-amino-3-methylcyclohexane-1-carboxylate

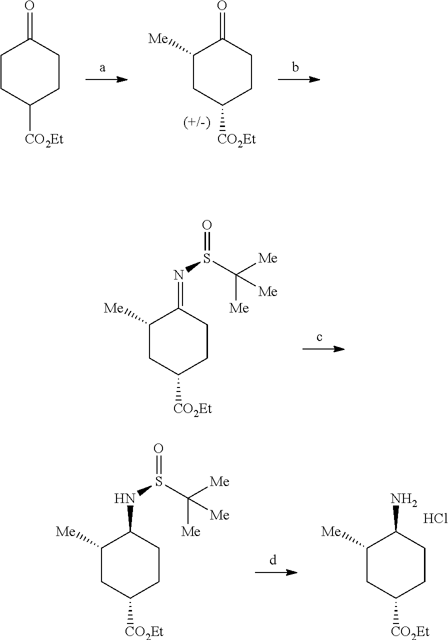

Step a: To ethyl 4-oxocyclohexane-1-carboxylate (120 g, 705 mmol) in THF (600 mL) at −70° C., was added 1.0 M LHMDS in THF (750 mL). The reaction mixture was stirred for 1 h. Next, Mel (211 g, 1.49 mol) was added and the mixture was warmed to 20° C. while stirring for 3 h. The reaction mixture was cooled to 0° C., quenched with aq. sat. NaHCO₃ solution (1 L), and partially concentrated under reduced pressure to remove volatile organics. This mixture was diluted with H₂O and extracted with EtOAc (500 mL×3). The combined organic extracts were dried and concentrated under reduced pressure to yield the crude product. The crude product was purified via silica gel chromatography (petroleum ether:EtOAc, 100:1 to 5:1). Desired fractions were combined and concentrated under reduced pressure to yield racemic cis-ethyl-3-methyl-4-oxocyclohexane-1-carboxylate (30 g) as a light yellow oil. ¹H NMR (400 MHz, CDCl₃) δ 4.17-4.04 (m, 2H), 2.82-2.70 (m, 1H), 2.45-2.21 (m, 5H), 1.89-1.71 (m, 1H), 1.59-1.46 (m, 1H), 1.25-1.16 (m, 3H), 1.03-0.97 (m, 3H).

Step b: To racemic cis-ethyl-3-methyl-4-oxocyclohexane-1-carboxylate (8.3 g, 45.1 mmol) and (S)-2-methylpropane-2-sulfinamide (6.83 g, 56.3 mmol) in THF (100 mL) at RT under N₂, was added tetraethoxytitanium (28.3 mL, 135 mmol). The reaction mixture was heated to 55° C. for 14 h, upon which time it was cooled to 0° C. and quenched with aq. sat. NaHCO₃ solution. The mixture was diluted with EtOAc, stirred intensely, and filtered. The solid was washed with EtOAc (40 mL×2) and the combined organic extracts were washed with aq. sat. NaCl solution (50 mL), dried over Na₂SO₄, and concentrated under reduced pressure to yield the crude product. The crude product was purified via silica gel chromatography (heptane:EtOAc, 100:0 to 40:60). Desired fractions were combined and concentrated under reduced pressure to yield the separated diastereomer ethyl (1S,3S,E)-4-(((S)-tert-butylsulfinyl)imino)-3-methylcyclohexane-1-carboxylate (Peak 1, eluting first, 4.44 g) as a colorless liquid. ¹H NMR (400 MHz, DMSO-d₆) δ 4.06 (q, J=7.1 Hz, 2H), 3.50 (dt, J=14.5, 3.8 Hz, 1H), 2.81 (tt, J=12.3, 3.8 Hz, 1H), 2.60 (dt, J=11.9, 6.0 Hz, 1H), 2.28-2.08 (m, 3H), 1.61-1.46 (m, 1H), 1.40-1.27 (m, 1H), 1.20-1.13 (m, 12H), 0.98 (d, J=6.4 Hz, 3H).

Step c: To ethyl (1S,3S,E)-4-(((S)-tert-butylsulfinyl)imino)-3-methylcyclohexane-1-carboxylate (Peak 1, 57 g. 198 mmol) in THF (570 mL) at 0° C., was added NaBH₄ (7.5 g, 198 mmol). The reaction mixture was stirred at 0° C. for 1 h, upon which time it was quenched with aq. sat. NaHCO₃ solution (300 mL), diluted with EtOAc (500 mL) and H₂O (300 mL), and stirred vigorously for 1 h. The resulting mixture was extracted with EtOAc (500 mL×2). The combined organic extracts were dried and concentrated under reduced pressure to yield the crude product. The crude product was purified via silica gel chromatography (petroleum ether:EtOAc, 100:1 to 5:1). Desired fractions were combined and concentrated under reduced pressure to yield ethyl (1S,3S,4S)-4-(((S)-tert-butylsulfinyl)amino)-3-methylcyclohexane-1-carboxylate (35 g) as a yellow oil. ¹H NMR (400 MHz, DMSO-d₆) δ 5.08-4.99 (m, 1H), 4.10-3.95 (m, 2H), 2.31-2.19 (m, 1H), 1.97-1.77 (m, 3H), 1.53-1.39 (m, 1H), 1.37-1.29 (m, 2H), 1.21-1.14 (m, 4H), 1.12-1.07 (m, 10H), 1.03-0.96 (m, 3H). MS m/z 290.1 [M+H]⁺.

Step d: To ethyl (1S,3S,4S)-4-(((S)-tert-butylsulfinyl)amino)-3-methylcyclohexane-1-carboxylate (45.0 g, 456 mmol) in 1,4-dioxane (270 mL) at 0° C., was added 4 M HCl solution in 1,4-dioxane (270 mL, 1.08 mol). The reaction mixture was stirred at 20° C. for 16 h, upon which time the reaction mixture was concentrated under reduced pressure to yield the crude material. This material was combined with another batch, triturated with petroleum ether (300 mL), and filtered. The solid was collected and dried under reduced pressure to yield ethyl (1S,3S,4S)-4-amino-3-methylcyclohexane-1-carboxylate hydrochloride salt (82 g) as a white solid. ¹H NMR (400 MHz, DMSO-d₆) δ 8.23 (s, 3H), 4.04 (m, J=7.1 Hz, 2H), 2.64 (s, 1H), 2.38-2.21 (m, 1H), 2.06 (d, J=2.7 Hz, 1H), 1.98-1.82 (m, 2H), 1.69-1.52 (m, 1H), 1.36 (t, J=10.5 Hz, 2H), 1.27-1.08 (m, 4H), 0.99 (d, J=6.5 Hz, 3H). MS m/z 186.2 [M+H]⁺.

### Intermediate 3

### methyl (2S,4S)-4-(4-fluorophenyl)-1-(1H-imidazole-1-carbonyl)-2-methyl piperidine-4-carboxylate

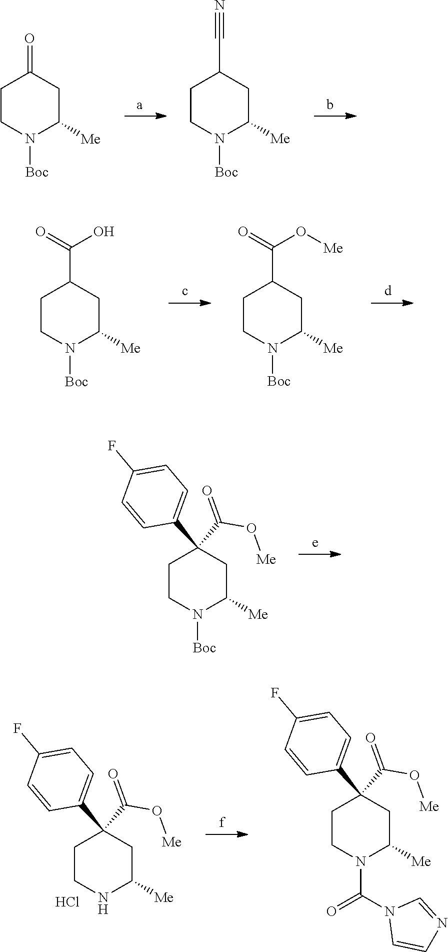

Step a: To KOtBu (421 g, 3.75 mol) in 1,4-dioxane (2.8 L) at 0° C. to −10° C., TosMIC (366 g, 1.88 mol) was added. The reaction mixture was stirred for 0.5 h, upon which time a solution of tert-butyl (S)-2-methyl-4-oxopiperidine-1-carboxylate (200 g, 938 mmol) in EtOH (86.4 g, 1.88 mol) and 1,4-dioxane (1.2 L) were slowly added via addition funnel at 0° C. to −10° C. The mixture was stirred at 25° C. for 16 h. The mixture was poured into aq. sat. NH₄Cl solution (4 L), extracted with MTBE (2 L×2), and the combined organic extracts were dried over Na₂SO₄, filtered, and concentrated under reduced pressure to yield the crude product. The crude product was purified via silica gel chromatography (petroleum ether:EtOAc, 10:1) to yield tert-butyl (2S)-4-cyano-2-methylpiperidine-1-carboxylate (114 g) as a yellow oil. ¹H NMR (400 MHz, CDCl₃) δ 4.50 (s, 1H), 4.03 (d, J=13.9 Hz, 1H), 2.88-2.69 (m, 2H), 2.09-1.99 (m, 1H), 1.92-1.86 (m, 2H), 1.71-1.65 (m, 1H), 1.45 (s, 9H), 1.13 (d, J=7.0 Hz, 3H).

Step b: To tert-butyl (2S)-4-cyano-2-methylpiperidine-1-carboxylate (150 g, 704 mmol) in EtOH/H₂O (750 mL/750 mL) at 25° C., was added KOH (237 g, 4.22 mol). The reaction mixture was stirred at 80° C. for 2 h, upon which time it was partially concentrated under reduced pressure to remove volatile organics. The residue was extracted with MTBE (500 mL×2) and the pH was lowered to 2-3 by the addition of citric acid (200 g). The mixture was diluted with H₂O (500 mL) and extracted with EtOAc (500 mL×2). The combined organic extracts were dried over Na₂SO₄, filtered, and concentrated under reduced pressure to yield (2S)-1-(tert-butoxycarbonyl)-2-methylpiperidine-4-carboxylic acid (120 g, crude) as a colorless oil.

Step c: To (2S)-1-(tert-butoxycarbonyl)-2-methylpiperidine-4-carboxylic acid (120 g, 493 mmol) in DMF (1.2 L) at 25° C., were added K₂CO₃ (136 g, 986 mmol) and Mel (105 g, 740 mmol). The reaction mixture was stirred at 25° C. for 6 h. The mixture was poured into H₂O (1 L) and extracted with MTBE (300 mL×3). The combined organic extracts were washed with aq. sat. NaCl solution (300 mL), dried over Na₂SO₄, filtered, and concentrated under reduced pressure to yield the crude product as a black oil. The crude product was purified via silica gel chromatography (petroleum ether:EtOAc, 5:1). Desired fractions were combined and concentrated under reduced pressure to yield 1-(tert-butyl) 4-methyl (2S)-2-methylpiperidine-1,4-dicarboxylate (100 g) as a yellow oil. ¹H NMR (400 MHz, CDCl₃) δ 4.47 (s, 1H), 4.00 (d, J=12.0 Hz, 1H), 3.66 (s, 3H), 2.82 (dt, J=2.5, 13.4 Hz, 1H), 2.68-2.52 (m, 1H), 1.91-1.83 (m, 1H), 1.77-1.70 (m, 2H), 1.50 (dd, J=4.6, 12.7 Hz, 1H), 1.44 (s, 9H), 1.12 (d, J=7.0 Hz, 3H).

Step d: To 1.0 M LDA solution in toluene (583 mL, 583 mmol) at −25° C., was added 1-(tert-butyl) 4-methyl (2S)-2-methylpiperidine-1,4-dicarboxylate (100 g, 389 mmol). The reaction mixture was warmed to 25° C. over 0.5 h. 1-Bromo-4-fluorobenzene (68 g, 389 mmol) was added followed by Pd(dba)₂ (8.94 g, 15.5 mmol) and TTBP-HBF₄ (9.02 g, 31.1 mmol). The reaction mixture was stirred at 25° C. for 16 h, upon which time it was poured into H₂O (1 L) and extracted with EtOAc (300 mL×2). The organic extracts were separated, dried over Na₂SO₄, filtered, and concentrated under reduced pressure to yield the crude product. The crude product was purified via silica gel chromatography (petroleum ether:EtOAc, 10:1), followed by preparative HPLC (column: Phenomenex luna C18 250 mm×100 mm, 10 μm; H₂O and CH₃CN with 0.1% formic acid, 55 to 75%) and then via spherical silica gel chromatography (petroleum ether:EtOAc, 0-8%) to yield 1-(tert-butyl) 4-methyl (2S,4S)-4-(4-fluorophenyl)-2-methylpiperidine-1,4-dicarboxylate (35.5 g) as a colorless oil. SFC-method: Cellulose-2 50×4.6 mm I.D., 3 μm; mobile phase: Phase A for CO₂ and Phase B for CH₃CN (0.05% DIPEA); gradient elution: CH₃CN (0.05% DIPEA) in CO₂ from 5% to 40%; flow rate: 3 mL/min; detector: PDA column temperature: 35° C.; back pressure: 100 bar; Rₜ=1.279 min. ¹H NMR (400 MHz, CDCl₃) δ 7.38-7.30 (m, 2H), 7.08-6.95 (m, 2H), 4.58-4.43 (m, 1H), 4.03 (d, J=12.6 Hz, 1H), 3.64 (s, 3H), 3.22-3.09 (m, 1H), 2.70-2.58 (m, 2H), 2.06 (dd, J=6.0, 13.8 Hz, 1H), 1.57 (dt, J=5.0, 13.3 Hz, 1H), 1.45 (s, 9H), 1.10 (d, J=7.1 Hz, 3H). MS m/z 296.0 [M-tBu]⁺.

Step e: To 1-(tert-butyl) 4-methyl (2S,4S)-4-(4-fluorophenyl)-2-methylpiperidine-1,4-dicarboxylate (120 g, 341 mmol) in 1,4-dioxane (1.2 L) at 25° C., was added 4 M HCl solution in 1,4-dioxane (512 mL). The reaction mixture was stirred at 25° C. for 16 h. The mixture was concentrated under reduced pressure to yield methyl (2S,4S)-4-(4-fluorophenyl)-2-methylpiperidine-4-carboxylate hydrochloride salt (98.3 g, crude). MS m/z 252.1 [M+H]⁺.

Step f: To methyl (2S,4S)-4-(4-fluorophenyl)-2-methylpiperidine-4-carboxylate hydrochloride salt (100 g, 348 mmol) in DCM (1 L) at 0° C., were added CDI (169 g, 1.04 mol) and DIPEA (270 g, 2.09 mol). The reaction mixture was stirred at 25° C. for 16 h, upon which time it was poured into cooled H₂O (1 L) and extracted with DCM (500 mL×3). The combined organic extracts were washed with aq. sat. NaCl solution (300 mL×3), dried over Na₂SO₄, filtered, and concentrated under reduced pressure to yield the crude product. The crude product was purified via silica gel chromatography (petroleum ether:EtOAc, 1:4) to yield methyl (2S,4S)-4-(4-fluorophenyl)-1-(1H-imidazole-1-carbonyl)-2-methylpiperidine-4-carboxylate (115 g) as a colorless oil that partially solidified over time. ¹H NMR (400 MHz, CDCl₃) δ 7.83 (s, 1H), 7.32 (dd, J=5.2, 7.5 Hz, 2H), 7.16 (s, 1H), 7.08 (s, 1H), 7.02 (t, J=8.2 Hz, 2H), 4.64-4.53 (m, 1H), 3.94 (d, J=13.9 Hz, 1H), 3.67 (s, 3H), 3.51 (t, J=13.3 Hz, 1H), 2.77 (t, J=11.1 Hz, 2H), 2.18 (dd, J=5.8, 14.0 Hz, 1H), 1.68 (dt, J=4.1, 13.4 Hz, 1H), 1.29 (d, J=7.2 Hz, 3H). MS m/z 346.0 [M+H]⁺. SFC-method: Chiralpak AD-3 50×4.6 mm I.D. 3 μm; mobile phase: Phase A for CO₂ and Phase B for MeOH (0.05% DIPEA); gradient elution: B in A from 5% to 40%; flow rate: 3 mL/min; detector: DAD; column temperature: 35° C.; back pressure: 100 bar, Rₜ=1.368 min.

### Intermediate 4

### 2-ethyl-6-fluoro-3-(((1r,4r)-4-((tetrahydro-2H-pyran-4-yl)oxy)cyclohexyl)methyl)-Z-indole

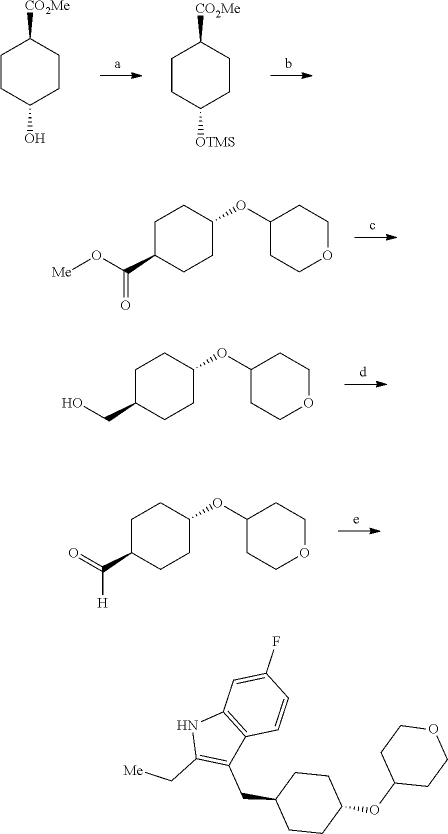

Step a: To methyl (1r,4r)-4-hydroxycyclohexane-1-carboxylate (100 g, 632 mmol) in THF (1.0 L) at 0° C. under N₂, were added NEt₃ (116 mL, 834 mmol) and TMSCI (105 mL, 821 mmol) drop-wise. The reaction mixture was stirred at 0° C. for 1 h, upon which time it was diluted with hexane (1.0 L) and filtered. The filtrate was concentrated under reduced pressure to yield the crude product. The crude product was purified via silica gel column chromatography (petroleum ether:EtOAc, 10:1). Desired fractions were combined and concentrated under reduced pressure to yield methyl (1r,4r)-4-[(trimethylsilyl)oxy]cyclohexane-1-carboxylate (130 g) as a yellow oil.

Step b: To methyl (1r,4r)-4-[(trimethylsilyl)oxy]cyclohexane-1-carboxylate (130 g, 564 mmol) in DCM (1.0 L) at −78° C. under N₂, were added tetrahydro-4H-pyran-4-one (103 mL, 1.03 mol), Et₃SiH (228 mL, 1.96 mol), and TMSOTf (145 mL, 652 mmol) drop-wise. The mixture was stirred at −78° C. for 5 min., then stirred at 0° C. for 1 h. The reaction mixture was quenched with aq. sat. NaHCO₃ solution (500 mL) and extracted with DCM (500 mL×3). The organic extracts were dried over Na₂SO₄, filtered, and concentrated under reduced pressure to yield the crude product. The crude product was purified via silica gel chromatography (petroleum ether:EtOAc, 2:1) to yield methyl (1r,4r)-4-(oxan-4-yloxy)cyclohexane-1-carboxylate (135 g) as a yellow oil. ¹H NMR (300 MHz, DMSO-d₆) δ 3.85-3.70 (m, 2H), 3.69-3.51 (m, 4H), 3.45-3.35 (m, 2H), 3.28-3.25 (m, 1H), 2.32-2.20 (m, 1H), 1.97-1.80 (m, 4H), 1.79-1.71 (m, 2H), 1.48-1.27 (m, 4H), 1.27-1.10 (m, 2H).

Step c: To methyl (1r,4r)-4-(oxan-4-yloxy)cyclohexane-1-carboxylate (140 g, 577 mmol) in THF (1.5 L) at 0° C. under N₂, was added a solution of 2.0 M LAH in THF (318 mL, 636 mmol) drop-wise. The reaction mixture was stirred at 0° C. for 0.5 h, upon which time it was quenched with aq. sat. NaHCO₃ solution (500 mL) and filtered. The filtrate was extracted with DCM (500 mL×3). The organic extracts were dried over Na₂SO₄, filtered, and concentrated under reduced pressure to yield the crude product. The crude product was purified via silica gel column chromatography (petroleum ether:EtOAc, 1:1). Desired fractions were combined and concentrated under reduced pressure to yield [(1r,4r)-4-(oxan-4-yloxy)cyclohexyl]methanol (110 g) as a yellow oil. ¹H NMR (300 MHz, DMSO-d₆) δ 4.38 (t, J=5.3 Hz, 1H), 3.83-3.73 (m, 2H), 3.64-3.53 (m, 1H), 3.37-3.34 (m, 1H) 3.31-3.24 (m, 2H), 3.19 (t, J=5.8 Hz, 2H), 1.98-1.85 (m, 2H), 1.83-1.66 (m, 4H), 1.43-1.21 (m, 3H), 1.18-1.02 (m, 2H), 0.96-0.76 (m, 2H).

Step d: To [(1r,4r)-4-(oxan-4-yloxy)cyclohexyl]methanol (50.0 g, 233 mmol) in DCM (1.0 L) at 0° C. under N₂, were added DIPEA (131 mL, 752 mmol) and pyridine sulfur trioxide complex (67.0 g, 421 mmol) in DMSO (300 mL). The reaction mixture was stirred for 1 h, upon which time the reaction mixture was quenched with aq. 1 M citric acid solution (1.0 L) and extracted with DCM (500 mL×3). The organic extracts were washed with aq. sat. NaCl solution, aq. 1 M citric acid solution (1.0 L), then again with aq. sat. NaCl solution. The combined organic extracts were dried over Na₂SO₄, filtered, and concentrated under reduced pressure to yield (1r,4r)-4-(oxan-4-yloxy)cyclohexane-1-carbaldehyde (49 g, crude) as a yellow oil. ¹H NMR (300 MHz, DMSO-d₆) δ 9.57 (d, J=1.1 Hz, 1H), 3.83-3.73 (m, 2H), 3.64-3.53 (m, 1H), 3.39-3.34 (m, 2H), 3.30-3.21 (m, 1H), 2.29-2.16 (m, 1H), 1.97-1.85 (m, 4H), 1.82-1.72 (m, 2H), 1.43-1.17 (m, 6H).

Step e: To (1r,4r)-4-(oxan-4-yloxy)cyclohexane-1-carbaldehyde (49 g, 231 mmol) in DCM (800 mL) at 0 C, were added 2-ethyl-6-fluoro-1H-indole (31.5 g, 193 mmol), Et₃SiH (156 mL, 966 mmol), and TFA (36.0 mL, 484 mmol) drop-wise. The reaction mixture was stirred at 0° C. for 1 h, upon which time it was quenched with aq. sat. NaHCO₃ solution (500 mL) and extracted with DCM. The combined organic extracts were washed with aq. sat. NaCl solution (500 mL), dried over Na₂SO₄, filtered, and concentrated under reduced pressure to yield the crude material. The crude material was purified via silica gel column chromatography (petroleum ether:EtOAc, 2:1). Desired fractions were combined and concentrated under reduced pressure. This material was further purified via crystallization (petroleum ether:EtOAc:DCM, 600:10:10) to yield 2-ethyl-6-fluoro-3-{[(1r,4r)-4-(oxan-4-yloxy)cyclohexyl]methyl}-1H-indole (50.0 g) as a white solid. MS m/z 358 [M−H]⁻. ¹H NMR (300 MHz, DMSO-d₆) δ 10.76 (s, 1H), 7.32 (dd, J=8.4, 5.7 Hz, 1H), 6.98 (dd, J=10.2, 2.1 Hz, 1H), 6.79-6.81 (m, 1H), 3.80-3.72 (m, 2H), 3.57-3.52 (m, 1H), 3.31-3.21 (m, 3H), 2.63 (dd, J=15.3, 7.8 Hz, 2H), 2.48-2.40 (m, 2H), 1.91-1.60 (m, 6H), 1.50-1.29 (m, 3H), 1.20 (t, J=7.8 Hz, 3H), 1.12-0.96 (m, 4H).

The following compounds of table 2 were synthesized using the above procedure or modifications to the above procedure using the corresponding functionalized indole and aldehyde/ketone intermediate.

TABLE 2

| Intermediate | | |
|---|---|---|
| ID | Structure | Analytical data |
| 4a | 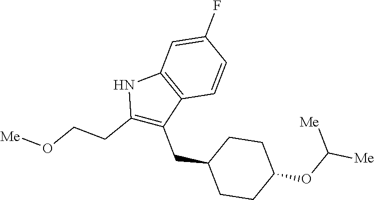 [SMILES: COCCc1[nH]c2cc(F)ccc2c1C[C@H]1CC[C@H](OC(C)C)CC1] | MS m/z [M − H]⁻ = 346.3 |
| 4b | 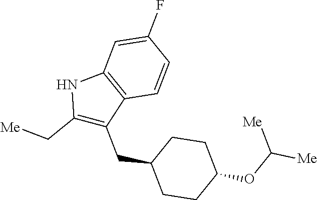 [SMILES: CCc1[nH]c2cc(F)ccc2c1C[C@H]1CC[C@H](OC(C)C)CC1] | MS m/z [M − H]⁻ = 316.4 |
| 4c | 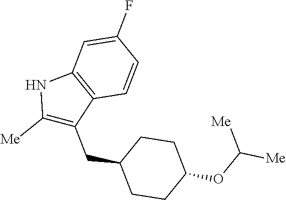 [SMILES: Cc1[nH]c2cc(F)ccc2c1C[C@H]1CC[C@H](OC(C)C)CC1] | MS m/z [M − H]⁻ = 302.2 |
| 4d | 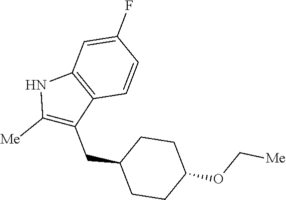 [SMILES: CCO[C@H]1CC[C@H](Cc2c(C)[nH]c3cc(F)ccc23)CC1] | MS m/z [M − H]⁻ = 288.3 |
| 4e | 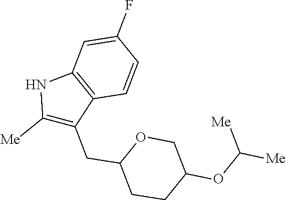 [SMILES: Cc1[nH]c2cc(F)ccc2c1CC1CCC(OC(C)C)CO1] | Derived from Intermediate 8b. Unknown absolute stereochemistry. Trans configuration. MS m/z [M + H]⁺ = 306.2 |
| 4f | 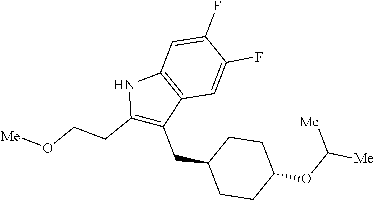 [SMILES: COCCc1[nH]c2cc(F)c(F)cc2c1C[C@H]1CC[C@H](OC(C)C)CC1] | MS m/z [M − H]⁻ = 364.05 |
| 4g | 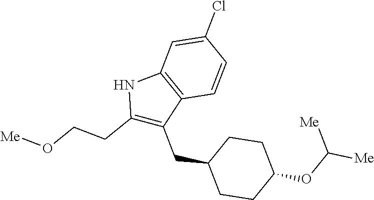 [SMILES: COCCc1[nH]c2cc(Cl)ccc2c1C[C@H]1CC[C@H](OC(C)C)CC1] | MS m/z [M + H]⁺ = 363.9 |
| 4h | 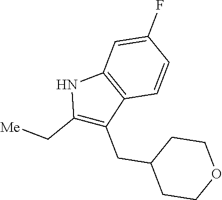 [SMILES: CCc1[nH]c2cc(F)ccc2c1CC1CCOCC1] | MS m/z [M − H]⁻ = 260.3 |
| 4i | 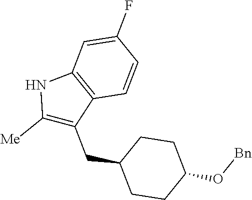 [SMILES: Cc1[nH]c2cc(F)ccc2c1C[C@H]1CC[C@H](OCc2ccccc2)CC1] | MS m/z [M + H]⁺ = 352.1 |

### Intermediate 5

### methyl 4-(4-fluorophenyl)piperidine-4-carboxylate

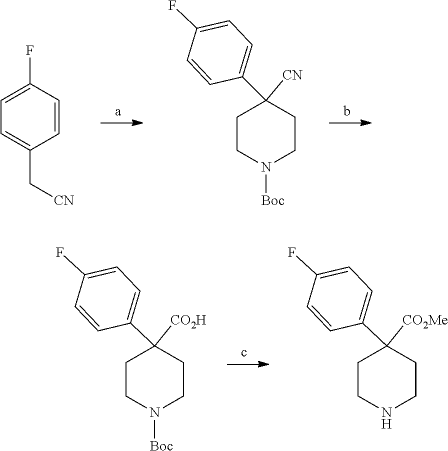 [SMILES: N#CCc1ccc(F)cc1 ; CC(C)(C)OC(=O)N1CCC(C#N)(c2ccc(F)cc2)CC1 ; CC(C)(C)OC(=O)N1CCC(C(=O)O)(c2ccc(F)cc2)CC1 ; COC(=O)C1(c2ccc(F)cc2)CCNCC1]

Step a: To 2-(4-fluorophenyl)acetonitrile (240 g, 1.78 mol) in DMF (1.2 L) at 0° C., were added tert-butyl bis(2-chloroethyl)carbamate (430 g, 1.78 mol) and NaH (156 g, 3.91 mol, 60 wt. %) in batches over 10 min. Next, the reaction mixture was heated to 60° C. and stirred for 4 h. After cooling to RT, the mixture was poured into a mixed solution of H₂O (3.6 L) and MTBE (3.6 L). The organic phase was separated and washed with aq. sat. NaCl solution (1.5 L×3), dried over Na₂SO₄, filtered, and concentrated under reduced pressure to yield the crude product. The crude product was triturated with heptane (2 L) and filtered to yield tert-butyl 4-cyano-4-(4-fluorophenyl)piperidine-1-carboxylate (440 g) as a light yellow solid. ¹H NMR (400 MHz, DMSO) δ 7.60 (m, 2H), 7.41-7.14 (m, 2H), 4.13 (d, J=12.6 Hz, 2H), 3.01 (s, 2H), 2.12 (d, J=13.1 Hz, 2H), 1.90 (td, J=13.2, 4.3 Hz, 2H), 1.61-1.22 (s, 9H).

Step b: To tert-butyl 4-cyano-4-(4-fluorophenyl)piperidine-1-carboxylate (638 g, 2.10 mol) in EtOH (3.2 L) at RT, was added a solution of NaOH (3.35 kg, 83.8 mol) in H₂O (3.2 L). Next, the reaction mixture was heated to 70° C. and stirred for 16 h. The mixture was cooled to 20° C., diluted with H₂O (6.4 L) and adjusted to pH=6-7 with citric acid at 5-10° C. This mixture was extracted with MTBE (3 L×2) and concentrated under reduced pressure to yield 1-(tert-butoxycarbonyl)-4-(4-fluorophenyl)piperidine-4-carboxylic acid (620 g, crude) as a white solid. MS m/z 322.2 [M−H]⁻

Step c: To 1-(tert-butoxycarbonyl)-4-(4-fluorophenyl)piperidine-4-carboxylic acid (620 g, 1.92 mol) in MeOH (6.2 L) at 10° C., was added SOCl₂ (684 g, 5.75 mol) drop-wise and the reaction mixture was heated to 70° C. while stirring for 16 h. The mixture was cooled to 20° C., concentrated under reduced pressure, and diluted with H₂O (3.1 L). The aq. phase was extracted with MTBE (1 L) and neutralized to pH=7-8 with Na₂CO₃ at 5-10° C. The organic extracts were filtered, washed with H₂O (1 L), and concentrated under reduced pressure. The filter cake was dissolved in DCM (1.5 L), dried over Na₂SO₄, filtered, and concentrated under reduced pressure to yield methyl 4-(4-fluorophenyl)piperidine-4-carboxylate (260 g, crude) as an off-white solid. ¹H NMR (400 MHz, CDCl₃) δ 7.43-7.31 (m, 2H), 7.09-6.96 (m, 2H), 3.66 (s, 3H), 3.07 (m, 2H), 2.88-2.69 (m, 2H), 2.64-2.43 (m, 2H), 1.82 (m, 2H), 1.56 (s, 1H). MS m/z 238.1 [M+H]⁺.

The following compounds of table 3 were synthesized using the above procedure or modifications to the above procedure using the corresponding phenyl acetonitrile.

TABLE 3

| Intermediate | | |
|---|---|---|
| ID | Structure | Analytical data |
| 5a | 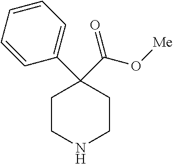 [SMILES: COC(=O)C1(c2ccccc2)CCNCC1] | MS m/z [M + H]⁺ = 220.1 |
| 5b | 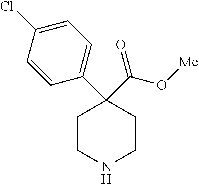 [SMILES: COC(=O)C1(c2ccc(Cl)cc2)CCNCC1] | MS m/z [M + H]⁺ = 254.1 |

### Intermediate 6

### ((1r,4r)-4-((tert-butyldimethylsilyl)oxy)cyclohexyl)methanol

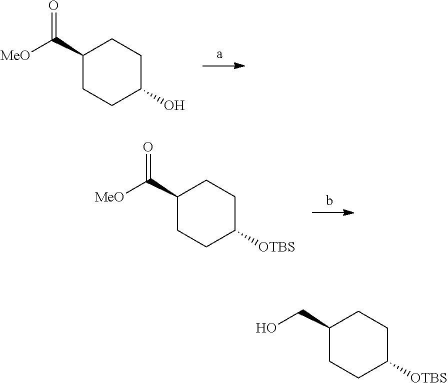

Step a: To methyl (1r,4r)-4-hydroxycyclohexane-1-carboxylate (200 g, 1.26 mol) in DMF (1.0 L) at RT, was added imidazole (129 g, 1.90 mol) followed by TBSCI (166 g, 1.1 mol). The reaction mixture was stirred at RT for 1.5 h, upon which time it was diluted with H₂O (2.0 L) and EtOAc (2.0 L). The layers were separated and the organic layer was washed with H₂O (500 mL×2), dried over Na₂SO₄, filtered, and concentrated under reduced pressure to yield the crude product. The crude product was purified via silica gel chromatography (petroleum ether:EtOAc, 10:0 to 20:1) to yield methyl (1r,4r)-4-((tert-butyldimethylsilyl)oxy)cyclohexane-1-carboxylate (295 g) as a colorless oil. ¹H NMR (400 MHz, DMSO-d₆) δ 3.47-3.72 (m, 4H), 2.15-2.33 (m, 1H), 1.23-1.41 (m, 4H), 1.16-1.46 (m, 4H), 0.85 (s, 9H), 0.04 (s, 6H).

Step b: To methyl (1r,4r)-4-((tert-butyldimethylsilyl)oxy)cyclohexane-1-carboxylate (180 g, 660 mmol) in THF (1.5 L) at 0° C. under N₂, was added LAH (25.1 g, 660 mmol) portion-wise and the reaction mixture was stirred for 1 h. The reaction mixture was quenched with H₂O (25 mL) under N₂ at 0° C., followed by aq. 10 wt. % NaOH solution (25 mL) and then H₂O (50 mL). Next, Na₂SO₄ (100 g) was added and the mixture was filtered. The filter cake was washed with EtOAc (500 mL×2) and the filtrate was concentrated under reduced pressure to yield ((1r,4r)-4-((tert-butyldimethylsilyl)oxy)cyclohexyl)methanol (121 g, crude) as a colorless oil. ¹H NMR (400 MHz, DMSO-d₆) δ 4.34 (t, J=5.3 Hz, 1H), 3.59-3.43 (m, 1H), 3.23-3.12 (m, 2H), 1.82-1.72 (m, 2H), 1.71-1.61 (m, 2H), 1.30-1.07 (m, 3H), 0.95-0.79 (m, 11H), 0.06-−0.03 (m, 6H).

### Intermediate 7a and Intermediate 7b

### (S)-3-((1r,4r)-4-(benzyloxy)cyclohexyl)-6-fluoroindoline and (R)-3-((1r,4r)-4-(benzyloxy)cyclohexyl)-6-fluoroindoline

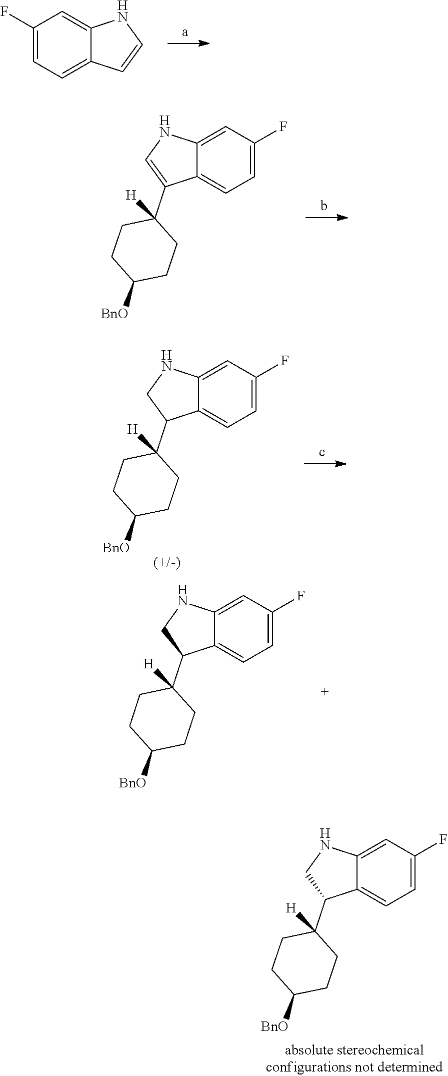 [SMILES: Fc1ccc2cc[nH]c2c1 ; Fc1ccc2c(c1)[nH]cc2[C@H]1CC[C@H](OCc2ccccc2)CC1 ; Fc1ccc2c(c1)NCC2[C@H]1CC[C@H](OCc2ccccc2)CC1 ; Fc1ccc2c(c1)NC[C@@H]2[C@H]1CC[C@H](OCc2ccccc2)CC1 ; Fc1ccc2c(c1)NC[C@H]2[C@H]1CC[C@H](OCc2ccccc2)CC1]

Step a: To 6-fluoro-1H-indole (150 g, 1.11 mol) in DCM (2.1 L) at RT, was added 4-(benzyloxy)cyclohexan-1-one (317 g, 1.55 mol). Next, Et₃SiH (516 g, 4.44 mol) was added followed by TFA (506 g, 4.44 mol). The reaction mixture was stirred at RT for 2 h, upon which time the reaction mixture was quenched with H₂O (1.1 L) and aq. sat. NaHCO₃ solution (500 mL). The layers were separated and the aq. layer was extracted with DCM (600 mL×3). The combined organic extracts were washed with aq. sat. NaCl solution (1 L), passed through a phase separator, and concentrated to yield the crude product. The crude product was stirred in petroleum ether (500 mL) and MeOH (175 mL) overnight. The solids were filtered and washed with MeOH until the filtrate was clear and colorless to yield 3-((1r,4r)-4-(benzyloxy)cyclohexyl)-6-fluoro-1H-indole (614 g, from 4 batches in parallel) as a white solid.

Step b: To 3-((1r,4r)-4-(benzyloxy)cyclohexyl)-6-fluoro-1H-indole (167 g, 518 mmol) at RT, were added TFA (1.18 kg, 10.3 mol) followed by Et₃SiH (580 g, 4.99 mol), and the reaction mixture was heated to 40° C. for 25 min., upon which time it was cooled and concentrated under reduced pressure to remove the TFA. The resulting solution was diluted with DCM (1.0 L). This mixture was poured into aq. sat. NaHCO₃ solution (1.0 L) to neutralize the remaining TFA. The neutralized solution was passed through a phase separator and concentrated under reduced pressure to yield the crude product. The crude product was triturated with MTBE (200 mL) and the filter cake was washed with additional MTBE (200 mL) and MeOH (800 mL) to yield 3-((1r,4r)-4-(benzyloxy)cyclohexyl)-6-fluoroindoline (266 g, from 4 batches in parallel) as a white solid. ¹H NMR (400 MHz, DMSO-d₆) δ 7.20-7.39 (m, 5H), 6.83-7.04 (m, 1H), 6.08-6.35 (m, 2H), 5.55-5.87 (m, 1H), 4.40-4.55 (m, 2H), 3.42-3.51 (m, 1H), 3.18-3.29 (m, 2H), 2.99-3.08 (m, 1H), 1.96-2.10 (m, 2H), 1.66-1.80 (m, 1H), 1.42-1.59 (m, 2H), 0.93-1.27 (m, 4H). MS m/z 326.2 [M+H]⁺.

Step c: 3-((1r,4r)-4-(benzyloxy)cyclohexyl)-6-fluoroindoline (350 g, 1.08 mol) was separated by chiral SFC providing (S)-3-((1r,4r)-4-(benzyloxy)cyclohexyl)-6-fluoroindoline and (R)-3-((1r,4r)-4-(benzyloxy)cyclohexyl)-6-fluoroindoline. SFC-method: DAICEL CHIRALPAK AD 250 mm×50 mm 10 μm; mobile phase: Phase A: 0.1% NH₃ in H₂O and Phase B for iPrOH (A:B, 70:30) to yield Peak 1 (eluting first, Intermediate 7a) and Peak 2 (eluting second, Intermediate 7b).

Peak 1 (110 g) was obtained as an off-white solid. ¹H NMR (400 MHz, DMSO-d₆) δ 7.14-7.44 (m, 5H), 6.84-7.04 (m, 1H), 6.11-6.33 (m, 2H), 5.59-5.79 (m, 1H), 4.42-4.55 (m, 2H), 3.40-3.53 (m, 1H), 3.17-3.31 (m, 2H), 2.98-3.10 (m, 1H), 1.94-2.12 (m, 2H), 1.65-1.81 (m, 1H), 1.42-1.59 (m, 2H), 0.94-1.22 (m, 4H). MS m/z 326.5 [M+H]⁺.

Peak 2 (110 g) was obtained as an off-white solid. ¹H NMR (400 MHz, DMSO-d₆) δ 7.18-7.41 (m, 5H), 6.85-7.00 (m, 1H), 6.11-6.31 (m, 2H), 5.61-5.78 (m, 1H), 4.36-4.61 (m, 2H), 3.40-3.51 (m, 1H), 3.17-3.31 (m, 2H), 2.97-3.11 (m, 1H), 1.96-2.12 (m, 2H), 1.66-1.80 (m, 1H), 1.42-1.59 (m, 2H), 0.98-1.20 (m, 4H). MS m/z 326.2 [M+H]⁺. Note: The absolute stereochemical configurations were not determined.

### Intermediate 8a and Intermediate 8b

### ((2R,5S)-5-(benzyloxy)tetrahydro-2H-pyran-2-yl)methanol and ((2S,5R)-5-(benzyloxy)tetrahydro-2H-pyran-2-yl)methanol

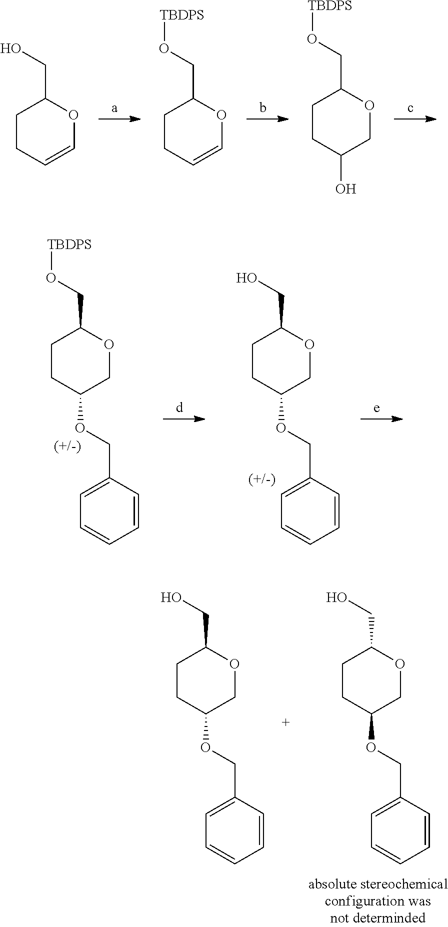

Step a: To (3,4-dihydro-2H-pyran-2-yl)methanol (200 g, 1.75 mol) in DMF (10 L) at 0° C., was added imidazole (298 g, 4.38 mol). tert-Butylchlorodiphenylsilane (530 g, 1.93 mol) was added and the reaction mixture was stirred at RT for 1.5 h, upon which time it was quenched with aq. sat. NaHCO₃ solution and extracted with EtOAc. The combined organic extracts were washed with aq. sat. NaCl solution, dried over Na₂SO₄, filtered, and concentrated under reduced pressure to yield the crude product. The crude product was purified via silica gel column chromatography (EtOAc:heptane, 0:100 to 20:80). Desired fractions were combined and concentrated under reduced pressure to yield tert-butyl((3,4-dihydro-2H-pyran-2-yl)methoxy)diphenylsilane (560 g) as a colorless oil. MS m/z 353 [M+H]⁺.

Step b: To tert-butyl((3,4-dihydro-2H-pyran-2-yl)methoxy)diphenylsilane (550 g, 1.56 mol) in anhydrous THF (6 L) at 0° C. under N₂, was added borane dimethyl sulfide complex (780 mL, 7.8 mol) drop-wise. The reaction mixture was warmed to RT and stirred at RT for 3 h. Next, aq. 5 N NaOH solution (1.87 L, 9.35 mol) was slowly added to the reaction mixture at RT over 2 h followed by aq. 30% H₂O₂ (1.38 L, 12.5 mol) drop-wise over 1.5 h. The reaction mixture was stirred at 55° C. for 2 h, upon which time it was quenched with aq. sat. NaHCO₃ solution, stirred at RT for 0.5 h, and extracted with EtOAc. The combined organic extracts were dried over Na₂SO₄, filtered, and concentrated under reduced pressure to yield the crude product. The crude product was purified via silica gel chromatography (petroleum ether:EtOAc, 5:1). Desired fractions were combined and concentrated under reduced pressure to yield 6-(((tert-butyldiphenylsilyl)oxy)methyl)tetrahydro-2H-pyran-3-ol (450 g) as a colorless oil. MS m/z 393.2 [M+Na]⁺.

Step c: To 6-(((tert-butyldiphenylsilyl)oxy)methyl)tetrahydro-2H-pyran-3-ol (430 g, 1.16 mol) in anhydrous THF (7 L) at 0° C., was added NaH (278 g, 6.96 mol, 60 wt. %) portion-wise and the resulting mixture was stirred at RT for 1 h. Next, (bromomethyl)benzene was added and the reaction mixture was stirred at RT for 5 h, upon which time it was quenched with aq. sat. NH₄Cl solution and diluted with EtOAc. The layers were separated and the aq. layer was extracted with EtOAc. The combined organic extracts were washed with aq. sat. NaCl solution, dried over Na₂SO₄, filtered, and concentrated under reduced pressure to yield the crude product. The crude product was purified via silica gel chromatography (petroleum ether:EtOAc, 200:1). Desired fractions were combined and concentrated under reduced pressure to yield the racemic mixture of trans ((5-(benzyloxy)tetrahydro-2H-pyran-2-yl)methoxy)(tert-butyl)diphenylsilane (200 g) as a colorless oil. ¹H NMR (300 MHz, CDCl₃) δ 7.73-7.56 (m, 4H), 7.41-7.14 (m, 11H), 4.60-4.38 (m, 2H), 4.12-3.94 (m, 1H), 3.64 (dd, J=9.0, 3.0 Hz, 1H), 3.53-3.22 (m, 3H), 3.10 (t, J=10.5 Hz, 1H), 2.22-2.06 (m, 1H), 1.90-1.76 (m, 1H), 1.49-1.13 (m, 2H), 0.99 (s, 9H). MS m/z 483.2 [M+Na]⁺.

Step d: To the racemic mixture of trans ((5-(benzyloxy)tetrahydro-2H-pyran-2-yl)methoxy)(tert-butyl)diphenylsilane (500 g, 1.08 mol) in MeOH (5 L) at 0° C., was added 12 M HCl (452 mL, 5.42 mol). The reaction mixture was stirred at RT for 5 h, upon which time it was concentrated under reduced pressure to yield the crude product. The crude product was purified via reverse phase C18 chromatography (CH₃CN and H₂O, 35:65) to yield the racemic mixture of trans_5-(benzyloxy)tetrahydro-2H-pyran-2-yl)methanol.

Step e: Single enantiomers of the racemic mixture of trans 5-(benzyloxy)tetrahydro-2H-pyran-2-yl)methanol were separated by preparative-SFC [chiral ART Amylose-C NEO, 5 cm×25 cm 5 μm; mobile phase A: CO₂, mobile phase B:iPrOH:hexane=2:1 (0.1% 2 M NH₃-MeOH); flow rate: 200 mL/min; gradient: isocratic 18% B; column temperature: 35° C.; back pressure: 100 bar; wavelength: 220 nm; sample solvent: EtOH; injection volume: 1.2 mL; number of runs: 250] to yield Peak 1 (Intermediate 8a, Rₜ=8.33 min) and Peak 2 (Intermediate 8b, Rₜ=9.80 min).

Peak 1: ((2S,5R)-5-(benzyloxy)tetrahydro-2H-pyran-2-yl)methanol or ((2R,5S)-5-(benzyloxy)tetrahydro-2H-pyran-2-yl)methanol (56.7 g) as a colorless oil. ¹H NMR (300 MHz, CDCl₃) δ 7.43-7.15 (m, 5H), 4.66-4.41 (m, 2H), 4.18-4.01 (m, 1H), 3.56 (dd, J=11.4, 3.3 Hz, 1H), 3.51-3.30 (m, 3H), 3.20 (t, J=10.5 Hz, 1H), 2.51 (s, 1H), 2.28-2.15 (m, 1H), 1.68-1.56 (m, 1H), 1.55-1.25 (m, 2H). MS m/z 223.2 [M+H]⁺.

Peak 2: ((2S,5R)-5-(benzyloxy)tetrahydro-2H-pyran-2-yl)methanol or ((2R,5S)-5-(benzyloxy)tetrahydro-2H-pyran-2-yl)methanol (54.4 g) as a colorless oil. ¹H NMR (300 MHz, CDCl₃) δ 7.44-7.14 (m, 5H), 4.65-4.43 (m, 2H), 4.18-4.01 (m, 1H), 3.55 (dd, J=11.4, 3.0 Hz, 1H), 3.50-3.29 (m, 3H), 3.20 (t, J=10.4 Hz, 1H), 2.64 (s, 1H), 2.27-2.13 (m, 1H), 1.69-1.55 (m, 1H), 1.55-1.26 (m, 2H). MS m/z 223.2 [M+H]⁺. Note: The absolute stereochemical configurations were not determined.

### Intermediate 9

### methyl 1-(2-(2-((tert-butyldimethylsilyl)oxy)ethyl)-6-fluoro-1H-indole-1-carbonyl)-4-(4-fluorophenyl)piperidine-4-carboxylate

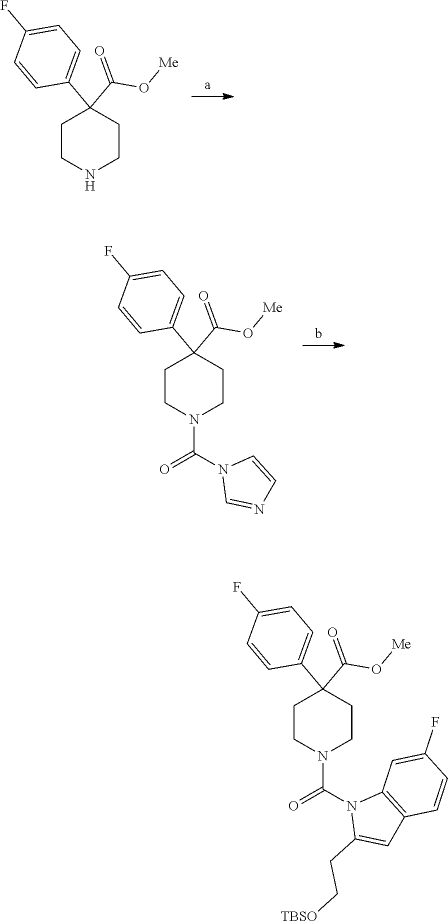 [SMILES: COC(=O)C1(c2ccc(F)cc2)CCNCC1 ; COC(=O)C1(c2ccc(F)cc2)CCN(C(=O)n2ccnc2)CC1 ; COC(=O)C1(c2ccc(F)cc2)CCN(C(=O)n2c(CCO[Si](C)(C)C(C)(C)C)cc3ccc(F)cc32)CC1]

Step a: To methyl 4-(4-fluorophenyl)piperidine-4-carboxylate (69.0 g, 291 mmol) in DCM (500 mL) at RT, was added CDI (70.7 g, 436 mmol) and the reaction mixture was stirred for 2 h. The resulting mixture was quenched with H₂O and extracted with DCM. The organic layers were washed with aq. sat. NaCl solution, dried over Na₂SO₄, filtered, and concentrated to yield methyl 4-(4-fluorophenyl)-1-(imidazole-1-carbonyl)piperidine-4-carboxylate (100 g, crude) as a white solid. ¹H NMR (300 MHz, DMSO-d₆) δ 8.02 (t, J=1.1 Hz, 1H), 7.51-7.34 (m, 3H), 7.28-7.14 (m, 2H), 7.06-6.99 (m, 1H), 3.84 (d, J=13.8 Hz, 2H), 3.64 (s, 3H), 3.30-3.21 (m, 2H), 2.49-2.41 (m, 2H), 2.20-1.95 (m, 2H). MS m/z 332 [M+H]⁺.

Step b: To 2-{2-[(tert-butyldimethylsilyl)oxy]ethyl}-6-fluoro-1H-indole (70.1 g, 239 mmol) in THF (800 mL) at −25° C. under N₂, was added 1.0 M LHMDS in THF (319 mL, 319 mmol) drop-wise followed by 2.0 M AlMe₃ solution in toluene (146 mL, 252 mmol). The mixture was stirred for another 30 min at −25° C. Next, methyl 4-(4-fluorophenyl)-1-(imidazole-1-carbonyl)piperidine-4-carboxylate (88.0 g, 266 mmol) was added at −25° C. The mixture was warmed to 60° C. and stirred for 2 h, followed by cooling to 0° C. and quenching by the addition of aq. sat. NH₄Cl solution at 0° C. The resulting mixture was diluted with H₂O and extracted with DCM. The combined organic extracts were washed with aq. sat. NaCl solution, dried over Na₂SO₄, filtered, and concentrated under reduced pressure to yield the crude product. The crude product was purified via silica gel chromatography (petroleum ether:DCM:EtOAc, 10:1:1). Desired fractions were combined and concentrated under reduced pressure to yield the crude material (102 g) as a yellow oil. The reaction was repeated with additional methyl 4-(4-fluorophenyl)-1-(imidazole-1-carbonyl)piperidine-4-carboxylate (35 g) to yield another 52.7 g crude product. The combined lot (155 g) was dried to yield methyl 1-(2-(2-((tert-butyldimethylsilyl)oxy)ethyl)-6-fluoro-1H-indole-1-carbonyl)-4-(4-fluorophenyl)piperidine-4-carboxylate (152 g) as a yellow solid. ¹H NMR (300 MHz, CDCl₃) δ 7.46-7.42 (m, 1H), 7.40-7.30 (m, 2H), 7.15-6.84 (m, 4H), 6.40 (s, 1H), 4.07-3.45 (m, 7H), 3.36-3.21 (m, 2H), 3.03 (m, J=7.3 Hz, 2H), 2.63 (t, J=16.9 Hz, 2H), 2.11-1.75 (m, 2H), 0.88 (d, J=21.2 Hz, 9H), 0.04 (d, J=24.3 Hz, 6H). MS m/z 557 [M+H]⁺.

The following compound of table 4 was synthesized using the above procedure or modifications to the above procedure using the corresponding functionalized piperidine and indole intermediate.

TABLE 4

| Intermediate | | |
|---|---|---|
| ID | Structure | Analytical data |
| 9a | 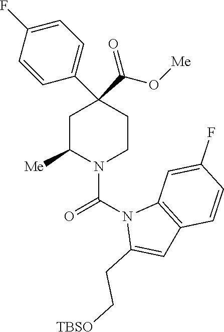 [SMILES: COC(=O)[C@@]1(c2ccc(F)cc2)CCN(C(=O)n2c(CCO[Si](C)(C)C(C)(C)C)cc3ccc(F)cc32)[C@@H](C)C1] | MS m/z [M + H]⁺ = 571.2 |

### Intermediate 10

### (3S,4R)-4-fluoro-1-methylpyrrolidin-3-amine

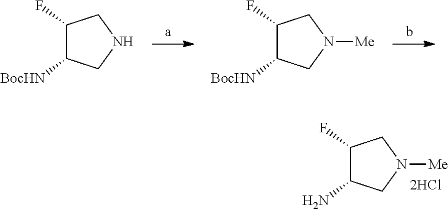 [SMILES: CC(C)(C)OC(=O)N[C@H]1CNC[C@H]1F ; CN1C[C@@H](F)[C@@H](NC(=O)OC(C)(C)C)C1 ; CN1C[C@@H](F)[C@@H](N)C1]

Step a: To tert-butyl (3S,4R)-4-fluoropyrrolidin-3-ylcarbamate (7.9 g, 38.6 mmol) in MeOH (79 mL) at 0° C., were added formaldehyde (1.27 g, 42.5 mol) and NaBH₄ (2.20 g, 58.0 mmol) portion-wise. The resulting reaction mixture was warmed to RT and stirred for 14 h. The resulting reaction mixture was concentrated under reduced pressure and extracted with EtOAc. Combined organic extracts were washed with aq. sat. NaCl solution, dried over Na₂SO₄, filtered, and concentrated under reduced pressure to obtain the crude material. The crude material was purified via silica gel chromatography (MeOH:DCM, 3:97 to 5:95). Desired fractions were combined and concentrated under reduced pressure to yield tert-butyl ((3S,4R)-4-fluoro-1-methylpyrrolidin-3-yl)carbamate (7.5 g) as a yellow liquid. ¹H NMR (400 MHz, DMSO-d₆) δ 6.92 (s, 1H), 4.99-4.83 (m, 1H), 3.98-3.92 (m, 1H), 3.12-3.01 (m, 1H), 2.77-2.73 (t, J=8 Hz, 1H), 2.54-2.53 (m, 1H), 2.49-2.37 (m, 1H), 2.23 (s, 3H), 1.38 (s, 9H).

Step b: To tert-butyl ((3S,4R)-4-fluoro-1-methylpyrrolidin-3-yl)carbamate (9.20 g, 0.0422 mol) in MeOH (9.2 mL) at 0° C., was added 4 M HCl solution in 1,4-dioxane (92.0 mL) drop-wise. The reaction mixture was warmed to RT and stirred for 3 h, upon which time it was concentrated under reduced pressure to yield the crude product. The crude product was dissolved in MeOH and stirred for 1 h at RT, during which time a solid precipitated. This mixture was filtered to obtain (3S,4R)-4-fluoro-1-methylpyrrolidin-3-amine hydrochloride salt (6.24 g) as an off-white solid. ¹H NMR (400 MHz, DMSO-d₆) δ 9.26 (s, 4H), 5.55-5.4 (d, J=52.8 Hz, 1H), 4.18 (s, 1H), 3.73-3.39 (m, 4H), 2.89 (s, 3H).

### Intermediate 11

### 5-bromo-1-((tetrahydro-2H-pyran-4-yl)methyl)-1H-indole-3-carboxylic acid

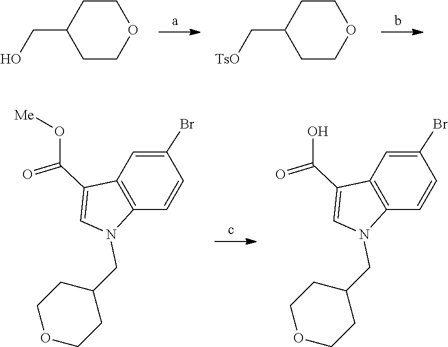 [SMILES: OCC1CCOCC1 ; Cc1ccc(S(=O)(=O)OCC2CCOCC2)cc1 ; COC(=O)c1cn(CC2CCOCC2)c2ccc(Br)cc12 ; O=C(O)c1cn(CC2CCOCC2)c2ccc(Br)cc12]

Step a: To tetrahydropyran-4-ylmethanol (10.0 g, 86.1 mmol) in DCM (60 mL) at 15° C., were added NEt₃ (17.4 g, 172 mmol), DMAP (1.05 g, 8.61 mmol), and 4-methylbenzenesulfonyl chloride (19.7 g, 103 mmol). The reaction mixture was stirred at 15° C. for 16 h, upon which time it was concentrated under reduced pressure to yield the crude product. The crude product was purified via silica gel chromatography (EtOAc:petroleum ether, 0:100 to 40:60). Desired fractions were combined and concentrated under reduced pressure to yield tetrahydropyran-4-ylmethyl 4-methylbenzenesulfonate (22.0 g) as a white solid.

Step b: To methyl 5-bromo-1H-indole-3-carboxylate (4.50 g, 17.7 mmol) in DMF (60 mL) at 15° C., were added tetrahydropyran-4-ylmethyl 4-methylbenzenesulfonate (14.4 g, 53.1 mmol) and K₂CO₃ (9.79 g, 70.8 mmol). The reaction mixture was stirred at 95° C. for 3 h, upon which time it was added to aq. sat. NaCl solution (500 mL), then extracted with EtOAc (400 mL×2). The combined organic extracts were washed with aq. sat. NaCl solution (400 mL×2), dried over Na₂SO₄, filtered, and concentrated under reduced pressure to yield the crude product. The crude product was purified via silica gel chromatography (EtOAc:petroleum ether, 0:100 to 35:65). Desired fractions were combined and concentrated under reduced pressure to yield methyl 5-bromo-1-(tetrahydropyran-4-ylmethyl)indole-3-carboxylate (6.00 g) as a white solid. MS m/z 351.9 [M+H]⁺.

Step c: To methyl 5-bromo-1-(tetrahydropyran-4-ylmethyl)indole-3-carboxylate (5.50 g, 15.6 mmol) in MeOH (30 mL), H₂O (15 mL), and THF (60 mL) at 15° C., was added NaOH (1.25 g, 31.2 mmol). The reaction mixture was stirred at 50° C. for 20 h. The solvent was concentrated under reduced pressure, THF (100 mL) was added, and the reaction mixture was concentrated under reduced pressure again. Next, 4 M HCl solution in 1,4-dioxane (30 mL) was added to the residue and the resulting mixture was concentrated to yield 5-bromo-1-(tetrahydropyran-4-ylmethyl)indole-3-carboxylic acid (7.5 g, crude) as a light red solid. MS m/z 339.9 [M+H]⁺.

### Intermediate 12

### 5-chloro-2-methyl-1-((tetrahydro-2H-pyran-4-yl)methyl)-1H-indole-3-carboxylic acid

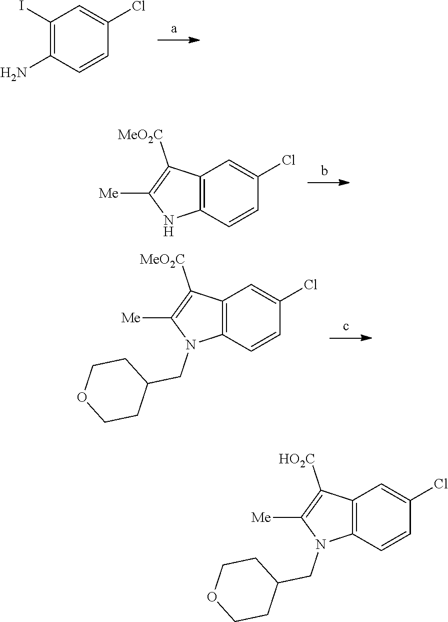

Step a: To 4-chloro-2-iodoaniline (4.00 g, 15.8 mmol) in DMSO (59 mL) and H₂O (20 mL) at RT under N₂, were added Cs₂CO₃ (5.14 g, 15.8 mmol) and copper(I) oxide (0.226 g, 1.58 mmol). Next, methyl 3-oxobutanoate (2.04 mL, 18.9 mmol) was added and the reaction mixture was stirred for 7 h at 100° C. The reaction mixture was cooled to RT, diluted with EtOAc (300 mL), and washed with aq. sat. NaCl solution (150 mL) and H₂O (150 mL). Single aq. layers were extracted with EtOAc (150 mL). The combined organic extracts were dried over Na₂SO₄, filtered, and concentrated under reduced pressure to yield the crude product as a dark brown oil. The crude product was purified via silica gel chromatography (EtOAc:heptane, 0:100 to 30:70). Desired fractions were combined and concentrated under reduced pressure to yield methyl 5-chloro-2-methyl-1H-indole-3-carboxylate (860 mg) as a brown solid. MS m/z 224.1 [M+H]⁺.

Step b: To methyl 5-chloro-2-methyl-1H-indole-3-carboxylate (0.760 g, 3.40 mmol) in DMF (17.9 mL) at RT under N₂, were added (tetrahydro-2H-pyran-4-yl)methyl 4-methyltosylate (1.38 g, 5.10 mmol) and K₂CO₃ (1.88 g, 13.6 mmol). The reaction mixture was stirred at 95° C. for 12 h, upon which time it was cooled to RT and a mixture of aq. sat. NaCl solution and H₂O (1:1, 75 mL) was added. The resulting mixture was extracted with EtOAc (150 mL and 75 mL). Combined organic extracts were washed with aq. sat. NaCl solution (75 mL), dried over Na₂SO₄, filtered, and concentrated under reduced pressure to yield the crude product. The crude product was purified via silica gel chromatography (EtOAc:heptane, 0:100 to 30:70). Desired fractions were combined and concentrated under reduced pressure to yield methyl 5-chloro-2-methyl-1-((tetrahydro-2H-pyran-4-yl)methyl)-1H-indole-3-carboxylate (952 mg). MS m/z 322.1 [M+H]⁺.

Step c: To methyl 5-chloro-2-methyl-1-((tetrahydro-2H-pyran-4-yl)methyl)-1H-indole-3-carboxylate (2.73 g, 8.48 mmol) in THF (37.7 mL) and MeOH (18.4 mL) at RT, was added aq. 3 N NaOH (8.48 mL, 25.5 mmol). The reaction mixture was stirred at 50° C. for 2 d, upon which time aq. 3 N NaOH (5.65 mL, 17.0 mmol) was added and the mixture was stirred for an additional 2 d. The reaction mixture was extracted with Et₂O to remove unreacted starting material. The aq. phase was acidified with aq. 1 N HCl to pH ˜1, which produced a thick suspension. This suspension was extracted with EtOAc (120 mL and 60 mL×2). The combined organic extracts were washed with aq. sat. NaCl solution, dried over Na₂SO₄, filtered, and concentrated under reduced pressure to yield 5-chloro-2-methyl-1-((tetrahydro-2H-pyran-4-yl)methyl)-1H-indole-3-carboxylic acid (2.13 g, crude) as a white solid. ¹H NMR (400 MHz, DMSO-d₆) δ 12.23 (s, 1H), 7.95 (d, J=2.1 Hz, 1H), 7.63 (d, J=8.7 Hz, 1H), 7.18 (dd, J=8.7, 2.2 Hz, 1H), 4.12 (d, J=7.5 Hz, 2H), 3.85-3.74 (m, 2H), 3.18 (td, J=11.2, 3.3 Hz, 2H), 2.73 (s, 3H), 2.07-1.99 (m, 1H), 1.41-1.32 (m, 4H). MS m/z 308.1 [M+H]⁺.

### Intermediate 13

### 5-chloro-2-formyl-1-((tetrahydro-2H-pyran-4-yl)methyl)-1H-indole-3-carboxylic acid

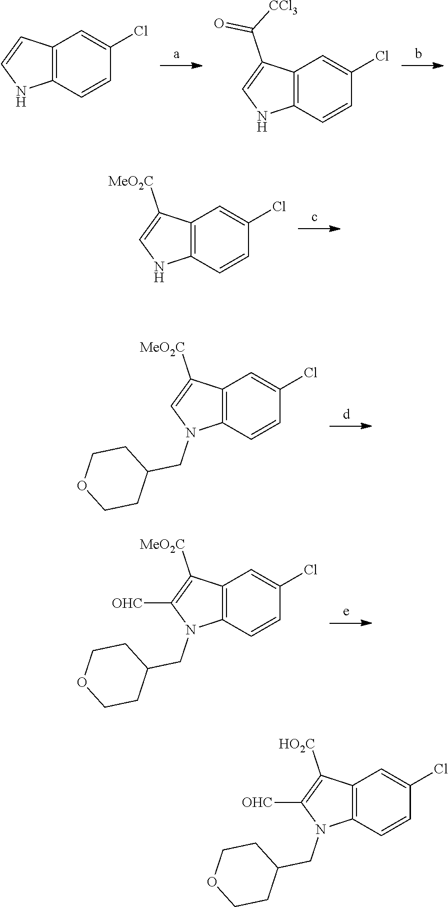

Step a: To 5-chloro-1H-indole (190 g, 1.25 mol) in anhydrous THF (950 mL) at 0° C. under N₂, was added pyridine (248 g, 3.13 mol). Next, a solution of 2,2,2-trichloroacetyl chloride (570 g, 3.13 mol) in anhydrous THF (400 mL) was added drop-wise over 2 h. The reaction mixture was warmed to 25° C. and stirred for 14 h, upon which time it was diluted with EtOAc (5.0 L) and washed with H₂O (5.0 L×2). The organic phase was dried over Na₂SO₄, filtered, and concentrated under reduced pressure to yield the crude product. The crude product was treated with petroleum ether:EtOAc (5:1, 600 mL) and filtered. The filter cake was dried to yield 2,2,2-trichloro-1-(5-chloro-1H-indol-3-yl)ethan-1-one (302 g) as an off-white solid. ¹H NMR (400 MHz, CDCl₃) δ 8.92 (br s, 1H), 8.45 (d, J=2.0 Hz, 1H), 8.37 (d, J=3.0 Hz, 1H), 7.40 (d, J=4.4 Hz, 1H), 7.32 (dd, J=2.0, 4.4 Hz, 1H). MS m/z 295.9 [M+H]⁺.

Step b: To 2,2,2-trichloro-1-(5-chloro-1H-indol-3-yl)ethan-1-one (252 g, 849 mmol) in MeOH (1.25 L) at RT, was added KOH (47.6 g, 849 mmol) in H₂O (48 mL) until pH=11. The reaction mixture was stirred at 80° C. for 5 h. The mixture was cooled to 25° C., neutralized with aq. 4 M HCl solution, and concentrated under reduced pressure. The resulting residue was dissolved in EtOAc (2 L) and washed with H₂O (2 L). The organic phase was dried over Na₂SO₄, filtered, and concentrated under reduced pressure to yield the crude product. The crude product was treated with petroleum ether:EtOAc (8:1, 200 mL) and filtered. The filter cake was collected and dried to yield methyl 5-chloro-1H-indole-3-carboxylate (131 g) as an off-white powder. MS m/z 210.0 [M+H]⁺.

Step c: To methyl 5-chloro-1H-indole-3-carboxylate (50 g, 238 mmol) in CH₃CN (250 mL) at RT, were added K₂CO₃ (65.0 g, 470 mmol) and 4-(bromomethyl)tetrahydro-2H-pyran (65.0 g, 363 mmol). The reaction mixture was stirred at 80° C. for 48 h, upon which time it was cooled to RT, filtered, and the filtrate was concentrated under reduced pressure to yield the crude product. The crude product was dissolved in EtOAc (1.0 L), washed with H₂O (1.0 L), dried over Na₂SO₄, filtered, and concentrated under reduced pressure to yield methyl 5-chloro-1-((tetrahydro-2H-pyran-4-yl)methyl)-1H-indole-3-carboxylate (73 g, crude) as a light yellow solid. ¹H NMR (400 MHz, CDCl₃) δ 8.17 (d, J=1.6 Hz, 1H), 7.80 (s, 1H), 7.24-7.30 (m, 2H), 4.02 (d, J=7.2 Hz, 2H), 3.94-4.00 (m, 2H), 3.93 (s, 3H), 3.30-3.33 (m, 2H), 2.06-2.12 (m, 1H), 1.39-1.51 (m, 4H). MS m/z 308.0 [M+H]⁺.

Step d: To iPr₂NH (36.0 g, 356 mmol) in anhydrous THF (200 mL) at −40° C., was added n-BuLi (2.5 M, 119 mL) drop-wise. The reaction mixture was warmed to 0° C. while stirring for 0.5 h, upon which time it was added drop-wise into a mixture of methyl 5-chloro-1-((tetrahydro-2H-pyran-4-yl)methyl)-1H-indole-3-carboxylate (73 g, 237 mmol) in anhydrous THF (700 mL) at −70° C. The reaction mixture was stirred at −70° C. for 0.5 h, then DMF (34.7 g, 474 mmol) was added. The reaction mixture was stirred at −70° C. for another 0.5 h, upon which time it was poured into aq. sat. NH₄Cl solution (2 L) and extracted with EtOAc (1.5 L). The organic extracts were washed with H₂O (1.5 L), dried over Na₂SO₄, filtered, and concentrated under reduced pressure to yield the crude product. The crude product was treated with petroleum ether:EtOAc (5:1, 400 mL) and filtered. The filter cake was collected and dried to yield methyl 5-chloro-2-formyl-1-((tetrahydro-2H-pyran-4-yl)methyl)-1H-indole-3-carboxylate (46 g) as a yellow solid. ¹H NMR (400 MHz, DMSO-d₆) δ 10.63 (s, 1H), 8.13 (s, 1H), 7.90 (d, J=8.8 Hz, 1H), 7.50 (d, J=2.0, 8.8 Hz, 1H), 4.50 (d, J=7.6 Hz, 2H), 3.94 (s, 3H), 3.76 (dd, J=2.8, 11.6 Hz, 2H), 3.76 (td, J=2.4, 11.2 Hz, 2H), 1.90-2.10 (m, 1H), 1.24-1.35 (m, 4H). MS m/z 336.1 [M+H]⁺.

Step e: To methyl 5-chloro-2-formyl-1-((tetrahydro-2H-pyran-4-yl)methyl)-1H-indole-3-carboxylate (20 g, 59.6 mmol) in THF (200 mL) and MeOH (100 mL) at RT, was added aq. 1 N NaOH solution (149 mL, 149 mmol) via addition funnel over 30 min. The reaction mixture was stirred at RT over the weekend. The reaction mixture was partially concentrated under reduced pressure to remove volatile organics. The resulting residue was diluted with H₂O and treated with aq. 1 N HCl solution until pH 1 was reached. The yellow suspension was filtered, washed with H₂O, and dried under a flow of N₂. The resulting material was resuspended in DCM and concentrated under reduced pressure several times to yield a free flowing yellow solid. The aq. phase from filtration was partitioned between EtOAc and H₂O and the organic phase was separated. The organic layer was washed with aq. sat. NaCl solution, dried over MgSO₄, filtered, and concentrated under reduced pressure to yield an orange solid. The solid from the filtration was combined with the solid from the extraction and triturated in heptane. The resulting suspension was filtered and the solid was further dried to yield 5-chloro-2-formyl-1-((tetrahydro-2H-pyran-4-yl)methyl)-1H-indole-3-carboxylic acid (22.2 g) as a yellow solid. ¹H NMR (400 MHz, DMSO-d₆) δ 11.01 (s, 1H), 8.42 (d, J=2.2 Hz, 1H), 7.69 (d, J=8.9 Hz, 1H), 7.35 (dd, J=8.9, 2.3 Hz, 1H), 4.43 (d, J=7.2 Hz, 2H), 3.81-3.73 (m, 2H), 3.19-3.09 (m, 2H), 2.04-1.86 (m, 1H), 1.42-1.17 (m, 4H).

The following compound of table 5 was synthesized using the above procedure or modifications to the above procedure using the corresponding indole and alkyl tosylate.

TABLE 5

| Intermediate ID | Structure | Analytical data |
|---|---|---|
| 13a | 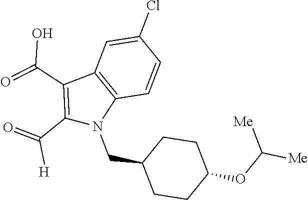 [SMILES: CC(C)O[C@H]1CC[C@H](Cn2c(C=O)c(C(=O)O)c3cc(Cl)ccc32)CC1] | MS m/z 376.2 [M − H]⁻ |

### Intermediate 14

### methyl 4-(6-chloropyridin-3-yl)piperidine-4-carboxylate

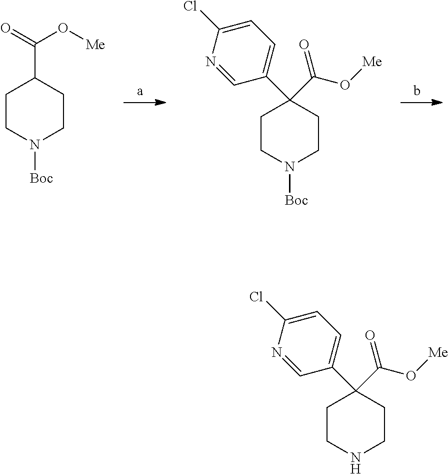 [SMILES: COC(=O)C1CCN(C(=O)OC(C)(C)C)CC1 ; COC(=O)C1(c2ccc(Cl)nc2)CCN(C(=O)OC(C)(C)C)CC1 ; COC(=O)C1(c2ccc(Cl)nc2)CCNCC1]

Step a: To 1-tert-butyl 4-methyl piperidine-1,4-dicarboxylate (3.00 g, 12.3 mmol) in toluene (15 mL) at 0° C., was added a solution of 0.6 M NaHMDS in toluene (25 mL, 15.0 mmol) drop-wise over 0.25 h. Next, a 1.9 M ZnCl₂ solution in 2-Me-THF (10 mL, 19.0 mmol) was added and the reaction mixture was stirred at 0° C. for 1 h. To the mixture was added 2-chloro-5-iodopyridine (2.95 g, 12.3 mmol), followed by [Pd(mu-Br)t-Bu₃P]₂ (CAS #185812-86-6, 0.9 g, 1.16 mmol). The reaction mixture was stirred at RT for 17 h, upon which time it was quenched with H₂O (10 mL), diluted with DCM, and Celite® was added. The mixture was then filtered through a plug of Celite®, which was rinsed with DCM. The filtrate was concentrated under reduced pressure to yield the crude product. The crude product was purified via silica gel chromatography (EtOAc:heptane, 25:75 to 40:60). Desired fractions were combined and concentrated under reduced pressure to yield 1-(tert-butyl) 4-methyl 4-(6-chloropyridin-3-yl)piperidine-1,4-dicarboxylate (0.9 g) as a powder. ¹H NMR (400 MHz, MeOD-d₄) δ 8.39 (d, J=2.7 Hz, 1H), 7.86 (dd, J=2.7, 8.5 Hz, 1H), 7.45 (dd, J=0.6, 8.5 Hz, 1H), 3.89-3.98 (m, 2H), 3.70 (s, 3H), 2.96-3.17 (m, 2H), 2.53 (d, J=13.5 Hz, 2H), 1.82-1.94 (m, 2H), 1.45 (s, 9H). MS m/z 299.0 [M-tBu+2H]⁺.

Step b: To 1-(tert-butyl) 4-methyl 4-(6-chloropyridin-3-yl)piperidine-1,4-dicarboxylate (630 mg, 1.78 mmol) in MeOH (3 mL) at RT, was added 4 M HCl solution in 1,4-dioxane (1.00 mL, 4.00 mmol) drop-wise. The reaction mixture was heated to 40° C. for 3.5 h, upon which time it was concentrated directly under reduced pressure to yield methyl 4-(6-chloropyridin-3-yl)piperidine-4-carboxylate (519 mg, crude) as an off-white solid. MS m/z 255.1 [M+H]⁺.

The following compound of table 6 were synthesized using the above procedure or modifications to the above procedure using the appropriately substituted halogenated aromatic.

TABLE 6

| Intermediate ID | Structure | Analytical data |
|---|---|---|
| 14a | 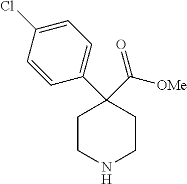 [SMILES: COC(=O)C1(c2ccc(Cl)cc2)CCNCC1] | MS m/z 254.1 [M + H]⁺ |

### Intermediate 15

### methyl (2S,4S)-4-(3,4-difluorophenyl)-1-(1H-imidazole-1-carbonyl)-2-methylpiperidine-4-carboxylate

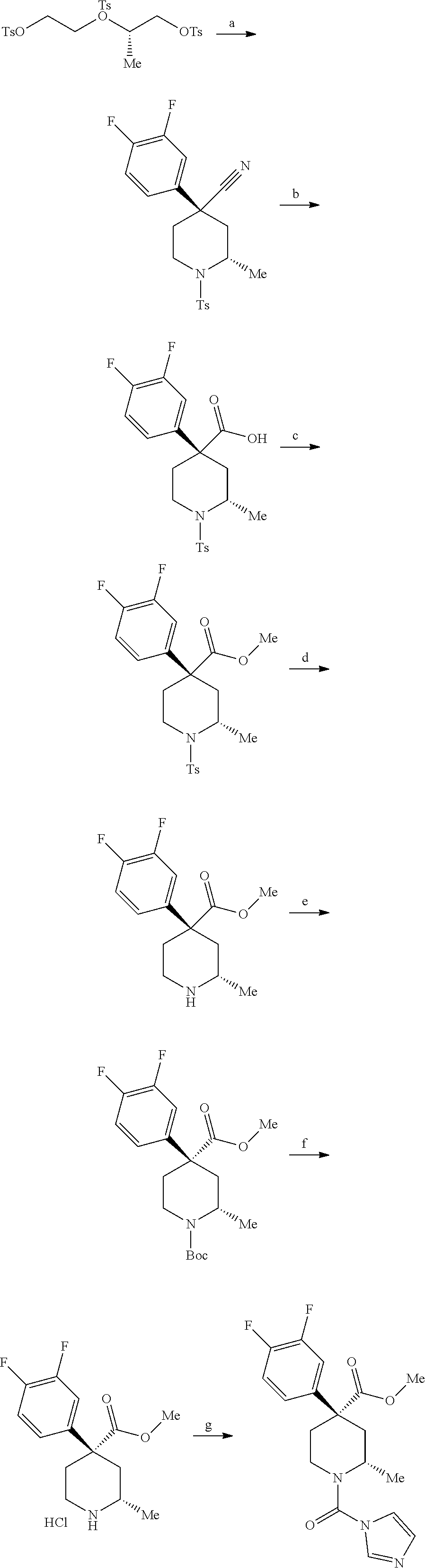

Step a: To 2-(4-fluorophenyl) acetonitrile (2.73 g, 17.8 mmol) in THF (25 mL) at RT, was added a 1.0 M LHMDS solution in THF (35.7 mL, 35.7 mmol) and the reaction mixture was stirred for 2 min. Next, a solution of (S)-2-((4-methyl-N-(1-(tosyloxy)propan-2-yl)phenyl)sulfonamido)ethyl 4-methylbenzenesulfonate (4.00 g, 6.87 mmol) in THF (15 mL) was added slowly at RT. The reaction mixture was stirred at 40° C. for 1 h, and then cooled to RT while stirring for an additional 12 h. The reaction mixture was quenched with H₂O (100 mL) and extracted with EtOAc (100 mL). The combined organic extracts were washed with H₂O (50 mL×2), dried over Na₂SO₄, filtered, and concentrated under reduced pressure to yield the crude material. The crude material was purified via silica gel chromatography (EtOAc:hexane, 10:90 to 15:85) to yield (2S)-4-(3,4-difluorophenyl)-2-methyl-1-tosylpiperidine-4-carbonitrile (4.0 g). MS m/z 392.1 [M+H]⁺.

Step b: To (2S)-4-(3,4-difluorophenyl)-2-methyl-1-tosylpiperidine-4-carbonitrile (4.00 g, 10.2 mmol) in EtOH and H₂O (1:1, 30 mL) at RT, was added KOH (11.4 g, 205 mmol). The reaction mixture was stirred at 110° C. for 4 d. The reaction mixture was cooled to RT, acidified with concentrated HCl (15 mL), and extracted with EtOAc (50 mL×2). The organic extracts were washed with aq. sat. NaCl solution (10 mL), dried over Na₂SO₄, filtered, and concentrated under reduced pressure to obtain (2S)-4-(3,4-difluorophenyl)-2-methyl-1-tosylpiperidine-4-carboxylic acid (4.5 g, crude). MS m/z 409.8 [M+H]⁺.

Step c: To (2S)-4-(3,4-difluorophenyl)-2-methyl-1-tosylpiperidine-4-carboxylic acid (4.5 g, 10.9 mmol) in DMF (45 mL) at RT, were added K₂CO₃ (14.9 g, 108 mmol) and Mel (4.7 mL, 74.8 mmol). The reaction mixture was stirred at 40° C. for 12 h. The reaction mixture was diluted with H₂O (50 mL) and extracted with EtOAc (100 mL×2). The combined organic extracts were washed with aq. sat. NaCl solution (20 mL), dried over Na₂SO₄, filtered, and concentrated under reduced pressure to yield methyl (2S)-4-(3,4-difluorophenyl)-2-methyl-1-tosylpiperidine-4-carboxylate (1.65 g, crude). MS m/z 423.9 [M+H]⁺.

Step d: To methyl (2S)-4-(3,4-difluorophenyl)-2-methyl-1-tosylpiperidine-4-carboxylate (1.45 g, 3.42 mmol) in MeOH (30 mL) at RT, was added magnesium metal (5.8 g). The reaction mixture was sonicated for 5 h. The reaction mixture was quenched with aq. sat. NH₄Cl solution (50 mL) and extracted with EtOAc (50 mL×2). The organic extracts were washed with aq. sat. NaCl solution (10 mL), dried over Na₂SO₄, filtered, and concentrated under reduced pressure to yield methyl (2S,4S)-4-(3,4-difluorophenyl)-2-methylpiperidine-4-carboxylate (450 mg, crude). MS m/z 270.1 [M+H]⁺.

Step e: To methyl (2S)-4-(3,4-difluorophenyl)-2-methylpiperidine-4-carboxylate (450 mg, 1.67 mmol) in DCM (10 mL) at 0° C., were added (Boc)₂O (0.806 mL, 3.51 mmol) and DIPEA (0.87 mL, 5.01 mmol). The reaction mixture was stirred at RT for 2 h, upon which time it was quenched with H₂O (10 mL), extracted with DCM (10 mL×2), and washed with aq. sat. NaCl solution (10 mL). The organic extracts were dried over Na₂SO₄, filtered, and concentrated under reduced pressure to yield 1-(tert-butyl) 4-methyl (2S,4S)-4-(3,4-difluorophenyl)-2-methylpiperidine-1,4-dicarboxylate (150 mg, crude).

Step f: To 1-(tert-butyl) 4-methyl (2S,4S)-4-(3,4-difluorophenyl)-2-methylpiperidine-1,4-dicarboxylate (150 mg, 0.406 mmol) in MeOH (5 mL) at 0° C., was added 4 M HCl solution in 1,4-dioxane (0.32 mL, 1.29 mmol). The reaction mixture was stirred at RT for 2 h. The reaction mixture was concentrated under reduced pressure to yield methyl (2S,4S)-4-(3,4-difluorophenyl)-2-methylpiperidine-4-carboxylate hydrochloride salt (110 mg, crude). MS m/z 270.5 [M+H]⁺.

Step g: To methyl (2S)-4-(3,4-difluorophenyl)-2-methylpiperidine-4-carboxylate hydrochloride salt (110 mg, 0.408 mmol) in DCM (5 mL) at 0° C., were added DIPEA (0.43 mL, 2.45 mmol) and CDI (205 mg, 1.27 mmol). The reaction mixture was stirred at RT for 4 h, upon which time it was diluted with DCM and washed with H₂O and aq. sat. NaCl solution. The organic layer was dried over Na₂SO₄, filtered, and concentrated under reduced pressure to yield the crude material. The crude material was purified via silica gel chromatography (EtOAc:hexane, 0:100 to 100:0) to obtain methyl (2S,4S)-4-(3,4-difluorophenyl)-1-(1H-imidazole-1-carbonyl)-2-methylpiperidine-4-carboxylate (120 mg). MS m/z 364.1 [M+H]⁺.

The following compound of table 7 was synthesized using the above procedure or modifications to the above procedure using the corresponding substituted phenyl acetonitrile.

TABLE 7

| Intermediate | | |
|---|---|---|
| ID | Structure | Analytical data |
| 15a | 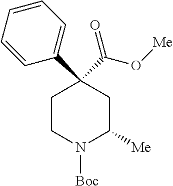 [SMILES: COC(=O)[C@@]1(c2ccccc2)CCN(C(=O)OC(C)(C)C)[C@@H](C)C1] | ¹H NMR (300 MHz, CDCl₃) δ 7.45 − 7.29 (m, 5H), 4.50 (s, 1H), 4.02 (d, J = 14.1 Hz, 1H), 3.61 (s, 3H), 3.17 (t, J = 13.1 Hz, 1H), 2.91 (s, 1H), 2.65 (d, J = 13.6 Hz, 2H), 2.11 (dd, J = 13.7, 6.1 Hz, 1H), 1.45 (s, 9H), 1.10 (d, J = 7.0 Hz, 3H). |

### Intermediate 16

### ethyl (1S,3S,4S)-4-amino-3-chlorocyclohexane-1-carboxylate

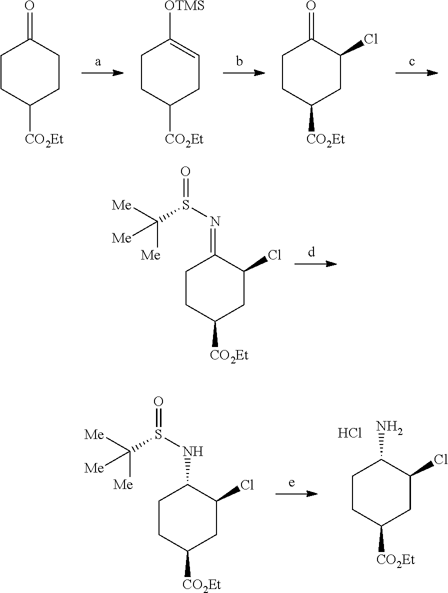

Step a: To ethyl 4-oxocyclohexane-1-carboxylate (4.68 mL, 29.4 mmol) in DMF (30 mL) at RT, were added NEt₃ (16.4 mL, 118 mmol) and TMSCI (7.51 mL, 58.8 mmol). A reflux condensor was fitted to the flask with a N₂ inlet and needle outlet, and the reaction mixture was heated to 120° C. for 5 h. The reaction mixture was cooled to 0° C. with an ice bath and diluted with H₂O and EtOAc until all solids had dissolved. The EtOAc layer was washed with aq. sat. NaCl solution (50 mL×2), dried over Na₂SO₄, filtered, and concentrated under reduced pressure to yield ethyl 4-((trimethylsilyl)oxy)cyclohex-3-ene-1-carboxylate (7.13 g, crude) as an orange oil.

Step b: To ethyl 4-((trimethylsilyl)oxy)cyclohex-3-ene-1-carboxylate (7.13 g) in acetone (50 mL) and H₂O (12.5 mL) at 0° C., was added sodium acetate (6.03 g, 73.5 mmol). Next, NCS (5.89 g, 44.1 mmol) was added in a single portion and the reaction mixture was stirred while warming to RT over 72 h. The reaction mixture was diluted with EtOAc and H₂O and stirred vigorously. The organic layer was partitioned and washed with aq. sat. NaCl solution (40 mL×2), dried over Na₂SO₄, filtered, and concentrated under reduced pressure to yield the crude product. The crude product was purified via silica gel chromatography (iPrOH:heptane, 0:100 to 50:50). Desired fractions were combined and concentrated under reduced pressure to yield ethyl (1S,3S)-3-chloro-4-oxocyclohexane-1-carboxylate (1.4 g).

Step c: To (1S,3S)-3-chloro-4-oxocyclohexane-1-carboxylate (1.4 g, 6.84 mmol) in THF (34.2 mL) at RT under N₂, was added (S)-2-methylpropane-2-sulfinamide (1.66 g, 13.7 mmol). Next, tetraethoxytitanium (4.30 mL, 20.5 mmol) was added rapidly and the reaction mixture was heated to 55° C. for 3 h. The reaction mixture was cooled to 0° C., and diluted carefully with aq. sat. NaHCO₃ solution, which produced a solid white precipitate. This material was extracted with EtOAc, the organic extracts were dried over Na₂SO₄, and concentrated under reduced pressure to yield the crude product. The crude product was purified via silica gel chromatography (EtOAc:heptane, 0:100 to 100:0), which yielded two peaks, Peak 1 (eluting first) and Peak 2 (eluting second). Desired fractions corresponding to Peak 1 were combined and concentrated under reduced pressure to yield ethyl (1S,3S,E)-4-(((S)-tert-butylsulfinyl)imino)-3-chlorocyclohexane-1-carboxylate (450 mg) as a colorless liquid. ¹H NMR (400 MHz, DMSO) δ 5.03 (dd, J=11.7, 5.1 Hz, 1H), 4.11 (m, J=7.1 Hz, 2H), 3.81-3.57 (m, 1H), 3.07-2.88 (m, 1H), 2.66 (d, J=13.5 Hz, 1H), 2.39 (td, J=13.3, 4.8 Hz, 1H), 2.16 (d, J=13.4 Hz, 1H), 2.06-1.89 (m, 1H), 1.62 (qd, J=12.8, 4.0 Hz, 1H), 1.21 (d, J=4.3 Hz, 12H).

Step d: To ethyl (1S,3S,E)-4-(((S)-tert-butylsulfinyl)imino)-3-chlorocyclohexane-1-carboxylate in THF (15.4 mL) and EtOH (5 mL) at 0° C. under N₂, was added NaBH₄ (270 mg, 7.15 mmol). The reaction mixture was stirred for 1 h, upon which time it was quenched with aq. sat. NaHCO₃ solution and diluted with DCM and H₂O while stirring vigorously for 30 min. The organic layer was passed through a phase separator and concentrated under reduced pressure to yield the crude product. The crude product was purified via silica gel chromatography (EtOAc:heptane, 0:100 to 100:0). Desired fractions were combined and concentrated under reduced pressure to yield ethyl (1S,3S,4S)-4-(((S)-tert-butylsulfinyl)amino)-3-chlorocyclohexane-1-carboxylate (301 mg) as a colorless oil. ¹H NMR (400 MHz, DMSO) δ 5.26 (d, J=6.1 Hz, 1H), 4.18-3.91 (m, 3H), 3.06 (td, J=10.7, 5.6 Hz, 1H), 2.51-2.36 (m, 2H), 2.03 (d, J=14.9 Hz, 1H), 1.96-1.88 (m, 1H), 1.78 (m, J=12.3 Hz, 1H), 1.54-1.39 (m, 2H), 1.20 (t, J=7.1 Hz, 3H), 1.13 (s, 9H).

Step e: To ethyl (1S,3S,4S)-4-(((S)-tert-butylsulfinyl)amino)-3-chlorocyclohexane-1-carboxylate in DCM (5 mL) at RT, was added 4 M HCl solution in 1,4-dioxane (0.607 mL, 2.43 mmol). The reaction mixture was stirred for 1 h during which time a white precipitate formed. The reaction mixture was concentrated under reduced pressure and the resulting material was triturated (2×) with heptane to yield ethyl (1S,3S,4S)-4-amino-3-chlorocyclohexane-1-carboxylate hydrochloride salt (180 mg) as a white solid. ¹H NMR (400 MHz, DMSO-d₆) δ 8.38 (s, 3H), 4.17 (ddd, J=12.0, 10.3, 4.3 Hz, 1H), 4.09 (m, J=7.1 Hz, 2H), 3.24 (s, 1H), 2.60-2.53 (m, 1H), 2.47-2.37 (m, 1H), 2.16 (dt, J=9.4, 4.0 Hz, 1H), 1.96 (d, J=8.1 Hz, 1H), 1.81 (m, J=12.4 Hz, 1H), 1.55-1.45 (m, 2H), 1.20 (t, J=7.1 Hz, 3H).

### Intermediate 17

### tert-butyl (1R,3S,4S)-3-amino-4-methylcyclopentane-1-carboxylate

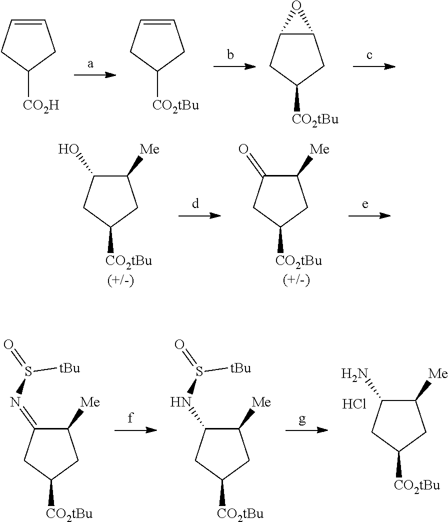

Step a: To cyclopent-3-ene-1-carboxylic acid (5.1 g, 45.5 mmol) in DCM (55 mL) and DMF (0.5 mL) at 0° C., were added oxalyl chloride (4.78 mL, 54.6 mmol) drop-wise and the reaction mixture was allowed to warm to RT overnight. The reaction mixture was concentrated under reduced pressure and the residue was dissolved in DCM (50 mL), upon which time it was cooled back to 0° C. A solution of tert-butanol (13.1 mL, 136 mmol) and NEt₃ (12.7 mL, 91 mmol) in DCM (20 mL) was added drop-wise. The reaction mixture was warmed to RT while stirring overnight, upon which time it was quenched with aq. sat. NaHCO₃ solution. The layers were separated and the aq. layer was extracted with DCM. The combined organic extracts were washed with aq. sat. NaHCO₃ solution, aq. sat. NaCl solution, dried over Na₂SO₄, and passed through a phase separator. The filtrate was concentrated under reduced pressure to yield tert-butyl cyclopent-3-ene-1-carboxylate (4.75 g, crude) as an oil.

Step b: To tert-butyl cyclopent-3-ene-1-carboxylate (4.75 g, 28.2 mmol) in DCM (100 mL) at 0° C., was added mCPBA (8.23 g, 36.7 mmol). The reaction mixture was stirred at RT overnight. The precipitate was filtered, and the filtrate was washed with sat. aq. NaHCO₃ solution (100 mL×2) followed by aq. sat. NaCl solution (100 mL). The resulting organic layer was dried over Na₂SO₄, filtered, and concentrated under reduced pressure (bath temperature 35° C.) to yield the crude product. The crude product was purified via silica gel chromatography (EtOAc:heptane, 0:100 to 100:0). Desired fractions were concentrated under reduced pressure to yield tert-butyl (1R,3s,5S)-6-oxabicyclo[3.1.0]hexane-3-carboxylate (3.6 g). ¹H NMR (400 MHz, DMSO-d₆) δ 5.67 (s, 2H), 3.03 (tt, J=9.4, 6.6 Hz, 1H), 2.58-2.50 (m, 4H), 1.43 (s, 9H).

Step c: To tert-butyl (1R,3s,5S)-6-oxabicyclo[3.1.0]hexane-3-carboxylate in THF (28 mL) at RT under N₂, was added CuI (0.154 g, 0.809 mmol) and the reaction mixture was cooled to −40° C. Next, a 3.0 M methylmagnesium chloride solution in THF (5.39 mL, 16.2 mmol) was added drop-wise and the reaction mixture was stirred at RT overnight, upon which time it was cooled to 0° C. and quenched with aq. sat. NH₄Cl solution and stirred for 10 min. The stirred mixture was diluted with EtOAc and transferred to a separatory funnel. The layers were separated and the aq. layer was extracted with EtOAc (2×). The combined organic extracts were washed with aq. sat. NaCl solution, dried over Na₂SO₄, filtered, and concentrated under reduced pressure to yield the crude product. The crude product was purified via silica gel chromatography (EtOAc:heptane, 0:100 to 50:50). Desired fractions were combined and concentrated under reduced pressure to yield tert-butyl (1R,3S,4S)-3-hydroxy-4-methylcyclopentane-1-carboxylate (1.12 g) as a clear oil. ¹H NMR (400 MHz, DMSO-d₆) δ 4.66 (s, 1H), 3.56 (m, J=6.2 Hz, 1H), 2.79 (qd, J=8.9, 6.7 Hz, 1H), 2.17-2.03 (m, 1H), 1.96 (dt, J=13.4, 6.8 Hz, 1H), 1.68 (dddd, J=28.6, 13.2, 9.4, 6.3 Hz, 2H), 1.41 (d, J=2.0 Hz, 9H), 1.25-1.19 (m, 1H), 0.95 (d, J=6.8 Hz, 3H).

Step d: To tert-butyl (1R,3S,4S)-3-hydroxy-4-methylcyclopentane-1-carboxylate (1.12 g, 5.59 mmol) in DCM (14.0 mL) at −25° C. under N₂, was added DIPEA (2.93 mL, 16.8 mmol). The temperature of the reaction mixture was held between −20 to −30° C. A solution of pyridine sulfur trioxide complex (1.16 g, 7.27 mmol) in DMSO (4.66 mL) was added drop-wise. The reaction mixture was then placed in an ice/H₂O bath while stirring for 2 h. The reaction mixture was quenched with aq. 1.0 M citric acid solution, the organic layer was passed through a phase separator, and the aq. layer was extracted with DCM (5 mL×3). The combined organic extracts were washed with aq. 1 M citric acid solution, aq. sat. NaCl solution, and then H₂O. The organics were passed through a phase separator and concentrated under reduced pressure to yield tert-butyl (1R,3S)-3-methyl-4-oxocyclopentane-1-carboxylate (1.03 g, crude) as a yellow crystalline solid.

Step e: To tert-butyl (1R,3S)-3-methyl-4-oxocyclopentane-1-carboxylate (1.03 g, 5.17 mmol) in THF (17 mL) at RT under N₂, was added (S)-2-methylpropane-2-sulfinamide (0.783 g, 6.46 mmol) in a single portion. Next, tetraethoxytitanium (3.54 g, 15.5 mmol) was added and the reaction mixture was heated to 55° C. for 18 h. The reaction mixture was cooled to 0° C., and quenched with aq. sat. NaHCO₃ solution, which produced a solid white precipitate. The reaction mixture was diluted with EtOAc and H₂O, and stirred vigorously. The solid precipitate was partitioned and washed with EtOAc (40 mL×2). The combined organic extracts were washed with aq. sat. NaCl solution (50 mL), dried over Na₂SO₄, filtered, and concentrated under reduced pressure to yield the crude product. The crude product was purified via silica gel chromatography (EtOAc:heptane, 0:100 to 60:40). Desired fractions corresponding to Peak 1 (eluting first) were combined and concentrated under reduced pressure to yield tert-butyl (1R,4S,Z)-3-(((S)-tert-butylsulfinyl)imino)-4-methylcyclopentane-1-carboxylate (270 mg) as an off-white solid. Note: The absolute stereochemistry was assigned on the basis of peak retention time in comparison to the product of step b en route to Intermediate 2. ¹H NMR (400 MHz, DMSO-d₆) δ 3.03-2.81 (m, 3H), 2.67 (dt, J=13.1, 6.9 Hz, 1H), 2.27 (dt, J=12.4, 7.2 Hz, 1H), 1.43 (s, 9H), 1.41-1.33 (m, 1H), 1.18 (s, 9H), 1.11 (d, J=6.8 Hz, 3H).

Step f: To tert-butyl (1R,4S,Z)-3-(((S)-tert-butylsulfinyl)imino)-4-methylcyclopentane-1-carboxylate (270 mg, 0.896 mmol) in THF (6 mL) at 0° C. under N₂, was added NaBH₄ (42.4 mg, 1.12 mmol) in a single portion. The reaction mixture was stirred at 0° C. for 1 h, upon which time it was quenched with aq. sat. NaHCO₃ solution, diluted with EtOAc and then H₂O, and stirred vigorously for 1 h. The resulting mixture was extracted with EtOAc (50 mL×2), and the combined organic extracts were washed with aq. sat. NaCl solution (40 mL), dried over Na₂SO₄, filtered, and concentrated under reduced pressure to yield the crude product. The crude product was purified via silica gel chromatography (EtOAc:heptane, 5:95 to 100:0). Desired fractions were combined and concentrated under reduced pressure to yield tert-butyl (1R,3S,4S)-3-(((S)-tert-butylsulfinyl)amino)-4-methylcyclopentane-1-carboxylate (89 mg) as a colorless oil. 1H NMR (400 MHz, DMSO-d₆) δ 5.17 (d, J=7.0 Hz, 1H), 3.08 (p, J=7.7 Hz, 1H), 2.77 (qd, J=8.8, 5.8 Hz, 1H), 2.21-1.99 (m, 2H), 1.97-1.73 (m, 2H), 1.40 (s, 9H), 1.29 (ddd, J=12.7, 9.9, 8.5 Hz, 1H), 1.12 (s, 9H), 1.04 (d, J=6.7 Hz, 3H).

Step g: To tert-butyl (1R,3S,4S)-3-(((S)-tert-butylsulfinyl)amino)-4-methylcyclopentane-1-carboxylate (90 mg, 0.297 mmol) in DCM (1.48 mL) at RT, was added 4 M HCl solution in 1,4-dioxane (185 μL, 0.741 mmol). The reaction mixture was stirred for 1 h, upon which time it was concentrated directly to yield a white solid. This material was triturated with Et₂O (20 mL), and the residual solvent was removed under reduced pressure to yield tert-butyl (1R,3S,4S)-3-amino-4-methylcyclopentane-1-carboxylate hydrochloride salt (52 mg) as a white solid. ¹H NMR (400 MHz, DMSO-d₆) δ 8.08 (s, 3H), 3.08 (s, 1H), 2.92 (p, J=8.6 Hz, 1H), 2.18 (ddd, J=13.0, 8.0, 6.6 Hz, 2H), 1.98 (dq, J=9.8, 7.0 Hz, 1H), 1.93-1.70 (m, 1H), 1.42 (s, 9H), 1.34 (dt, J=12.8, 9.7 Hz, 1H), 1.07 (d, J=6.6 Hz, 3H).

### Intermediate 18

### (2S,3S)-1,2-dimethylazetidin-3-amine

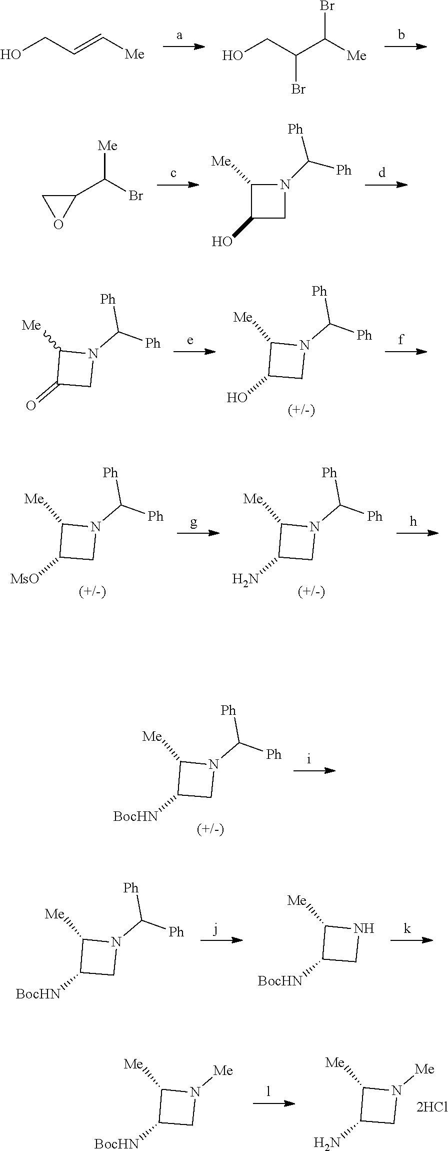

Step a: To (E)-but-2-en-1-ol (11.8 mL, 139 mmol) in DCM (350 mL) at RT, was added bromine (7.14 mL, 139 mmol) slowly. The reaction mixture was stirred for 2 h, upon which time it was treated with sat. aq. Na₂S₂O₃ solution. The organic layer was separated, dried over Na₂SO₄, filtered, and concentrated under reduced pressure to yield 2,3-dibromobutan-1-ol (crude) as a colorless oil. ¹H NMR (400 MHz, CDCl₃) δ 4.39 (dq, J=9.3, 6.6 Hz, 1H), 4.26 (ddd, J=9.2, 4.8, 3.6 Hz, 1H), 4.11-4.07 (m, 2H), 2.41-2.35 (m, 1H), 1.91 (d, J=6.6 Hz, 3H).

Step b: To 2,3-dibromobutan-1-ol (33 g, 142 mmol) in Et₂O (100 mL) at RT, was added KOH (7.98 g, 142 mmol) solution in H₂O (100 mL) and the reaction mixture was stirred at RT for 18 h. The layers were separated and the organic layer was washed with aq. sat. NaCl solution, dried over Na₂SO₄, filtered, and concentrated under reduced pressure to yield 2-(1-bromoethyl)oxirane (crude). ¹H NMR (400 MHz, CDCl₃) δ 3.87 (p, J=6.9 Hz, 1H), 3.24 (ddd, J=6.8, 3.8, 2.5 Hz, 1H), 2.96 (dd, J=4.8, 3.8 Hz, 1H), 2.75 (dd, J=4.8, 2.5 Hz, 1H), 1.72 (d, J=6.9 Hz, 3H).

Step c: To 2-(1-bromoethyl)oxirane (22 g, 146 mmol) in MeOH at RT, was added diphenylmethanamine (25.1 mL, 146 mmol) and the reaction mixture was stirred for 18 h and then heated to reflux for 24 h. The mixture was concentrated under reduced pressure, and the crude product was extracted with Et₂O, and the organic layer was washed with aq. sat. NaHCO₃ solution, dried over Na₂SO₄, filtered, and concentrated under reduced pressure to yield the crude product. To confirm the structure, a small quantity of the material was purified via silica gel chromatography (EtOAc:heptane, 30:70). Desired fractions were combined and concentrated under reduced pressure to yield trans-1-benzhydryl-2-methylazetidin-3-ol as an oil. ¹H NMR (400 MHz, DMSO) δ 7.40 (dt, J=8.1, 1.7 Hz, 4H), 7.26 (ddd, J=10.3, 8.5, 6.8 Hz, 4H), 7.17 (td, J=7.2, 1.8 Hz, 2H), 5.28 (d, J=6.4 Hz, 1H), 4.35 (s, 1H), 3.66 (p, J=6.5 Hz, 1H), 3.43 (t, J=6.7 Hz, 1H), 2.91 (p, J=6.1 Hz, 1H), 2.42 (t, J=7.0 Hz, 1H), 0.61 (d, J=6.1 Hz, 3H).

Step d: To trans-1-benzhydryl-2-methylazetidin-3-ol (8.43 g, 29.9 mmol) in DCM (200 mL) at 0° C., were added DMSO (21.3 mL, 299 mmol) and NEt₃ (16.7 mL, 120 mmol). Next, pyridine sulfur trioxide complex (19.1 g, 120 mmol) was added and the reaction mixture was stirred for 2 h and then warmed to RT. The reaction mixture was diluted with aq. sat. NaCl solution, and the crude product was extracted with EtOAc. The organic extracts were dried over Na₂SO₄, filtered, and concentrated under reduced pressure to yield the crude product. The crude product was purified via silica gel chromatography (EtOAc:heptane, 20:80). Desired fractions were combined and concentrated under reduced pressure to yield 1-benzhydryl-2-methylazetidin-3-one (4.9 g) as a white solid. ¹H NMR (400 MHz, DMSO-d₆) δ 7.50 (ddd, J=7.9, 4.1, 1.3 Hz, 4H), 7.31 (td, J=7.6, 5.9 Hz, 4H), 7.25-7.18 (m, 2H), 4.82 (s, 1H), 4.26-4.01 (m, 2H), 3.82 (d, J=15.9 Hz, 1H), 0.76 (d, J=6.8 Hz, 3H).

Step e: To 1-benzhydryl-2-methylazetidin-3-one (4.9 g, 19.5 mmol) in THF (100 mL) at −78° C., was added a 1.0 M L-Selectride solution in THF (29.2 mL, 29.2 mmol). The reaction mixture was stirred for 30 min., upon which time the cooling bath was removed and the reaction mixture was stirred at RT for 24 h. The reaction mixture was cooled to −78° C. and H₂O (5 mL), EtOH (5 mL), 30% aq. H₂O₂ (5 mL) and aq. 1 N NaOH solution (5 mL) were added. The cooling bath was removed and the mixture was stirred at RT for 1 h. Solid Na₂S₂O₃ was added and the crude product was extracted with EtOAc. The organic layer was separated, dried over Na₂SO₄, filtered, and concentrated under reduced pressure to yield the crude product. The crude product was purified via silica gel chromatography (EtOAc:heptane, 50:50). Desired fractions were combined and concentrated under reduced pressure to yield cis-1-benzhydryl-2-methylazetidin-3-ol (4.9 g) as a white solid. ¹H NMR (400 MHz, DMSO-d₆) δ 7.40 (m, 4H), 7.26 (m, 4H), 7.16 (m, 2H), 5.03 (d, J=5.6 Hz, 1H), 4.46 (s, 1H), 4.21 (m, 1H), 3.38 (m, 1H), 3.02 (m, 1H), 2.95 (m, 1H), 0.67 (d, J=6.5 Hz, 3H).

Step f: To cis-1-benzhydryl-2-methylazetidin-3-ol (4.41 g, 16.2 mmol) in DCM (100 mL) at 0° C., were added NEt₃ (7.28 mL, 52.2 mmol) and methanesulfonyl chloride (2.04 mL, 26.1 mmol). The cooling bath was removed and the reaction mixture was stirred at RT for 3 h, upon which time it was diluted with DCM and washed with H₂O (200 mL×3). The organic layer was dried over Na₂SO₄, filtered, and concentrated under reduced pressure to yield the crude product. The crude product was purified via silica gel chromatography (EtOAc:heptane with 1% NEt₃, 0:100 to 50:50). Desired fractions were combined and concentrated under reduced pressure to yield cis-1-benzhydryl-2-methylazetidin-3-yl methanesulfonate (3.65 g). ¹H NMR (400 MHz, CDCl₃) δ 7.50-7.39 (m, 4H), 7.35-7.19 (m, 6H), 5.18 (td, J=6.1, 2.2 Hz, 1H), 4.43 (s, 1H), 3.69 (td, J=6.4, 1.4 Hz, 1H), 3.57 (dt, J=10.1, 1.9 Hz, 1H), 3.19 (dd, J=10.1, 6.1 Hz, 1H), 3.06 (s, 3H), 0.86 (d, J=6.4 Hz, 3H).

Step g: To cis-1-benzhydryl-2-methylazetidin-3-yl methanesulfonate (5.36 g, 16.2 mmol) in iPrOH (100 mL) at RT, was added 30% aq. NH₄OH (21.0 mL, 162 mmol). The reaction mixture was heated to 60° C. for 2 h under refluxing conditions, upon which time it was cooled to RT and concentrated under reduced pressure. The residue was extracted with DCM, washed with aq. sat. NaHCO₃ solution, and aq. sat. NaCl solution. The organic extracts were dried over Na₂SO₄, filtered, and concentrated under reduced pressure to yield trans-1-benzhydryl-2-methylazetidin-3-amine (crude). ¹H NMR (400 MHz, DMSO-d₆) δ 7.40 (td, J=7.7, 1.3 Hz, 4H), 7.26 (dt, J=12.2, 7.5 Hz, 4H), 7.20-7.13 (m, 2H), 4.43 (s, 1H), 3.42-3.29 (m, 2H), 2.95 (t, J=7.3 Hz, 1H), 2.85 (dd, J=7.7, 3.1 Hz, 1H), 0.61 (d, J=6.2 Hz, 3H).

Step h: To trans-1-benzhydryl-2-methylazetidin-3-amine (3.2 g, 12.7 mmol) in DCM (100 mL) at RT, were added Boc₂O (4.42 mL, 19.0 mmol) and DIPEA (6.64 mL, 38.0 mmol). The reaction mixture was stirred for 2 h, upon which time it was concentrated under reduced pressure. The residue was diluted with EtOAc, and washed with aq. sat. NaHCO₃ solution followed by aq. sat. NaCl solution. The organic layer was dried over Na₂SO₄, filtered, and concentrated under reduced pressure to yield the crude product. The crude product was purified via silica gel chromatography (EtOAc:heptane, 20:80). Desired fractions were combined and concentrated under reduced pressure to yield racemic tert-butyl ((2S,3S)-1-benzhydryl-2-methylazetidin-3-yl)carbamate (3.5 g). ¹H NMR (400 MHz, DMSO) δ 7.44-7.37 (m, 4H), 7.32-7.22 (m, 5H), 7.19-7.13 (m, 2H), 4.10-3.99 (m, 1H), 3.43 (t, J=6.5 Hz, 1H), 3.32 (s, 1H), 3.12 (dd, J=8.0, 3.6 Hz, 1H), 2.98 (t, J=7.9 Hz, 1H), 1.36 (s, 9H), 0.57 (d, J=6.5 Hz, 3H).

Step i: Single enantiomers were separated by chiral SFC: ChiralPak IG21×2 50 mm, 80 g per minute, 15% MeOH with 10 mM ammonia to yield tert-butyl ((2S,3S)-1-benzhydryl-2-methylazetidin-3-yl)carbamate (1.77 g, Peak 1, eluting first) and tert-butyl ((2R,3R)-1-benzhydryl-2-methylazetidin-3-yl)carbamate (1.7 g, Peak 2, eluting second), respectively.

Step j: To tert-butyl ((2S,3S)-1-benzhydryl-2-methylazetidin-3-yl)carbamate (5.49 g, 15.6 mmol) in EtOH (75 mL) at RT under N₂, was added palladium hydroxide (1.09 g, 1.56 mmol). The reaction mixture was stirred under H₂ (1 atm., balloon) for 2 h, upon which time it was purged with N₂ and filtered. The filtrate was concentrated under reduced pressure to yield tert-butyl ((2S,3S)-2-methylazetidin-3-yl)carbamate (crude). ¹H NMR (400 MHz, DMSO-d₆) δ 4.35-4.22 (m, 1H), 3.83-3.70 (m, 1H), 3.53-3.43 (m, 1H), 3.40-3.34 (m, 1H), 1.37 (s, 9H), 1.04 (d, J=6.6 Hz, 3H).

Step k: To tert-butyl ((2S,3S)-2-methylazetidin-3-yl)carbamate (2.9 g, 15.6 mmol) in DCM (100 mL) at RT, were added formaldehyde (8.58 mL, 31.1 mmol) and sodium triacetoxyborohydride (6.60 g, 31.1 mmol). The reaction mixture was stirred at RT for 1 h, upon which time it was diluted with DCM. The organic layer was washed with aq. sat. NaHCO₃ solution and aq. sat. NaCl solution. The resulting organics were dried over Na₂SO₄, filtered, and concentrated under reduced pressure to yield the crude product. The crude product was purified via silica gel chromatography (MeOH:DCM with 1% NH₄OH, 20:80). Desired fractions were combined and concentrated under reduced pressure to yield tert-butyl ((2S,3S)-1,2-dimethylazetidin-3-yl)carbamate (2.67 g) as a white solid. ¹H NMR (400 MHz, DMSO-d₆) δ 7.26 (d, J=7.8 Hz, 1H), 3.98 (qd, J=7.5, 3.0 Hz, 1H), 3.21-3.05 (m, 2H), 3.01 (t, J=7.5 Hz, 1H), 2.13 (s, 3H), 1.37 (s, 9H), 0.91 (d, J=6.4 Hz, 3H).

Step I: To tert-butyl ((2S,3S)-1,2-dimethylazetidin-3-yl)carbamate (2.67 g, 13.3 mmol) in MeOH (30 mL) at RT, was added 4 M HCl solution in 1,4-dioxane (33.3 mL, 133 mmol). The reaction mixture was stirred for 2 h, upon which time it was concentrated under reduced pressure to yield (2S,3S)-1,2-dimethylazetidin-3-amine hydrochloride salt (2.4 g, crude) as an oil that solidified over time.

### Intermediate 19

### ((1r,4r)-4-isopropoxycyclohexyl)methyl 4-methylbenzenesulfonate

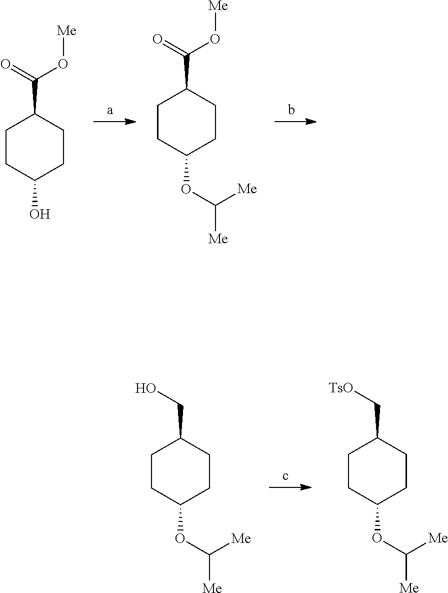 [SMILES: COC(=O)[C@H]1CC[C@H](O)CC1 ; COC(=O)[C@H]1CC[C@H](OC(C)C)CC1 ; CC(C)O[C@H]1CC[C@H](CO)CC1 ; Cc1ccc(S(=O)(=O)OC[C@H]2CC[C@H](OC(C)C)CC2)cc1]

Step a: To methyl (1r,4r)-4-hydroxycyclohexane-1-carboxylate (31.9 g, 202 mmol) in THF (400 mL) at 0° C. under N₂, were added NEt₃ (30.9 mL, 222 mmol) and TMSCI (26.0 mL, 204 mmol) drop-wise. The reaction mixture was stirred at 0° C. for 30 min., upon which time it was diluted with 25 mL heptane, stirred vigorously, and filtered. The resulting clear filtrate was concentrated under reduced pressure to yield the crude silyl ether intermediate as a colorless oil. The crude silyl ether was dissolved in anhydrous DCM (400 mL) at RT and under N₂. The resulting solution was treated with acetone (17.8 mL, 242 mmol), and the reaction mixture was cooled to −78° C. Next, Et₃SiH (38.6 mL, 242 mmol) was added followed by TMSOTf (14.6 mL, 81 mmol) drop-wise. The reaction mixture was stirred for 5 min. at −78° C., and then warmed to 0° C. The reaction mixture was stirred at 0° C. for an additional 1 h, upon which time it was neutralized with aq. sat. NaHCO₃ solution (20 mL) and stirred vigorously for 5 min. The resulting mixture was extracted with DCM (40 mL), and the organic extracts were combined, dried over Na₂SO₄, filtered, and concentrated under reduced pressure to yield the crude product. The crude product was purified via silica gel chromatography (EtOAc:heptane, 0:100 to 80:20). Desired fractions were combined and concentrated under reduced pressure to yield methyl (1r,4r)-4-isopropoxycyclohexane-1-carboxylate (37.7 g) as a colorless oil. ¹H NMR (400 MHz, CDCl₃) δ 3.80-3.66 (m, 4H), 3.29 (tt, J=10.6, 3.9 Hz, 1H), 2.37-2.22 (m, 1H), 2.12-1.96 (m, 4H), 1.57-1.42 (m, 2H), 1.34-1.27 (m, 2H), 1.16 (d, J=6.1 Hz, 6H).

Step b: To methyl (1r,4r)-4-isopropoxycyclohexane-1-carboxylate (14.2 g, 70.9 mmol) in THF (250 mL) at 0° C. under N₂, was added 1.0 M LAH in THF (78 mL, 78 mmol) drop-wise. The reaction mixture was stirred at 0° C. for 10 min., upon which time it was carefully quenched with aq. sat. NaHCO₃ solution at 0° C. under N₂. The mixture was diluted with EtOAc (100 mL) and filtered. The separated organic phase of the filtrate was concentrated under reduced pressure to yield ((1r,4r)-4-isopropoxycyclohexyl)methanol (11.9 g, crude) as a colorless oil. ¹H NMR (400 MHz, DMSO-d₆) δ 4.38 (t, J=5.3 Hz, 1H), 3.69 (hept, J=6.1 Hz, 1H), 3.26-3.14 (m, 3H), 1.96-1.82 (m, 2H), 1.81-1.63 (m, 2H), 1.36-1.21 (m, 1H), 1.16-1.09 (m, 1H), 1.06 (d, J=6.1 Hz, 7H), 0.90 (tdd, J=13.3, 11.7, 3.3 Hz, 2H).

Step c: To ((1r,4r)-4-isopropoxycyclohexyl)methanol (5.00 g, 29.0 mmol) in DCM (145 mL) at 0° C., were added NEt₃ (8.05 mL, 58.0 mmol) and TsCl (6.6 g, 34.8 mmol). The reaction mixture was warmed to RT while stirring for 72 h. The reaction mixture was diluted with H₂O and DCM, and the organic layer was passed through a phase separator. The filtrate was concentrated under reduced pressure to yield the crude product. The crude product was purified via silica gel chromatography (EtOAc:heptane, 0:100 to 80:20). Desired fractions were combined and concentrated under reduced pressure to yield ((1r,4r)-4-isopropoxycyclohexyl)methyl 4-methylbenzenesulfonate (7.45 g) as a colorless oil that solidified over time. MS m/z 327.3 [M+H]⁺.

### Intermediate 20

### (1r,4r)-4-isopropoxycyclohexane-1-carbaldehyde

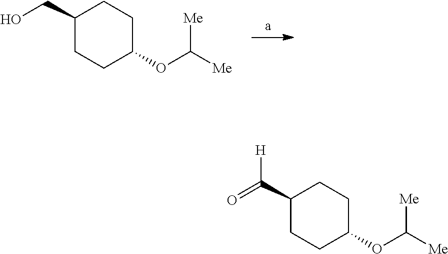 [SMILES: CC(C)O[C@H]1CC[C@H](CO)CC1 ; CC(C)O[C@H]1CC[C@H](C=O)CC1]

Step a: To ((1r,4r)-4-isopropoxycyclohexyl)methanol (6.49 g, 37.7 mmol) in DCM (77 mL) at −19° C. under N₂, was added DIPEA (19.7 mL, 113 mmol). Next, pyridine sulfur trioxide complex (7.79 g, 49.0 mmol) in DMSO (48.3 mL) was added drop-wise at such a rate to keep the temperature below −9° C. The reaction mixture was warmed to 0° C. and stirred for 30 min., upon which time it was poured into a separatory funnel containing H₂O (150 mL) and DCM (150 mL). The organic layer was partitioned and separated. The aq. layer was extracted with DCM (50 mL×2). The combined organic extracts were washed with aq. sat. NaCl solution, passed through a phase separator, and concentrated under reduced pressure to yield the crude product. The crude product was enriched via silica gel chromatography (EtOAc:heptane, 10:90 to 50:50). Desired fractions were combined and concentrated under reduced pressure. This material was purified via silica gel chromatography (EtOAc:heptane, 0:100 to 20:100). Desired fractions were combined and concentrated under reduced pressure to yield (1r,4r)-4-(benzyloxy)cyclohexane-1-carbaldehyde (4.21 g) as a clear and yellow oil. ¹H NMR (400 MHz, CDCl₃) δ 9.66 (d, J=1.5 Hz, 1H), 4.01-3.47 (m, 1H), 3.47-3.11 (m, 1H), 2.36-2.14 (m, 1H), 2.14-1.80 (m, 4H), 1.42-1.24 (m, 4H), 1.23-1.08 (m, 6H).

### Intermediate 21

### 5-chloro-2-formyl-1-(((1r,4r)-4-isopropoxycyclohexyl)methyl)-1H-pyrrolo[2,3-b]pyridine-3-carboxylic acid

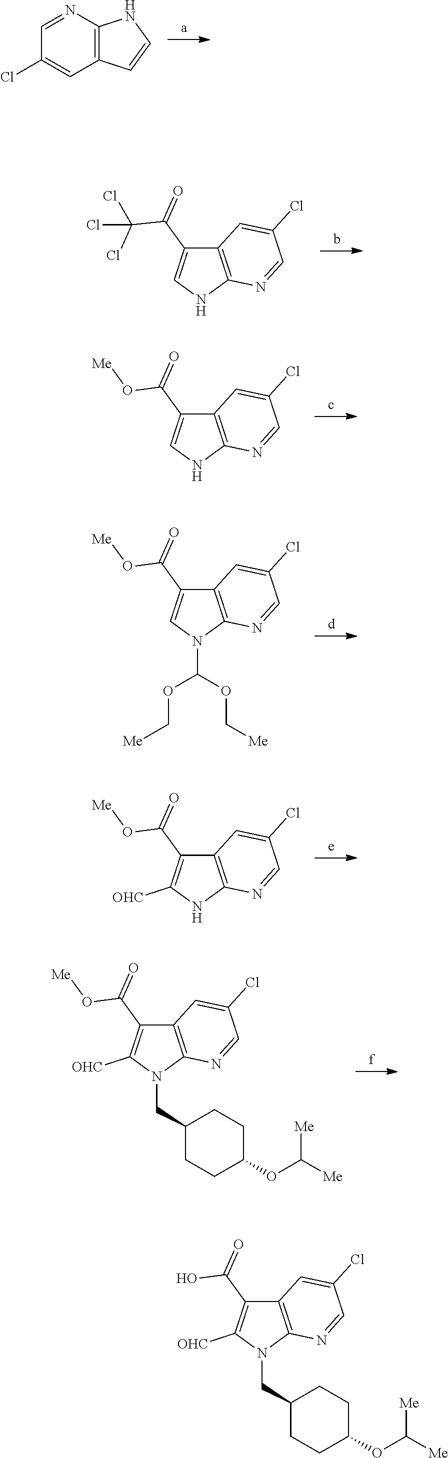

Step a: To 5-chloro-1H-pyrrolo[2,3-b]pyridine (90 g, 592 mmol) in DCM (900 mL) at RT, was added AlCl₃ (395 g, 2.96 mol). The reaction mixture was stirred for 10 min. at RT, upon which time 2,2,2-trichloroacetyl chloride (162 g, 888 mmol) was added drop-wise over 20 min. The reaction mixture was stirred for another 3 h, upon which time it was quenched by addition of H₂O (1 L) at 0° C. and extracted with EtOAc (1 L×3). The combined organic extracts were dried over Na₂SO₄, filtered, and concentrated under reduced pressure to yield the crude product. The crude product was purified via trituration with MeOH to yield 2,2,2-trichloro-1-(5-chloro-1H-pyrrolo[2,3-b]pyridin-3-yl)ethan-1-one (160 g) as a yellow solid. MS m/z 297 [M+H]⁺.

Step b: To 2,2,2-trichloro-1-(5-chloro-1H-pyrrolo[2,3-b]pyridin-3-yl)ethan-1-one (160 g, 541 mmol) in MeOH (1.6 L) at RT, was added KOH (33.3 g, 595 mmol). The reaction mixture was stirred at RT for 2.5 h, upon which time the pH was adjusted to 7 with aq. 1 N HCl solution. The resulting precipitate was collected by filtration. The filter cake was washed with H₂O (500 mL) and MeOH (300 mL×3), and dried under reduced pressure to yield methyl 5-chloro-1H-pyrrolo[2,3-b]pyridine-3-carboxylate (80 g, crude) as a yellow solid. ¹H NMR (400 MHz, DMSO-d₆) δ 12.78 (s, 1H), 8.34-8.28 (m, 3H), 3.84 (m, 3H).

Step c: To methyl 5-chloro-1H-pyrrolo[2,3-b]pyridine-3-carboxylate (80 g, 381 mmol) at RT, was added ethyl orthoformate (320 mL). The reaction mixture was stirred at 180° C. for 3 h, upon which time it was cooled to 0° C. and heptane (300 mL) was added. The resulting mixture was stirred for 30 min. and the precipitate was collected by filtration. The filter cake was dried under reduced pressure to yield methyl 5-chloro-1-(diethoxymethyl)-1H-pyrrolo[2,3-b]pyridine-3-carboxylate (70 g, crude) as a yellow solid. ¹H NMR (400 MHz, DMSO-d₆) δ 8.44 (d, J=2.4 Hz, 1H), 8.26 (d, J=2.4 Hz, 1H), 8.14-8.12 (s, 1H), 6.80 (s, 1H), 3.86 (s, 3H), 3.72-3.68 (m, 2H), 3.66-3.62 (m, 2H), 1.15 (t, J=7.0 Hz, 6H).

Step d: To methyl 5-chloro-1-(diethoxymethyl)-1H-pyrrolo[2,3-b]pyridine-3-carboxylate (70 g, 224 mmol) in THF (700 mL) at −78° C., was added LDA (48.1 g, 449 mmol) drop-wise. The reaction mixture was stirred for 1 h, upon which time methyl formate (53.8 g, 897 mmol) was added drop-wise. The reaction mixture was slowly warmed to RT over ˜1 h. The reaction mixture was quenched by addition of aq. sat. NH₄Cl solution (800 mL) at 0° C. and extracted with EtOAc (800 mL×3). The combined organic extracts were dried over Na₂SO₄, filtered, and concentrated under reduced pressure to yield the crude product. The crude product was purified via trituration with THF (300 mL) to yield methyl 5-chloro-2-formyl-1H-pyrrolo[2,3-b]pyridine-3-carboxylate (11 g) as a brown solid. MS m/z 239 [M+H]⁺.

Step e: To methyl 5-chloro-2-formyl-1H-pyrrolo[2,3-b]pyridine-3-carboxylate (300 mg, 1.26 mmol) in DMF (6.29 mL) at RT, were added ((1r,4r)-4-isopropoxycyclohexyl)methyl 4-methylbenzenesulfonate (410 mg, 1.26 mmol) and K₂CO₃ (695 mg, 5.03 mmol). The reaction mixture was heated to 100° C. for 3 h, upon which time it was diluted with EtOAc and washed with H₂O (20 mL×3) and aq. sat. NaCl solution. The resulting organic layer was dried over MgSO₄, filtered, and concentrated under reduced pressure to yield the crude product. The crude product was diluted with DCM, adsorbed onto Celite®, evaporated to dryness, and purified via silica gel chromatography (EtOAc:heptane, 0:100 to 100:0). Desired fractions were combined and concentrated under reduced pressure to yield methyl 5-chloro-2-formyl-1-(((1r,4r)-4-isopropoxycyclohexyl)methyl)-1H-pyrrolo[2,3-b]pyridine-3-carboxylate (402 mg). ¹H NMR (400 MHz, CDCl₃) δ 10.87 (s, 1H), 8.52 (d, J=2.4 Hz, 1H), 8.49 (d, J=2.4 Hz, 1H), 4.63 (d, J=7.4 Hz, 2H), 4.03 (s, 3H), 3.75-3.60 (m, 1H), 3.31-3.15 (m, 1H), 1.96-1.87 (m, 2H), 1.87-1.75 (m, 1H), 1.54-1.49 (m, 2H), 1.34-1.29 (m, 1H), 1.18-1.12 (m, 3H), 1.11 (s, 3H), 1.10 (s, 3H). MS m/z 393.1 [M+H]⁺.

Step f: To methyl 5-chloro-2-formyl-1-(((1r,4r)-4-isopropoxycyclohexyl)methyl)-1H-pyrrolo[2,3-b]pyridine-3-carboxylate (402 mg, 1.023 mmol) in THF (5.12 mL) and MeOH (5.12 mL) at RT, was added aq. 2 N LiOH solution (5.12 mL, 10.2 mmol). The reaction mixture was heated to 50° C. for 1.5 h, upon which time it was cooled to RT, diluted with aq. 1 M Na₂S₂O₃ solution and then extracted with DCM (10 mL×3). The combined organic extracts were passed through a phase separator and concentrated under reduced pressure to yield 5-chloro-2-formyl-1-(((1r,4r)-4-isopropoxycyclohexyl)methyl)-1H-pyrrolo[2,3-b]pyridine-3-carboxylic acid (crude). MS m/z 379.2 [M+H]⁺.

### Intermediate 22

### 5-chloro-2-(difluoromethyl)-1-((tetrahydro-2H-pyran-4-yl)methyl)-1H-indole-3-carboxylic acid

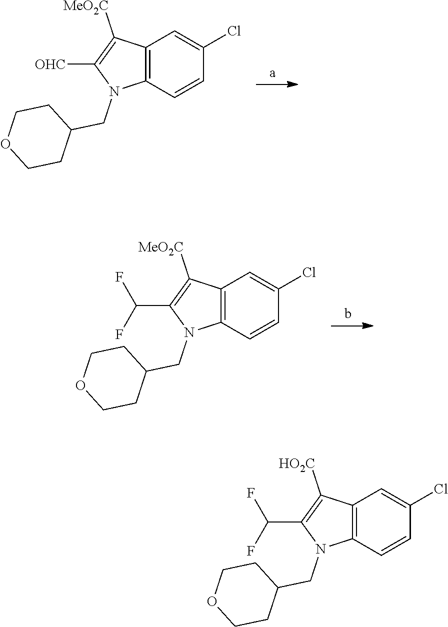 [SMILES: COC(=O)c1c(C=O)n(CC2CCOCC2)c2ccc(Cl)cc12 ; COC(=O)c1c(C(F)F)n(CC2CCOCC2)c2ccc(Cl)cc12 ; O=C(O)c1c(C(F)F)n(CC2CCOCC2)c2ccc(Cl)cc12]

Step a: To methyl 5-chloro-2-formyl-1-((tetrahydro-2H-pyran-4-yl)methyl)-1H-indole-3-carboxylate (49 mg, 0.146 mmol) in DCM (0.75 mL) at 0° C., was added deoxofluor (0.135 mL, 0.730 mmol). The reaction mixture was stirred for 5 min., upon which time it was warmed to RT while stirring for 2 h. Additional deoxofluor (0.135 mL, 0.730 mmol) was added and the reaction mixture was stirred at RT for 1 h. The reaction mixture was cooled to 0° C. and quenched via addition of ice cold aq. sat. NaHCO₃ solution and partitioned between DCM and H₂O. The aq. phase was washed with DCM, and the combined organics were washed with aq. sat. NaHCO₃ solution and aq. sat. NaCl solution. The combined organics were dried over MgSO₄, filtered, and concentrated under reduced pressure to yield methyl 5-chloro-2-(difluoromethyl)-1-((tetrahydro-2H-pyran-4-yl)methyl)-1H-indole-3-carboxylate (88.4 mg, crude) as a tan solid.

Step b: To methyl 5-chloro-2-(difluoromethyl)-1-((tetrahydro-2H-pyran-4-yl)methyl)-1H-indole-3-carboxylate (52.2 mg, 0.146 mmol) in THF (0.50 mL) and MeOH (0.25 mL) at RT, was added aq. 1 N NaOH solution (0.729 mL, 0.729 mmol). The reaction mixture was heated to 50° C. for 1.5 h, upon which time it was concentrated under reduced pressure to remove the organic solvent. The residue was diluted with H₂O, then treated with aq. 1 N HCl solution until pH 1 was reached. The resulting suspension was diluted with EtOAc and the layers were separated. The aq. layer was extracted with EtOAc, the combined organics were washed with aq. sat. NaCl solution, dried over MgSO₄, filtered, and concentrated under reduced pressure to yield 5-chloro-2-(difluoromethyl)-1-((tetrahydro-2H-pyran-4-yl)methyl)-1H-indole-3-carboxylic acid (35.1 mg, crude) as a yellow solid. ¹H NMR (400 MHz, DMSO-d₆) δ 13.28 (s, 1H), 8.09 (d, J=2.2 Hz, 1H), 7.87 (d, J=9.0 Hz, 1H), 7.42 (dd, J=9.0, 2.2 Hz, 1H), 4.33 (d, J=7.5 Hz, 2H), 3.88-3.76 (m, 2H), 3.17 (td, J=11.7, 2.3 Hz, 2H), 2.26-2.10 (m, 1H), 1.47-1.20 (m, 5H). MS m/z 342.3 [M−H]⁻.

### Intermediate 23

### 5-chloro-2-cyano-1-((tetrahydro-2H-pyran-4-yl)methyl)-1H-indole-3-carboxylic acid

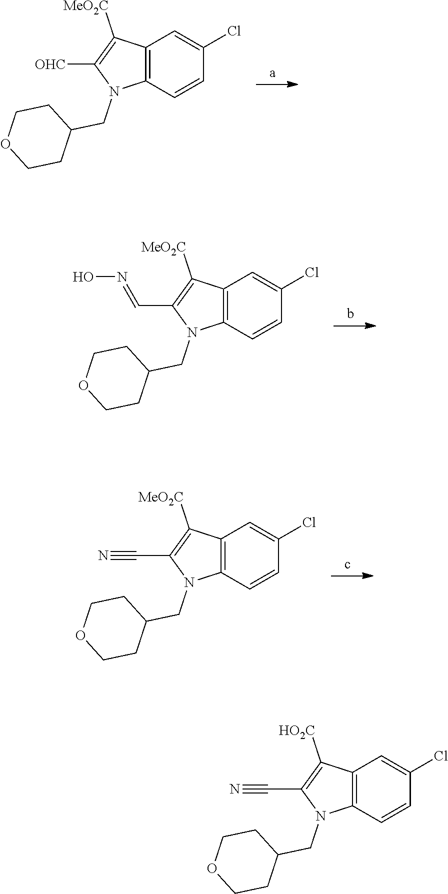 [SMILES: COC(=O)c1c(C=O)n(CC2CCOCC2)c2ccc(Cl)cc12 ; COC(=O)c1c(/C=N/O)n(CC2CCOCC2)c2ccc(Cl)cc12 ; COC(=O)c1c(C#N)n(CC2CCOCC2)c2ccc(Cl)cc12 ; N#Cc1c(C(=O)O)c2cc(Cl)ccc2n1CC1CCOCC1]

Step a: To methyl 5-chloro-2-formyl-1-((tetrahydro-2H-pyran-4-yl)methyl)-1H-indole-3-carboxylate (110 mg, 0.329 mmol) in EtOH (1.65 mL) at RT, were added pyridine (0.053 mL, 0.658 mmol) and hydroxylamine hydrochloride salt (27.4 mg, 0.395 mmol). The reaction mixture was heated overnight at 80° C., upon which time the reaction mixture was cooled to RT and concentrated under reduced pressure. The resulting material was dissolved in H₂O/EtOAc and the layers were separated. The organic phase was washed with aq. sat. NaCl solution, dried over MgSO₄, filtered, and concentrated under reduced pressure to yield methyl (E)-5-chloro-2-((hydroxyimino)methyl)-1-((tetrahydro-2H-pyran-4-yl)methyl)-1H-indole-3-carboxylate (110 mg, crude) as a white solid. ¹H NMR (400 MHz, DMSO-d₆) δ 12.03 (s, 1H), 8.97 (s, 1H), 8.02 (d, J=2.0 Hz, 1H), 7.78 (d, J=9.0 Hz, 1H), 7.35 (dd, J=8.9, 2.2 Hz, 1H), 4.52 (d, J=7.2 Hz, 2H), 3.88 (s, 3H), 3.83-3.74 (m, 2H), 3.21-3.10 (m, 2H), 2.13-1.98 (m, 1H), 1.43-1.22 (m, 4H). MS m/z 351.3 [M+H]⁺.

Step b: To methyl (E)-5-chloro-2-((hydroxyimino)methyl)-1-((tetrahydro-2H-pyran-4-yl)methyl)-1H-indole-3-carboxylate (109 mg, 0.311 mmol) in NEt₃ (1.5 mL) at RT, was added Ac₂O (0.059 mL, 0.621 mmol). The reaction mixture was heated to 90° C. for 2 h, upon which time the reaction mixture was cooled to RT and partitioned between EtOAc and H₂O. The aq. phase was washed with EtOAc. The combined organic extracts were washed with aq. sat. NaCl solution, dried over MgSO₄, filtered, and concentrated under reduced pressure to yield the crude product. The crude product was purified via silica gel chromatography (EtOAc:heptane, 0:100 to 80:20). Desired fractions were combined and concentrated under reduced pressure to yield methyl 5-chloro-2-cyano-1-((tetrahydro-2H-pyran-4-yl)methyl)-1H-indole-3-carboxylate (85 mg) as a tan solid. ¹H NMR (400 MHz, DMSO-d₆) δ 8.08 (dd, J=2.1, 0.6 Hz, 1H), 7.95 (dd, J=9.0, 0.7 Hz, 1H), 7.55 (dd, J=9.0, 2.1 Hz, 1H), 4.36 (d, J=7.5 Hz, 2H), 3.93 (s, 3H), 3.87-3.77 (m, 2H), 3.24-3.15 (m, 2H), 2.18-2.04 (m, 1H), 1.41-1.29 (m, 4H). MS m/z 333.4 [M+H]⁺.

Step c: To methyl 5-chloro-2-cyano-1-((tetrahydro-2H-pyran-4-yl)methyl)-1H-indole-3-carboxylate (83.7 mg, 0.252 mmol) in THF (958 μL) and MeOH (479 μL) at RT, was added aq. 1 N NaOH solution (528 μL, 0.528 mmol). The reaction mixture was stirred overnight at RT upon which time the reaction mixture was concentrated under reduced pressure to remove the organic solvent. The residue was diluted with H₂O and treated with aq. 1 N HCl solution until pH 1 was reached. The resulting suspension was diluted with EtOAc and the layers were separated. The aq. layer was extracted with EtOAc and the combined organics were washed with aq. sat. NaCl solution, dried over MgSO₄, filtered, and concentrated under reduced pressure to yield 5-chloro-2-cyano-1-((tetrahydro-2H-pyran-4-yl)methyl)-1H-indole-3-carboxylic acid (71 mg, crude) as a solid. ¹H NMR (400 MHz, DMSO-d₆) δ 13.45 (s, 1H), 8.11 (d, J=2.1 Hz, 1H), 7.91 (d, J=9.0 Hz, 1H), 7.51 (dd, J=9.0, 2.2 Hz, 1H), 4.33 (d, J=7.4 Hz, 2H), 3.86-3.77 (m, 2H), 3.27-3.13 (m, 2H), 2.17-2.03 (m, 1H), 1.44-1.31 (m, 4H). MS m/z 317.5 [M+H]⁺.

### Intermediate 24

### methyl 4-phenylpiperidine-4-carboxylate

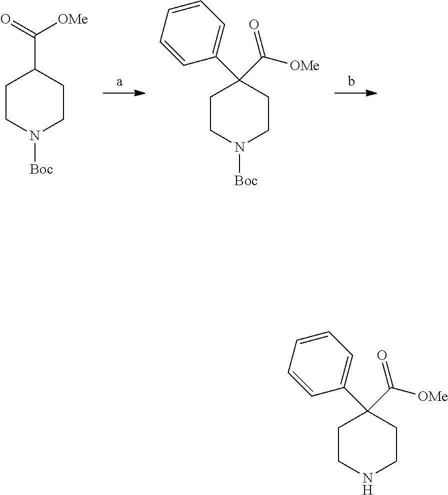 [SMILES: COC(=O)C1CCN(C(=O)OC(C)(C)C)CC1 ; COC(=O)C1(c2ccccc2)CCN(C(=O)OC(C)(C)C)CC1 ; COC(=O)C1(c2ccccc2)CCNCC1]

Step a: To 1-tert-butyl 4-methyl piperidine-1,4-dicarboxylate (3.63 g, 14.9 mmol) in toluene (38.2 mL) under N₂ at RT, was added bromobenzene (1.21 mL, 11.5 mmol). This mixture was purged with N₂ for −10 min. Next, bis(tri-tert-butylphosphine)palladium(0) (CAS #53199-31-8, 0.246 g, 0.481 mmol) was added followed by 1.0 M LHMDS solution in toluene (20.6 mL, 20.6 mmol). The reaction mixture was stirred at RT for 24 h, upon which time it was quenched with 2 mL AcOH, filtered, and concentrated under reduced pressure to yield the crude product. The crude product was purified via silica gel chromatography (EtOAc:heptane, 0:100 to 40:60). Desired fractions were combined and concentrated under reduced pressure to yield 1-(tert-butyl) 4-methyl 4-phenylpiperidine-1,4-dicarboxylate (2.09 g) as a white solid. ¹H NMR (400 MHz, MeOD-d₄) δ 7.47-7.33 (m, 4H), 7.31-7.23 (m, 1H), 3.95 (dtd, J=13.7, 4.5, 1.3 Hz, 2H), 3.68 (s, 3H), 3.05 (s, 2H), 2.53 (dp, J=13.6, 2.2 Hz, 2H), 1.86 (ddd, J=13.4, 11.4, 4.2 Hz, 2H), 1.48 (s, 9H). MS m/z 342.2 [M+Na]⁺.

Step b: To 1-(tert-butyl) 4-methyl 4-phenylpiperidine-1,4-dicarboxylate (2.15 g, 6.73 mmol) in MeOH (16.8 mL) exposed to air at RT, was added 4 M HCl solution in 1,4-dioxane (3.37 mL, 13.5 mmol) drop-wise. The reaction mixture was stirred for 8 h, upon which time it was poured into aq. sat. NaHCO₃ solution (50 mL). The material was extracted with EtOAc (20 mL×3), and the combined organics were washed with aq. sat. NaCl solution (20 mL×2), dried over Na₂SO₄, filtered, and concentrated under reduced pressure to yield methyl 4-phenylpiperidine-4-carboxylate (crude) as a white solid. MS m/z 220.4 [M+H]⁺.

### Intermediate 25a and Intermediate 25b

### 3-(((2S,5R)-5-ethoxytetrahydro-2H-pyran-2-yl)methyl)-6-fluoro-2-methyl-1H-indole and 3-(((2R,5S)-5-ethoxytetrahydro-2H-pyran-2-yl)methyl)-6-fluoro-2-methyl-1H-indole

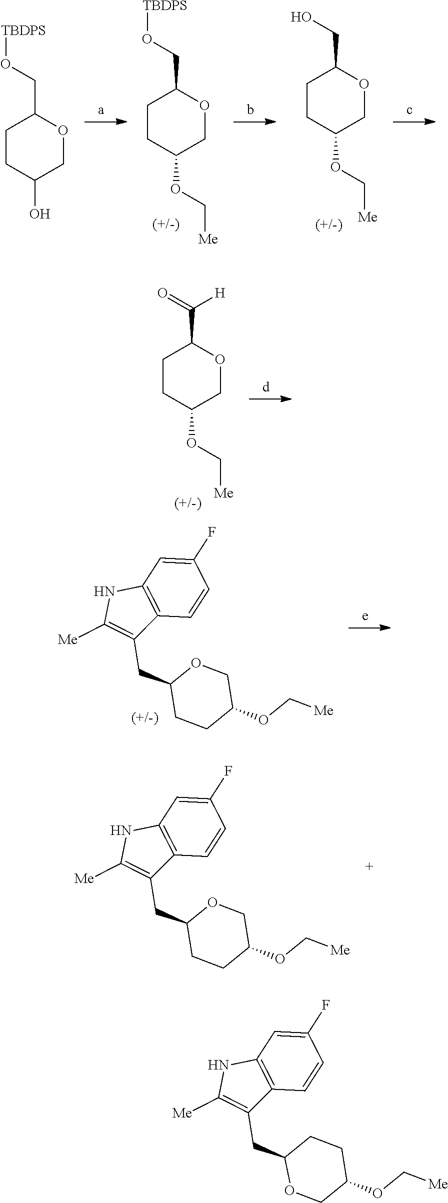

Step a: To 6-(((tert-butyldiphenylsilyl)oxy)methyl)tetrahydro-2H-pyran-3-ol (Intermediate 8b, 16.0 g, 43.18 mmol) in THF (200 mL) at 0° C., were added NaH (3.4 g, 86.4 mmol) and ethyl iodide (10.4 mL, 130 mmol). The reaction mixture was stirred at RT for 2 h, upon which time it was cooled to 0° C., quenched with cooled H₂O, and extracted with EtOAc. The organic extracts were washed with H₂O, aq. sat. NaCl solution, dried over Na₂SO₄, filtered, and concentrated under reduced pressure to yield the crude product. The crude product was purified via silica gel chromatography (EtOAc:hexane, 2:98 to 5:95). Desired fractions were combined and concentrated under reduced pressure to yield racemic trans tert-butyl((5-ethoxytetrahydro-2H-pyran-2-yl)methoxy)diphenylsilane (6.8 g, eluting first) as a colorless oil. ¹H NMR (400 MHz, CDCl₃) δ 7.69-7.63 (m, 4H), 7.42-7.32 (m, 6H), 4.10-4.05 (m, 1H), 3.74-3.68 (m, 1H), 3.60-3.48 (m, 3H), 3.37-3.30 (m, 2H), 3.15-3.08 (m, 1H), 2.20-2.15 (m, 1H), 1.84-1.80 (m, 1H), 1.40-1.30 (m, 2H), 1.20 (t, J=7.2 Hz, 3H), 1.05 (s, 9H).

Step b: To racemic trans tert-butyl((5-ethoxytetrahydro-2H-pyran-2-yl)methoxy)diphenylsilane (6.8 g, 17.1 mmol) in THF (100 mL) at 0° C., was added a 1.0 M TBAF solution in THF (34.1 mL, 34.1 mmol). The reaction mixture was stirred for 2 h at RT, upon which time it was quenched with aq. sat. NH₄Cl solution and extracted with a MeOH:DCM mixture (9:1, 250 mL). Combined organic extracts were washed with aq. sat. NaCl solution, dried over Na₂SO₄, filtered, and concentrated under reduced pressure to yield the crude product. The crude product was purified via silica gel chromatography (EtOAc:hexane, 80:20). Desired fractions were combined and concentrated under reduced pressure to yield racemic trans (5-ethoxytetrahydro-2H-pyran-2-yl)methanol (2.5 g) as a colorless oil. ¹H NMR (300 MHz, DMSO-d₆) δ 4.58 (t, J=6.0 Hz, 1H), 3.98-3.88 (m, 1H), 3.52-3.30 (m, 2H), 3.30-3.12 (m, 4H), 2.94 (t, J=10.5 Hz, 1H), 2.10-2.04 (m, 1H), 1.66-1.60 (m, 1H), 1.26-1.12 (m, 2H), 1.05 (t, J=7.0 Hz, 3H).

Step c: To oxalyl chloride (2.97 g, 23.4 mmol) in DCM (80 mL) at −78° C., were added DMSO (2.77 mL, 39.0 mmol) in DCM (10 mL), racemic trans (5-ethoxytetrahydro-2H-pyran-2-yl)methanol (2.50 g, 15.6 mmol) in DCM (10 mL), and triethylamine (13.0 mL, 93.6 mmol). The reaction mixture was stirred at −78° C. for 2 h, upon which time it was quenched with aq. sat. NH₄Cl solution (25 mL) and extracted with DCM (100 mL). The combined organic extracts were washed with aq. sat. NaHCO₃ solution, dried over Na₂SO₄, filtered, and concentrated under reduced pressure to yield racemic trans 5-ethoxytetrahydro-2H-pyran-2-carbaldehyde (2.5 g, crude) as a light brown oil.

Step d: To 6-fluoro-2-methyl-1H-indole (2.30 g, 15.4 mmol) in DCM (100 mL) at 0° C., were added racemic trans 5-ethoxytetrahydro-2H-pyran-2-carbaldehyde (2.44 g, 15.4 mmol) and Et₃SiH (4.9 mL, 92.6 mmol). The reaction mixture was stirred for 15 min., upon which time trifluoroacetic acid (4.71 mL, 61.7 mmol) was added drop-wise. The resulting reaction mixture was stirred at 0° C. for 1.5 h, upon which time it was diluted with DCM (100 mL) and washed with aq. sat. NaHCO₃ solution (100 mL) followed by aq. sat. NaCl solution. The combined organic extracts were dried over Na₂SO₄, filtered, and concentrated under reduced pressure to yield the crude product. The crude product was purified via silica gel chromatography (EtOAc:hexane, 30:70). The desired fractions were combined and concentrated under reduced pressure to yield racemic trans 3-((5-ethoxytetrahydro-2H-pyran-2-yl)methyl)-6-fluoro-2-methyl-1H-indole (2.6 g) as a light brown solid. ¹H NMR (300 MHz, DMSO-d₆) δ 10.79 (s, 1H), 7.36-7.28 (m, 1H), 6.97 (dd, J=5.4 and 9.9 Hz, 1H), 6.76-6.70 (m, 1H), 3.95-3.90 (m, 1H), 3.48-3.40 (m, 2H), 3.38-3.20 (m, 2H), 2.91 (t, J=10.5 Hz, 1H), 2.75-2.60 (m, 2H), 2.25 (s, 3H), 2.05-1.95 (m, 1H), 1.60-1.51 (m, 1H), 1.25-1.10 (m, 2H), 1.03 (t, J=6.6 Hz, 3H).

Step e: Racemic trans 3-((5-ethoxytetrahydro-2H-pyran-2-yl)methyl)-6-fluoro-2-methyl-1H-indole (2.6 g) was separated via the following method providing 3-(((2S,5R)-5-ethoxytetrahydro-2H-pyran-2-yl)methyl)-6-fluoro-2-methyl-1H-indole and 3-(((2R,5S)-5-ethoxytetrahydro-2H-pyran-2-yl)methyl)-6-fluoro-2-methyl-1H-indole. Chiral separation: LUX AMYLOSE-1, 250 mm×21.2 mm 5 μm; mobile phase: Phase A for CH₃CN, Phase B for 0.1% DIPEA in EtOH:MeOH, 1:1; flow: 15 mL; isocratic Phase A: Phase B=50:50.

Peak 1 (eluting first, Intermediate 25a): 1.2 g; MS m/z 292.2 [M+H]⁺.

Peak 2 (eluting second, Intermediate 25b): 1.2 g; MS m/z 292.1 [M+H]⁺.

### Intermediate 26

### ethyl (1S,4S)-4-amino-3,3-dimethylcyclohexane-1-carboxylate

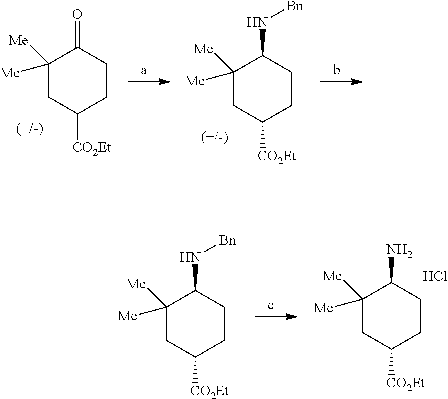 [SMILES: CCOC(=O)C1CCC(=O)C(C)(C)C1 ; CCOC(=O)[C@H]1CC[C@H](NCc2ccccc2)C(C)(C)C1 ; CCOC(=O)[C@H]1CC[C@H](NCc2ccccc2)C(C)(C)C1 ; CCOC(=O)[C@H]1CC[C@H](N)C(C)(C)C1 ; Cl]

Step a: To racemic ethyl 3,3-dimethyl-4-oxocyclohexane-1-carboxylate (1.9 g, 9.58 mmol, obtained as a byproduct from Step a in the route to Intermediate 2) in anhydrous THF (25 mL) at RT under N₂, were added benzylamine (1.31 mL, 12.0 mmol) and sodium triacetoxyborohydride (3.05 g, 14.4 mmol) in two portions. The white suspension was vigorously stirred at RT for 6.5 h. The reaction mixture was quenched via addition of aq. sat. NaHCO₃ solution (50 mL) and stirred for 30 min. before allowing to settle at RT overnight. The resulting mixture was partitioned between EtOAc (25 mL) and aq. sat. NaHCO₃ solution (25 mL), and the aq. phase was extracted with EtOAc (25 mL×2). The combined organic extracts were washed with aq. sat. NaCl solution (25 mL), and dried over Na₂SO₄. The mixture was filtered and the filtrate was concentrated under reduced pressure to yield the crude product. The crude product was purified via reverse phase C18 chromatography (CH₃CN:H₂O with 0.1% NH₄OH, 10:90 to 0:100). Desired fractions were combined, partially concentrated under reduced pressure, and then extracted with EtOAc (25 mL×3). The combined organic extracts were dried over Na₂SO₄, filtered, and concentrated under reduced pressure to yield racemic trans-ethyl-4-(benzylamino)-3,3-dimethylcyclohexane-1-carboxylate (1.6 g). ¹H NMR (400 MHz, CDCl₃) δ 7.34-6.99 (m, 5H), 3.97 (m, J=7.1 Hz, 2H), 3.81 (d, J=13.2 Hz, 1H), 2.32 (tt, J=12.6, 3.5 Hz, 1H), 2.05 (dd, J=11.6, 3.7 Hz, 1H), 1.89-1.77 (m, 2H), 1.51 (ddd, J=13.4, 3.5, 2.3 Hz, 1H), 1.33-1.02 (m, 7H), 0.81 (d, J=41.0 Hz, 7H).

Step b: Racemic trans-ethyl-4-(benzylamino)-3,3-dimethylcyclohexane-1-carboxylate (1.6 g, 5.53 mmol) was purified via preparative chiral SFC separation: Chiralpak IG (CPC071) 21 mm×250 mm; 5 to 25% MeOH and 10 mM NH₃/CO₂; 125 bar over 6.2 min.; multiple runs yielded ethyl (1S,4S)-4-(benzylamino)-3,3-dimethylcyclohexane-1-carboxylate (Peak 1, eluting first, 0.55 g). ¹H NMR (400 MHz, CDCl₃) δ 7.46-7.20 (m, 6H), 4.12 (m, J=7.1 Hz, 2H), 3.97 (d, J=13.2 Hz, 1H), 3.72 (d, J=13.2 Hz, 1H), 2.55-2.40 (m, 1H), 2.21 (dd, J=11.5, 3.7 Hz, 1H), 1.99 (ddt, J=11.4, 6.4, 3.3 Hz, 2H), 1.66 (ddd, J=13.4, 3.6, 2.3 Hz, 1H), 1.52-1.31 (m, 2H), 1.26 (t, J=7.1 Hz, 4H), 1.01 (s, 3H), 0.92 (s, 3H).

Step c: To ethyl (1S,4S)-4-(benzylamino)-3,3-dimethylcyclohexane-1-carboxylate (0.55 g, 1.90 mmol) in EtOH (10 mL) at RT under N₂, was added Pd/C (0.202 g, 0.190 mmol). The vial was purged by performing two reduced pressure-to-N₂ cycles, and the final reduced pressure purge was broken with H₂. The reaction mixture was vigorously stirred under the H₂ atm. overnight. Then the reaction mixture was filtered through a pad of Celite® while washing with EtOH and then EtOAc. The filtrate was concentrated under reduced pressure to dryness, the residue was redissolved in EtOAc and the mixture was concentrated under reduced pressure again. The residue was dissolved in Et₂O (50 mL), and the solution was treated drop-wise with 2.5 M HCl solution in EtOH (1.06 mL, 2.66 mmol) under N₂, which produced a white precipitate. Additional Et₂O (50 mL) was added and the resulting mixture was sonicated and then stirred in an ice bath for 30 min. The solids were collected under reduced pressure filtration and washed with Et₂O. The solid wet cake was then dried under a stream of N₂ through the solids for >1 h to yield ethyl (1S,4S)-4-amino-3,3-dimethylcyclohexane-1-carboxylate hydrochloride salt (0.324 g, crude) as a white solid. ¹H NMR (400 MHz, MeOD-d₄) δ 4.14 (m, J=7.1 Hz, 2H), 2.99 (dd, J=12.3, 4.2 Hz, 1H), 2.56 (tt, J=12.6, 3.7 Hz, 1H), 2.14-2.04 (m, 1H), 1.96-1.85 (m, 1H), 1.80 (ddd, J=13.7, 3.5, 2.4 Hz, 1H), 1.74-1.61 (m, 1H), 1.61-1.40 (m, 2H), 1.26 (t, J=7.1 Hz, 3H), 1.14 (s, 3H), 1.02 (s, 3H).

### Example 1

### (1S,3S,4S)-4-(1-(6-fluoro-3-(((1r,4S)-4-isopropoxycyclohexyl)methyl)-2-methyl-1H-indole-1-carbonyl)-4-(4-fluorophenyl)piperidine-4-carboxamido)-3-methylcyclohexane-1-carboxylic acid

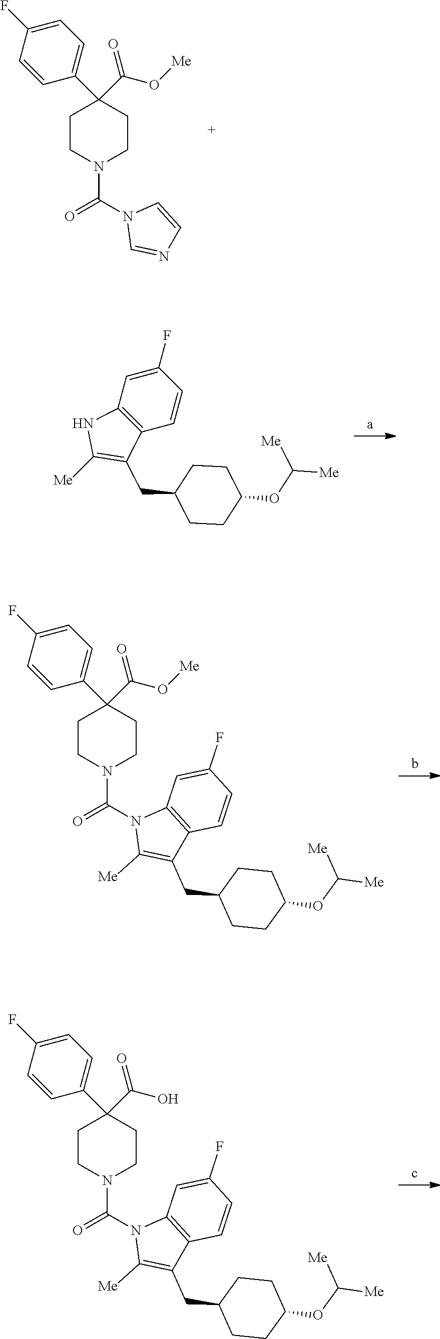 [SMILES: COC(=O)C1(c2ccc(F)cc2)CCN(C(=O)n2ccnc2)CC1 ; Cc1[nH]c2cc(F)ccc2c1C[C@H]1CC[C@H](OC(C)C)CC1 ; COC(=O)C1(c2ccc(F)cc2)CCN(C(=O)n2c(C)c(C[C@H]3CC[C@H](OC(C)C)CC3)c3ccc(F)cc32)CC1 ; Cc1c(C[C@H]2CC[C@H](OC(C)C)CC2)c2ccc(F)cc2n1C(=O)N1CCC(C(=O)O)(c2ccc(F)cc2)CC1]

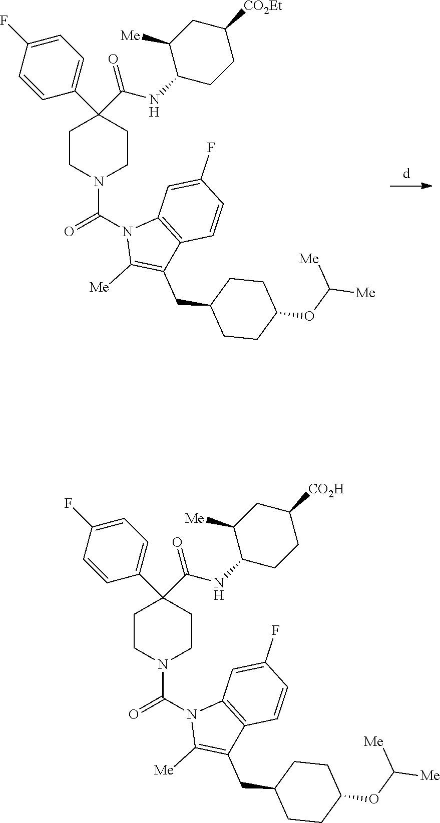 [SMILES: CCOC(=O)[C@H]1CC[C@H](NC(=O)C2(c3ccc(F)cc3)CCN(C(=O)n3c(C)c(C[C@H]4CC[C@H](OC(C)C)CC4)c4ccc(F)cc43)CC2)[C@@H](C)C1 ; Cc1c(C[C@H]2CC[C@H](OC(C)C)CC2)c2ccc(F)cc2n1C(=O)N1CCC(C(=O)N[C@H]2CC[C@H](C(=O)O)C[C@@H]2C)(c2ccc(F)cc2)CC1]

Step a: To 6-fluoro-3-(((1r,4r)-4-isopropoxycyclohexyl)methyl)-2-methyl-1H-indole (8.2 g, 27.0 mmol) in THF (100 mL) at −40° C. under N₂, was added 1.5 M LHMDS solution in toluene (20.7 mL, 31.1 mmol) drop-wise. The reaction mixture was stirred for 10 min., upon which time 2.0 M AlMe₃ solution in toluene (14.9 mL, 29.7 mmol) was added drop-wise. The reaction mixture was stirred at −40° C. for 40 min., upon which time 0.5 M methyl 4-(4-fluorophenyl)-1-(1H-imidazole-1-carbonyl)piperidine-4-carboxylate solution in THF (64.9 mL) was added rapidly. The cooling bath was removed and immediately heated to 40° C. for 1 h, upon which time the solution was cooled to 0° C. and quenched with aq. sat. NaHCO₃ solution (50 mL). The resulting heterogeneous mixture was diluted with DCM (150 mL) and stirred vigorously until partitioning of the white aluminum salts and the organic layer occurred. The resulting mixture was filtered and concentrated under reduced pressure to yield the crude product. The crude product was purified via silica gel chromatography (EtOAc:heptane, 0:100 to 100:0). Desired fractions were combined and concentrated under reduced pressure to yield (1S,3S,4S)-4-(1-(6-fluoro-3-(((1r,4S)-4-isopropoxycyclohexyl)methyl)-2-methyl-1H-indole-1-carbonyl)-4-(4-fluorophenyl)piperidine-4-carboxamido)-3-methylcyclohexane-1-carboxylic acid (15.1 g) as a colorless foam. ¹H NMR (400 MHz, DMSO-d₆) δ 7.46 (dtd, J=12.0, 6.4, 2.7 Hz, 3H), 7.31-7.15 (m, 3H), 7.05-6.89 (m, 1H), 4.11-3.45 (m, 6H), 3.42-3.16 (m, 3H), 2.55 (s, 2H), 2.44 (d, J=13.6 Hz, 1H), 2.29 (d, J=4.9 Hz, 3H), 2.11 (s, 1H), 1.87 (s, 3H), 1.69 (s, 2H), 1.48 (s, 1H), 1.27 (s, 1H), 1.04 (dd, J=6.1, 2.4 Hz, 10H). MS m/z 567.3 [M+H]⁺.

Step b: To (1S,3S,4S)-4-(1-(6-fluoro-3-(((1r,4S)-4-isopropoxycyclohexyl)methyl)-2-methyl-1H-indole-1-carbonyl)-4-(4-fluorophenyl)piperidine-4-carboxamido)-3-methylcyclohexane-1-carboxylic acid (10.9 g, 19.2 mmol) in iPrOH (48.1 mL) and THF (48.1 mL) at RT exposed to air, was added aq. 1 N NaOH solution (77 mL, 77 mmol). The reaction mixture was capped and stirred at 65° C. for 4 h, upon which time it was cooled to RT and left to stir overnight. The resulting mixture was acidified with formic acid (2.95 mL, 77 mmol) and diluted with DCM (100 mL) and aq. sat. NaCl solution, and stirred vigorously. The layers were separated and the organic layer was passed through a phase separator and the filtrate concentrated under reduced pressure to yield 1-(6-fluoro-3-(((1r,4r)-4-isopropoxycyclohexyl)methyl)-2-methyl-1H-indole-1-carbonyl)-4-(4-fluorophenyl)piperidine-4-carboxylic acid (10.6 g, crude) as an off-white solid. MS m/z 553.7 [M+H]⁺.

Step c: To 1-(6-fluoro-3-(((1r,4r)-4-isopropoxycyclohexyl)methyl)-2-methyl-1H-indole-1-carbonyl)-4-(4-fluorophenyl)piperidine-4-carboxylic acid (15.5 g, 28.0 mmol) in CH₃CN (100 mL) at RT, were added ethyl (1S,3S,4S)-4-amino-3-methylcyclohexane-1-carboxylate hydrochloride salt (6.20 g, 28.0 mmol) and HATU (13.8 g, 36.4 mmol). This mixture was stirred for 15 min., upon which time DIPEA (24.4 mL, 140 mmol) was added slowly. The resulting mixture was stirred at RT for 1 h. The reaction mixture was concentrated under reduced pressure, diluted with EtOAc, and washed with aq. 1 N HCl solution, aq. sat. NaHCO₃ solution, followed by aq. sat. NaCl solution. The organic layer was separated and dried over Na₂SO₄, filtered, and concentrated under reduced pressure to yield the crude product. The crude product was purified via silica gel chromatography (EtOAc:heptane, 0:100 to 50:50). Desired fractions were combined and concentrated under reduced pressure to yield ethyl (1S,3S,4S)-4-(1-(6-fluoro-3-(((1r,4S)-4-isopropoxycyclohexyl)methyl)-2-methyl-1H-indole-1-carbonyl)-4-(4-fluorophenyl)piperidine-4-carboxamido)-3-methylcyclohexane-1-carboxylate (19.3 g) as a white solid. ¹H NMR (400 MHz, DCM-d₂) δ 7.42-7.30 (m, 3H), 7.16-7.03 (m, 2H), 7.01-6.82 (m, 2H), 4.88 (dd, J=17.2, 8.9 Hz, 1H), 4.06 (qd, J=7.1, 2.5 Hz, 2H), 3.72-3.31 (m, 5H), 3.22 (ddt, J=14.6, 10.1, 4.2 Hz, 1H), 2.54-1.72 (m, 18H), 1.62-1.10 (m, 13H), 1.00-0.85 (m, 4H), 0.70 (t, J=6.4 Hz, 3H). MS m/z 720.3 [M+H]⁺.

Step d: To ethyl (1S,3S,4S)-4-(1-(6-fluoro-3-(((1r,4S)-4-isopropoxycyclohexyl)methyl)-2-methyl-1H-indole-1-carbonyl)-4-(4-fluorophenyl)piperidine-4-carboxamido)-3-methylcyclohexane-1-carboxylate (19.3 g, 26.8 mmol) in THF (80 mL) and MeOH (40 mL) at RT, was added aq. 2 N NaOH solution (40.2 mL, 80 mmol). The reaction mixture was stirred at RT overnight, upon which time it was partially concentrated under reduced pressure and purified via reverse phase C18 chromatography (CH₃CN:H₂O with 0.1% NH₄OH, 10:90 to 50:50). Desired fractions were collected and partially concentrated (30° C. bath temperature). The resulting solution was lyophilized to yield (1S,3S,4S)-4-(1-(6-fluoro-3-(((1r,4S)-4-isopropoxycyclohexyl)methyl)-2-methyl-1H-indole-1-carbonyl)-4-(4-fluorophenyl)piperidine-4-carboxamido)-3-methylcyclohexane-1-carboxylic acid (sodium salt, 16.0 g) as a white powder. ¹H NMR (400 MHz, DMSO-d₆) δ 7.51-7.36 (m, 3H), 7.28 (dd, J=18.1, 8.7 Hz, 1H), 7.22-7.09 (m, 3H), 6.94 (qd, J=9.3, 2.4 Hz, 1H), 3.84-3.40 (m, 3H), 3.40-3.14 (m, 6H), 2.71-2.52 (m, 2H), 2.26 (d, J=4.0 Hz, 3H), 2.04-1.51 (m, 10H), 1.51-1.41 (m, 1H), 1.28 (qd, J=10.7, 5.1 Hz, 1H), 1.22-0.85 (m, 13H), 0.56 (dd, J=11.2, 6.4 Hz, 3H). HRMS for C₄₀H₅₂F₂N₃O₅: mass calculated 692.3870 [M+H]⁺; mass observed 692.3900 [M+H]⁺. Potency (μM): biochemical qualified AC₅₀: 3.3E-04; NanoBiT qualified absolute AC₅₀: 0.10; cell proliferation qualified AC₅₀: 0.11.

The following compounds of table 8 were synthesized using the above procedure or modifications to the above procedure using the corresponding functionalized piperidine, indole intermediate, and amine. The protonated carboxylate can be obtained directly when formic acid is used to neutralize the crude sodium carboxylate salt prior to purification.

TABLE 8

| Example | | | Potency |
|---|---|---|---|
| ID | Structure | Analytical data | (μM) |
| 1a | 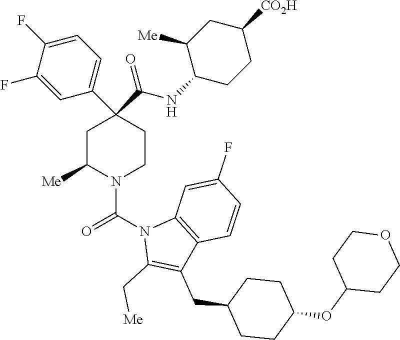 [SMILES: CCc1c(C[C@H]2CC[C@H](OC3CCOCC3)CC2)c2ccc(F)cc2n1C(=O)N1CC[C@@](C(=O)N[C@H]2CC[C@H](C(=O)O)C[C@@H]2C)(c2ccc(F)c(F)c2)C[C@@H]1C] | ¹H NMR (400 MHz, DMSO-d₆) δ 12.02 (s, 1H), 7.49 (td, J = 8.5, 5.4 Hz, 1H), 7.42 − 7.25 (m, 5H), 7.03 − 6.89 (m, 1H), 3.83 − 3.72 (m, 2H), 3.57 (td, J = 9.2, 4.4 Hz, 1H), 3.34 (s, 2H), 3.28 − 3.08 (m, 3H), 2.92 − 2.70 (m, 2H), 2.10 (d, J = 12.5 Hz, 2H), 2.08 − 1.95 (m, 1H), 1.94 − 1.71 (m, 6H), 1.63 (d, J = 25.6 Hz, 4H), 1.47 (s, 3H), 1.39 − 1.20 (m, 10H), 1.06 (m, J = 7.7 Hz, 9H), 0.65 (dd, J = 9.5, 6.4 Hz, 3H). MS m/z 780.3 [M + H]⁺ | Biochemical qualified AC₅₀: <2.8E−04 NanoBIT qualified absolute AC₅₀: 0.16 Cell proliferation qualified AC₅₀: 0.010 |
| 1b | 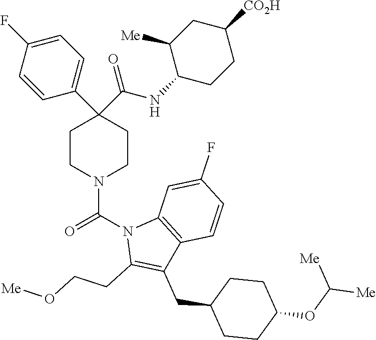 [SMILES: COCCc1c(C[C@H]2CC[C@H](OC(C)C)CC2)c2ccc(F)cc2n1C(=O)N1CCC(C(=O)N[C@H]2CC[C@H](C(=O)O)C[C@@H]2C)(c2ccc(F)cc2)CC1] | ¹H NMR (400 MHz, DMSO-d₆) δ 7.72 (dd, J = 25.0, 8.7 Hz, 1H), 7.50 (dd, J = 8.6, 5.5 Hz, 1H), 7.45 − 7.32 (m, 2H), 7.25 − 6.89 (m, 4H), 3.95 (t, J = 11.5 Hz, 1H), 3.84 − 3.53 (m, 3H), 3.45 (d, J = 5.9 Hz, 4H), 3.14 (d, J = 25.0 Hz, 8H), 2.54 (d, J = 2.6 Hz, 2H), 2.32 (s, 1H), 2.03 − 1.39 (m, 11H), 1.30 − 1.14 (m, 2H), 1.02 (dd, J = 6.1, 1.1 Hz, 11H). Sodium salt MS m/z 756.9 [M + H]⁺ | Biochemical qualified AC₅₀: <2.8E−04 NanoBiT qualified absolute AC₅₀: 0.180 Cell proliferation qualified AC₅₀: 0.062 |
| 1c | 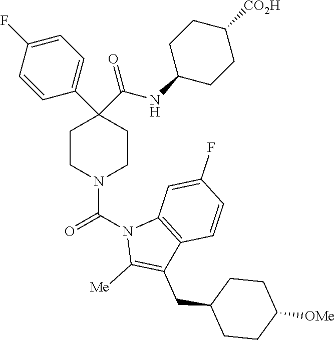 [SMILES: CO[C@H]1CC[C@H](Cc2c(C)n(C(=O)N3CCC(C(=O)N[C@H]4CC[C@H](C(=O)O)CC4)(c4ccc(F)cc4)CC3)c3cc(F)ccc23)CC1] | ¹H NMR (400 MHz, DMSO-d₆) δ 0.97 − 0.99 (m, 3H), 1.20 (s, 3H), 1.25 − 1.32 (m, 2H), 1.45 − 1.50 (m, 2H), 1.63 (m, 5H), 1.93 (m, 5H), 2.01 − 2.10 (m, 1H), 2.20 − 2.25 (m, 3H), 2.45 − 2.48 (m, 1H), 2.50 − 2.55 (m, 2H), 3.01 − 3.07 (m, 3H), 3.14 − 3.20 (m, 5H), 3.52 − 3.68 (m, 2H), 6.95 − 6.99 (m, 1H), 7.11 − 7.15 (m, 3H), 7.32 − 7.39 (m, 3H), 7.44 − 7.48 (m, 1H) ppm, 12.06 (br s, 1H). MS m/z 650.5 [M + H]⁺ | Biochemical qualified AC₅₀: 1.3E−03 NanoBiT qualified absolute AC₅₀: 0.39 Cell proliferation qualified AC₅₀: 0.94 |
| 1d | 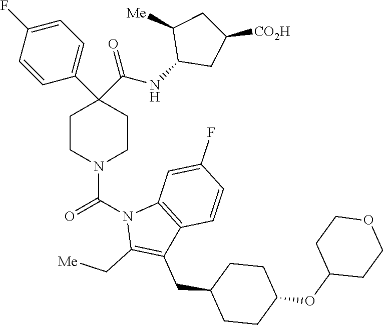 [SMILES: CCc1c(C[C@H]2CC[C@H](OC3CCOCC3)CC2)c2ccc(F)cc2n1C(=O)N1CCC(C(=O)N[C@H]2C[C@H](C(=O)O)C[C@@H]2C)(c2ccc(F)cc2)CC1] | ¹H NMR (400 MHz, DMSO-d₆) δ 7.64 − 7.37 (m, 4H), 7.23 (dq, J = 17.0, 8.7 Hz, 3H), 7.01 (m, J = 8.9 Hz, 1H), 4.12 − 3.51 (m, 5H), 3.27 (s, 4H), 2.79 (s, 4H), 2.55 (s, 6H), 2.06 (d, J = 11.2 Hz, 3H), 1.93 (s, 2H), 1.89 − 1.67 (m, 5H), 1.58 (dd, J = 22.7, 11.7 Hz, 2H), 1.35 (dd, J = 23.4, 11.8 Hz, 3H), 1.12 (d, J = 7.8 Hz, 7H), 0.83 (dd, J = 16.7, 6.5 Hz, 3H). Sodium salt MS m/z 734.4 [M + H]⁺ | Biochemical qualified AC₅₀: <2.8E−04 NanoBiT qualified absolute AC₅₀: 0.30 Cell proliferation qualified AC₅₀. 0.11 |
| 1e | 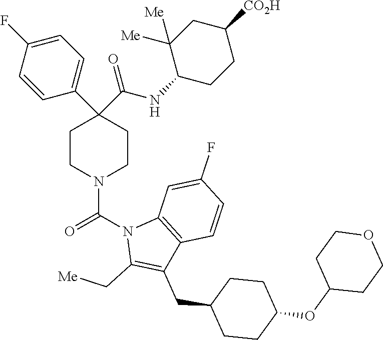 [SMILES: CCc1c(C[C@H]2CC[C@H](OC3CCOCC3)CC2)c2ccc(F)cc2n1C(=O)N1CCC(C(=O)N[C@H]2CC[C@H](C(=O)O)CC2(C)C)(c2ccc(F)cc2)CC1] | ¹H NMR (400 MHz, DMSO-d₆) δ 7.80 − 7.54 (m, 3H), 7.46 − 7.27 (m, 3H), 7.15 (m, J = 10.1 Hz, 1H), 6.97 (dd, J = 44.9, 9.1 Hz, 1H), 4.27 − 3.59 (m, 10H), 2.89 (d, J = 25.7 Hz, 5H), 2.36 − 1.80 (m, 11H), 1.73 − 1.46 (m, 6H), 1.46 − 1.19 (m, 10H), 0.84 − 0.70 (m, 6H). Sodium salt MS m/z 762.8 [M + H]⁺ | Biochemical qualified AC₅₀. <2.8E−04 NanoBIT qualified absolute AC₅₀: 0.019 Cell proliferation qualified AC₅₀. 0.070 |
| 1f | 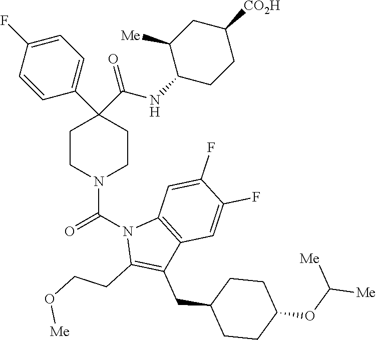 [SMILES: COCCc1c(C[C@H]2CC[C@H](OC(C)C)CC2)c2cc(F)c(F)cc2n1C(=O)N1CCC(C(=O)N[C@H]2CC[C@H](C(=O)O)C[C@@H]2C)(c2ccc(F)cc2)CC1] | ¹H NMR (400 MHz, DMSO-d₆) δ 12.06 (s, 1H), 7.54 (dd, J = 11.0, 7.9 Hz, 1H), 7.50 − 7.27 (m, 4H), 7.18 (dt, J = 14.5, 8.9 Hz, 2H), 3.65 (pd, J = 6.1, 2.3 Hz, 4H), 3.42 (m, J = 4.5 Hz, 3H), 3.12 (d, J = 40.6 Hz, 9H), 2.74 − 2.53 (m, 3H), 2.34 (s, 1H), 2.13 (dt, J = 11.6, 6.0 Hz, 2H), 1.85 (s, 4H), 1.60 (d, J = 30.9 Hz, 3H), 1.53 − 1.19 (m, 4H), 1.03 (dd, J = 6.1, 1.8 Hz, 10H), 0.59 (d, J = 6.8 Hz, 3H). MS m/z 754.8 [M + H]⁺ | Biochemical qualified AC₅₀: <2.8E−04 NanoBiT qualified absolute AC₅₀: 0.14 Cell proliferation qualified AC₅₀: 0.052 |
| 1g | 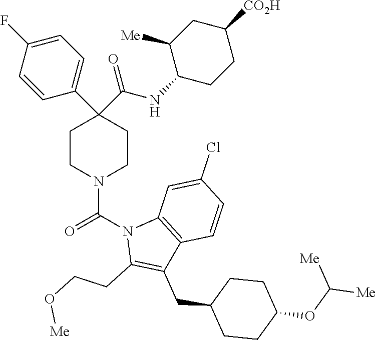 [SMILES: COCCc1c(C[C@H]2CC[C@H](OC(C)C)CC2)c2ccc(Cl)cc2n1C(=O)N1CCC(C(=O)N[C@H]2CC[C@H](C(=O)O)C[C@@H]2C)(c2ccc(F)cc2)CC1] | ¹H NMR (400 MHz, DMSO-d₆) δ 12.06 (s, 2H), 7.52 (d, J = 8.4 Hz, 1H), 7.48 − 7.27 (m, 4H), 7.25 − 7.09 (m, 3H), 3.64 (ddd, J = 11.5, 6.8, 3.7 Hz, 3H), 3.43 (d, J = 5.4 Hz, 2H), 3.24 (d, J = 56.2 Hz, 10H), 2.54 (s, 4H), 2.33 (s, 2H), 2.20 − 1.93 (m, 2H), 1.85 (s, 4H), 1.63 (s, 3H), 1.43 (d, J = 39.5 Hz, 2H), 1.26 (d, J = 20.3 Hz, 2H), 1.19 − 0.92 (m, 9H), 0.61 (dd, J = 15.1, 6.4 Hz, 2H). MS m/z 753.1 [M + H]⁺ | Biochemical qualified AC₅₀: <2.8E−04 NanoBIT qualified absolute AC₅₀: 0.21 Cell proliferation qualified AC₅₀: 0.093 |
| 1h | 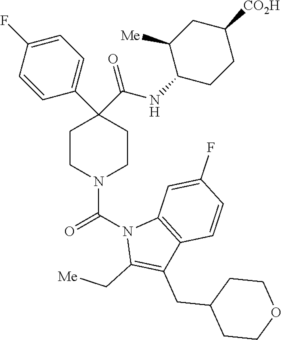 [SMILES: CCc1c(CC2CCOCC2)c2ccc(F)cc2n1C(=O)N1CCC(C(=O)N[C@H]2CC[C@H](C(=O)O)C[C@@H]2C)(c2ccc(F)cc2)CC1] | ¹H NMR (400 MHz, DMSO-d₆) δ 11.98 (s, 1H), 7.45 (dd, J = 8.6, 5.4 Hz, 1H), 7.40 − 7.21 (m, 3H), 7.18 − 6.99 (m, 3H), 6.89 (qd, J = 9.5, 2.3 Hz, 1H), 4.42 − 3.28 (m, 4H), 3.26 − 3.06 (m, 5H), 2.79 − 2.45 (m, 5H), 2.42 (s, 1H), 2.15 − 1.57 (m, 6H), 1.57 − 1.37 (m, 3H), 1.10 (dtd, J = 76.4, 7.8, 3.8 Hz, 9H), 0.52 (t, J = 8.1 Hz, 3H). MS m/z 650.8 [M + H]⁺ | Biochemical qualified AC₅₀: <2.8E−04 NanoBiT qualified absolute AC₅₀: 0.055 Cell proliferation qualified AC₅₀: 0.016 |
| 1i | 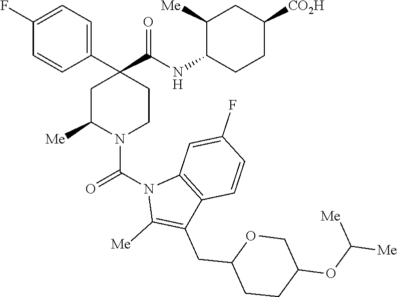 [SMILES: Cc1c(CC2CCC(OC(C)C)CO2)c2ccc(F)cc2n1C(=O)N1CC[C@@](C(=O)N[C@H]2CC[C@H](C(=O)O)C[C@@H]2C)(c2ccc(F)cc2)C[C@@H]1C] | ¹H NMR (400 MHz, MeOD-d₄) δ 7.58 − 7.38 (m, 3H), 7.27 (d, J = 8.5 Hz, 1H), 7.15 − 6.83 (m, 4H), 4.50 (d, J = 57.8 Hz, 1H), 3.93 (dt, J = 10.5, 3.6 Hz, 1H), 3.73 (pd, J = 6.1, 2.0 Hz, 1H), 3.67 − 3.37 (m, 4H), 3.02 − (t, J = 10.4 Hz, 1H), 2.97 − 2.65 (m, 4H), 2.41 − 2.00 (m, 6H), 2.00 − 1.65 (m, 5H), 1.55 − 1.02 (m, 16H), 0.71 (t, J = 6.5 Hz, 3H). Sodium salt Single trans isomer MS m/z 708.4 [M + H]⁺ | Biochemical qualified AC₅₀: <2.8E−04 NanoBiT qualified absolute AC₅₀: 0.031 Cell proliferation qualified AC₅₀: 0.015 |
| 1j | 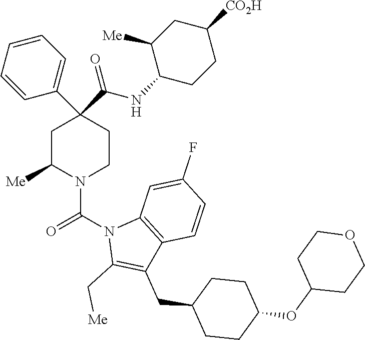 [SMILES: CCc1c(C[C@H]2CC[C@H](OC3CCOCC3)CC2)c2ccc(F)cc2n1C(=O)N1CC[C@@](C(=O)N[C@H]2CC[C@H](C(=O)O)C[C@@H]2C)(c2ccccc2)C[C@@H]1C] | ¹H NMR (400 MHz, DMSO-d₆) δ 12.02 (s, 1H), 7.49 (td, J = 8.5, 5.4 Hz, 1H), 7.40 − 7.18 (m, 6H), 7.16 − 6.83 (m, 2H), 4.59 (s, 1H), 3.85 − 3.68 (m, 2H), 3.57 (td, J = 9.2, 4.4 Hz, 1H), 3.44 − 3.34 (m, 1H), 3.28 − 3.07 (m, 2H), 2.97 − 2.70 (m, 2H), 2.34 (s, 1H), 2.10 (d, J = 12.5 Hz, 2H), 2.05 − 1.93 (m, 1H), 1.93 − 1.53 (m, 10H), 1.47 (s, 3H), 1.39 − 1.14 (m, 10H), 1.06 (m, J = 7.7 Hz, 9H), 0.65 (dd, J = 9.5, 6.4 Hz, 3H). MS m/z 744.05 [M + H]+ | Biochemical qualified AC₅₀: <2.8E−04 NanoBiT qualified absolute AC₅₀: 0.030 Cell proliferation qualified AC₅₀: 0.029 |
| 1k | 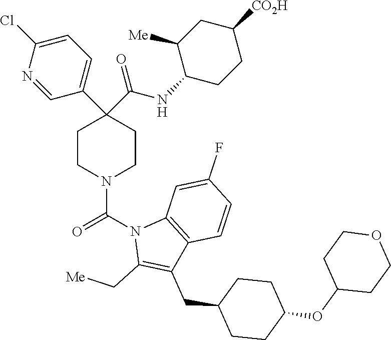 [SMILES: CCc1c(C[C@H]2CC[C@H](OC3CCOCC3)CC2)c2ccc(F)cc2n1C(=O)N1CCC(C(=O)N[C@H]2CC[C@H](C(=O)O)C[C@@H]2C)(c2ccc(Cl)nc2)CC1] | ¹H NMR (400 MHz, DMSO-d₆) δ 8.34 (dd, J = 21.9, 2.7 Hz, 1H), 7.74 (ddd, J = 19.2, 8.5, 2.7 Hz, 1H), 7.56 − 7.33 (m, 3H), 7.10 (ddd, J = 22.9, 10.0, 2.3 Hz, 1H), 6.88 (qd, J = 9.5, 2.3 Hz, 1H), 4.20 − 2.88 (m, 12H), 2.66 (s, 4H), 2.36 (s, 1H), 2.08 (s, 1H), 1.94 − 1.33 (m, 12H), 1.35 − 0.77 (m, 13H), 0.51 (t, J = 7.2 Hz, 3H). Sodium salt MS m/z 765.8 [M + H]⁺ | Biochemical qualified AC₅₀: 4.8E−04 NanoBiT qualified absolute AC₅₀: 0.25 Cell proliferation qualified AC₅₀: 0.033 |
| 1l | 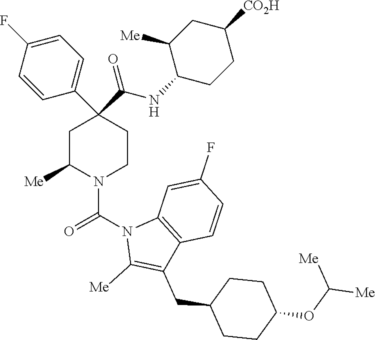 [SMILES: Cc1c(C[C@H]2CC[C@H](OC(C)C)CC2)c2ccc(F)cc2n1C(=O)N1CC[C@@](C(=O)N[C@H]2CC[C@H](C(=O)O)C[C@@H]2C)(c2ccc(F)cc2)C[C@@H]1C] | ¹H NMR (400 MHz, DMSO-d₆) δ 7.40 (dt, J = 8.8, 5.3 Hz, 1H), 7.32 (td, J = 9.3, 5.4 Hz, 2H), 7.22 (d, J = 8.4 Hz, 1H), 7.15 − 6.79 (m, 4H), 4.27 (d, J = 62.2 Hz, 1H), 3.58 (pd, J = 6.1, 1.9 Hz, 1H), 3.22 − 2.97 (m, 3H), 2.68 (q, J = 13.8 Hz, 2H), 2.46 (d, J = 3.4 Hz, 1H), 2.16 (s, 4H), 1.90 − 1.36 (m, 11H), 1.36 − 1.24 (m, 1H), 1.20 (d, J = 7.1 Hz, 1H), 1.17 0.76 (m, 16H), 0.51 (dd, J = 10.1, 6.4 Hz, 3H). Sodium salt MS m/z 706.5 [M + H]⁺ | Biochemical qualified AC₅₀: <2.8E−04 NanoBiT qualified absolute AC₅₀: 0.27 Cell proliferation qualified AC₅₀: 0.018 |

### Example 2

### (1S,3S,4S)-4-(1-(2-ethyl-6-fluoro-3-(((1r,4S)-4-isopropoxycyclohexyl)methyl)-1H-indole-1-carbonyl)-4-(4-fluorophenyl)piperidine-4-carboxamido)-3-methylcyclohexane-1-carboxylic acid

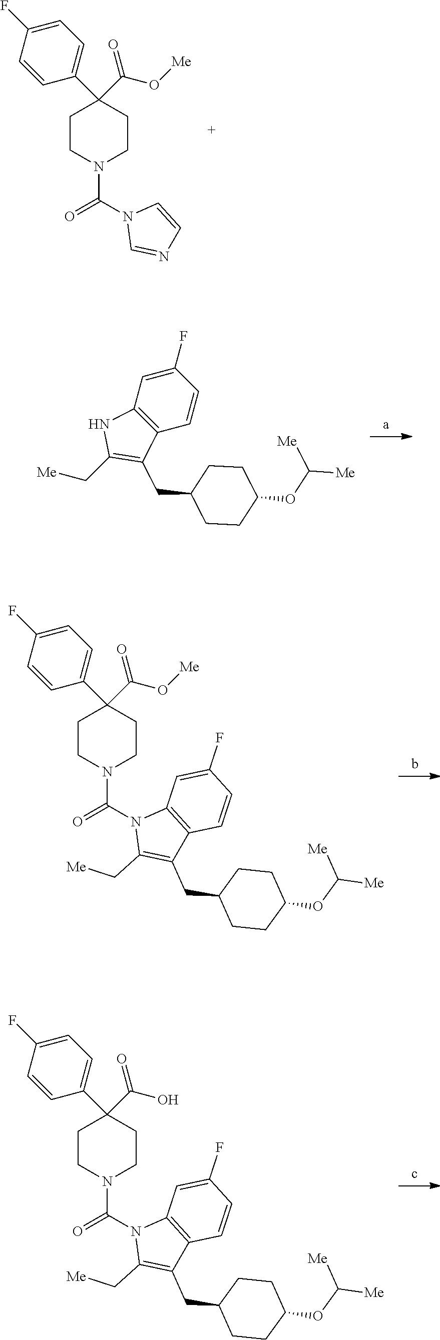 [SMILES: COC(=O)C1(c2ccc(F)cc2)CCN(C(=O)n2ccnc2)CC1 ; CCc1[nH]c2cc(F)ccc2c1C[C@H]1CC[C@H](OC(C)C)CC1 ; CCc1c(C[C@H]2CC[C@H](OC(C)C)CC2)c2ccc(F)cc2n1C(=O)N1CCC(C(=O)OC)(c2ccc(F)cc2)CC1 ; CCc1c(C[C@H]2CC[C@H](OC(C)C)CC2)c2ccc(F)cc2n1C(=O)N1CCC(C(=O)O)(c2ccc(F)cc2)CC1]

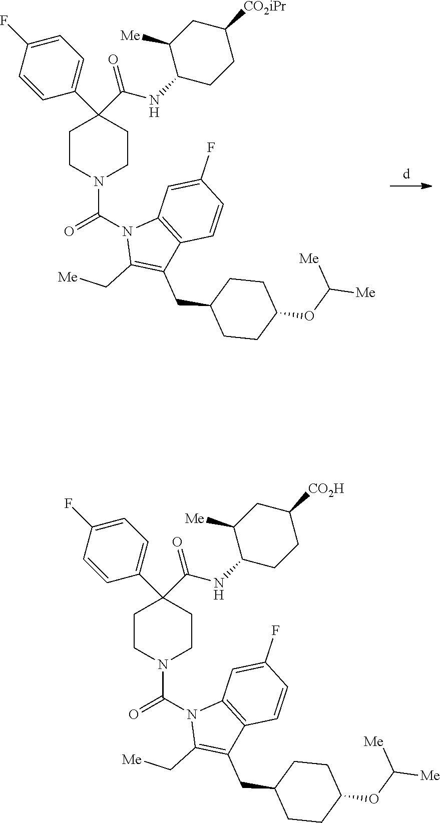

Step a: To 2-ethyl-6-fluoro-3-(((1r,4r)-4-isopropoxycyclohexyl)methyl)-1H-indole in THF (52.9 mL) at −40° C. under N₂, was added 1.0 M LHMDS solution in toluene (31.8 mL, 31.8 mmol) drop-wise. The reaction mixture was stirred for 10 min., upon which time 2.0 M AlMe₃ solution in toluene (15.4 mL, 30.7 mmol) was added drop-wise. The resulting solution was stirred at −40° C. for 40 min., upon which time 0.5 M methyl 4-(4-fluorophenyl)-1-(1H-imidazole-1-carbonyl)piperidine-4-carboxylate solution in THF (42.3 mL) was added drop-wise. The cooling bath was removed and the reaction mixture was immediately heated to 60° C. for 2.5 h, upon which time it was cooled to 0° C. and quenched carefully with aq. sat. NaHCO₃ solution (30 mL). The resulting mixture was diluted with DCM (100 mL) at 0° C. and stirred vigorously until partitioning of the white aluminum salts and the organic layer occurred (˜10 min). The resulting mixture was filtered and concentrated under reduced pressure to yield the crude product. The crude product was purified via silica gel chromatography (EtOAc:heptane, 0:100 to 50:50). Desired fractions were combined and concentrated under reduced pressure to yield methyl 1-(2-ethyl-6-fluoro-3-(((1r,4r)-4-isopropoxycyclohexyl)methyl)-1H-indole-1-carbonyl)-4-(4-fluorophenyl)piperidine-4-carboxylate (8.0 g) as a colorless foam. MS m/z 581.5 [M+H]⁺.

Step b: To methyl 1-(2-ethyl-6-fluoro-3-(((1r,4r)-4-isopropoxycyclohexyl)methyl)-1H-indole-1-carbonyl)-4-(4-fluorophenyl)piperidine-4-carboxylate (8.0 g, 13.8 mmol) in MeOH (26 mL) and THF (36 mL) at RT, was added aq. 1 N NaOH solution (27.6 mL). The reaction mixture was capped and stirred at 60° C. for 5 h, upon which time the reaction mixture was cooled to RT. The reaction mixture was diluted with DCM and aq. sat. NaCl solution while stirring vigorously. This mixture was passed through a phase separator and concentrated under reduced pressure to yield 1-(2-ethyl-6-fluoro-3-(((1r,4r)-4-isopropoxycyclohexyl)methyl)-1H-indole-1-carbonyl)-4-(4-fluorophenyl)piperidine-4-carboxylic acid (sodium salt, 8.13 g, crude) as a white solid. MS m/z 567.7 [M+H]⁺.

Step c: To 1-(2-ethyl-6-fluoro-3-(((1r,4r)-4-isopropoxycyclohexyl)methyl)-1H-indole-1-carbonyl)-4-(4-fluorophenyl)piperidine-4-carboxylic acid (sodium salt, 1.16 g, 1.97 mmol) in DMF (9.84 mL) at RT, was added DIPEA (1.37 mL, 7.87 mmol). Next, isopropyl (1S,3S,4S)-4-amino-3-methylcyclohexane-1-carboxylate hydrochloride salt (0.510 g, 2.16 mmol) was added followed by HATU (1.50 g, 3.93 mmol). The reaction mixture was stirred for 30 min., upon which time the reaction mixture was diluted with EtOAc and poured into a separatory funnel containing aq. sat. NaHCO₃ solution and H₂O (1:1). The mixture was extracted with EtOAc (20 mL×2) and the combined organics were washed with aq. sat. NaCl solution (20 mL×2). The resulting organic extracts were dried over Na₂SO₄, filtered, and concentrated under reduced pressure to yield the crude product. The crude product was purified via silica gel chromatography (EtOAc:heptane, 0:100 to 100:0). Desired fractions were combined and concentrated under reduced pressure to yield isopropyl (1S,3S,4S)-4-(1-(2-ethyl-6-fluoro-3-(((1r,4S)-4-isopropoxycyclohexyl)methyl)-1H-indole-1-carbonyl)-4-(4-fluorophenyl)piperidine-4-carboxamido)-3-methylcyclohexane-1-carboxylate (1.13 g) as a colorless foam. MS m/z 749.0 [M+H]⁺.

Step d: To isopropyl (1S,3S,4S)-4-(1-(2-ethyl-6-fluoro-3-(((1r,4S)-4-isopropoxycyclohexyl)methyl)-1H-indole-1-carbonyl)-4-(4-fluorophenyl)piperidine-4-carboxamido)-3-methylcyclohexane-1-carboxylate (1.13 g, 1.51 mmol) in MeOH (3.78 mL) and THF (3.78 mL) at RT, was added aq. 1 N NaOH solution (3.78 mL, 3.78 mmol). The reaction mixture was stirred for 2 h at 55° C., upon which time it was cooled to RT. Volatile solvent was partially removed under reduced pressure and the resulting mixture was purified directly via reverse phase column chromatography over C18 (CH₃CN:H₂O with 0.1% NH₄OH, 10:90 to 100:0). Desired fractions were combined and lyophilized to yield (1S,3S,4S)-4-(1-(2-ethyl-6-fluoro-3-(((1r,4S)-4-isopropoxycyclohexyl)methyl)-1H-indole-1-carbonyl)-4-(4-fluorophenyl)piperidine-4-carboxamido)-3-methylcyclohexane-1-carboxylic acid (sodium salt, 0.91 g) as a white powder. ¹H NMR (400 MHz, DMSO-d₆) δ 7.53-7.27 (m, 4H), 7.22-7.08 (m, 3H), 6.95 (qd, J=9.3, 2.3 Hz, 1H), 3.88-2.99 (m, 9H), 2.87-2.53 (m, 3H), 2.49 (s, 1H), 2.11 (dddd, J=14.0, 8.4, 6.8, 2.6 Hz, 2H), 1.93-0.95 (m, 26H), 0.59 (dd, J=10.5, 6.5 Hz, 3H). MS m/z 706.3 [M+H]⁺. Potency (μM): biochemical qualified AC₅₀: <2.8E-04; NanoBiT qualified absolute AC₅₀: 0.19; cell proliferation qualified AC₅₀: 0.11.

### Example 3

### (1S,3S,4S)-4-(1-(6-fluoro-3-(((1r,4S)-4-isopropoxycyclohexyl)methyl)-2-(2-methoxyethyl)-1H-indole-1-carbonyl)-4-(4-fluorophenyl)piperidine-4-carboxamido)-3-methylcyclohexane-1-carboxylic acid

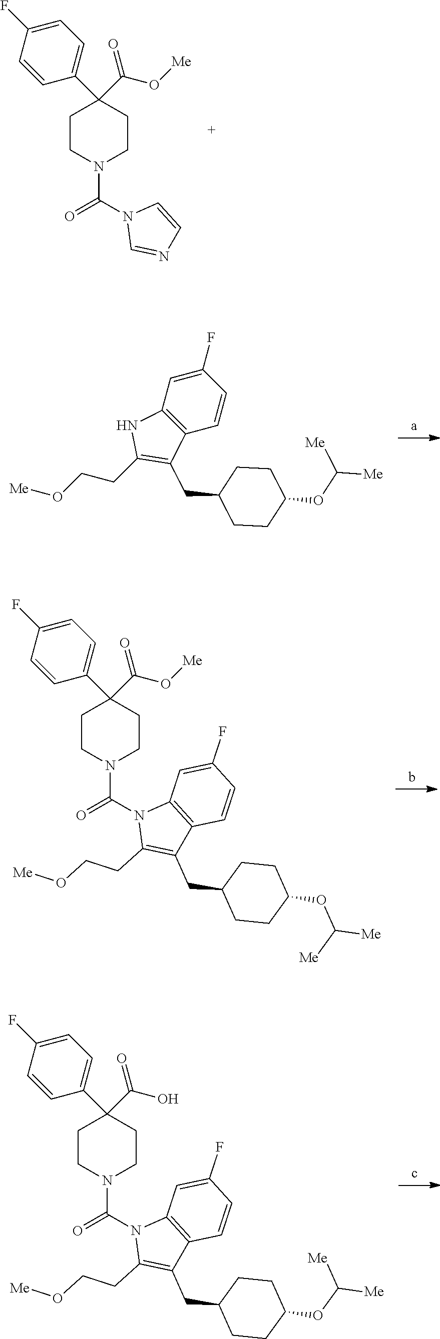 [SMILES: COC(=O)C1(c2ccc(F)cc2)CCN(C(=O)n2ccnc2)CC1 ; COCCc1[nH]c2cc(F)ccc2c1C[C@H]1CC[C@H](OC(C)C)CC1 ; COCCc1c(C[C@H]2CC[C@H](OC(C)C)CC2)c2ccc(F)cc2n1C(=O)N1CCC(C(=O)OC)(c2ccc(F)cc2)CC1 ; COCCc1c(C[C@H]2CC[C@H](OC(C)C)CC2)c2ccc(F)cc2n1C(=O)N1CCC(C(=O)O)(c2ccc(F)cc2)CC1]

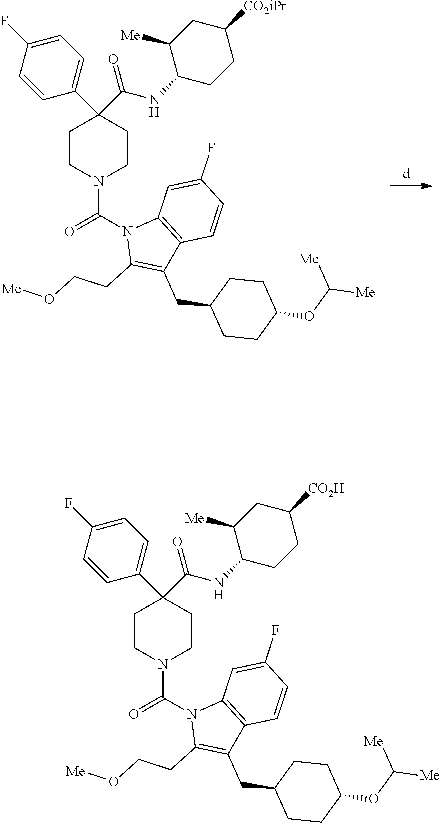

Step a: To 6-fluoro-3-(((1r,4r)-4-isopropoxycyclohexyl)methyl)-2-(2-methoxyethyl)-1H-indole (2.7 g, 5.83 mmol) in THF (29.1 mL) at −40° C. under N₂, was added 1.5 M LHMDS solution in toluene (5.83 mL, 8.74 mmol) drop-wise. The reaction mixture was stirred for 10 min., upon which time 2.0 M AlMe₃ solution in toluene (4.23 mL, 8.45 mmol) was added drop-wise. The reaction mixture was stirred at −40° C. for 40 min., upon which time 0.5 M methyl 4-(4-fluorophenyl)-1-(1H-imidazole-1-carbonyl)piperidine-4-carboxylate solution in THF (16.3 mL, 8.16 mmol) was added drop-wise. The cooling bath was removed and immediately heated to 60° C. for 2.5 h, upon which time it was cooled to 0° C. and quenched carefully with aq. sat. NaHCO₃ (30 mL) solution. The resulting heterogeneous mixture was diluted with DCM (100 mL) at 0° C. and stirred vigorously until partitioning of the white aluminum salts and the organic layer occurred (˜10 min). The resulting mixture was filtered and concentrated under reduced pressure to yield the crude product. The crude product was purified via silica gel chromatography (EtOAc:heptane, 0:100 to 100:0). Desired fractions were combined and concentrated under reduced pressure to yield methyl 1-(6-fluoro-3-(((1r,4r)-4-isopropoxycyclohexyl)methyl)-2-(2-methoxyethyl)-1H-indole-1-carbonyl)-4-(4-fluorophenyl)piperidine-4-carboxylate (4.0 g) as a colorless foam. MS m/z 611.5 [M+H]⁺.

Step b: To methyl 1-(6-fluoro-3-(((1r,4r)-4-isopropoxycyclohexyl)methyl)-2-(2-methoxyethyl)-1H-indole-1-carbonyl)-4-(4-fluorophenyl)piperidine-4-carboxylate (4.0 g, 4.91 mmol) in iPrOH (12.3 mL) and THF (12.3 mL) at RT, was added aq. 1 N NaOH solution (12.3 mL, 12.3 mmol). The reaction mixture was capped and stirred at 40° C. for 14 h, upon which time the reaction mixture was cooled to RT. The reaction mixture was diluted with DCM and aq. sat. NaCl solution, and then neutralized with formic acid while stirring vigorously. This mixture was passed through a phase separator and concentrated under reduced pressure to yield the crude product. The crude product was purified via reverse phase column chromatography over C18 (CH₃CN:H₂O with 0.1% NH₄OH, 0:100 to 100:0). Desired fractions were combined and lyophilized to yield 1-(6-fluoro-3-(((1r,4r)-4-isopropoxycyclohexyl)methyl)-2-(2-methoxyethyl)-1H-indole-1-carbonyl)-4-(4-fluorophenyl)piperidine-4-carboxylic acid (2.41 g) as a white solid. MS m/z 597.7 [M+H]⁺.

Step c: To 1-(6-fluoro-3-(((1r,4r)-4-isopropoxycyclohexyl)methyl)-2-(2-methoxyethyl)-1H-indole-1-carbonyl)-4-(4-fluorophenyl)piperidine-4-carboxylic acid (700 mg, 1.17 mmol) in DMF (5.87 mL) at RT, was added DIPEA (1.02 mL, 5.87 mmol). Next, ethyl (1S,3S,4S)-4-amino-3-methylcyclohexane-1-carboxylate hydrochloride salt (312 mg, 1.41 mmol) was added followed by HATU (558 mg, 1.47 mmol) in a single portion. The reaction mixture was stirred for 30 min., upon which time the reaction mixture was diluted with EtOAc and poured into a separatory funnel containing aq. sat. NaHCO₃ solution and H₂O (1:1). The mixture was extracted with EtOAc (20 mL×2) and combined organics were washed with aq. sat. NaCl solution (20 mL×2). The resulting organics were dried over Na₂SO₄, filtered, and concentrated under reduced pressure to yield the crude product. The crude product was purified via silica gel chromatography (EtOAc:heptane, 0:100 to 100:0). Desired fractions were combined and concentrated under reduced pressure to yield ethyl (1S,3S,4S)-4-(1-(6-fluoro-3-(((1r,4S)-4-isopropoxycyclohexyl)methyl)-2-(2-methoxyethyl)-1H-indole-1-carbonyl)-4-(4-fluorophenyl)piperidine-4-carboxamido)-3-methylcyclohexane-1-carboxylate (896 mg) as a colorless foam. MS m/z 764.9 [M+H]⁺.

Step d: To ethyl (1S,3S,4S)-4-(1-(6-fluoro-3-(((1r,4S)-4-isopropoxycyclohexyl)methyl)-2-(2-methoxyethyl)-1H-indole-1-carbonyl)-4-(4-fluorophenyl)piperidine-4-carboxamido)-3-methylcyclohexane-1-carboxylate (870 mg, 1.14 mmol) in MeOH (4.50 mL) and THF (4.50 mL) at RT, was added aq. 1 N NaOH solution (4.56 mL). The reaction mixture was stirred for 30 min. at 40° C., upon which time the reaction mixture was cooled to RT and formic acid was added until pH 5 was reached. The reaction mixture was concentrated partially and diluted with CH₃CN, H₂O, and MeOH. This mixture was purified via reverse phase column chromatography over C18 (CH₃CN:H₂O with 0.1% NH₄OH, 0:100 to 100:0). Desired fractions were combined and lyophilized to yield (1S,3S,4S)-4-(1-(6-fluoro-3-(((1r,4S)-4-isopropoxycyclohexyl)methyl)-2-(2-methoxyethyl)-1H-indole-1-carbonyl)-4-(4-fluorophenyl)piperidine-4-carboxamido)-3-methylcyclohexane-1-carboxylic acid (610 mg) as a white powder. ¹H NMR (400 MHz, DMSO-d₆) δ 11.58 (s, 1H), 7.54-7.27 (m, 4H), 7.24-7.08 (m, 3H), 6.96 (qd, J=9.0, 2.3 Hz, 1H), 3.65 (pd, J=6.1, 2.3 Hz, 2H), 3.47-3.18 (m, 10H), 3.08 (s, 2H), 3.00 (s, 2H), 2.59 (s, 2H), 2.25-1.97 (m, 2H), 1.91-1.00 (m, 23H), 0.60 (t, J=6.5 Hz, 3H). MS m/z 736.9 [M+H]⁺. Potency (μM): biochemical qualified AC₅₀: 5.0E-04; NanoBiT qualified absolute AC₅₀: 0.046; cell proliferation qualified AC₅₀: 0.060.

### Example 4

### (1S,3S,4S)-4-((2S,4S)-1-(6-fluoro-3-((1R,4S)-4-isopropoxycyclohexyl)indoline-1-carbonyl)-4-(4-fluorphenyl)-2-methylpiperidine-4-carboxamido)-3-methylcyclohexane-1-carboxylic acid

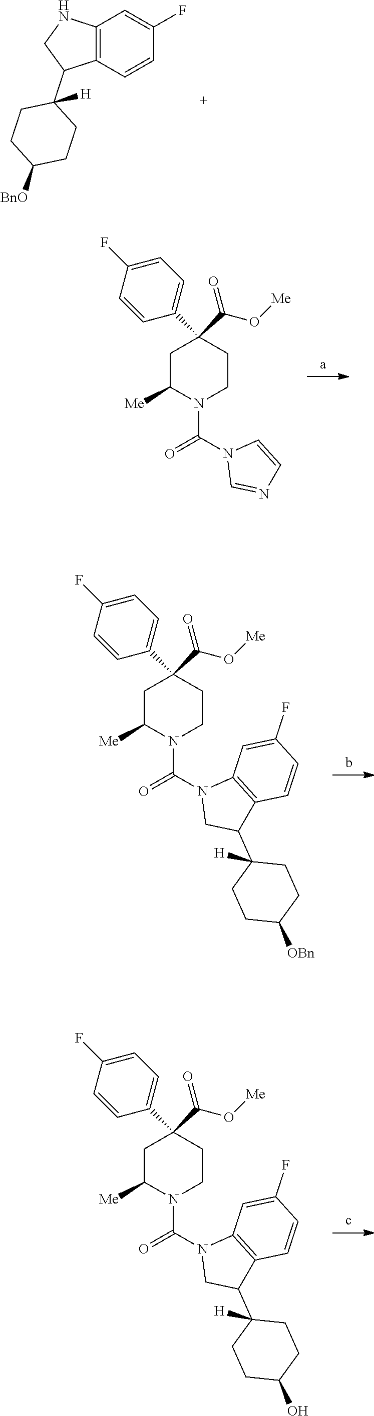 [SMILES: Fc1ccc2c(c1)NCC2[C@H]1CC[C@H](OCc2ccccc2)CC1 ; COC(=O)[C@@]1(c2ccc(F)cc2)CCN(C(=O)n2ccnc2)[C@@H](C)C1 ; COC(=O)[C@@]1(c2ccc(F)cc2)CCN(C(=O)N2CC([C@H]3CC[C@H](OCc4ccccc4)CC3)c3ccc(F)cc32)[C@@H](C)C1 ; COC(=O)[C@@]1(c2ccc(F)cc2)CCN(C(=O)N2CC([C@H]3CC[C@H](O)CC3)c3ccc(F)cc32)[C@@H](C)C1]

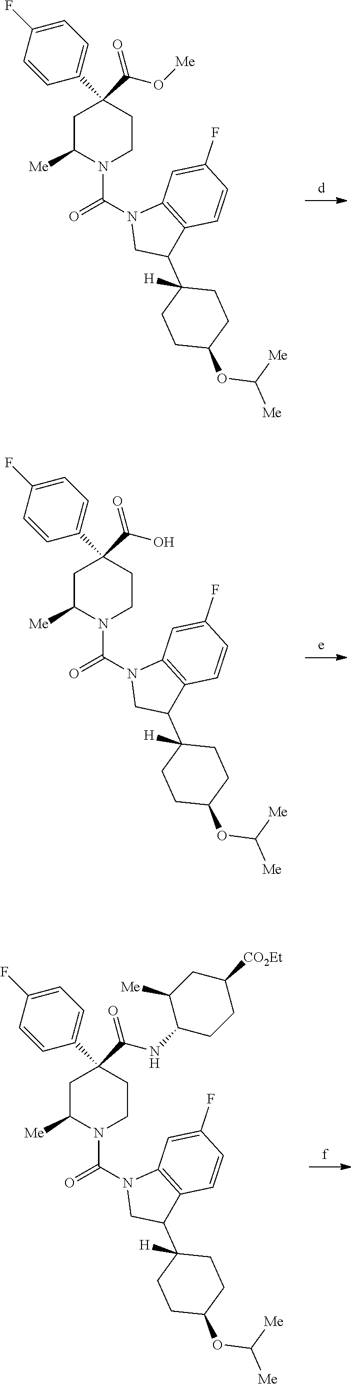 [SMILES: COC(=O)[C@@]1(c2ccc(F)cc2)CCN(C(=O)N2CC([C@H]3CC[C@H](OC(C)C)CC3)c3ccc(F)cc32)[C@@H](C)C1 ; CC(C)O[C@H]1CC[C@H](C2CN(C(=O)N3CC[C@@](C(=O)O)(c4ccc(F)cc4)C[C@@H]3C)c3cc(F)ccc32)CC1 ; CCOC(=O)[C@H]1CC[C@H](NC(=O)[C@@]2(c3ccc(F)cc3)CCN(C(=O)N3CC([C@H]4CC[C@H](OC(C)C)CC4)c4ccc(F)cc43)[C@@H](C)C2)[C@@H](C)C1]

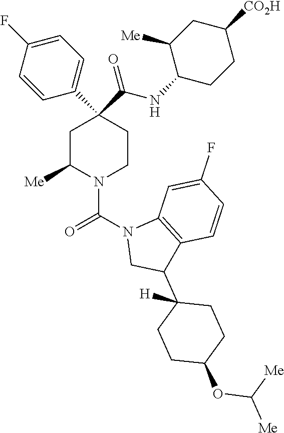 [SMILES: CC(C)O[C@H]1CC[C@H](C2CN(C(=O)N3CC[C@@](C(=O)N[C@H]4CC[C@H](C(=O)O)C[C@@H]4C)(c4ccc(F)cc4)C[C@@H]3C)c3cc(F)ccc32)CC1]

Step a: To 3-((1r,4r)-4-(benzyloxy)cyclohexyl)-6-fluoroindoline (Intermediate 7b, 4.41 g, 12.7 mmol) in THF (30 mL) at −30° C. under N₂, was added 1.0 M LHMDS solution in THF (31 mL, 31.0 mmol) drop-wise. The reaction mixture was stirred for 10 min., upon which time 2.0 M AlMe₃ solution in toluene (14.9 mL, 29.7 mmol) was added drop-wise. The resulting solution was stirred at −30° C. for 40 min., upon which time 1.0 M methyl (2S,4S)-4-(4-fluorophenyl)-1-(1H-imidazole-1-carbonyl)-2-methylpiperidine-4-carboxylate solution in THF (16 mL) was added. The cooling bath was removed and the reaction mixture was immediately heated to 60° C. overnight, upon which time it was cooled to 0° C. and then quenched with Rochelle's salt. The resulting heterogeneous mixture was diluted with EtOAc (50 mL) and stirred vigorously until partitioning of the white aluminum salts and the organic layer occurred. The resulting biphasic mixture was transferred to a separatory funnel to resolve organic and aq. layers. The aq. phase was extracted with EtOAc (3×). The combined organic extracts were concentrated under reduced pressure to yield the crude product. The crude product was purified via silica gel chromatography (EtOAc:heptane, 0:100 to 100:0). Desired fractions were combined and concentrated under reduced pressure to yield methyl (2S,4S)-1-(3-((1r,4S)-4-(benzyloxy)cyclohexyl)-6-fluoroindoline-1-carbonyl)-4-(4-fluorophenyl)-2-methylpiperidine-4-carboxylate (5.33 g) as a white foam. MS m/z 603.5 [M+H]⁺.

Step b: To methyl (2S,4S)-1-(3-((1r,4S)-4-(benzyloxy)cyclohexyl)-6-fluoroindoline-1-carbonyl)-4-(4-fluorophenyl)-2-methylpiperidine-4-carboxylate (5.33 g, 8.84 mmol) in EtOAc (90 mL) at RT under N₂, was added Pd/C (10 wt. %, 4.71 g, 4.42 mmol), and the flask was purged with N₂ (3×).

The reaction mixture was placed under a balloon of H₂ and stirred at RT overnight, upon which time it was filtered through Celite® and rinsed thoroughly with EtOAc. The resulting filtrate was concentrated under reduced pressure and purified via silica gel chromatography (EtOAc:heptane, 15:85 to 100:0). Desired fractions were collected and concentrated under reduced pressure to yield methyl (2S,4S)-1-((S)-6-fluoro-3-((1r,4S)-4-hydroxycyclohexyl)indoline-1-carbonyl)-4-(4-fluorophenyl)-2-methylpiperidine-4-carboxylate (4.50 g) as a white foam. MS m/z 513.4 [M+H]⁺.

Step c: To methyl (2S,4S)-1-((S)-6-fluoro-3-((1r,4S)-4-hydroxycyclohexyl)indoline-1-carbonyl)-4-(4-fluorophenyl)-2-methylpiperidine-4-carboxylate (2.00 g, 3.90 mmol) in DCM (40 mL) at RT, was added acetone (1.13 g, 19.5 mmol) and the reaction mixture was cooled to −78° C. Et₃SiH (2.27 g, 19.5 mmol) was added followed by TMSOTf (3.82 g, 17.2 mmol), upon which time the flask was warmed directly to 0° C. and stirred for 2 h. The reaction mixture was quenched with aq. sat. Na₂CO₃ solution, transferred to a separatory funnel, and the layers were partitioned. The aq. phase was extracted with DCM (3×). The combined organic extracts were passed through a phase separator and concentrated under reduced pressure to yield the crude product. The crude product was purified via silica gel chromatography (EtOAc:heptane, 0:100 to 100:0). Desired fractions were combined and concentrated under reduced pressure to yield methyl (2S,4S)-1-(6-fluoro-3-((1r,4S)-4-isopropoxycyclohexyl)indoline-1-carbonyl)-4-(4-fluorophenyl)-2-methylpiperidine-4-carboxylate (2.12 g) as a white foam. MS m/z 555.2 [M+H]⁺.

Step d: To methyl (2S,4S)-1-(6-fluoro-3-((1r,4S)-4-isopropoxycyclohexyl)indoline-1-carbonyl)-4-(4-fluorophenyl)-2-methylpiperidine-4-carboxylate (2.12 g, 3.82 mmol) in THF (19 mL) and iPrOH (40 mL) at RT, was added aq. 2 N NaOH solution (19.1 mL, 38.2 mmol). The reaction mixture was stirred at RT for 48 h, upon which time the reaction mixture was acidified to pH 3-4 with aq. 1 N HCl solution. The acidified mixture was partially concentrated under reduced pressure, diluted with EtOAc (20 mL), and transferred to a separatory funnel. The layers were separated, and the aq. phase was extracted with EtOAc (3×). The combined organic extracts were washed with aq. sat. NaCl solution and passed through a phase separator. The resulting solution was concentrated under reduced pressure to yield (2S,4S)-1-(6-fluoro-3-((1r,4S)-4-isopropoxycyclohexyl)indoline-1-carbonyl)-4-(4-fluorophenyl)-2-methylpiperidine-4-carboxylic acid (2.06 g, crude) as a white foam. MS m/z 541.2 [M+H]⁺.

Step e: To (2S,4S)-1-(6-fluoro-3-((1r,4S)-4-isopropoxycyclohexyl)indoline-1-carbonyl)-4-(4-fluorophenyl)-2-methylpiperidine-4-carboxylic acid (2.00 g, 3.90 mmol) in CH₃CN (40 mL) at RT, was added ethyl (1S,3S,4S)-4-amino-3-methylcyclohexane-1-carboxylate hydrochloride salt (1.01 g, 4.57 mmol). The flask was purged with N₂ (3×) and DIPEA (2.46 g, 19.1 mmol) was added. The reaction mixture was stirred at RT for 5 min., upon which time HATU (2.90 g, 7.62 mmol) was added in one portion. The reaction mixture was stirred at RT overnight, upon which time it was quenched with aq. 5% NaCl solution (100 mL) and EtOAc (25 mL). The separated aq. phase was extracted with EtOAc (50 mL×3). The combined organic extracts were washed with aq. sat. NaCl solution, passed through a phase separator, and concentrated under reduced pressure to yield the crude product. The crude product was purified via silica gel chromatography (EtOAc:heptane, 0:100 to 65:35). Desired fractions were combined and concentrated under reduced pressure to yield ethyl (1S,3S,4S)-4-((2S,4S)-1-(6-fluoro-3-((1r,4S)-4-isopropoxycyclohexyl)indoline-1-carbonyl)-4-(4-fluorophenyl)-2-methylpiperidine-4-carboxamido)-3-methylcyclohexane-1-carboxylate (2.43 g) as a white foam. MS m/z 708.2 [M+H]⁺.

Step f: To methyl (2S,4S)-1-(6-fluoro-3-((1r,4S)-4-isopropoxycyclohexyl)indoline-1-carbonyl)-4-(4-fluorophenyl)-2-methylpiperidine-4-carboxylate (2.43 g, 3.43 mmol) in THF (17 mL) and iPrOH (17 mL) at RT, was added aq. 2 N NaOH solution (17.2 mL, 34.3 mmol). The reaction mixture was stirred at 50° C. overnight, upon which time it was partially concentrated under reduced pressure to remove volatile solvent. The resulting mixture was purified via reverse phase column chromatography over C18 (CH₃CN:H₂O with 0.1% NH₄OH, 0:100 to 65:35). Desired fractions were collected and lyophilized to yield (1S,3S,4S)-4-((2S,4S)-1-(6-fluoro-3-((1r,4S)-4-isopropoxycyclohexyl)indoline-1-carbonyl)-4-(4-fluorophenyl)-2-methylpiperidine-4-carboxamido)-3-methylcyclohexane-1-carboxylic acid (sodium salt, 2.19 g) as a white solid. ¹H NMR (400 MHz, MeOD-d₄) δ 7.57-7.40 (m, 2H), 7.21-7.02 (m, 4H), 6.78 (dd, J=10.3, 2.4 Hz, 1H), 6.70-6.58 (m, 1H), 4.16 (m, J=5.4 Hz, 1H), 4.02 (dd, J=10.9, 9.1 Hz, 1H), 3.88-3.64 (m, 2H), 3.56-3.43 (m, 2H), 3.40-3.32 (m, 1H), 3.25 (m, J=5.2 Hz, 1H), 3.18-3.07 (m, 1H), 2.79-2.56 (m, 2H), 2.34 (dd, J=13.9, 5.3 Hz, 1H), 2.09 (tt, J=12.2, 3.4 Hz, 1H), 2.03-1.79 (m, 5H), 1.74 (dt, J=12.9, 3.6 Hz, 2H), 1.61-1.36 (m, 4H), 1.29 (d, J=6.9 Hz, 3H), 1.27-1.04 (m, 12H), 0.71 (d, J=6.4 Hz, 3H). HRMS for C₃₉H₅₁F₂N₃O₅: mass calculated 680.3870 [M+H]⁺; mass observed 680.3891 [M+H]⁺. Potency (μM): biochemical qualified AC₅₀: 4.7E-04; NanoBiT qualified absolute AC₅₀: 0.020; cell proliferation qualified AC₅₀: 0.0030.

The following compounds of table 9 were synthesized using the above procedure or modifications to the above procedure using the corresponding ketone. The protonated carboxylate can be obtained directly when formic acid is used to neutralize the crude sodium carboxylate salt prior to purification.

TABLE 9

| Example | | | |
|---|---|---|---|
| ID | Structure | Analytical data | Potency (μM) |
| 4a | 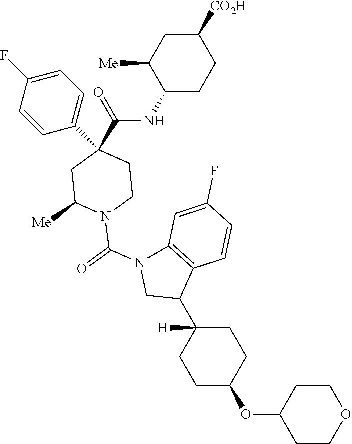 [SMILES: C[C@H]1C[C@@H](C(=O)O)CC[C@@H]1NC(=O)[C@@]1(c2ccc(F)cc2)CCN(C(=O)N2CC([C@H]3CC[C@H](OC4CCOCC4)CC3)c3ccc(F)cc32)[C@@H](C)C1] | ¹H NMR (400 MHz, MeOD-d₄) δ 7.34 (ddd, J = 9.3, 5.5, 2.9 Hz, 2H), 7.11 (d, J = 8.6 Hz, 1H), 7.06 (dd, J = 8.3, 5.6 Hz, 1H), 6.98 (t, J = 8.7 Hz, 2H), 6.68 (dd, J = 10.3, 2.4 Hz, 1H), 6.55 (td, J = 8.7, 2.4 Hz, 1H), 4.10 − 4.02 (m, 1H), 3.92 (dd, J = 10.8, 9.0 Hz, 1H), 3.77 (dt, J = 11.8, 4.3 Hz, 2H), 3.56 (ddd, J = 19.0, 10.0, 3.8 Hz, 2H), 3.33 (tdd, J = 11.8, 8.4, 2.8 Hz, 4H), 3.01 (m, J = 3.8 Hz, 1H), 2.62 − 2.47 (m, 2H), 2.25 (dd, J = 13.9, 5.3 Hz, 1H), 2.08 (ddd, J = 12.2, 8.8, 3.1 Hz, 1H), 1.93 − 1.59 (m, 10H), 1.47 − 1.26 (m, 7H), 1.19 (d, J = 6.9 Hz, 3H), 1.05 (td, J = 22.8, 10.3 Hz, 6H), 0.63 (d, J = 6.4 Hz, 3H). Sodium salt Single stereoisomer HRMS m/z 722.4056 [M + H]⁺ Calculated HRMS m/z 722.3975 | Biochemical qualified AC₅₀. 8.7E−04 NanoBiT qualified absolute AC₅₀: 0.024 Cell proliferation qualified AC₅₀: 0.0040 |
| 4b | 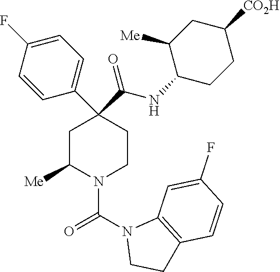 [SMILES: C[C@H]1C[C@@H](C(=O)O)CC[C@@H]1NC(=O)[C@@]1(c2ccc(F)cc2)CCN(C(=O)N2CCc3ccc(F)cc32)[C@@H](C)C1] | ¹H NMR (400 MHz, MeOD-d₄) δ 7.39 − 7.29 (m, 2H), 7.09 − 6.93 (m, 3H), 6.71 (dd, J = 10.3, 2.4 Hz, 1H), 6.51 (ddd, J = 10.5, 8.4, 2.4 Hz, 1H), 4.14 − 4.01 (m, 1H), 3.84 (h, J = 9.9 Hz, 2H), 3.52 − 3.29 (m, 2H), 3.29 − 3.23 (m, 1H), 2.91 (t, J = 8.2 Hz, 2H), 2.66 − 2.45 (m, 2H), 2.20 (dd, J = 14.1, 5.2 Hz, 1H), 2.07 − 1.92 (m, 1H), 1.88 − 1.71 (m, 3H), 1.64 (dd, J = 12.7, 3.6 Hz, 1H), 1.47 − 1.27 (m, 2H), 1.22 (d, J = 7.0 Hz, 3H), 1.15 − 0.96 (m, 2H), 0.62 (d, J = 6.4 Hz, 3H). Sodium salt | Biochemical qualified AC₅₀: 3.2E−04 NanoBiT qualified absolute AC₅₀. 0.26 Cell proliferation qualified AC₅₀: 0.22 |
| | | MS m/z 540.4 [M + H]⁺ | |
| 4c | 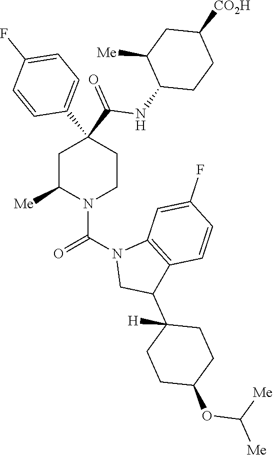 [SMILES: CC(C)O[C@H]1CC[C@H](C2CN(C(=O)N3CC[C@@](C(=O)N[C@H]4CC[C@H](C(=O)O)C[C@@H]4C)(c4ccc(F)cc4)C[C@@H]3C)c3cc(F)ccc32)CC1] | ¹H NMR (400 MHz, DMSO) δ 12.08 (s, 1H), 7.40 (ddd, J = 8.8, 5.4, 2.6 Hz, 2H), 7.27 (d, J = 8.4 Hz, 1H), 7.21 − 7.07 (m, 3H), 6.76 (dd, J = 10.7, 2.5 Hz, 1H), 6.66 (ddd, J = 9.2, 8.2, 2.5 Hz, 1H), 4.07 − 3.94 (m, 1H), 3.94 − 3.81 (m, 1H), 3.72 − 3.55 (m, 2H), 3.20 (ddt, J = 18.9, 8.8, 3.2 Hz, 5H), 2.77 − 2.56 (m, 2H), 2.23 − 2.02 (m, 2H), 2.02 − 1.69 (m, 5H), 1.70 − 1.41 (m, 4H), 1.41 − 0.93 (m, 17H), 0.62 (d, J = 6.4 Hz, 3H). Single stereoisomer Derived from intermediate 7a MS m/z 680.9 [M + H]⁺ | Biochemical qualified AC₅₀: 0.0039 NanoBiT qualified absolute AC₅₀: 1.2 Cell proliferation qualified AC₅₀: 0.41 |

### Example 5

### (1S,3S,4S)-4-(1-(2-ethyl-6-fluoro-3-(((1r,4S)-4-((tetrahydro-2H-pyran-4-yl)oxy)cyclohexyl)methyl)-1H-indole-1-carbonyl)-4-(4-fluorophenyl)piperidine-4-carboxamido)-3-methylcyclohexane-1-carboxylic acid

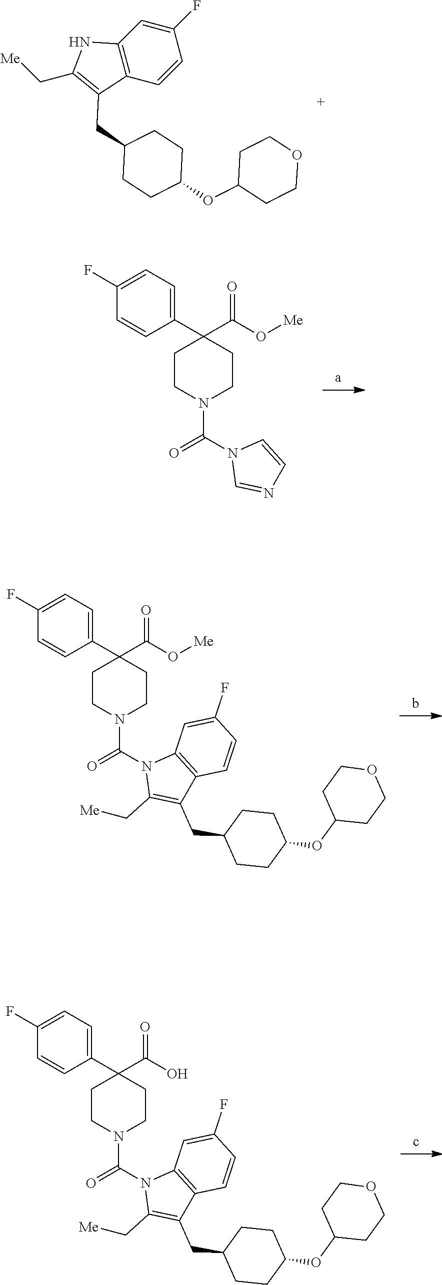 [SMILES: CCc1[nH]c2cc(F)ccc2c1C[C@H]1CC[C@H](OC2CCOCC2)CC1 ; COC(=O)C1(c2ccc(F)cc2)CCN(C(=O)n2ccnc2)CC1 ; CCc1c(C[C@H]2CC[C@H](OC3CCOCC3)CC2)c2ccc(F)cc2n1C(=O)N1CCC(C(=O)OC)(c2ccc(F)cc2)CC1 ; CCc1c(C[C@H]2CC[C@H](OC3CCOCC3)CC2)c2ccc(F)cc2n1C(=O)N1CCC(C(=O)O)(c2ccc(F)cc2)CC1]

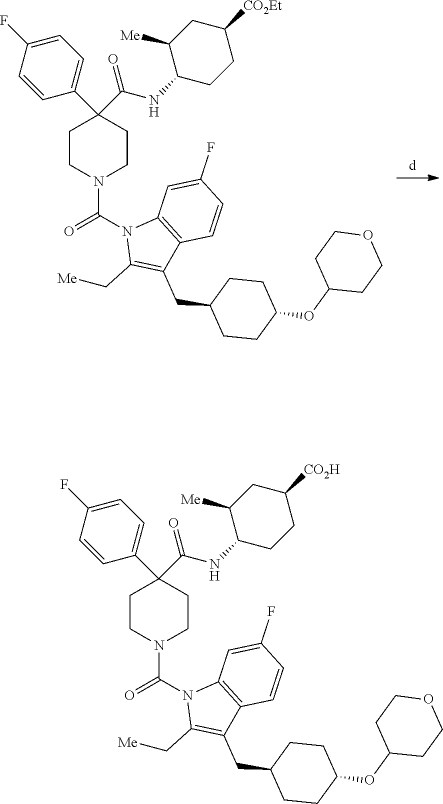 [SMILES: CCOC(=O)[C@H]1CC[C@H](NC(=O)C2(c3ccc(F)cc3)CCN(C(=O)n3c(CC)c(C[C@H]4CC[C@H](OC5CCOCC5)CC4)c4ccc(F)cc43)CC2)[C@@H](C)C1 ; CCc1c(C[C@H]2CC[C@H](OC3CCOCC3)CC2)c2ccc(F)cc2n1C(=O)N1CCC(C(=O)N[C@H]2CC[C@H](C(=O)O)C[C@@H]2C)(c2ccc(F)cc2)CC1]

Step a: To 2-ethyl-6-fluoro-3-(((1 r,4r)-4-((tetrahydro-2H-pyran-4-yl)oxy)cyclohexyl)methyl)-1H-indole (5.02 g, 14.0 mmol) in THF (100 mL) at −30° C. under N₂, was added 1.0 M LHMDS solution in toluene (21.0 mL, 21.0 mmol) drop-wise, and the reaction mixture was stirred for 10 min. Next, 2.0 M AlMe₃ solution in toluene (11.2 mL, 22.3 mmol) was added drop-wise and the reaction mixture was stirred for 30 min. Methyl 4-(4-fluorophenyl)-1-(1H-imidazole-1-carbonyl)piperidine-4-carboxylate (5.09 g, 15.4 mmol) in THF (50 mL) was added and the reaction mixture was stirred at RT for 30 min., upon which time it was heated to 60° C. for 2 h. The reaction mixture was cooled with −30° C. and quenched with Rochelle's salt solution. The layers were separated and the aq. layer was extracted with DCM. The combined organic extracts were passed through a phase separator and concentrated under reduced pressure to yield the crude product. The crude product was purified via silica gel chromatography (EtOAc:heptane, 0:100 to 100:0). Desired fractions were combined and concentrated under reduced pressure to yield methyl 1-(2-ethyl-6-fluoro-3-(((1R,4R)-4-((tetrahydro-2H-pyran-4-yl)oxy)cyclohexyl)methyl)-1H-indole-1-carbonyl)-4-(4-fluorophenyl)piperidine-4-carboxylate (8.1 g) as a glassy solid. ¹H NMR (400 MHz, DCM-d₂) δ 7.44-7.30 (m, 3H), 7.13-7.00 (m, 2H), 6.99-6.84 (m, 2H), 3.91-3.82 (m, 2H), 3.68 (d, J=21.2 Hz, 3H), 3.61-3.51 (m, 1H), 3.42-3.21 (m, 5H), 2.88-2.48 (m, 6H), 2.06 (d, J=12.0 Hz, 1H), 1.97-1.70 (m, 7H), 1.62-1.39 (m, 4H), 1.34-1.20 (m, 2H), 1.19-1.05 (m, 6H). MS m/z 623.3 [M+H]⁺.

Step b: To methyl 1-(2-ethyl-6-fluoro-3-(((1R,4R)-4-((tetrahydro-2H-pyran-4-yl)oxy)cyclohexyl)methyl)-1H-indole-1-carbonyl)-4-(4-fluorophenyl)piperidine-4-carboxylate (8.1 g, 13.0 mmol) in MeOH (50 mL) and THF (25.0 mL) at RT, was added aq. 2 N NaOH solution (32.5 mL, 65.0 mmol). The resulting solution was heated to 70° C. for 2 h, upon which time the reaction mixture was concentrated under reduced pressure and then diluted with EtOAc (200 mL). The mixture was cooled to 0° C. and aq. 1 N HCl solution (130 mL, 130 mmol) and aq. sat. NaCl solution (70 mL) were added until a pH of 2-3 was reached. The separated aq. fraction was extracted with EtOAc. The combined organics were concentrated under reduced pressure to yield 1-(2-ethyl-6-fluoro-3-(((1R,4R)-4-((tetrahydro-2H-pyran-4-yl)oxy)cyclohexyl)methyl)-1H-indole-1-carbonyl)-4-(4-fluorophenyl)piperidine-4-carboxylic acid (7.9 g, crude) as a white foam solid. MS m/z 609.2 [M+H]⁺.

Step c: To 1-(2-ethyl-6-fluoro-3-(((1R,4R)-4-((tetrahydro-2H-pyran-4-yl)oxy)cyclohexyl)methyl)-1H-indole-1-carbonyl)-4-(4-fluorophenyl)piperidine-4-carboxylic acid (7.9 g, 13.0 mmol) in DMF (100 mL) at RT, were added ethyl (1S,3S,4S)-4-amino-3-methylcyclohexane-1-carboxylate (3.17 g, 14.3 mmol) and DIPEA (11.3 mL, 64.9 mmol). The resulting mixture was stirred at RT for 5 min., upon which time HATU (6.42 g, 16.9 mmol) was added. The reaction mixture was stirred at RT for 2 h. Additional ethyl (1S,3S,4S)-4-amino-3-methylcyclohexane-1-carboxylate (0.1 equiv.) was added. The reaction mixture was stirred at RT for 1 h, upon which time the reaction mixture was concentrated under reduced pressure and diluted with EtOAc. The resulting mixture was washed with aq. 1 N HCl solution, aq. sat. NaHCO₃ solution, and aq. sat. NaCl solution. The resulting organic layer was dried over MgSO₄, filtered, and concentrated under reduced pressure to yield the crude product. The crude product was purified via silica gel chromatography (EtOAc:heptane, 0:100 to 100:0). Desired fractions were combined and concentrated under reduced pressure to yield ethyl (1S,3S,4S)-4-(1-(2-ethyl-6-fluoro-3-(((1r,4S)-4-((tetrahydro-2H-pyran-4-yl)oxy)cyclohexyl)methyl)-1H-indole-1-carbonyl)-4-(4-fluorophenyl)piperidine-4-carboxamido)-3-methylcyclohexane-1-carboxylate (9.4 g) as a foamy white solid. MS m/z 776.3 [M+H]⁺.

Step d: To ethyl (1S,3S,4S)-4-(1-(2-ethyl-6-fluoro-3-(((1r,4S)-4-((tetrahydro-2H-pyran-4-yl)oxy)cyclohexyl)methyl)-1H-indole-1-carbonyl)-4-(4-fluorophenyl)piperidine-4-carboxamido)-3-methylcyclohexane-1-carboxylate (9.4 g, 12.1 mmol) in MeOH (100 mL) at RT, was added aq. 2 N NaOH solution (30.3 mL, 60.6 mmol). The reaction mixture was heated to 70° C. for 1 h, upon which time it was cooled to RT and purified directly via reverse phase column chromatography over C18 (CH₃CN:H₂O with 0.1% NH₄OH, 0:100 to 100:0). Desired fractions were combined and lyophilized to yield (1S,3S,4S)-4-(1-(2-ethyl-6-fluoro-3-(((1r,4S)-4-((tetrahydro-2H-pyran-4-yl)oxy)cyclohexyl)methyl)-1H-indole-1-carbonyl)-4-(4-fluorophenyl)piperidine-4-carboxamido)-3-methylcyclohexane-1-carboxylic acid (sodium salt, 9.3 g) as an off white powder. ¹H NMR (400 MHz, DMSO-d₆) δ 7.52-7.08 (m, 7H), 6.95 (qd, J=9.3, 2.3 Hz, 1H), 3.75 (dd, J=10.6, 5.2 Hz, 2H), 3.56 (dtd, J=9.3, 6.0, 3.2 Hz, 1H), 3.41-3.10 (m, 10H), 2.84-2.59 (m, 3H), 2.17-0.81 (m, 27H), 0.56 (dd, J=10.9, 6.2 Hz, 3H). MS m/z 748.6 [M+H]⁺. Potency (μM): biochemical qualified AC₅₀: 7.0E-04; NanoBiT qualified absolute AC₅₀: 0.066; cell proliferation qualified AC₅₀: 0.024.

### Example 6

### (1S,3S,4S)-4-((2S,4S)-1-(2-ethyl-6-fluoro-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxamido)-3-methylcyclohexane-1-carboxylic acid

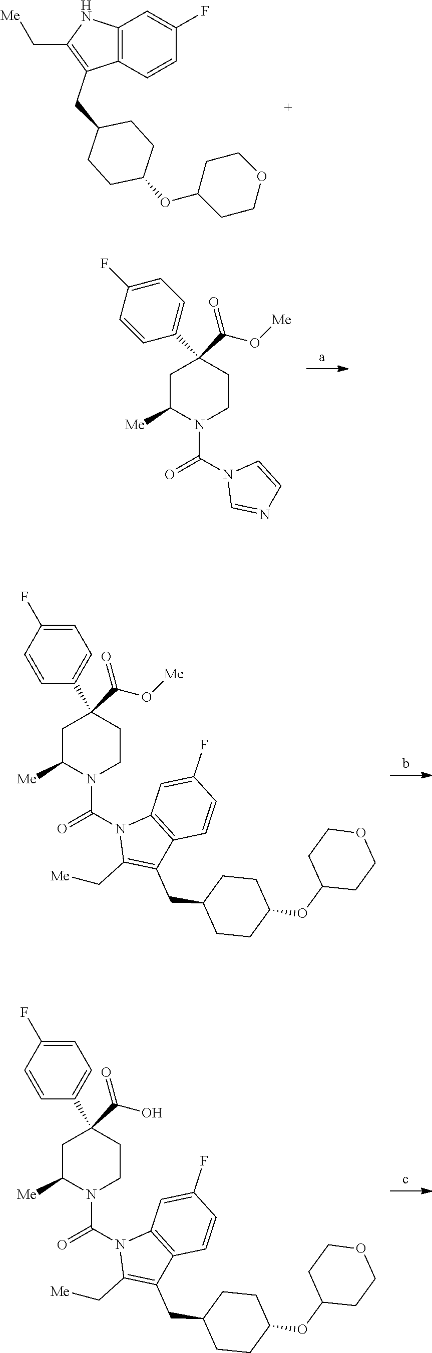 [SMILES: CCc1[nH]c2cc(F)ccc2c1C[C@H]1CC[C@H](OC2CCOCC2)CC1 ; COC(=O)[C@@]1(c2ccc(F)cc2)CCN(C(=O)n2ccnc2)[C@@H](C)C1 ; CCc1c(C[C@H]2CC[C@H](OC3CCOCC3)CC2)c2ccc(F)cc2n1C(=O)N1CC[C@@](C(=O)OC)(c2ccc(F)cc2)C[C@@H]1C ; CCc1c(C[C@H]2CC[C@H](OC3CCOCC3)CC2)c2ccc(F)cc2n1C(=O)N1CC[C@@](C(=O)O)(c2ccc(F)cc2)C[C@@H]1C]

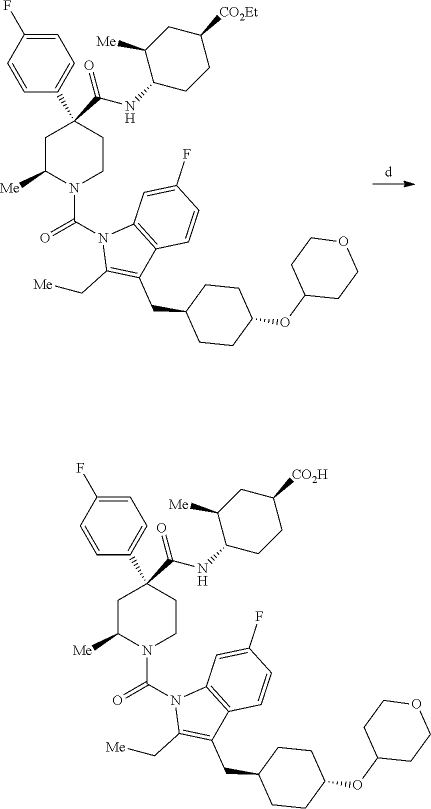 [SMILES: CCOC(=O)[C@H]1CC[C@H](NC(=O)[C@@]2(c3ccc(F)cc3)CCN(C(=O)n3c(CC)c(C[C@H]4CC[C@H](OC5CCOCC5)CC4)c4ccc(F)cc43)[C@@H](C)C2)[C@@H](C)C1 ; CCc1c(C[C@H]2CC[C@H](OC3CCOCC3)CC2)c2ccc(F)cc2n1C(=O)N1CC[C@@](C(=O)N[C@H]2CC[C@H](C(=O)O)C[C@@H]2C)(c2ccc(F)cc2)C[C@@H]1C]

Step a: To 2-ethyl-6-fluoro-3-(((1r,4r)-4-((tetrahydro-2H-pyran-4-yl)oxy)cyclohexyl)methyl)-1H-indole (0.600 g, 1.67 mmol) in THF (20 mL) at −30° C. under N₂, was added 1.0 M LHMDS solution in THF (2.50 mL, 2.50 mmol) drop-wise. The reaction mixture was stirred for 10 min. Next, 2.0 M AlMe₃ solution in toluene (1.34 mL, 2.67 mmol) was added drop-wise and the reaction mixture was stirred for 1 h. Methyl (2S,4S)-4-(4-fluorophenyl)-1-(1H-imidazole-1-carbonyl)-2-methylpiperidine-4-carboxylate (0.703 g, 2.04 mmol) in THF (10 mL) was added and the reaction mixture was heated to 60° C. for 2.5 h. The reaction mixture was cooled to 0° C. and quenched with Rochelle's salt solution. The layers were separated and the aq. layer was extracted with DCM (3×). The combined organic extracts were passed through a phase separator and concentrated under reduced pressure to yield the crude product. The crude product was purified via reverse phase column chromatography over C18 (CH₃CN:H₂O with 0.1% NH₄OH, 10:90 to 100:0). Desired fractions were combined and lyophilized to yield methyl (2S,4S)-1-(2-ethyl-6-fluoro-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxylate (0.696 g) as a glassy solid. MS m/z 637 [M+H]⁺.

Step b: To methyl (2S,4S)-1-(2-ethyl-6-fluoro-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxylate (0.696 g, 1.09 mmol) in THF (7.3 mL) and iPrOH (3.6 mL) at 0° C., was added aq. 1 N NaOH solution (10.9 mL, 10.9 mmol). The reaction mixture was heated to 70° C. MeOH (3 mL) was added and the reaction mixture was cooled to 0° C. and acidified with aq. 1 N HCl solution (12 mL). The reaction mixture was extracted with DCM (3×). The combined organic extracts were passed through a phase separator and concentrated under reduced pressure to yield (2S,4S)-1-(2-ethyl-6-fluoro-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxylic acid (0.681 g, crude) as an off-white foam. MS m/z 623 [M+H]⁺.

Step c: To (2S,4S)-1-(2-ethyl-6-fluoro-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxylic acid (0.681 g, 1.09 mmol) in CH₃CN (10.9 mL) at RT under N₂, were added ethyl (1S,3S,4S)-4-amino-3-methylcyclohexane-1-carboxylate (0.291 g, 1.31 mmol), HATU (0.624 g, 1.64 mmol), and DIPEA (1.14 mL, 6.56 mmol). The reaction mixture was stirred at RT overnight, upon which time the reaction mixture was concentrated under reduced pressure to yield the crude product. The crude product was purified via silica gel chromatography (EtOAc:heptane, 0:100 to 100:0). Desired fractions were combined and concentrated under reduced pressure to yield ethyl (1S,3S,4S)-4-((2S,4S)-1-(2-ethyl-6-fluoro-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxamido)-3-methylcyclohexane-1-carboxylate (740 mg) as a white solid. MS m/z 791.0 [M+H]⁺.

Step d: To ethyl (1S,3S,4S)-4-((2S,4S)-1-(2-ethyl-6-fluoro-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxamido)-3-methylcyclohexane-1-carboxylate (740 mg, 0.937 mmol) in MeOH (3.1 mL) and THF (6.2 mL) at RT, was added aq. 1 N NaOH solution (4.7 mL, 4.7 mmol). The reaction mixture was stirred at 40° C. for 1 h, upon which time it was partially concentrated under reduced pressure to remove volatile solvent. The resulting mixture was purified via reverse phase column chromatography over C18 (CH₃CN:H₂O with 0.1% NH₄OH, 10:90 to 100:0). Desired fractions were combined and lyophilized to yield (1S,3S,4S)-4-((2S,4S)-1-(2-ethyl-6-fluoro-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxamido)-3-methylcyclohexane-1-carboxylic acid (sodium salt, 0.640 g) as a white powder. ¹H NMR (400 MHz, MeOD-d₄) δ 7.50-7.08 (m, 3H), 7.05-6.67 (m, 4H), 4.68-4.37 (m, 1H), 3.89-3.66 (m, 2H), 3.66-3.49 (m, 2H), 3.49-3.27 (m, 5H), 2.92-2.38 (m, 6H), 2.38-2.09 (m, 1H), 2.09-1.21 (m, 19H), 1.21-0.84 (m, 9H), 0.74-0.47 (m, 3H). HRMS for C₄₄H₅₇F₂N₃O₆: mass calculated 762.4288 [M+H]⁺; mass observed 762.4353 [M+H]⁺. Potency (μM): biochemical qualified AC₅₀: 9.8E-04; NanoBiT qualified absolute AC₅₀: 0.072; cell proliferation qualified AC₅₀: 0.023.

### Example 7

### (1S,3S,4S)-4-(1-(3-(((1r,4S)-4-ethoxycyclohexyl)methyl)-6-fluoro-2-methyl-1H-indole-1-carbonyl)-4-(4-fluorophenyl)piperidine-4-carboxamido)-3-methyl cyclohexane-1-carboxylic acid

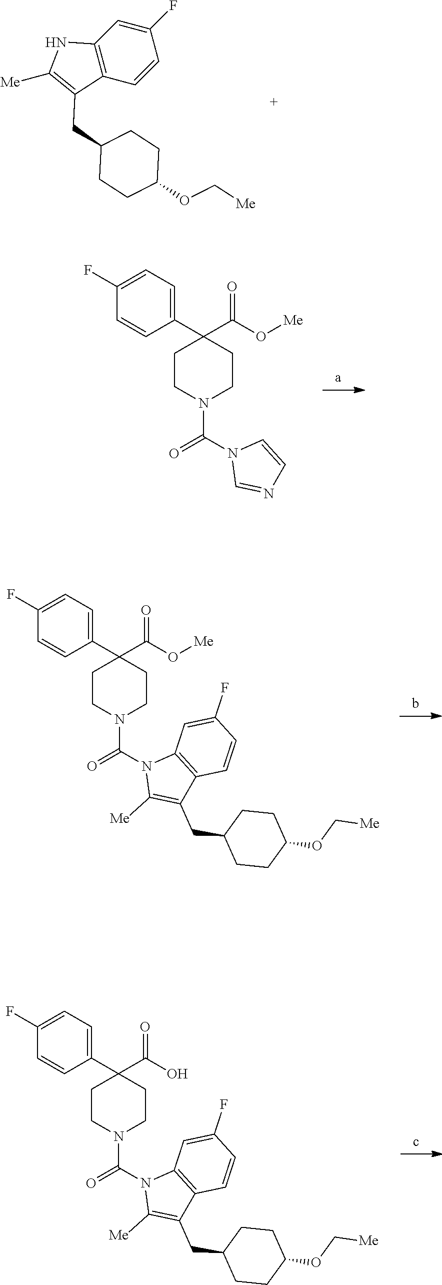 [SMILES: CCO[C@H]1CC[C@H](Cc2c(C)[nH]c3cc(F)ccc23)CC1 ; COC(=O)C1(c2ccc(F)cc2)CCN(C(=O)n2ccnc2)CC1 ; CCO[C@H]1CC[C@H](Cc2c(C)n(C(=O)N3CCC(C(=O)OC)(c4ccc(F)cc4)CC3)c3cc(F)ccc23)CC1 ; CCO[C@H]1CC[C@H](Cc2c(C)n(C(=O)N3CCC(C(=O)O)(c4ccc(F)cc4)CC3)c3cc(F)ccc23)CC1]

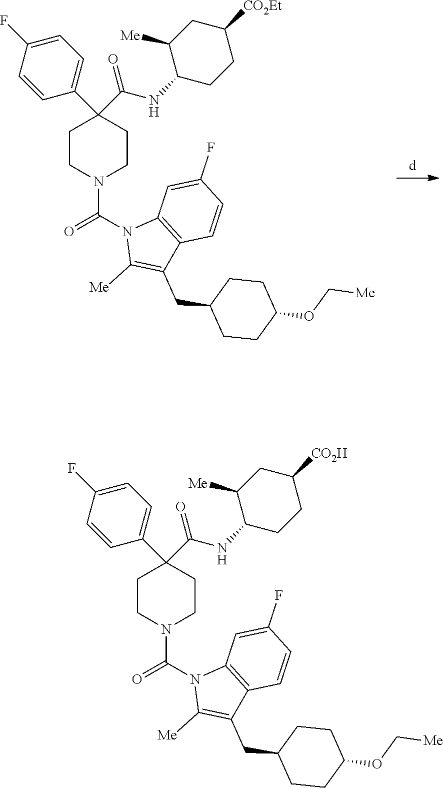 [SMILES: CCOC(=O)[C@H]1CC[C@H](NC(=O)C2(c3ccc(F)cc3)CCN(C(=O)n3c(C)c(C[C@H]4CC[C@H](OCC)CC4)c4ccc(F)cc43)CC2)[C@@H](C)C1 ; CCO[C@H]1CC[C@H](Cc2c(C)n(C(=O)N3CCC(C(=O)N[C@H]4CC[C@H](C(=O)O)C[C@@H]4C)(c4ccc(F)cc4)CC3)c3cc(F)ccc23)CC1]

Step a: To 3-(((1r,4r)-4-ethoxycyclohexyl)methyl)-6-fluoro-2-methyl-1H-indole (3.7 g, 12.8 mmol) in THF (60 mL) at −40° C. under N₂, was added 1.0 M LHMDS solution in toluene (16.0 mL, 16.0 mmol) drop-wise. The reaction mixture was stirred for 10 min., upon which time 2.0 M AlMe₃ solution in toluene (7.67 mL, 15.3 mmol) was added drop-wise. The reaction mixture was stirred at −40° C. for 1 h. Methyl 4-(4-fluorophenyl)-1-(1H-inidazole-1-carbonyl)piperidine-4-carboxylate (5.08 g, 15.3 mmol) in THF (30.7 mL) was added drop-wise and the reaction mixture was stirred for 10 min. The cooling bath was removed and the reaction mixture was heated to 40° C. for 1.5 h, upon which time it was cooled to 0° C. and quenched with aq. sat. NaHCO₃ solution (20 mL). The resulting heterogeneous mixture was diluted with DCM (60 mL) at 0° C. and stirred vigorously until partitioning of the white aluminum salts and the organic layer occurred (˜10 min.). The resulting mixture was filtered and concentrated under reduced pressure to yield the crude product. The crude product was purified via silica gel chromatography (EtOAc:heptane, 5:95 to 100:0). Desired fractions were combined and concentrated under reduced pressure to yield methyl 1-(3-(((1r,4r)-4-ethoxycyclohexyl)methyl)-6-fluoro-2-methyl-1H-indole-1-carbonyl)-4-(4-fluorophenyl)piperidine-4-carboxylate (4.75 g) as a colorless foam. MS m/z 553.5 [M+H]⁺.

Step b: To methyl 1-(3-(((1r,4r)-4-ethoxycyclohexyl)methyl)-6-fluoro-2-methyl-1H-indole-1-carbonyl)-4-(4-fluorophenyl)piperidine-4-carboxylate (4.75 g, 8.59 mmol) in iPrOH (40 mL) and THF (40 mL) at RT, was added aq. 1 N NaOH solution (34.4 mL, 34.4 mmol). The reaction mixture was stirred at 60° C. for 1.5 h. Volatile organics were partially removed under reduced pressure. The mixture was diluted with CHCl₃ (100 mL) and aq. 5% NaCl solution (100 mL), and neutralized with formic acid while stirring vigorously. The resulting organic layer was dried over Na₂SO₄, filtered, and concentrated under reduced pressure to yield 1-(3-(((1r,4r)-4-ethoxycyclohexyl)methyl)-6-fluoro-2-methyl-1H-indole-1-carbonyl)-4-(4-fluorophenyl)piperidine-4-carboxylic acid (4.7 g, crude) as an off-white solid. MS m/z 539.6 [M+H]⁺.

Step c: To 1-(3-(((1r,4r)-4-ethoxycyclohexyl)methyl)-6-fluoro-2-methyl-1H-indole-1-carbonyl)-4-(4-fluorophenyl)piperidine-4-carboxylic acid (2.9 g, 5.38 mmol) in CH₃CN (60 mL) and DMF (6 mL) at RT under N₂, were added ethyl (1S,3S,4S)-4-amino-3-methylcyclohexane-1-carboxylate hydrochloride salt (1.43 g, 6.46 mmol), DIPEA (5.64 mL, 32.3 mmol), and HATU (3.07 g, 8.08 mmol). The reaction mixture was stirred at RT for 2 h. The resulting mixture was diluted with EtOAc (150 mL) and washed with aq. 5% NaCl solution (100 mL×2). The combined organic extracts were dried over Na₂SO₄, filtered, and concentrated under reduced pressure to yield the crude product. The crude product was purified via silica gel chromatography (EtOAc:heptane, 5:95 to 100:0). Desired fractions were combined and concentrated under reduced pressure to yield ethyl (1S,3S,4S)-4-(1-(3-(((1r,4S)-4-ethoxycyclohexyl)methyl)-6-fluoro-2-methyl-1H-indole-1-carbonyl)-4-(4-fluorophenyl)piperidine-4-carboxamido)-3-methylcyclohexane-1-carboxylate (3.67 g) as a white solid. MS m/z 706.7 [M+H]⁺.

Step d: To ethyl (1S,3S,4S)-4-(1-(3-(((1r,4S)-4-ethoxycyclohexyl)methyl)-6-fluoro-2-methyl-1H-indole-1-carbonyl)-4-(4-fluorophenyl)piperidine-4-carboxamido)-3-methylcyclohexane-1-carboxylate (3.67 g, 5.20 mmol) in MeOH (80 mL) and THF (80 mL) at RT, was added aq. 1 N NaOH solution (31.2 mL, 31.2 mmol). The reaction mixture was stirred at 40° C. for 24 h, upon which time it was cooled to RT and neutralized with formic acid. Volatile organics were partially removed under reduced pressure and the remaining crude was diluted with CHCl₃ (150 mL) and washed with aq. 5% NaCl solution (120 mL). The resulting organic layer was dried over Na₂SO₄, filtered, and concentrated under reduced pressure to yield the crude product. This crude product was suspended in EtOH and concentrated under reduced pressure to yield (1S,3S,4S)-4-(1-(3-(((1r,4S)-4-ethoxycyclohexyl)methyl)-6-fluoro-2-methyl-1H-indole-1-carbonyl)-4-(4-fluorophenyl)piperidine-4-carboxamido)-3-methylcyclohexane-1-carboxylic acid (3.50 g) as a white solid. ¹H NMR (400 MHz, DMSO-d₆) δ 12.09 (s, 1H), 7.52-7.29 (m, 4H), 7.25-7.10 (m, 3H), 6.96 (qd, J=9.4, 2.3 Hz, 1H), 3.80-3.53 (m, 1H), 3.52-3.19 (m, 8H), 3.15 (s, 1H), 2.60-2.49 (m, 2H), 2.27 (s, 3H), 2.17-2.09 (m, 1H), 1.99-1.59 (m, 9H), 1.53-1.11 (m, 3H), 1.23-0.97 (m, 9H), 0.61 (dd, J=10.6, 6.4 Hz, 3H). MS m/z 678.8 [M+H]⁺. Potency (μM): biochemical qualified AC₅₀: 3.3E-04; NanoBiT qualified absolute AC₅₀: 0.080; cell proliferation qualified AC₅₀: 0.096.

### Example 8

### (1S,3S,4S)-4-((2S,4S)-1-(3-((trans-5-ethoxytetrahydro-2H-pyran-2-yl)methyl)-6-fluoro-2-methyl-1H-indole-1-carbonyl)-4-(4-fluorophenyl)-2-methylpiperidine-4-carboxamido)-3-methylcyclohexane-1-carboxylic acid (single trans isomer)

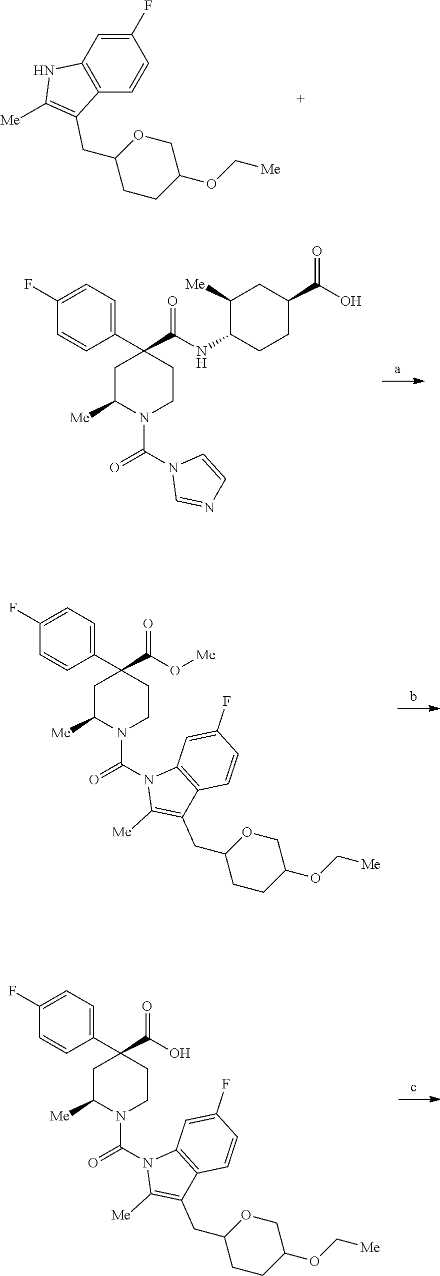 [SMILES: CCOC1CCC(Cc2c(C)[nH]c3cc(F)ccc23)OC1 ; C[C@H]1C[C@@H](C(=O)O)CC[C@@H]1NC(=O)[C@@]1(c2ccc(F)cc2)CCN(C(=O)n2ccnc2)[C@@H](C)C1 ; CCOC1CCC(Cc2c(C)n(C(=O)N3CC[C@@](C(=O)OC)(c4ccc(F)cc4)C[C@@H]3C)c3cc(F)ccc23)OC1 ; CCOC1CCC(Cc2c(C)n(C(=O)N3CC[C@@](C(=O)O)(c4ccc(F)cc4)C[C@@H]3C)c3cc(F)ccc23)OC1]

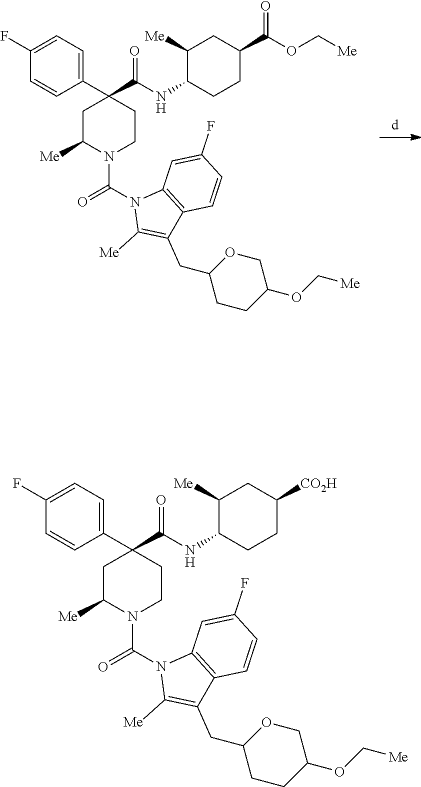 [SMILES: CCOC(=O)[C@H]1CC[C@H](NC(=O)[C@@]2(c3ccc(F)cc3)CCN(C(=O)n3c(C)c(CC4CCC(OCC)CO4)c4ccc(F)cc43)[C@@H](C)C2)[C@@H](C)C1 ; CCOC1CCC(Cc2c(C)n(C(=O)N3CC[C@@](C(=O)N[C@H]4CC[C@H](C(=O)O)C[C@@H]4C)(c4ccc(F)cc4)C[C@@H]3C)c3cc(F)ccc23)OC1]

Step a: To trans-3-((5-ethoxytetrahydro-2H-pyran-2-yl)methyl)-6-fluoro-2-methyl-1H-indole (Intermediate 25b, 550 mg, 1.89 mmol) in THF (10 mL) at −40° C., was added 1.0 M LHMDS solution in THF (2.83 mL, 2.83 mmol) drop-wise. The resulting mixture was stirred for 10 min., upon which time 2.0 M AlMe₃ solution in toluene (1.51 mL, 3.02 mmol) was added drop-wise. The resulting mixture was stirred at −40° C. for 40 min., then a solution of methyl (2S,4S)-4-(4-fluorophenyl)-1-(1H-imidazole-1-carbonyl)-2-methylpiperidine-4-carboxylate (700 mg, 2.03 mmol) in THF (10 mL) was added drop-wise. The cooling bath was removed and the mixture was heated to 70° C. for 2 h. The mixture was cooled to 0° C., quenched with aq. sat. Rochelle's salt solution, and extracted with EtOAc (50 mL×3). The organic layer was dried over Na₂SO₄, filtered, and concentrated under reduced pressure to yield the crude product. The crude product was purified via silica gel chromatography (EtOAc:heptane, 0:100 to 100:0). Desired fractions were combined and concentrated under reduced pressure to yield methyl (2S,4S)-1-(3-((trans-5-ethoxytetrahydro-2H-pyran-2-yl)methyl)-6-fluoro-2-methyl-1H-indole-1-carbonyl)-4-(4-fluorophenyl)-2-methylpiperidine-4-carboxylate (929 mg) as a beige solid. MS m/z 569 [M+H]⁺.

Step b: To methyl (2S,4S)-1-(3-((trans-5-ethoxytetrahydro-2H-pyran-2-yl)methyl)-6-fluoro-2-methyl-1H-indole-1-carbonyl)-4-(4-fluorophenyl)-2-methylpiperidine-4-carboxylate (0.926 g, 1.63 mmol) in THF (15 mL) and MeOH (5.0 mL) at RT, was added aq. 1 N NaOH solution (16.3 mL, 16.3 mmol). The reaction mixture was heated to 60° C. for 5 h. The reaction mixture was cooled to RT, acidified with aq. 1 N HCl solution (24.4 mL, 24.4 mmol), and extracted with DCM. The organic layer was passed through a phase separator and concentrated under reduced pressure to yield (2S,4S)-1-(3-((trans-5-ethoxytetrahydro-2H-pyran-2-yl)methyl)-6-fluoro-2-methyl-1H-indole-1-carbonyl)-4-(4-fluorophenyl)-2-methylpiperidine-4-carboxylic acid (990 mg, crude) as a beige solid. MS m/z 554 [M+H]⁺.

Step c: To (2S,4S)-1-(3-((trans-5-ethoxytetrahydro-2H-pyran-2-yl)methyl)-6-fluoro-2-methyl-1H-indole-1-carbonyl)-4-(4-fluorophenyl)-2-methylpiperidine-4-carboxylic acid (990 mg, 1.61 mmol) in DCM (20 mL) at RT, were added ethyl (1S,3S,4S)-4-amino-3-methylcyclohexane-1-carboxylate (356 mg, 1.61 mmol), HATU (1.36 g, 3.57 mmol), and DIPEA (1.40 mL, 8.03 mmol). The reaction mixture was stirred at RT for 1 h, upon which time it was partially concentrated and purified via silica gel chromatography (EtOAc:heptane, 0:100 to 60:40). Desired fractions were combined and concentrated under reduced pressure to yield ethyl (1S,3S,4S)-4-((2S,4S)-1-(3-((trans-5-ethoxytetrahydro-2H-pyran-2-yl)methyl)-6-fluoro-2-methyl-1H-indole-1-carbonyl)-4-(4-fluorophenyl)-2-methylpiperidine-4-carboxamido)-3-methylcyclohexane-1-carboxylate (1.1 g) as a beige solid. MS m/z 722 [M+H]⁺.

Step d: To ethyl (1S,3S,4S)-4-((2S,4S)-1-(3-((trans-5-ethoxytetrahydro-2H-pyran-2-yl)methyl)-6-fluoro-2-methyl-1H-indole-1-carbonyl)-4-(4-fluorophenyl)-2-methylpiperidine-4-carboxamido)-3-methylcyclohexane-1-carboxylate (1.1 g, 1.52 mmol) in THF (15 mL) and MeOH (5.0 mL) at RT, was added aq. 1 N NaOH solution (7.62 mL, 7.62 mmol). The reaction mixture was stirred at RT for 18 h, upon which time it was partially concentrated under reduced pressure and purified via reverse phase column chromatography over C18 (CH₃CN:H₂O with 0.1% NH₄OH, 0:100 to 30:70). Desired fractions were combined and lyophilized to yield (1S,3S,4S)-4-((2S,4S)-1-(3-((trans-5-ethoxytetrahydro-2H-pyran-2-yl)methyl)-6-fluoro-2-methyl-1H-indole-1-carbonyl)-4-(4-fluorophenyl)-2-methylpiperidine-4-carboxamido)-3-methylcyclohexane-1-carboxylic acid (sodium salt, 988 mg) as a white solid. Note: The absolute stereochemistry of the trans-pyran fragment was not determined. ¹H NMR (400 MHz, MeOD-d₄) δ 7.47 (ddt, J=20.0, 11.1, 5.5 Hz, 3H), 7.26 (d, J=9.9 Hz, 1H), 7.09 (td, J=8.7, 5.8 Hz, 2H), 7.05-6.84 (m, 2H), 4.50 (d, J=51.6 Hz, 1H), 4.07-3.97 (m, 1H), 3.63-3.46 (m, 5H), 3.06 (t, J=10.4 Hz, 1H), 2.96-2.64 (m, 4H), 2.32 (dd, J=9.9, 4.0 Hz, 4H), 2.18-2.03 (m, 2H), 1.88 (s, 2H), 1.74 (s, 3H), 1.49-1.00 (m, 13H), 0.71 (m, J=7.7 Hz, 3H). HRMS for C₃₉H₄₉F₂N₃O₆: mass calculated 694.3662 [M+H]⁺; mass observed 694.3706 [M+H]⁺. Potency (μM): biochemical qualified AC₅₀: <2.8E-04; NanoBiT qualified absolute AC₅₀: 0.031; cell proliferation qualified AC₅₀: 0.015.

The following compound of table 10 was synthesized using the above procedure or modifications to the above procedure using the corresponding ketone. The protonated carboxylate can be obtained directly when formic acid is used to neutralize the crude sodium carboxylate salt prior to purification.

TABLE 10

| Example | | | |
|---|---|---|---|
| ID | Structure | Analytical data | Potency (μM) |
| 8a | 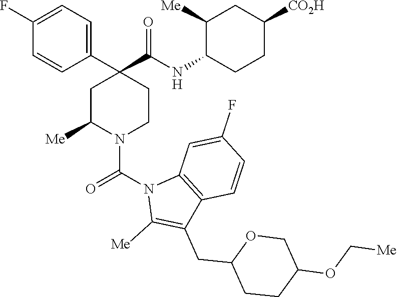 [SMILES: CCOC1CCC(Cc2c(C)n(C(=O)N3CC[C@@](C(=O)N[C@H]4CC[C@H](C(=O)O)C[C@@H]4C)(c4ccc(F)cc4)C[C@@H]3C)c3cc(F)ccc23)OC1] | ¹H NMR (400 MHz, MeOD-d₄) δ 7.53 − 7.25 (m, 4H), 7.13 − 6.84 (m, 4H), 4.49 (d, J = 62.9 Hz, 1H), 4.01 (dtd, J = 8.9, 4.4, 2.1 Hz, 1H), 3.63 − 3.42 (m, 5H), 3.29 − 3.17 (m, 1H), 3.13 − 2.95 (m, 1H), 2.95 − 2.67 (m, 4H), 2.40 − 2.09 (m, 6H), 1.92 (d, J = 13.3 Hz, 2H), 1.89 − 1.63 (m, 3H), 1.46 − 1.26 (m, 8H), 1.14 (td, J = 7.0, 1.4 Hz, 5H), 0.71 (dd, J = 9.9, 6.4 Hz, 3H). Sodium salt Single stereoisomer Derived from intermediate 25a HRMS m/z [M + H]⁺ 694.3663 Calculated HRMS m/z 694.3662 | Biochemical qualified AC₅₀. <2.8E−04 NanoBIT qualified absolute AC₅₀: 0.028 Cell proliferation qualified AC₅₀. 0.013 |

### Example 9

### (1S,3S,4S)-4-((2S,4S)-1-(6-fluoro-2-methyl-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxamido)-3-methylcyclohexane-1-carboxylic acid

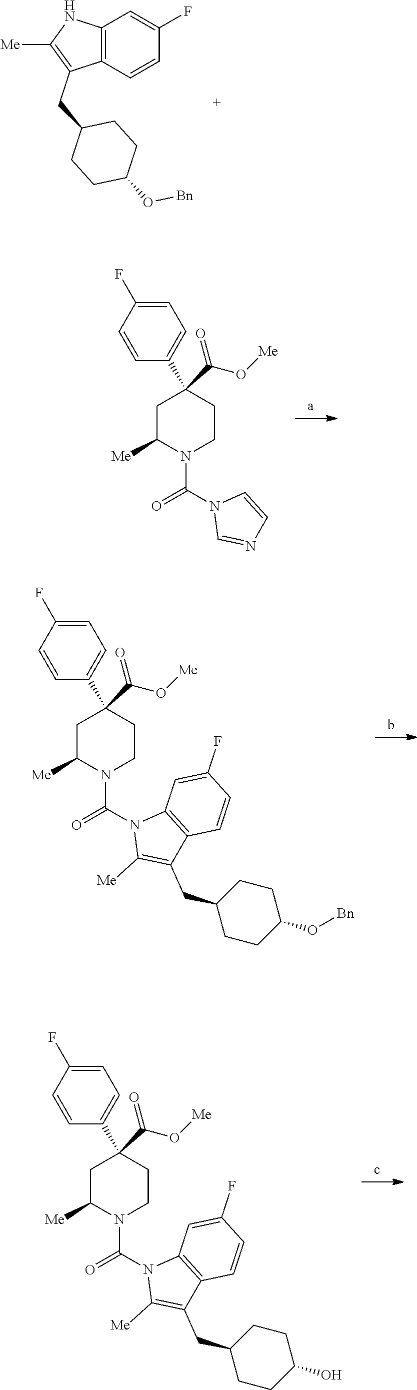 [SMILES: Cc1[nH]c2cc(F)ccc2c1C[C@H]1CC[C@H](OCc2ccccc2)CC1 ; COC(=O)[C@@]1(c2ccc(F)cc2)CCN(C(=O)n2ccnc2)[C@@H](C)C1 ; COC(=O)[C@@]1(c2ccc(F)cc2)CCN(C(=O)n2c(C)c(C[C@H]3CC[C@H](OCc4ccccc4)CC3)c3ccc(F)cc32)[C@@H](C)C1 ; COC(=O)[C@@]1(c2ccc(F)cc2)CCN(C(=O)n2c(C)c(C[C@H]3CC[C@H](O)CC3)c3ccc(F)cc32)[C@@H](C)C1]

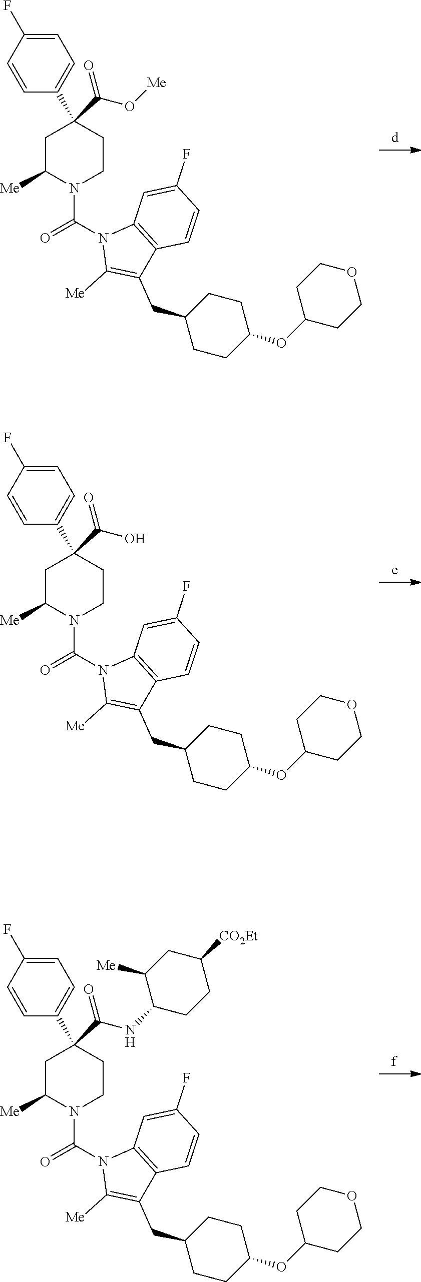 [SMILES: COC(=O)[C@@]1(c2ccc(F)cc2)CCN(C(=O)n2c(C)c(C[C@H]3CC[C@H](OC4CCOCC4)CC3)c3ccc(F)cc32)[C@@H](C)C1 ; Cc1c(C[C@H]2CC[C@H](OC3CCOCC3)CC2)c2ccc(F)cc2n1C(=O)N1CC[C@@](C(=O)O)(c2ccc(F)cc2)C[C@@H]1C ; CCOC(=O)[C@H]1CC[C@H](NC(=O)[C@@]2(c3ccc(F)cc3)CCN(C(=O)n3c(C)c(C[C@H]4CC[C@H](OC5CCOCC5)CC4)c4ccc(F)cc43)[C@@H](C)C2)[C@@H](C)C1]

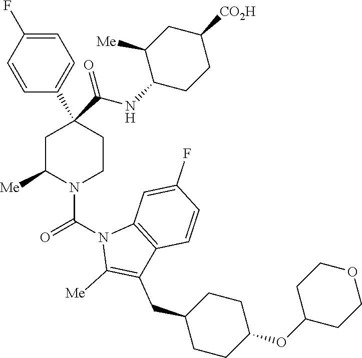 [SMILES: Cc1c(C[C@H]2CC[C@H](OC3CCOCC3)CC2)c2ccc(F)cc2n1C(=O)N1CC[C@@](C(=O)N[C@H]2CC[C@H](C(=O)O)C[C@@H]2C)(c2ccc(F)cc2)C[C@@H]1C]

Step a: To 3-(((1r,4r)-4-(benzyloxy)cyclohexyl)methyl)-6-fluoro-2-methyl-1H-indole (1.34 g, 3.82 mmol) in THF (20 mL) at −20° C. under N₂, was added 1.0 M LHMDS solution in THF (5.10 mL, 5.10 mmol) drop-wise. The reaction mixture was stirred for 10 min. Next, 2.0 M AlMe₃ solution in toluene (2.55 mL, 5.10 mmol) was added drop-wise and the reaction mixture was stirred for 1 h. Methyl (2S,4S)-4-(4-fluorophenyl)-1-(1H-imidazole-1-carbonyl)-2-methylpiperidine-4-carboxylate (1.10 g, 3.19 mmol) in THF (6.0 mL) was added and the reaction mixture was heated to 60° C. for 3.5 h. The reaction mixture was cooled to 0° C. and quenched with Rochelle's salt solution. The resulting mixture was extracted with DCM (25 mL×3). The combined organic extracts were passed through a phase separator and concentrated under reduced pressure to yield the crude product. The crude product was purified via reverse phase column chromatography over C18 (CH₃CN:H₂O with 0.1% NH₄OH, 10:90 to 100:0). Desired fractions were combined and concentrated under reduced pressure to yield methyl (2S,4S)-1-(3-(((1r,4S)-4-(benzyloxy)cyclohexyl)methyl)-6-fluoro-2-methyl-1H-indole-1-carbonyl)-4-(4-fluorophenyl)-2-methylpiperidine-4-carboxylate (1.75 g) as a white foam. MS m/z 629.3 [M+H]⁺.

Step b: To methyl (2S,4S)-1-(3-(((1r,4S)-4-(benzyloxy)cyclohexyl)methyl)-6-fluoro-2-methyl-1H-indole-1-carbonyl)-4-(4-fluorophenyl)-2-methylpiperidine-4-carboxylate (1.75 g, 2.79 mmol) in EtOAc (30 mL) at RT under N₂, was added Pd/C (1.48 g, 10 wt. %, 1.40 mmol) in a single portion. The reaction was stirred under H₂ (1 atm., balloon) overnight. The flask was purged with N₂ and filtered. To the filtrate was added additional Pd/C (1.00 g, 10 wt. %) and the reaction was stirred for 1 h under H₂ (1 atm., balloon). The flask was purged with N₂ and the reaction mixture was filtered through Celite®. The filtrate was concentrated under reduced pressure to yield methyl (2S,4S)-1-(6-fluoro-3-(((1r,4S)-4-hydroxycyclohexyl)methyl)-2-methyl-1H-indole-1-carbonyl)-4-(4-fluorophenyl)-2-methylpiperidine-4-carboxylate (1.50 g, crude) as a white foam. MS m/z 539.2 [M+H]⁺.

Step c: To methyl (2S,4S)-1-(6-fluoro-3-(((1r,4S)-4-hydroxycyclohexyl)methyl)-2-methyl-1H-indole-1-carbonyl)-4-(4-fluorophenyl)-2-methylpiperidine-4-carboxylate (1.50 g, 2.80 mmol) in DCM (30 mL) at RT, were added tetrahydro-4H-pyran-4-one (1.30 mL, 13.9 mmol) and Et₃SiH (2.23 mL, 13.9 mol). The reaction mixture was placed under N₂ and cooled to −78° C., upon which time TMSOTf (2.27 mL, 12.6 mmol) was added drop-wise. The reaction mixture was stirred for 2 min., then stirred at 0° C. for 1 h. The reaction mixture was quenched with aq. sat. NaHCO₃ solution and extracted with DCM (30 mL×3). The organic extracts were washed with aq. sat. NaCl solution and passed through a phase separator. Celite® was added and the solution was concentrated under reduced pressure to yield the crude product. The crude product was purified via silica gel chromatography (EtOAc:heptane, 0:100 to 100:0). Desired fractions were combined and concentrated under reduced pressure to yield methyl (2S,4S)-1-(6-fluoro-2-methyl-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxylate (2.56 g) as a yellow oil. MS m/z 623.3 [M+H]⁺.

Step d: To methyl (2S,4S)-1-(6-fluoro-2-methyl-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxylate (1.74 g, 2.79 mmol) in THF (10 mL) and MeOH (10 mL) at RT, was added aq. 2 N NaOH (14.0 mL, 27.9 mmol). The reaction mixture was heated to 70° C. for 2 h, upon which time it was cooled to RT, acidified with aq. 1 N HCl (28 mL), and extracted with DCM (40 mL×3). The combined organic extracts were passed through a phase separator and concentrated under reduced pressure to yield (2S,4S)-1-(6-fluoro-2-methyl-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxylic acid (2.33 g, crude) as a yellow oil. MS m/z 609.3 [M+H]⁺.

Step e: To (2S,4S)-1-(6-fluoro-2-methyl-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxylic acid (1.70 g, 2.79 mmol) in CH₃CN (90 mL) at RT under N₂, were added ethyl (1S,3S,4S)-4-amino-3-methylcyclohexane-1-carboxylate (0.742 g, 3.35 mmol), DIPEA (2.43 mL, 14.0 mmol), and HATU (2.12 g, 5.58 mmol). The reaction mixture was stirred at RT overnight, upon which time the reaction mixture was concentrated under reduced pressure to yield the crude product. The crude product was purified via reverse phase column chromatography over C18 (CH₃CN:H₂O with 0.1% NH₄OH; 10:90 to 100:0). Desired fractions were combined and concentrated under reduced pressure to yield ethyl (1S,3S,4S)-4-((2S,4S)-1-(6-fluoro-2-methyl-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxamido)-3-methylcyclohexane-1-carboxylate (1.48 g) as a white foam. MS m/z 776.4 [M+H]⁺.

Step f: To ethyl (1S,3S,4S)-4-((2S,4S)-1-(6-fluoro-2-methyl-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxamido)-3-methylcyclohexane-1-carboxylate (1.47 g, 1.9 mmol) in MeOH (9.5 mL) and THF (9.5 mL) at RT under N₂, was added aq. 2 N NaOH solution (9.5 mL, 19.0 mmol). The reaction mixture was stirred at RT overnight, upon which time it was partially concentrated under reduced pressure to remove volatile organics. The resulting mixture was purified via reverse phase column chromatography over C18 (CH₃CN:H₂O with 0.1% NH₄OH, 10:100 to 100:0). Desired fractions were combined and lyophilized to yield (1S,3S,4S)-4-((2S,4S)-1-(6-fluoro-2-methyl-3-(((1r,4S)-4-((tetrahydro-2H-pyran-4-yl)oxy)cyclohexyl)methyl)-1H-indole-1-carbonyl)-4-(4-fluorophenyl)-2-methylpiperidine-4-carboxamido)-3-methylcyclohexane-1-carboxylic acid (sodium salt, 1.21 g) as a white powder. ¹H NMR (400 MHz, MeOD) δ 7.40-7.28 (m, 3H), 7.03-6.75 (m, 4H), 4.39 (d, J=58.4 Hz, 1H), 3.78 (dt, J=11.8, 4.2 Hz, 2H), 3.56 (ttd, J=9.2, 4.2, 2.2 Hz, 1H), 3.50-3.26 (m, 5H), 2.80-2.56 (m, 2H), 2.48 (dd, J=7.1, 4.1 Hz, 2H), 2.29-2.10 (m, 4H), 2.05-1.55 (m, 11H), 1.55-1.18 (m, 9H), 1.15-0.91 (m, 6H), 0.60 (dd, J=8.3, 6.4 Hz, 3H). MS m/z 748.3 [M+H]⁺. Potency (μM): biochemical qualified AC₅₀: 4.4E-04; NanoBiT qualified absolute AC₅₀: 0.026; cell proliferation qualified AC₅₀: 0.0059.

### Example 10

### (1S,3S,4S)-4-((2S,4S)-1-(7-fluoro-1-(tetrahydro-2H-pyran-4-yl)-1,3,4,5-tetrahydropyrano[4,3-b]indole-5-carbonyl)-4-(4-fluorophenyl)-2-methylpiperidine-4-carboxamido)-3-methylcyclohexane-1-carboxylic acid (single isomer)

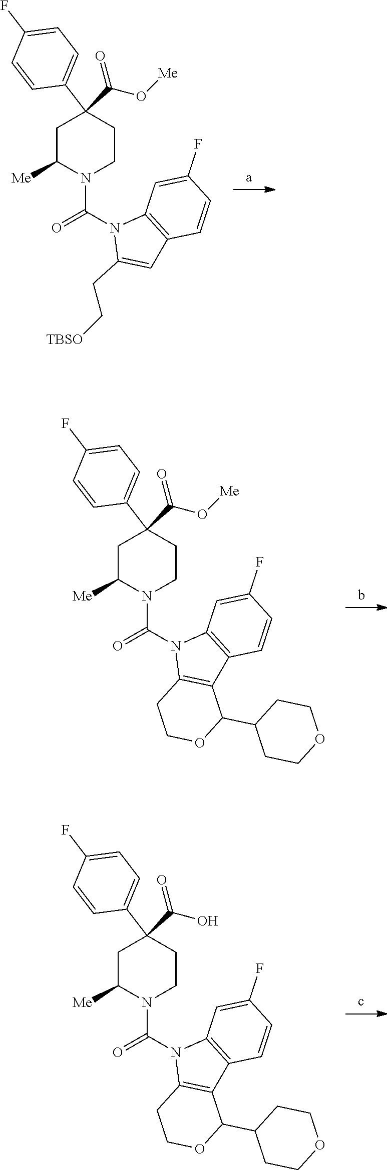 [SMILES: COC(=O)[C@@]1(c2ccc(F)cc2)CCN(C(=O)n2c(CCO[Si](C)(C)C(C)(C)C)cc3ccc(F)cc32)[C@@H](C)C1 ; COC(=O)[C@@]1(c2ccc(F)cc2)CCN(C(=O)n2c3c(c4ccc(F)cc42)C(C2CCOCC2)OCC3)[C@@H](C)C1 ; C[C@H]1C[C@](C(=O)O)(c2ccc(F)cc2)CCN1C(=O)n1c2c(c3ccc(F)cc31)C(C1CCOCC1)OCC2]

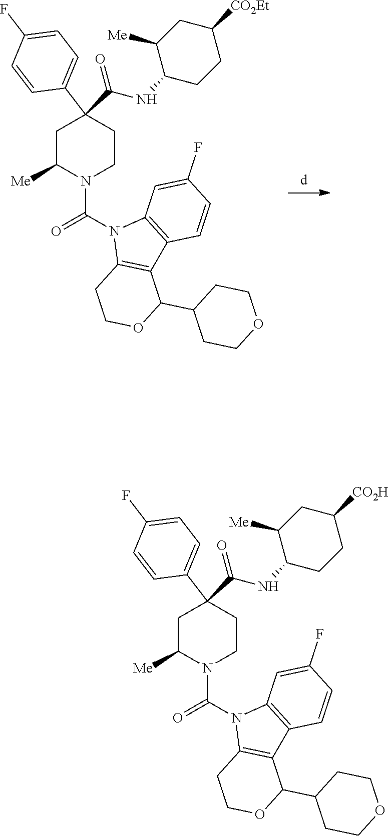 [SMILES: CCOC(=O)[C@H]1CC[C@H](NC(=O)[C@@]2(c3ccc(F)cc3)CCN(C(=O)n3c4c(c5ccc(F)cc53)C(C3CCOCC3)OCC4)[C@@H](C)C2)[C@@H](C)C1 ; C[C@H]1C[C@@H](C(=O)O)CC[C@@H]1NC(=O)[C@@]1(c2ccc(F)cc2)CCN(C(=O)n2c3c(c4ccc(F)cc42)C(C2CCOCC2)OCC3)[C@@H](C)C1]

Step a: To methyl (2S,4S)-1-(2-(2-((tert-butyldimethylsilyl)oxy)ethyl)-6-fluoro-1H-indole-1-carbonyl)-4-(4-fluorophenyl)-2-methylpiperidine-4-carboxylate in DCM (40 mL) at −40° C., were added tetrahydro-2H-pyran-4-carbaldehyde (1.03 g, 9.04 mmol) and TMSOTf (1.63 mL, 9.04 mmol). The reaction mixture was stirred at −40° C. for 1 h, upon which time it was removed from the cooling bath, quenched with aq. sat NaHCO₃ solution, and then stirred for 10 min. The resulting mixture was passed through a phase separator and concentrated under reduced pressure to yield the crude product. The crude product was purified via silica gel chromatography (EtOAc:heptane, 0:100 to 50:50). Desired fractions were combined and concentrated under reduced pressure to yield methyl (2S,4S)-1-(7-fluoro-1-(tetrahydro-2H-pyran-4-yl)-1,3,4,5-tetrahydropyrano[4,3-b]indole-5-carbonyl)-4-(4-fluorophenyl)-2-methylpiperidine-4-carboxylate (2.8 g). The sample was further purified via chiral SFC: (S,S) Whelk-O1 21 mm×250 mm 5 μm (CPC104); flow rate: 80 g/min.; co-solvent: 25% 3:1 CH₃CN:iPrOH in CO₂; detection: 269 nm; BPR pressure: 125 bar; injection size: 46 mg (23.0 mg/mL in CH₃CN:iPrOH, 20:1) to yield methyl (2S,4S)-1-(7-fluoro-1-(tetrahydro-2H-pyran-4-yl)-1,3,4,5-tetrahydropyrano[4,3-b]indole-5-carbonyl)-4-(4-fluorophenyl)-2-methylpiperidine-4-carboxylate.

Peak 1 (eluting first): 879 mg. Peak 2 (eluting second): 1.68 g.

Step b: To methyl (2S,4S)-1-(7-fluoro-1-(tetrahydro-2H-pyran-4-yl)-1,3,4,5-tetrahydropyrano[4,3-b]indole-5-carbonyl)-4-(4-fluorophenyl)-2-methylpiperidine-4-carboxylate (Peak 1, 879 mg, 1.59 mmol) in THF (15 mL) and iPrOH (7.50 mL) at 0° C., was added aq. 1 N NaOH solution (15.9 mL, 15.9 mmol). The resulting mixture was stirred for 5 min., upon which time it was warmed to RT and stirred for 30 min. The reaction mixture was heated to 45° C. for 24 h, and the temperature was increased to 50° C. for 30 min. The reaction mixture was cooled to 0° C., acidified with aq. 1 N HCl solution (23.9 mL, 23.9 mmol), and extracted with DCM. The organic extracts were passed through a phase separator and concentrated under reduced pressure to yield (2S,4S)-1-(7-fluoro-1-(tetrahydro-2H-pyran-4-yl)-1,3,4,5-tetrahydropyrano[4,3-b]indole-5-carbonyl)-4-(4-fluorophenyl)-2-methylpiperidine-4-carboxylic acid (857 mg, crude) as a beige solid. MS m/z 539.2 [M+H]⁺.

Step c: To (2S,4S)-1-(7-fluoro-1-(tetrahydro-2H-pyran-4-yl)-1,3,4,5-tetrahydropyrano[4,3-b]indole-5-carbonyl)-4-(4-fluorophenyl)-2-methylpiperidine-4-carboxylic acid (857 mg, 1.59 mmol) in DCM (40 mL) at RT, were added ethyl (1S,3S,4S)-4-amino-3-methylcyclohexane-1-carboxylate hydrochloride salt (353 mg, 1.59 mmol), HATU (1.21 g, 3.18 mmol), and DIPEA (1.39 mL, 7.96 mmol). The reaction mixture was stirred at RT for 2 h, upon which time it was partially concentrated under reduced pressure and purified via silica gel chromatography (EtOAc:heptane, 0:100 to 100:0). Desired fractions were combined and concentrated under reduced pressure to yield ethyl (1S,3S,4S)-4-((2S,4S)-1-(7-fluoro-1-(tetrahydro-2H-pyran-4-yl)-1,3,4,5-tetrahydropyrano[4,3-b]indole-5-carbonyl)-4-(4-fluorophenyl)-2-methylpiperidine-4-carboxamido)-3-methylcyclohexane-1-carboxylate (1.05 g). MS m/z 706.3 [M+H]⁺.

Step d: To ethyl (1S,3S,4S)-4-((2S,4S)-1-(7-fluoro-1-(tetrahydro-2H-pyran-4-yl)-1,3,4,5-tetrahydropyrano[4,3-b]indole-5-carbonyl)-4-(4-fluorophenyl)-2-methylpiperidine-4-carboxamido)-3-methylcyclohexane-1-carboxylate (1.05 g, 1.49 mmol) in THF (15 mL) and iPrOH (7.50 mL) at RT, was added aq. 1 N NaOH solution (14.9 mL, 14.9 mmol). The reaction mixture was stirred at RT for 18 h, upon which time it was partially concentrated under reduced pressure (temperature 40° C.). The crude material was purified via reverse phase column chromatography over C18 (CH₃CN:H₂O with 0.1% NH₄OH, 10:90 to 60:40). Desired fractions were combined, partially concentrated under reduced pressure and lyophilized to yield (1S,3S,4S)-4-((2S,4S)-1-(7-fluoro-1-(tetrahydro-2H-pyran-4-yl)-1,3,4,5-tetrahydropyrano[4,3-b]indole-5-carbonyl)-2-methyl-4-phenylpiperidine-4-carboxamido)-3-methylcyclohexane-1-carboxylic acid (sodium salt, 879 mg) as a white solid. ¹H NMR (400 MHz, MeOD-d₄) δ 7.58-7.51 (m, 1H), 7.50-7.41 (m, 2H), 7.25 (t, J=8.9 Hz, 1H), 7.17-7.04 (m, 3H), 6.96 (tdd, J=9.0, 6.6, 2.4 Hz, 1H), 4.85 (s, 1H), 4.44 (d, J=48.7 Hz, 1H), 4.31-4.16 (m, 1H), 4.04 (dd, J=11.3, 4.3 Hz, 1H), 3.88 (d, J=11.3 Hz, 1H), 3.76 (dtd, J=14.1, 10.4, 4.1 Hz, 1H), 3.66-3.45 (m, 3H), 2.82 (ddt, J=58.7, 42.4, 14.1 Hz, 5H), 2.54-2.18 (m, 2H), 2.16-1.96 (m, 2H), 1.94-1.64 (m, 5H), 1.56 (qd, J=12.7, 4.7 Hz, 1H), 1.49-1.29 (m, 5H), 1.26-0.97 (m, 4H), 0.72 (dd, J=6.4, 5.0 Hz, 3H). HRMS for C₃₈H₄₄F₂N₃O₆: mass calculated 678.3349 [M+H]⁺; mass observed 678.3339 [M+H]⁺. Potency (μM): biochemical qualified AC₅₀: <2.8E-04; NanoBiT qualified absolute AC₅₀: 0.28; cell proliferation qualified AC₅₀: 0.040.

The following compound of table 11 was synthesized using the above procedure or modifications to the above procedure using the corresponding aldehyde. The protonated carboxylate can be obtained directly when formic acid is used to neutralize the crude sodium carboxylate salt prior to purification.

TABLE 11

| Example | | | |
|---|---|---|---|
| ID | Structure | Analytical data | Potency (μM) |
| 10a | 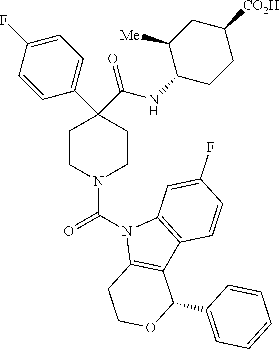 [SMILES: C[C@H]1C[C@@H](C(=O)O)CC[C@@H]1NC(=O)C1(c2ccc(F)cc2)CCN(C(=O)n2c3c(c4ccc(F)cc42)[C@H](c2ccccc2)OCC3)CC1] | ¹H NMR (400 MHz, MeOD- d₄) δ 7.42 − 7.30 (m, 2H), 7.30 − 7.13 (m, 6H), 7.11 − 6.96 (m, 3H), 6.70 − 6.51 (m, 2H), 5.74 (dt, J = 5.7, 2.0 Hz, 1H), 4.16 − 4.01 (m, 1H), 3.85 (dtd, J = 11.3, 8.6, 4.2 Hz, 1H), 3.68 (d, J = 14.9 Hz, 1H), 3.51 − 3.41 (m, 1H), 3.41 − 3.27 (m, 2H), 3.12 − 2.93 (m, 1H), 2.85 − 2.69 (m, 1H), 2.52 (dt, J = 31.2, 14.3 Hz, 2H), 2.24 − 1.91 (m, 3H), 1.91 − 1.74 (m, 3H), 1.74 − 1.59 (m, 1H), 1.35 (ddq, J = 27.3, 14.0, 4.4 Hz, 2H), 1.23 − 1.01 (m, 2H), 0.59 (dd, J = 6.4, 3.6 Hz, 3H). Sodium salt Derived from peak 1, Step a HRMS m/z 656.2936 | Biochemical qualified AC₅₀: 3.5E−04 NanoBiT qualified absolute AC₅₀: 0.088 Cell proliferation qualified AC₅₀: 0.077 |
| | | [M + H]⁺ | |
| | | Calculated HRMS m/z | |
| | | 656.2931 | |
| 10b | 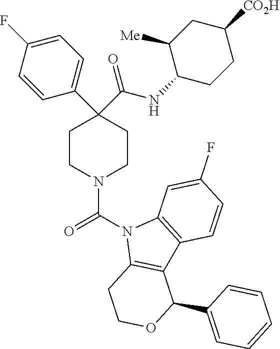 [SMILES: C[C@H]1C[C@@H](C(=O)O)CC[C@@H]1NC(=O)C1(c2ccc(F)cc2)CCN(C(=O)n2c3c(c4ccc(F)cc42)[C@@H](c2ccccc2)OCC3)CC1] | ¹H NMR (400 MHz, MeOD) δ 7.53 − 7.41 (m, 2H), 7.41 − 7.22 (m, 6H), 7.22 − 7.04 (m, 3H), 6.83 − 6.57 (m, 2H), 5.84 (dt, J = 4.4, 2.0 Hz, 1H), 4.30 − 4.12 (m, 1H), 3.95 (dtd, J = 11.7, 8.4, 4.2 Hz, 2H), 3.80 (d, J = 13.9 Hz, 1H), 3.59 − 3.38 (m, 3H), 3.18 − 3.03 (m, 1H), 2.86 (d, J = 16.7 Hz, 1H), 2.63 (q, J = 15.5 Hz, 2H), 2.26 − 2.08 (m, 2H), 2.04 − 1.89 (m, 3H), 1.79 (ddd, J = 13.6, 10.0, 3.6 Hz, 1H), 1.56 − 1.35 (m, 2H), 1.26 − 1.12 (m, 2H), 0.69 (dd, J = 14.1, 6.4 Hz, 3H). Sodium salt Derived from peak 2, Step a MS m/z 656.2 [M + H]⁺ | Biochemical qualified AC₅₀: 0.037 NanoBiT qualified absolute AC₅₀: 12 Cell proliferation qualified AC₅₀: 10 |

### Example 11

### (R)-1-(5-bromo-1-((tetrahydro-2H-pyran-4-yl)methyl)-1H-indole-3-carbonyl)-4-(4-fluorophenyl)-N-(1-methylpyrrolidin-3-yl)piperidine-4-carboxamide

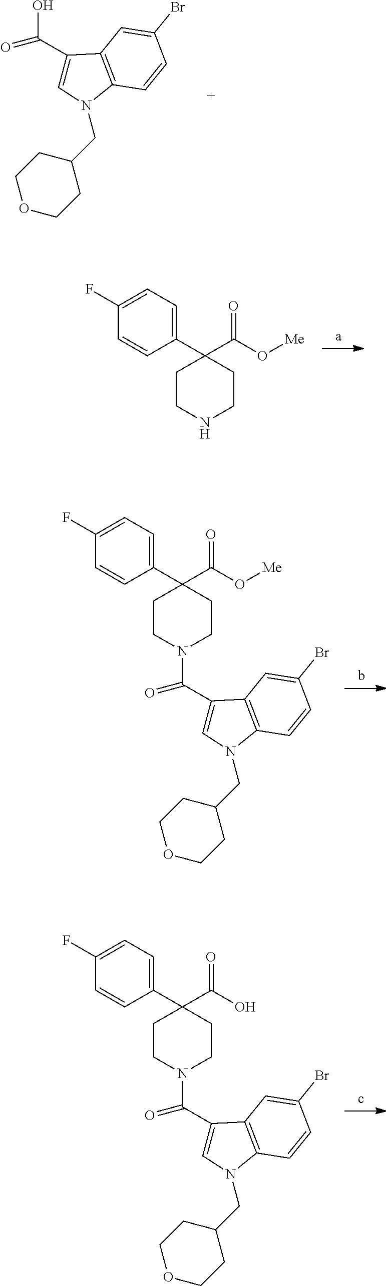 [SMILES: O=C(O)c1cn(CC2CCOCC2)c2ccc(Br)cc12 ; COC(=O)C1(c2ccc(F)cc2)CCNCC1 ; COC(=O)C1(c2ccc(F)cc2)CCN(C(=O)c2cn(CC3CCOCC3)c3ccc(Br)cc23)CC1 ; O=C(c1cn(CC2CCOCC2)c2ccc(Br)cc12)N1CCC(C(=O)O)(c2ccc(F)cc2)CC1]

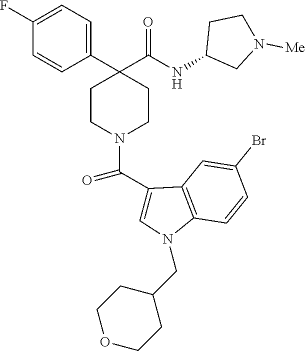 [SMILES: CN1CC[C@@H](NC(=O)C2(c3ccc(F)cc3)CCN(C(=O)c3cn(CC4CCOCC4)c4ccc(Br)cc34)CC2)C1]

Step a: To methyl 4-(4-fluorophenyl)piperidine-4-carboxylate hydrochloride salt (3.72 g, 13.6 mmol) in DCM (50 mL) at RT, were added HATU (7.76 g, 20.4 mmol), 5-bromo-1-((tetrahydro-2H-pyran-4-yl)methyl)-1H-indole-3-carboxylic acid (4.60 g, 13.6 mmol), and DIPEA (8.79 g, 68.0 mmol). The reaction mixture was stirred at RT for 4 h, upon which time it was diluted with EtOAc (1.0 L), washed with H₂O (500 mL), and aq. sat. NaCl solution (500 mL×2). The combined organic extracts were dried over Na₂SO₄, filtered, and concentrated under reduced pressure to yield the crude product. The crude product was purified via silica gel chromatography (petroleum ether:EtOAc, 50:50 to 0:100). Desired fractions were combined and concentrated under reduced pressure to yield methyl 1-(5-bromo-1-((tetrahydro-2H-pyran-4-yl)methyl)-1H-indole-3-carbonyl)-4-(4-fluorophenyl)piperidine-4-carboxylate (5.0 g) as an off-white solid. MS m/z 559.1 [M+H]⁺.

Step b: To methyl 1-(5-bromo-1-((tetrahydro-2H-pyran-4-yl)methyl)-1H-indole-3-carbonyl)-4-(4-fluorophenyl)piperidine-4-carboxylate (4.80 g, 8.61 mmol) in MeOH (15.0 mL), H₂O (3.00 mL), and THF (30.0 mL) at RT, was added NaOH (689 mg, 17.2 mmol) in one portion. The mixture was stirred at 50° C. for 7 h. The solvent was concentrated under reduced pressure, then THF (100 mL) was added. The reaction mixture was concentrated under reduced pressure once again. Lastly, 4.0 M HCl in 1,4-dioxane (20 mL) was added to the residue and the resulting mixture was concentrated under reduced pressure to yield 1-[5-bromo-1-(tetrahydropyran-4-ylmethyl)indole-3-carbonyl]-4-(4-fluorophenyl)piperidine-4-carboxylic acid (6.00 g, crude) as a white solid. MS m/z 543.1 [M+H]⁺.

Step c: To 1-[5-bromo-1-(tetrahydropyran-4-ylmethyl)indole-3-carbonyl]-4-(4-fluorophenyl)piperidine-4-carboxylic acid (1.25 g, 2.30 mmol) in DCM (20 mL) under N₂, was added HATU (1.53 g, 4.03 mmol) and the reaction mixture was stirred for 5 min. Next, (3R)-1-methylpyrrolidin-3-amine (0.230 g, 2.30 mmol) in DCM (3 mL) was added slowly to the reaction mixture followed by DIPEA (1.61 mL, 9.20 mmol). The reaction mixture was stirred overnight, upon which time it was diluted with H₂O (10 mL) and stirred for 1 h. The organic phase was separated and the aq. layer was extracted with DCM. The combined organic extracts were washed with aq. sat. NaHCO₃ solution, dried over Na₂SO₄, filtered, and concentrated under reduced pressure to yield the crude product. The crude product was purified via silica gel chromatography (DCM:MeOH, 0:100 to 15:85). Desired fractions were concentrated under reduced pressure. EtOH (10 mL) was added and the mixture was concentrated under reduced pressure once again. Lastly, the material was dissolved in EtOH (10 mL), concentrated under reduced pressure almost to dryness, and diluted with H₂O and CH₃CN. The material was lyophilized to yield (R)-1-(5-bromo-1-((tetrahydro-2H-pyran-4-yl)methyl)-1H-indole-3-carbonyl)-4-(4-fluorophenyl)-N-(1-methylpyrrolidin-3-yl)piperidine-4-carboxamide (870 mg) as a white solid. ¹H NMR (400 MHz, MeOD-d₄) δ 7.86 (d, J=1.8 Hz, 1H), 7.69 (s, 1H), 7.51-7.42 (m, 3H), 7.37 (dd, J=8.8, 1.9 Hz, 1H), 7.15-7.08 (m, 2H), 4.40 (tt, J=9.4, 4.8 Hz, 1H), 4.15 (d, J=7.3 Hz, 4H), 3.93 (dd, J=11.6, 2.5 Hz, 2H), 3.59-3.44 (m, 2H), 3.41-3.34 (m, 2H), 2.85 (dd, J=10.1, 7.5 Hz, 2H), 2.57 (d, J=9.9 Hz, 4H), 2.43 (s, 3H), 2.28 (ddd, J=17.1, 8.5, 4.1 Hz, 1H), 2.16 (ddd, J=11.3, 7.4, 4.1 Hz, 1H), 2.09-1.96 (m, 2H), 1.72-1.60 (m, 1H), 1.53-1.33 (m, 4H). MS m/z 625.2 [M+H]⁺. Potency (μM): biochemical qualified AC₅₀: 1.8E-02; NanoBiT qualified absolute AC₅₀: 4.9; cell proliferation qualified AC₅₀: 2.7.

The following compounds of table 11 were synthesized using the above procedure or modifications to the above procedure using the corresponding functionalized piperidine, indole intermediate, and amine. In cases where an acid is present in the final structure, saponification of the corresponding ethyl ester was performed in the final step. The protonated acid can be obtained directly when formic acid is used to neutralize the crude sodium carboxylate salt prior to purification.

TABLE 12

| Example | | | |
|---|---|---|---|
| ID | Structure | Analytical data | Potency (μM) |
| 11a | 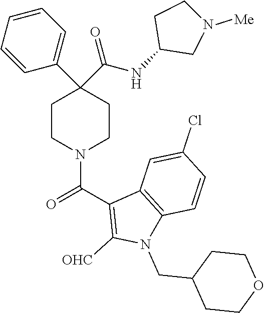 [SMILES: CN1CC[C@@H](NC(=O)C2(c3ccccc3)CCN(C(=O)c3c(C=O)n(CC4CCOCC4)c4ccc(Cl)cc34)CC2)C1] | ¹H NMR (400 MHz, DMSO- d₆) δ 9.97 (s, 1H), 7.85 (d, J = 9.1 Hz, 1H), 7.77 − 7.66 (m, 1H), 7.63 − 7.57 (m, 1H), 7.49 − 7.42 (m, 1H), 7.39 − 7.31 (m, 4H), 7.27 − 7.19 (m, 1H), 4.47 (d, J = 7.1 Hz, 2H), 4.39 − 4.11 (m, 2H), 3.85 − 3.70 (m, 2H), 3.46 − 3.36 (m, 1H), 3.25 − 3.14 (m, 4H), 2.69 − 2.53 (m, 2H), 2.47 − 2.33 (m, 2H), 2.32 − 2.20 (m, 2H), 2.17 (s, 3H), 2.07 − 1.87 (m, 3H), 1.84 − 1.43 (m, 2H), 1.38 − 1.18 (m, 4H). HRMS m/z 591.2730 [M + H]⁺ Calculated HRMS m/z 591.2738 | Biochemical qualified AC₅₀: 8.7E−04 Cell proliferation qualified AC₅₀: 0.30 |
| 11b | 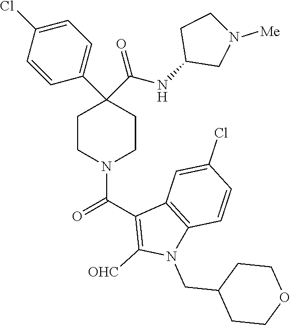 [SMILES: CN1CC[C@@H](NC(=O)C2(c3ccc(Cl)cc3)CCN(C(=O)c3c(C=O)n(CC4CCOCC4)c4ccc(Cl)cc34)CC2)C1] | ¹H NMR (400 MHz, MeOD- d₄) δ 9.91 (s, 1H), 7.64 − 7.51 (m, 2H), 7.39 − 7.22 (m, 6H), 4.53 − 4.19 (m, 4H), 3.80 (dt, J = 11.3, 3.2 Hz, 2H), 3.54 − 3.29 (m, 3H), 2.59 (m, J = 7.9 Hz, 3H), 2.31 (m, J = 8.1 Hz, 11H), 1.41 (d, J = 54.2 Hz, 6H). MS m/z 625.4 [M + H]⁺ | Biochemical qualified AC₅₀: 6.5E−04 NanoBiT qualified absolute AC₅₀: 0.56 Cell proliferation qualified AC₅₀: 0.22 |
| 11c | 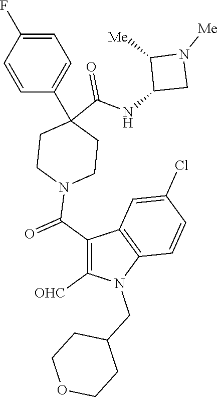 [SMILES: C[C@H]1[C@@H](NC(=O)C2(c3ccc(F)cc3)CCN(C(=O)c3c(C=O)n(CC4CCOCC4)c4ccc(Cl)cc34)CC2)CN1C] | ¹H NMR (400 MHz, DMSO- d₆) δ 9.95 (s, 1H), 7.97 (m, 1H), 7.86 (d, J = 9.0 Hz, 1H), 7.67 (m, 1H), 7.47 (m, 3H), 7.18 (m, 2H), 4.47 (d, J = 7.1 Hz, 2H), 4.19 (m, 2H), 3.80 (d, J = 10.8 Hz, 2H), 3.09 (m, 7H), 2.74 (m, 2H), 2.11 (s, 3H), 1.80 (m, 3H), 1.34 (m, 5H), 0.56 (m, 3H). HRMS m/z 609.2637 [M + H]⁺ Calculated HRMS m/z 609.2638 | Biochemical qualified AC₅₀: 1.0E−04 Cell proliferation qualified AC₅₀: 0.017 |
| 11d | 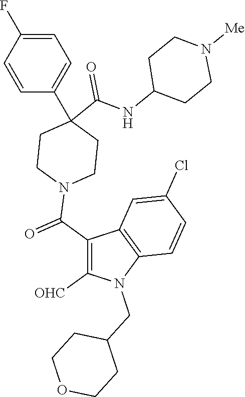 [SMILES: CN1CCC(NC(=O)C2(c3ccc(F)cc3)CCN(C(=O)c3c(C=O)n(CC4CCOCC4)c4ccc(Cl)cc34)CC2)CC1] | ¹H NMR (400 MHz, DMSO- d₆) δ 10.04 − 9.87 (m, 1H), 9.13 − 8.88 (m, 1H), 7.87 (d, J = 9.1 Hz, 1H), 7.76 − 7.60 (m, 2H), 7.47 (dd, J = 9.0, 2.0 Hz, 1H), 7.44 − 7.32 (m, 2H), 7.24 − 7.11 (m, 2H), 4.48 (d, J = 7.2 Hz, 2H), 4.31 (s, 1H), 3.91 − 3.72 (m, 3H), 3.23 − 3.11 (m, 4H), 3.06 − 2.90 (m, 2H). 2.71 (s, 3H), 2.62 − 2.54 (m, 1H), 2.37 − 2.28 (m, 2H), 2.07 − 1.90 (m, 2H), 1.88 − 1.64 (m, 4H), 1.60 − 1.46 (m, 2H), 1.41 − 1.22 (m, 4H). HRMS m/z 623.2792 [M + H]⁺ Calculated HRMS m/z 623.2795 | Cell proliferation qualified : 0.43 |
| 11e | 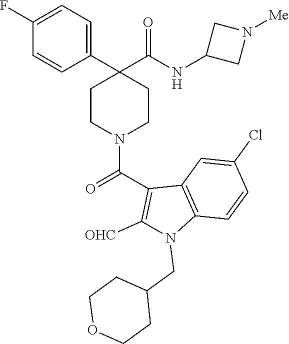 [SMILES: CN1CC(NC(=O)C2(c3ccc(F)cc3)CCN(C(=O)c3c(C=O)n(CC4CCOCC4)c4ccc(Cl)cc34)CC2)C1] | ¹H NMR (400 MHz, DMSO- d₆) δ 9.97 (s, 1H), 8.08 − 7.89 (m, 1H), 7.84 (d, J = 9.0 Hz, 1H), 7.71 (s, 1H), 7.59 − 7.31 (m, 3H), 7.17 (t, J = 8.7 Hz, 2H), 4.66 − 4.08 (m, 4H), 3.80 (d, J = 11.4 Hz, 2H), 3.45 (s, 2H), 3.25 − 3.09 (m, 4H), 2.84 (s, 2H), 2.33 (s, 1H), 2.20 (s, 3H), 1.86 (d, J = 115.3 Hz, 4H), 1.33 (d, J = 19.6 Hz, 5H). HRMS m/z 595.2492 [M + H]⁺ Calculated HRMS m/z 595.2482 | Cell proliferation qualified AC₅₀: 0.29 |
| 11f | 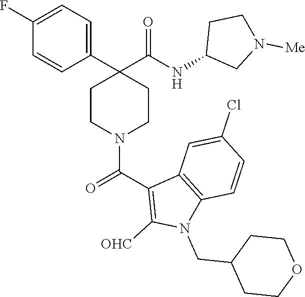 [SMILES: CN1CC[C@@H](NC(=O)C2(c3ccc(F)cc3)CCN(C(=O)c3c(C=O)n(CC4CCOCC4)c4ccc(Cl)cc34)CC2)C1] | ¹H NMR (400 MHz, DMSO- d₆) δ 9.97 (s, 1H), 7.85 (d, J = 9.1 Hz, 1H), 7.71 (d, J = 2.1 Hz, 1H), 7.63 (s, 1H), 7.55 − 7.32 (m, 3H), 7.17 (t, J = 8.7 Hz, 2H), 4.47 (d, J = 7.3 Hz, 2H), 4.22 (s, 2H), 3.80 (d, J = 11.1 Hz, 2H), 3.41 (s, 1H), 3.30 − 3.09 (m, 5H), 2.60 (s, 1H), 2.33 (s, 7H), 2.01 (s, 3H), 1.61 (d, J = 65.5 Hz, 2H), 1.44 − 1.18 (m, 4H). MS m/z 609.2 [M + H]⁺ | Cell proliferation qualified AC₅₀: 0.14 |
| 11g | 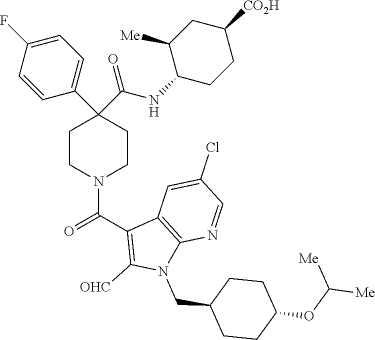 [SMILES: CC(C)O[C@H]1CC[C@H](Cn2c(C=O)c(C(=O)N3CCC(C(=O)N[C@H]4CC[C@H](C(=O)O)C[C@@H]4C)(c4ccc(F)cc4)CC3)c3cc(Cl)cnc32)CC1] | ¹H NMR (400 MHz, DMSO- d₆) δ 12.00 (s, 1H), 10.03 (s, 1H), 8.60 (d, J = 2.3 Hz, 1H), 8.35 (d, J = 2.4 Hz, 1H), 7.39 (dd, J = 8.7, 5.4 Hz, 2H), 7.32 (d. J = 8.6 Hz, 1H), 7.16 (t, J = 8.9 Hz, 2H), 4.47 (d, J = 7.3 Hz, 2H), 4.33 (s, 1H), 3.64 (p, J = 6.1 Hz, 1H), 3.44 (s, 1H), 3.31 − 3.12 (m, 4H), 2.70 − 2.55 (m, 1H), 2.46 − 2.29 (m, 1H), 2.22 −2.07 (m, 1H), 2.07 − 1.91 (m, 1H), 1.91 − 1.79 (m, 4H), 1.79 − 1.71 (m, 1H), 1.66 (s, 2H), 1.52 − 1.32 (m, 3H), 1.32 − 1.21 (m, 1H), 1.19 − 1.04 (m, 4H), 1.02 (s, 3H), 1.01 (s, 3H), 1.01 − 0.90 (m, 2H), 0.60 (d, J = 23.1 Hz, 3H). HRMS m/z 723.3298 [M + H]⁺ Calculated HRMS m/z | Biochemical qualified AC₅₀: 3.6E−04 NanoBiT qualified absolute AC₅₀: 0.27 Cell proliferation qualified AC₅₀: 0.61 |
| | | 723.3319 | |
| 11h | 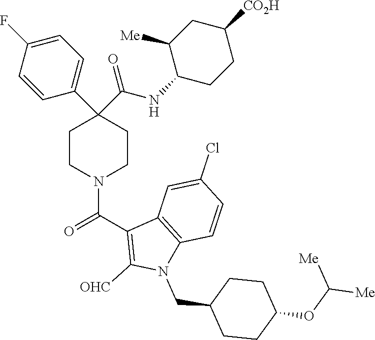 [SMILES: CC(C)O[C@H]1CC[C@H](Cn2c(C=O)c(C(=O)N3CCC(C(=O)N[C@H]4CC[C@H](C(=O)O)C[C@@H]4C)(c4ccc(F)cc4)CC3)c3cc(Cl)ccc32)CC1] | ¹H NMR (400 MHz, DMSO- d₆) δ 9.97 (s, 1H), 7.80 (d, J = 9.1 Hz, 1H), 7.75 − 7.64 (m, 1H), 7.54 − 7.26 (m, 4H), 7.18 (t, J = 8.6 Hz, 2H), 4.44 (s, 3H), 3.66 (p, J = 6.1 Hz, 1H), 3.41 (s. 2H), 3.22 (dt, J = 12.0, 6.2 Hz, 3H), 2.67 (d, J = 18.7 Hz, 1H), 2.37 (d, J = 20.0 Hz, 1H), 2.20 − 1.54 (m, 9H), 1.54 − 0.94 (m, 16H), 0.63 (s, 3H). Sodium salt MS m/z 722.8 [M + H]⁺ | Biochemical qualified AC₅₀: <2.8E−04 NanoBiT qualified absolute AC₅₀: 0.054 Cell proliferation qualified AC₅₀: 0.12 |
| 11i | 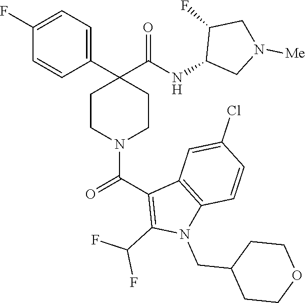 [SMILES: CN1C[C@@H](F)[C@@H](NC(=O)C2(c3ccc(F)cc3)CCN(C(=O)c3c(C(F)F)n(CC4CCOCC4)c4ccc(Cl)cc34)CC2)C1] | ¹H NMR (400 MHz, DMSO- d₆) δ 7.79 (dd, J = 8.9, 6.2 Hz, 1H), 7.65 − 7.49 (m, 2H), 7.38 (tdd, J = 15.5, 8.0, 4.3 Hz, 4H), 7.24 − 7.10 (m, 2H), 4.99 (dd, J = 56.3, 13.9 Hz, 1H), 4.26 (dd, J = 19.6, 7.3 Hz, 4H), 3.87 − 3.76 (m, 2H), 3.36 (s, 1H), 3.29 − 3.06 (m, 4H), 2.93 (d, J = 30.3 Hz, 1H), 2.73 − 2.51 (m, 4H), 2.44 − 1.51 (m, 7H), 1.47 − 1.23 (m, 4H). MS m/z 649.4 [M + H]⁺ | Biochemical qualified AC₅₀: 7.2E−04 Cell proliferation qualified AC₅₀: 0.054 |
| 11j | 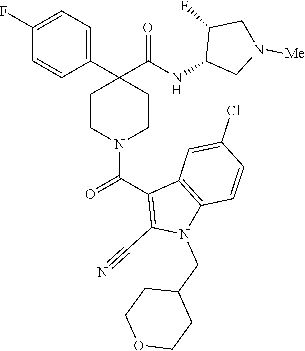 [SMILES: CN1C[C@@H](F)[C@@H](NC(=O)C2(c3ccc(F)cc3)CCN(C(=O)c3c(C#N)n(CC4CCOCC4)c4ccc(Cl)cc34)CC2)C1] | ¹H NMR (400 MHz, DMSO- d₆) δ 7.86 (d, J = 9.0 Hz, 1H), 7.75 − 7.63 (m, 2H), 7.49 (dd, J = 9.0, 2.1 Hz, 1H), 7.47 − 7.36 (m, 2H), 7.17 (t, J = 8.9 Hz, 2H), 5.03 (d, J = 55.6 Hz, 1H), 4.28 (d, J = 7.3 Hz, 3H), 3.83 (dd, J = 10.8, 3.9 Hz, 3H), 3.30 − 3.13 (m, 5H), 3.01 (s, 1H), 2.84 − 2.55 (m, 3H), 2.43 − 2.16 (m, 5H), 2.16 − 2.03 (m, 1H), 1.84 (s, 2H), 1.50 − 1.23 (m, 4H). MS m/z 624.3 [M + H]⁺ | Biochemical qualified AC₅₀: 9.7E−04 Cell proliferation qualified AC₅₀: 0.73 |

### Example 12

### 1-(5-chloro-2-methyl-1-((tetrahydro-2H-pyran-4-yl)methyl)-1H-indole-3-carbonyl)-N-((3S,4R)-4-fluoro-1-methylpyrrolidin-3-yl)-4-(4-fluorophenyl)piperidine-4-carboxamide

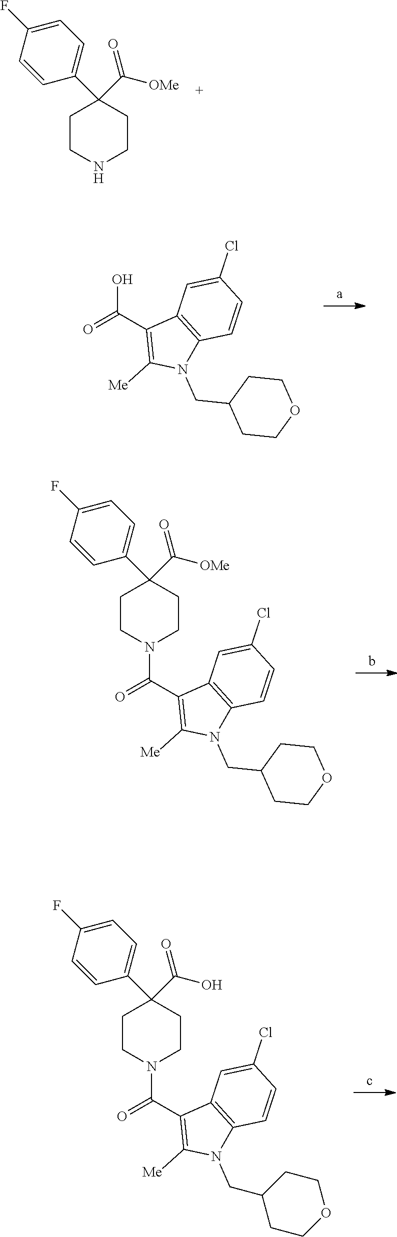 [SMILES: COC(=O)C1(c2ccc(F)cc2)CCNCC1 ; Cc1c(C(=O)O)c2cc(Cl)ccc2n1CC1CCOCC1 ; COC(=O)C1(c2ccc(F)cc2)CCN(C(=O)c2c(C)n(CC3CCOCC3)c3ccc(Cl)cc23)CC1 ; Cc1c(C(=O)N2CCC(C(=O)O)(c3ccc(F)cc3)CC2)c2cc(Cl)ccc2n1CC1CCOCC1]

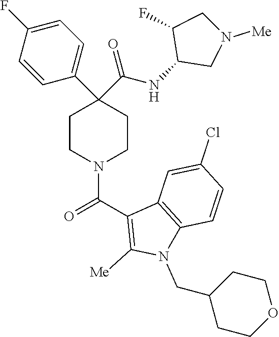 [SMILES: Cc1c(C(=O)N2CCC(C(=O)N[C@H]3CN(C)C[C@H]3F)(c3ccc(F)cc3)CC2)c2cc(Cl)ccc2n1CC1CCOCC1]

Step a: To methyl 4-(4-fluorophenyl)piperidine-4-carboxylate (3.40 g, 14.3 mmol) in DCM (54.6 mL) at RT, were added 5-chloro-2-methyl-1-((tetrahydro-2H-pyran-4-yl)methyl)-1H-indole-3-carboxylic acid (4.20 g, 13.7 mmol), EDC (4.71 g, 24.6 mmol), and 1-hydroxy-7-azabenzotriazole (3.71 g, 27.3 mmol). After stirring for 30 min., DIPEA (11.9 mL, 68.2 mmol) was added and the reaction mixture was stirred at RT for 18 h. The reaction mixture was diluted with EtOAc (300 mL) and washed with aq. sat. NaHCO₃ solution (150 mL), H₂O (150 mL), and aq. sat. NaCl solution (150 mL). Single aq. layers were extracted with EtOAc (150 mL). The combined organic extracts were dried over Na₂SO₄, filtered, and concentrated under reduced pressure to yield the crude product. The crude product was purified via silica gel chromatography (MeOH:DCM, 0:100 to 5:95). Desired fractions were combined and concentrated under reduced pressure to yield methyl 1-(5-chloro-2-methyl-1-((tetrahydro-2H-pyran-4-yl)methyl)-1H-indole-3-carbonyl)-4-(4-fluorophenyl)piperidine-4-carboxylate (6.12 g) as a foam. MS m/z 527.3 [M+H]⁺.

Step b: To methyl 1-(5-chloro-2-methyl-1-((tetrahydro-2H-pyran-4-yl)methyl)-1H-indole-3-carbonyl)-4-(4-fluorophenyl)piperidine-4-carboxylate (6.12 g, 10.8 mmol) in THF (24.7 mL), MeOH (12.3 mL), and H₂O (6.17 mL) at RT, was added NaOH (4.32 g, 108 mmol). The reaction mixture was stirred at 50° C. for 18 h, upon which time it was partially concentrated. The oily residue was diluted with H₂O (50 mL) followed by treatment with aq. 5 N HCl solution to adjust the pH to ˜1. The resulting milky suspension was diluted with EtOAc (100 mL), layers were separated, and the aq. layer was extracted with EtOAc (100 mL). The combined organic extracts were dried and concentrated under reduced pressure to yield 1-(5-chloro-2-methyl-1-((tetrahydro-2H-pyran-4-yl)methyl)-1H-indole-3-carbonyl)-4-(4-fluorophenyl)piperidine-4-carboxylic acid (5.50 g, crude) as a cream solid. MS m/z 513.2 [M+H]⁺.

Step c: To 1-(5-chloro-2-methyl-1-((tetrahydro-2H-pyran-4-yl)methyl)-1H-indole-3-carbonyl)-4-(4-fluorophenyl)piperidine-4-carboxylic acid (800 mg, 1.56 mmol) in DCM (15.6 mL) at RT, were added (3S,4R)-4-fluoro-1-methylpyrrolidin-3-amine hydrochloride salt (358 mg, 1.871 mmol), HATU (1.19 g, 3.12 mmol), and DIPEA (1.36 mL, 7.80 mmol). The reaction mixture was stirred at RT overnight, upon which time it was diluted with DCM (20 mL) and washed with H₂O and aq. sat. NaCl solution. The combined organic extracts were dried over Na₂SO₄, filtered, and concentrated under reduced pressure to yield the crude product. The crude product was purified via silica gel chromatography (MeOH:DCM, 0:100 to 10:90). Desired fractions were combined and concentrated under reduced pressure. The resulting material was dissolved in CH₃CN/H₂O (1/3, 80 mL) and lyophilized to yield 1-(5-chloro-2-methyl-1-((tetrahydro-2H-pyran-4-yl)methyl)-1H-indole-3-carbonyl)-N-((3S,4R)-4-fluoro-1-methylpyrrolidin-3-yl)-4-(4-fluorophenyl)piperidine-4-carboxamide (750 mg) as a white solid. ¹H NMR (400 MHz, MeOD-d₄) δ 7.53-7.32 (m, 4H), 7.21-7.02 (m, 3H), 5.25-4.97 (m, 1H), 4.59-4.20 (m, 2H), 4.10 (d, J=7.3 Hz, 2H), 3.94 (d, J=11.7 Hz, 2H), 3.83-3.37 (m, 5H), 3.05-2.73 (m, 3H), 2.57 (dd, J=55.0, 6.4 Hz, 6H), 2.38 (d, J=3.8 Hz, 3H), 2.26-1.67 (m, 3H), 1.59-1.40 (m, 4H). HRMS for C₃₃H₄₀ClF₂N₄O₃: mass calculated 613.2757 [M+H]⁺; mass observed 613.2784 [M+H]⁺. Potency (μM): biochemical qualified AC₅₀: 1.3E-03; NanoBiT qualified absolute AC₅₀: 0.35; cell proliferation qualified AC₅₀: 0.27.

### Example 13

### 1-(5-chloro-2-formyl-1-((tetrahydro-2H-pyran-4-yl)methyl)-1H-indole-3-carbonyl)-N-((3S,4R)-4-fluoro-1-methylpyrrolidin-3-yl)-4-(4-fluorophenyl)piperidine-4-carboxamide

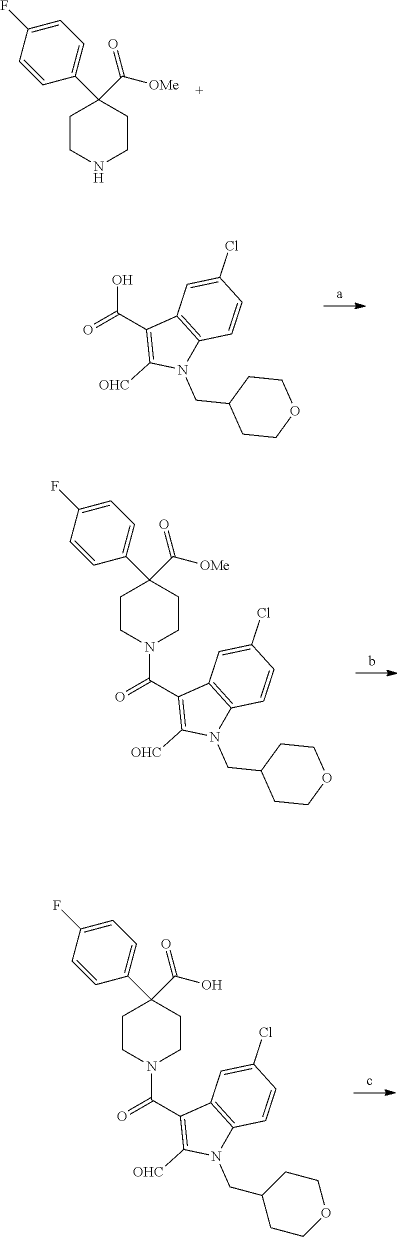 [SMILES: COC(=O)C1(c2ccc(F)cc2)CCNCC1 ; O=Cc1c(C(=O)O)c2cc(Cl)ccc2n1CC1CCOCC1 ; COC(=O)C1(c2ccc(F)cc2)CCN(C(=O)c2c(C=O)n(CC3CCOCC3)c3ccc(Cl)cc23)CC1 ; O=Cc1c(C(=O)N2CCC(C(=O)O)(c3ccc(F)cc3)CC2)c2cc(Cl)ccc2n1CC1CCOCC1]

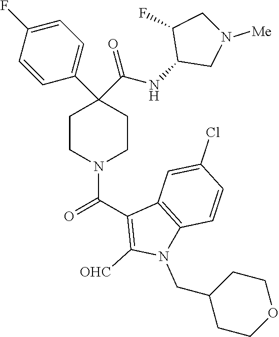 [SMILES: CN1C[C@@H](F)[C@@H](NC(=O)C2(c3ccc(F)cc3)CCN(C(=O)c3c(C=O)n(CC4CCOCC4)c4ccc(Cl)cc34)CC2)C1]

Step a: To 5-chloro-2-formyl-1-((tetrahydro-2H-pyran-4-yl)methyl)-1H-indole-3-carboxylic acid in DCM (50 mL) at RT, were added methyl 4-(4-fluorophenyl)piperidine-4-carboxylate (2.63 g, 11.1 mmol), HOAt (2.51 g, 18.5 mmol), and EDC (3.19 g, 16.6 mmol). The reaction mixture was stirred at RT for 45 min., upon which time DIPEA (8.06 mL, 46.2 mmol) was added and it was stirred for 1.5 h. The reaction mixture was diluted with EtOAc and washed with aq. sat. NaHCO₃ solution. The aq. phase was extracted with EtOAc and the combined organics were washed with aq. sat. NaCl solution, dried over MgSO₄, filtered, and concentrated under reduced pressure to yield the crude product. The crude product was purified via silica gel chromatography (EtOAc:heptane, 0:100 to 100:0). Desired fractions were combined and concentrated under reduced pressure to yield methyl 1-(5-chloro-2-formyl-1-((tetrahydro-2H-pyran-4-yl)methyl)-1H-indole-3-carbonyl)-4-(4-fluorophenyl)piperidine-4-carboxylate (4.66 g) as a solid. ¹H NMR (400 MHz, DMSO-d₆) δ 9.97 (s, 1H), 7.85 (d, J=9.1 Hz, 1H), 7.73 (d, J=2.0 Hz, 1H), 7.50-7.33 (m, 3H), 7.20 (t, J=8.6 Hz, 2H), 4.48 (d, J=7.2 Hz, 2H), 3.80 (d, J=11.3 Hz, 2H), 3.63 (s, 3H), 3.36-3.09 (m, 9H), 1.99 (s, 2H), 1.44-1.23 (m, 4H).

Step b: To methyl 1-(5-chloro-2-formyl-1-((tetrahydro-2H-pyran-4-yl)methyl)-1H-indole-3-carbonyl)-4-(4-fluorophenyl)piperidine-4-carboxylate (4.41 g, 8.15 mmol) in THF (28 mL) and MeOH (14 mL) at RT, was added aq. 1 N NaOH solution (20.4 mL, 20.4 mmol). The reaction mixture was stirred overnight at RT followed by stirring at 50° C. for 1.5 h. The reaction mixture was partially concentrated under reduced pressure. The residue was diluted with H₂O, then treated with aq. 1 N HCl solution until the pH 1 was reached. The suspension was diluted with EtOAc and the layers were separated. The aq. layer was extracted with EtOAc. The combined organic extracts were washed with aq. sat. NaCl solution, dried over MgSO₄, filtered, and concentrated under reduced pressure to yield 1-(5-chloro-2-formyl-1-((tetrahydro-2H-pyran-4-yl)methyl)-1H-indole-3-carbonyl)-4-(4-fluorophenyl)piperidine-4-carboxylic acid (4.17 g, crude). ¹H NMR (400 MHz, DMSO-d₆) δ 12.74 (s, 1H), 9.97 (s, 1H), 7.84 (d, J=9.1 Hz, 1H), 7.74 (d, J=10.0 Hz, 1H), 7.49-7.38 (m, 3H), 7.19 (t, J=8.7 Hz, 2H), 4.48 (d, J=7.2 Hz, 3H), 3.87-3.68 (m, 2H), 3.45 (s, 1H), 3.27-3.08 (m, 5H), 2.33 (p, J=1.9 Hz, 1H), 1.99 (s, 2H), 1.75 (s, 1H), 1.46-1.22 (m, 4H).

Step c: To 1-(5-chloro-2-formyl-1-((tetrahydro-2H-pyran-4-yl)methyl)-1H-indole-3-carbonyl)-4-(4-fluorophenyl)piperidine-4-carboxylic acid (3.29 g, 6.24 mmol) in DCM (62.4 mL) at RT, were added (3S,4R)-4-fluoro-1-methylpyrrolidin-3-amine hydrochloride salt (1.31 g, 6.87 mmol) and HATU (4.75 g, 12.5 mmol). The reaction mixture was stirred at RT for 15 min., upon which time DIPEA (4.36 mL, 25.0 mmol) was added. The reaction mixture was stirred at RT for 2.5 h. The resulting mixture was diluted with DCM and washed with H₂O, aq. sat. Na₂CO₃ solution (3×), and aq. sat. NaCl solution. The combined organic extracts were dried over MgSO₄, filtered, and concentrated under reduced pressure to yield the crude product. The crude product was purified via silica gel chromatography (MeOH:DCM, 0:100 to 20:80). Desired fractions were combined and concentrated under reduced pressure. The resulting solid was dissolved in EtOAc and washed with aq. sat. Na₂CO₃ solution (3×). The combined organics were dried over MgSO₄, filtered, and concentrated under reduced pressure. The resulting solid was dissolved in CH₃CN:H₂O (50:50) and lyophilized. This material was purified via reverse phase column chromatography over C18 (CH₃CN:H₂O, 0:100 to 90:10). Desired fractions were concentrated under reduced pressure to remove CH₃CN, then partitioned between EtOAc and H₂O. The aq. phase was washed with EtOAc and the combined organics were washed with aq. sat. NaCl solution, dried over MgSO₄, filtered, and concentrated under reduced pressure. The resulting material was dissolved in DCM/MeOH and concentrated under reduced pressure to yield 1-(5-chloro-2-formyl-1-((tetrahydro-2H-pyran-4-yl)methyl)-1H-indole-3-carbonyl)-N-((3S,4R)-4-fluoro-1-methylpyrrolidin-3-yl)-4-(4-fluorophenyl)piperidine-4-carboxamide (2.33 g) as a white solid. ¹H NMR (400 MHz, DMSO-d₆) δ 9.97 (s, 1H), 7.85 (d, J=9.1 Hz, 1H), 7.64 (m, 2H), 7.46 (dd, J=9.0, 2.1 Hz, 1H), 7.40 (s, 2H), 7.16 (t, J=8.6 Hz, 2H), 5.00 (d, J=63.0 Hz, 1H), 4.47 (d, J=7.2 Hz, 2H), 4.40-4.11 (m, 2H), 3.80 (d, J=11.1 Hz, 2H), 3.41 (s, 1H), 3.27-3.08 (m, 4H), 3.01-2.83 (m, 1H), 2.72-2.51 (m, 4H), 2.49-2.35 (m, 1H), 2.23 (s, 3H), 2.12-1.87 (m, 2H), 1.71 (s, 1H), 1.41-1.22 (m, 4H). HRMS for C₃₃H₃₈ClF₂N₄O₄: mass calculated 627.2550 [M+H]⁺; mass observed 627.2566 [M+H]⁺. Potency (μM): biochemical qualified AC₅₀: 4.8E-04; NanoBiT qualified absolute AC₅₀: 0.056; cell proliferation qualified AC₅₀: 0.054.

### Example 14

### 1-(5-chloro-2-formyl-1-((tetrahydro-2H-pyran-4-yl)methyl)-1H-indole-3-carbonyl)-4-(4-fluorophenyl)-N-((3S,4R)-4-fluoropyrrolidin-3-yl)piperidine-4-carboxamide

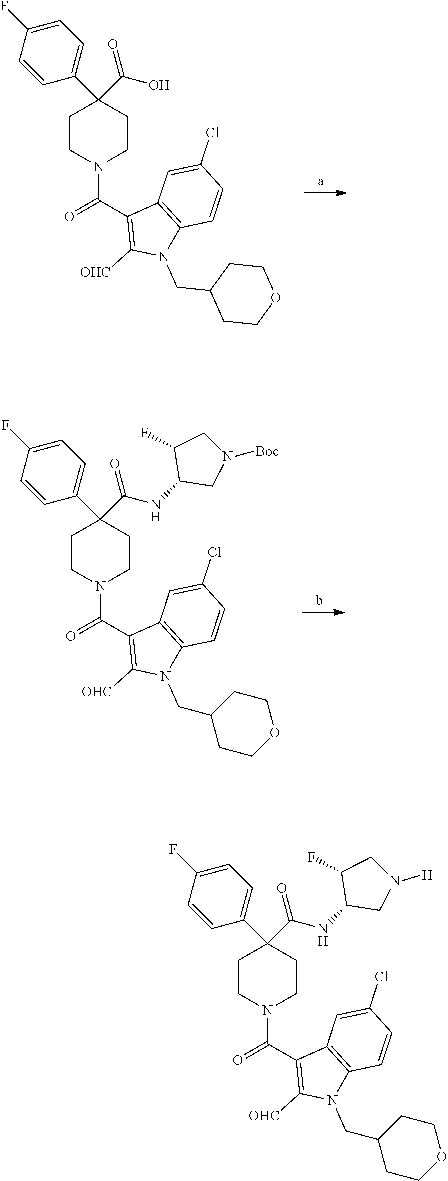 [SMILES: O=Cc1c(C(=O)N2CCC(C(=O)O)(c3ccc(F)cc3)CC2)c2cc(Cl)ccc2n1CC1CCOCC1 ; CC(C)(C)OC(=O)N1C[C@@H](F)[C@@H](NC(=O)C2(c3ccc(F)cc3)CCN(C(=O)c3c(C=O)n(CC4CCOCC4)c4ccc(Cl)cc34)CC2)C1 ; O=Cc1c(C(=O)N2CCC(C(=O)N[C@H]3CNC[C@H]3F)(c3ccc(F)cc3)CC2)c2cc(Cl)ccc2n1CC1CCOCC1]

Step a: To 1-(5-chloro-2-formyl-1-((tetrahydro-2H-pyran-4-yl)methyl)-1H-indole-3-carbonyl)-4-(4-fluorophenyl)piperidine-4-carboxylic acid (150 mg, 0.285 mmol) in DCM (1.42 mL) at RT, were added (3S,4R)-tert-butyl 3-amino-4-fluoropyrrolidine-1-carboxylate (87 mg, 0.427 mmol), HOAT (77 mg, 0.569 mmol), and EDC (82 mg, 0.427 mmol). The reaction mixture was stirred at RT for 2 min., upon which time DIPEA (249 μL, 1.42 mmol) was added and it was stirred at RT overnight. The reaction mixture was diluted with DCM, adsorbed onto silica gel, and concentrated under reduced pressure to dryness. The crude product was purified via silica gel chromatography (EtOAc:heptane, 0:100 to 100:0, then MeOH:DCM, 0:100 to 20:80). Desired fractions were combined and concentrated under reduced pressure to yield tert-butyl (3S,4R)-3-(1-(5-chloro-2-formyl-1-((tetrahydro-2H-pyran-4-yl)methyl)-1H-indole-3-carbonyl)-4-(4-fluorophenyl)piperidine-4-carboxamido)-4-fluoropyrrolidine-1-carboxylate (155 mg) as a yellow foamy solid. MS m/z 713.0 [M+H]⁺.

Step b: To tert-butyl (3S,4R)-3-(1-(5-chloro-2-formyl-1-((tetrahydro-2H-pyran-4-yl)methyl)-1H-indole-3-carbonyl)-4-(4-fluorophenyl)piperidine-4-carboxamido)-4-fluoropyrrolidine-1-carboxylate (24 mg, 0.034 mmol) in DCM (500 μL) at 0° C., was added TFA (51.9 μL, 0.673 mmol). The reaction mixture was stirred at RT for 5 h, upon which time it was diluted with CH₃CN (1 mL), filtered, and purified via reverse phase HPLC (CH₃CN:H₂O with 0.1% formic acid, 25:75 to 50:50). Desired fractions were combined with material from another batch and lyophilized together to yield 1-(5-chloro-2-formyl-1-((tetrahydro-2H-pyran-4-yl)methyl)-1H-indole-3-carbonyl)-4-(4-fluorophenyl)-N-((3S,4R)-4-fluoropyrrolidin-3-yl)piperidine-4-carboxamide (33 mg) as a white solid. ¹H NMR (400 MHz, DMSO-d₆) δ 9.97 (s, 1H), 7.85 (d, J=9.1 Hz, 1H), 7.79-7.58 (m, 2H), 7.57-7.30 (m, 3H), 7.28-7.08 (m, 2H), 5.06 (d, J=55.2 Hz, 1H), 4.58-4.11 (m, 4H), 3.93-3.35 (m, 4H), 3.24-3.03 (m, 5H), 2.97-2.80 (m, 1H), 2.73-2.59 (m, 2H), 2.42-2.25 (m, 2H), 2.22-1.50 (m, 3H), 1.49-1.13 (m, 4H). HRMS for C₃₂H₃₆ClF₂N₄O₄: mass calculated 613.2388 [M+H]⁺; mass observed 613.2362 [M+H]⁺. Potency (μM): biochemical qualified AC₅₀: 4.8E-04; cell proliferation qualified AC₅₀: 0.12.

### Example 15

### 1-(6-((R)-3-(1-(5-chloro-1-((tetrahydro-2H-pyran-4-yl)methyl)-1H-indole-3-carbonyl)-4-(4-fluorophenyl)piperidine-4-carboxamido)pyrrolidin-1-yl)-6-oxohexyl)-3,3-dimethyl-5-sulfo-2-((1E,3E)-5-((E)-1,3,3-trimethyl-5-sulfoindolin-2-ylidene)penta-1,3-dien-1-yl)-3H-indol-1-ium

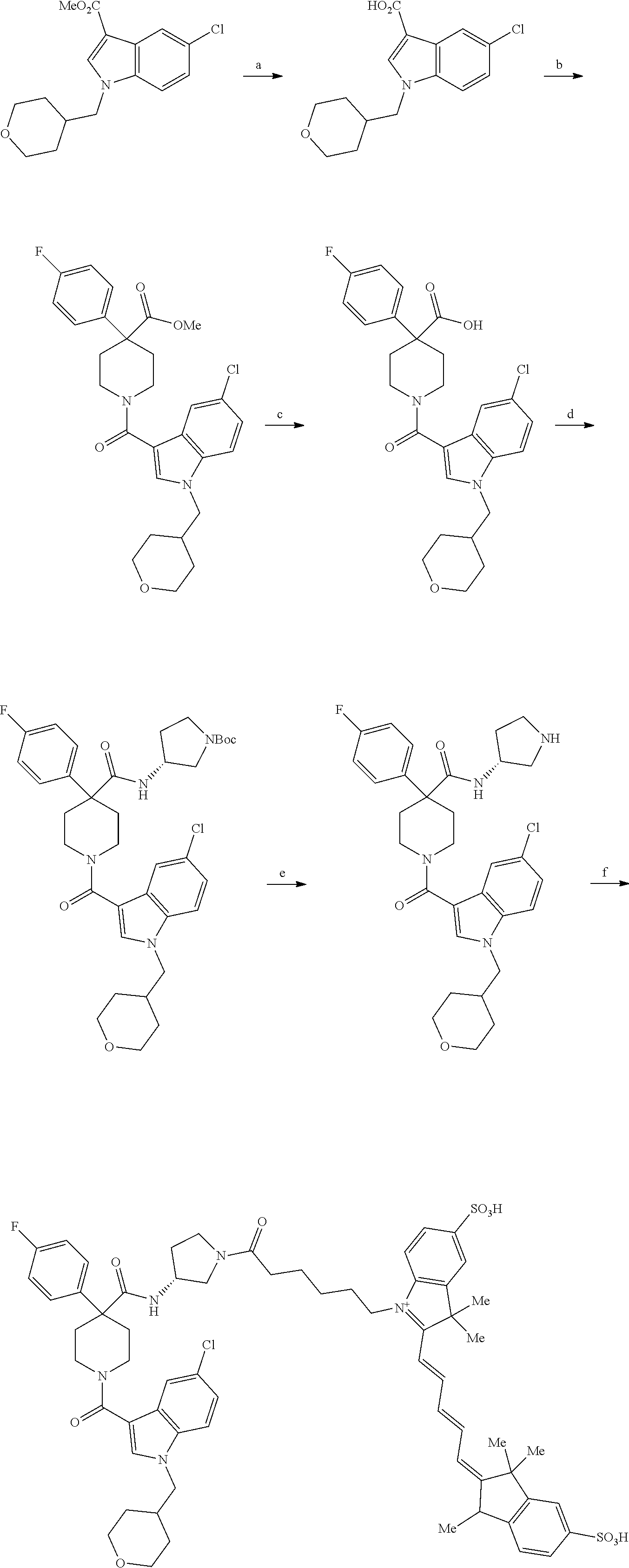

Step a: To methyl 5-chloro-1-((tetrahydro-2H-pyran-4-yl)methyl)-1H-indole-3-carboxylate (2.50 g, 8.12 mmol) in THF (13.5 mL) and MeOH (6.75 mL) at RT, was added aq. 2 N NaOH solution (8.12 mL, 16.3 mmol). The reaction mixture was stirred to homogenize, then split equally between two vials and each heated at 50° C. overnight. The reaction mixtures were combined and concentrated under reduced pressure. The resulting material was diluted with H₂O, washed with DCM, and the organic layer was extracted with H₂O. The combined aq. layers were acidified with aq. 1 N HCl solution and then extracted with DCM (50 mL) and EtOAc (50 mL×2). The combined organic extracts were dried over MgSO₄, filtered, and concentrated under reduced pressure to yield the crude product. The crude product was suspended in DCM, sonicated, and concentrated under reduced pressure. The resulting material was then suspended in Et₂O, sonicated, and collected by filtration. This solid was washed with Et₂O (2×) and dried to yield 5-chloro-1-((tetrahydro-2H-pyran-4-yl)methyl)-1H-indole-3-carboxylic acid (1.95 g, crude) as a white solid. ¹H NMR (400 MHz, DMSO-d₆) δ 12.19 (s, 1H), 8.12 (s, 1H), 7.97 (d, J=2.2 Hz, 1H), 7.69 (d, J=8.8 Hz, 1H), 7.25 (dd, J=8.8, 2.2 Hz, 1H), 4.16 (d, J=7.3 Hz, 2H), 3.89-3.72 (m, 2H), 3.19 (td, J=11.4, 2.9 Hz, 2H), 2.14-2.00 (m, 1H), 1.40-1.21 (m, 4H). MS m/z 294.0 [M+H]⁺.

Step b: To 5-chloro-1-((tetrahydro-2H-pyran-4-yl)methyl)-1H-indole-3-carboxylic acid (1.00 g, 3.40 mmol) in DCM (17 mL) at RT, were added EDC (979 mg, 5.11 mmol) and HOAT (927 mg, 6.81 mmol). The reaction mixture was stirred at RT for 10 min., upon which time methyl 4-(4-fluorophenyl)piperidine-4-carboxylate (889 mg, 3.74 mmol) was added followed by stirring for 10 min. To the reaction mixture was added DIPEA (2.97 mL, 17.0 mmol), and the reaction mixture was stirred at RT overnight. The reaction mixture was added to aq. sat. NaHCO₃ solution and extracted with DCM (50 mL×2). The combined organic extracts were dried over MgSO₄, filtered, adsorbed onto silica gel, and concentrated under reduced pressure to dryness. The crude product was purified via silica gel chromatography (EtOAc:heptane, 40:60 to 100:0). Desired fractions were combined and concentrated under reduced pressure. The material was sonicated with MeOH (7.5 mL), at which point it became a white solid. The organic solvent was removed under reduced pressure to yield methyl 1-(5-chloro-1-((tetrahydro-2H-pyran-4-yl)methyl)-1H-indole-3-carbonyl)-4-(4-fluorophenyl)piperidine-4-carboxylate (1.50 g, crude). 1H NMR (400 MHz, MeOD-d₄) δ 7.78-7.64 (m, 2H), 7.53 (d, J=8.8 Hz, 1H), 7.50-7.42 (m, 2H), 7.24 (dd, J=8.8, 2.1 Hz, 1H), 7.17-7.04 (m, 2H), 4.29 (d, J=13.6 Hz, 2H), 4.15 (d, J=7.3 Hz, 2H), 3.99-3.89 (m, 2H), 3.72 (s, 3H), 3.46-3.34 (m, 4H), 2.64 (d, J=13.5 Hz, 2H), 2.22-2.10 (m, 1H), 2.06-1.93 (m, 2H), 1.54-1.35 (m, 4H). MS m/z 513.0 [M+H]⁺.

Step c: To methyl 1-(5-chloro-1-((tetrahydro-2H-pyran-4-yl)methyl)-1H-indole-3-carbonyl)-4-(4-fluorophenyl)piperidine-4-carboxylate (500 mg, 0.975 mmol) in THF (2.44 mL) and MeOH (244 mL) at RT, was added aq. 2 N NaOH solution (1.22 mL, 2.44 mmol). The reaction mixture was heated to 50° C. over −3 days, upon which time it was acidified to pH 2 with aq. 1 N HCl solution and extracted with EtOAc (50 mL×3). The combined organic extracts were dried over MgSO₄, filtered, and concentrated under reduced pressure to yield 1-(5-chloro-1-((tetrahydro-2H-pyran-4-yl)methyl)-1H-indole-3-carbonyl)-4-(4-fluorophenyl)piperidine-4-carboxylic acid (561 mg, crude). MS m/z 499.0 [M+H]⁺.

Step d: To 1-(5-chloro-1-((tetrahydro-2H-pyran-4-yl)methyl)-1H-indole-3-carbonyl)-4-(4-fluorophenyl)piperidine-4-carboxylic acid (486 mg, 0.974 mmol) in DCM (4.87 mL) at RT, were added (R)-tert-butyl 3-aminopyrrolidine-1-carboxylate (327 mg, 1.75 mmol), HOAT (265 mg, 1.95 mmol), and EDC (280 mg, 1.46 mmol). The reaction mixture was stirred at RT for 2 min., upon which time DIPEA (851 μL, 4.87 mmol) was added and the reaction mixture was stirred at RT overnight. The reaction mixture was diluted with DCM, adsorbed onto silica gel, and evaporated to dryness. The crude product was purified via silica gel chromatography (MeOH:DCM, 0:100 to 10:90). Desired fractions were combined and concentrated under reduced pressure to yield tert-butyl (R)-3-(1-(5-chloro-1-((tetrahydro-2H-pyran-4-yl)methyl)-1H-indole-3-carbonyl)-4-(4-fluorophenyl)piperidine-4-carboxamido)pyrrolidine-1-carboxylate (640 mg). MS m/z 667.0 [M+H]⁺.

Step e: To tert-butyl (R)-3-(1-(5-chloro-1-((tetrahydro-2H-pyran-4-yl)methyl)-1H-indole-3-carbonyl)-4-(4-fluorophenyl)piperidine-4-carboxamido)pyrrolidine-1-carboxylate (640 mg, 0.959 mmol) in MeOH (1.6 mL) at RT, was added 4 M HCl solution in 1,4-dioxane (6.0 mL, 24.0 mmol). The reaction mixture was stirred at RT overnight, upon which time it was concentrated under reduced pressure to yield a brown oil. To the oil was added Et₂O and the mixture was concentrated under reduced pressure. To the residue was added DCM and the mixture was concentrated under reduced pressure (2×) to yield (R)-1-(5-chloro-1-((tetrahydro-2H-pyran-4-yl)methyl)-1H-indole-3-carbonyl)-4-(4-fluorophenyl)-N-(pyrrolidin-3-yl)piperidine-4-carboxamide hydrochloride salt (658 mg, crude) as a pale brown solid. MS m/z 565.3 [M−H]⁻.

Step f: To (R)-1-(5-chloro-1-((tetrahydro-2H-pyran-4-yl)methyl)-1H-indole-3-carbonyl)-4-(4-fluorophenyl)-N-(pyrrolidin-3-yl)piperidine-4-carboxamide hydrochloride salt (6.4 mg, 10.6 μmol) in DMF (500 μL) at RT, were added potassium 1-(6-((2,5-dioxopyrrolidin-1-yl)oxy)-6-oxohexyl)-3,3-dimethyl-2-((1E,3E,5E)-5-(1,3,3-trimethyl-5-sulfonatoindolin-2-ylidene)penta-1,3-dien-1-yl)-3H-indol-1-ium-5-sulfonate (sulfo-cyanine5 NHS ester, Lumiprobe, CAS #2230212-27-6, 7 mg, 9.0 μmol) and DIPEA (18.5 μL, 0.106 mmol). The reaction mixture was stirred at RT for 3 h while protected from light. The reaction mixture was diluted with MeOH (1 mL), filtered, and purified via reverse phase HPLC (CH₃CN:H₂O with 0.1% formic acid, 25:75 to 50:50). Desired fractions were combined, lyophilized, and the resulting material was purified once more under the same conditions. Desired fractions were combined and lyophilized to yield 1-(6-((R)-3-(1-(5-chloro-1-((tetrahydro-2H-pyran-4-yl)methyl)-1H-indole-3-carbonyl)-4-(4-fluorophenyl)piperidine-4-carboxamido)pyrrolidin-1-yl)-6-oxohexyl)-3,3-dimethyl-5-sulfo-2-((1E,3E)-5-((E)-1,3,3-trimethyl-5-sulfoindolin-2-ylidene)penta-1,3-dien-1-yl)-3H-indol-1-ium (1.3 mg) as a bright blue powder. ¹H NMR (400 MHz, DMSO-d₆) δ 8.36 (t, J=13.1 Hz, 2H), 7.87 (d, J=2.6 Hz, 1H), 7.84-7.75 (m, 3H), 7.71 (d, J=2.1 Hz, 1H), 7.69-7.57 (m, 3H), 7.39 (ddd, J=9.0, 5.4, 2.0 Hz, 2H), 7.36-7.27 (m, 2H), 7.24-7.11 (m, 3H), 6.58 (s, 2H), 6.29 (dd, J=19.8, 13.9 Hz, 2H), 4.34-4.00 (m, 6H), 3.80 (d, J=11.1 Hz, 2H), 3.59 (s, 3H), 3.48-3.39 (m, 1H), 3.33 (s, 5H), 3.19 (dd, J=12.9, 10.0 Hz, 4H), 3.10-2.95 (m, 1H), 2.63-2.55 (m, 2H), 2.23-1.93 (m, 4H), 1.81 (d, J=13.6 Hz, 3H), 1.68 (t, J=2.2 Hz, 12H), 1.56-1.41 (m, 2H), 1.41-1.17 (m, 7H). HRMS for C₆₃H₇₃ClFN₆O₁₀S₂: mass calculated 1191.4497 [M]⁺; mass observed 1191.4431 [M]⁺.

### Assays

The utility of the compounds of the invention described herein can be evidenced by testing in the following assays. Potency is demonstrated using the biochemical assay (qualified AC₅₀—Example 16), the NanoBiT assay (qualified absolute AC₅₀—Example 17), and the cell proliferation assay (qualified AC₅₀—Example 18). Compounds of the invention are further studied for their anti-tumor activity and tolerability in WM793 BRAF^{V600E} tumor xenografts in nude mice and nude rats.

### Biochemical Assay

The competition assays were performed at RT in a 384-well white polystyrene, flat bottom plate (Greiner #784075) using a final reaction volume of 15 μL and the following assay buffer conditions: 50 mM MOPS, pH 7.2, 10 mM MgCl₂, 0.5 mM TCEP, 0.01% Triton-X-100. 75 nL of a 10 mM compound solution in DMSO or 100% DMSO (12 point titration, final concentration: 50 μM-300 pM; final DMSO: 0.5%) was added using an acoustic liquid handler (Echo, Beckman Coulter) to wells containing 7.5 μL of 1-(6-((R)-3-(1-(5-chloro-1-((tetrahydro-2H-pyran-4-yl)methyl)-1H-indole-3-carbonyl)-4-(4-fluorophenyl)piperidine-4-carboxamido)pyrrolidin-1-yl)-6-oxohexyl)-3,3-dimethyl-5-sulfo-2-((1E,3E)-5-((E)-1,3,3-trimethyl-5-sulfoindolin-2-ylidene)penta-1,3-dien-1-yl)-3H-indol-1-ium (Example 15); 7.5 μL 400 pM SA-Tb (Cisbio, 610SATLB) and 400 pM ERK2[2-360(p-T185/p-Y187)]-Avi was added to the plate and incubated overnight at RT. HTRF signal was measured using a microplate reader (PheraStar, BMG Labtech) with excitation and emission wavelengths of 665 and 620 nm, respectively. The inhibitor dose-response curves were analyzed from the HTRF ratio signal using normalized IC₅₀ regression curve fitting with control-based normalization.

### Nanobit Assay

Cancer cell media was prepared by adding 500 mL of RPMI (ThermoFisher cat ##22400-089) and 10% Fetal Bovine Serum (Gibco) for WM793 cells (ATCC Cat #CRL-2806, RRID: CVCL 8787) and F12K (Thermo Fisher cat #21127-022, 500 mL; or 21127-030, 10×500 mL)+10% FBS for PC3 (ATCC CRL-1435) cells. The entire media with supplements was filter sterilized through a 0.4 μm bottle top filter system. Cancer cell lines were detached from traditional cell culture flasks by washing three times with 1×PBS and adding an appropriate amount of 0.25% TrypLE™ Express Enzyme (1×), no phenol red (ThermoFisher cat #12604013) to create a thin layer over the cells. Trypsin was neutralized with media containing serum and proceeded to count the cells.

A nanobit stable cell line was generated using WM793 cells with nanobit-large-bit-ERK2 and nanobit-small-bit-PEA15. The cell solution concentration was adjusted to 62.5×10⁵ in order to seed a total of 5000 cells per well in 30 μL of phenol red-free RPMI1640 (ThermoFisher cat #11835-030, 500 mL; or 11835-055, 10×500 mL)+10% FBS+Puromycin (1 μg/mL)+Hygromycin (100 μg/mL) and MT cell viability substrate (Promega #G9711, 1:3000 at a final dilution) into 384-well tissue culture treated plates (Greiner). In a biological safety cabinet, cells were seeded using a sterilized small stainless steel tipped cassette on a Multi-drop Combi. Between different cell lines, the tubing of the cassette was flushed with 1× sterile PBS for 10 s. Alternatively, cells can be seeded using a Multi-flow located within a Hepa filtered robotics room using either a sterilized small sapphire jewel tipped cassette and the peristatic pump, or the attached syringe pump, depending on volume of plates per cell line needed. The plates were placed in an incubator set at 37° C., with 5% CO₂ and 95% relative humidity. A 10-point, 3-fold serial dilution of compounds was prepared in 100% DMSO on a liquid handling robot with a 10 mM top concentration. 90 nl/well was dispensed in three assay plates/1 compound source plate, with the highest concentration at 30 μM. After addition of compound was complete, the plates were placed into an incubator at 37° C., with 5% CO₂ and 95% relative humidity for overnight. Luminescence was captured on a plate luminometer such as an Envision or View Lux to measure Day 0 time point both select cell lines. LUM plus, gain 3500, 0.1 s and focal height 12.5 [mm] on PheraStar reader. The raw data was extracted from the instrument and normalized all fields containing test compounds to the average of all wells containing DMSO alone as neutral control. From this value, the percent growth inhibition was calculated for each compound. Dose response curves and inhibitory IC₅₀s were generated by graphing the normalized data values against the compound concentration using either a program such as GraphPad™ or Excel Fit.

### Cell Proliferation Assay

After counting the cells, the concentration of the cell solution was adjusted to seed a total of 750 cells per well in 50 μL of complete media into 384-well tissue culture treated plates. In a biological safety cabinet, cells were seeded using a sterilized small stainless steel tipped cassette on a Multi-drop Combi. Between different cell lines, the tubing of the cassette was flushed with 1× sterile PBS for 10 seconds. Alternatively, cells can be seeded using a Multi-flow located within a Hepa filtered robotics room using either a sterilized small sapphire jewel tipped cassette and the peristatic pump, or the attached syringe pump, depending on volume of plates per cell line needed. The plates were placed in an incubator set at 37° C., with 5% CO₂ and 95% relative humidity. A 10-point, 3-fold serial dilutions of compounds in 100% DMSO was prepared on a liquid handling robot with a 10 mM top concentration. 100 nL/well in three assay plates/1 compound was dispensed to source plate, with the highest concentration at 20 μM. After addition of compound was complete, plates were placed into an incubator at 37° C., with 5% CO₂ and 95% relative humidity for 3 d. An adequate volume of CellTiter-Glo® (Promega #G7571) detection reagent was pre-warmed to RT or in a 37° C. H₂O bath. 25 μL of the reagent was added to each well using either a Multi-drop Combi or MultiFlo and incubate at RT for at least 15 min. Luminesce was captured on a plate luminometer such as an Envision or View Lux to measure Day 0 time point both select cell lines. LUM plus, gain 3500, 0.1 s and focal height 12.5 mm on PheraStar reader. At Day 3, an adequate volume of CellTiter-Glo® detection reagent was pre-warmed at RT or in a 37° C. H₂O bath. A total of 25 μL of the reagent was added to each well using either a Multi-drop Combi or MultiFlo and incubate at RT for at least 15 min. Luminesce was captured on a plate luminometer such as an Envision or View Lux to measure Day 3 time point both select cell lines. LUM plus, gain 3500, 0.1 s and focal height 12.5 mm on PheraStar reader. Raw data was extracted from the instrument and all fields containing test compounds were normalized to the average of all wells containing DMSO alone from Day 3 as neutral control and Day 0 starting numbers. From this value, percent growth inhibition of each compound was calculated. Dose response curves and growth inhibitory IC₅₀s were generated by graphing the normalized data values against the compound concentration using a program such as GraphPad™ or Excel Fit.

### Anti-Tumor Activity and Tolerability in WM793 BRAF^{V600E} Tumor Xenograft in Nude Mice.

SCID beige female mice (Charles River, 8-9 weeks old) were acclimatized into animal facility and implanted subcutaneously in the right flank with 20-40 mg WM793^{V600E} tumor fragment from a previous passage with sterile techniques under isoflurane anesthesia using a 10-gauge trocar (n=8/group). Body weights were monitored at least once a week and calculated as (BWcurrent−BWinitial)/(BWinitial)×100%. After 1-3 weeks when palpable tumors developed, tumor volumes were monitored at least once a week by caliper measurements. Tumor volumes (mm³) were calculated as (length×width×width)/2. Mice were randomized and selected for study based on tumor sizes (200-250 mm³) and appropriate health conditions. A compound of Formula I, formulated in 0.1% Tween 80+0.5% MC at 3, 7.5, 15 and 30 mg/ml was dosed at 10 ml/kg per os. Animals were euthanized based on poor health conditions, tumor volumes exceeding 1500 mm³, or have reached the end of study. T/C % is calculated by 100×ΔT/ΔC if ΔT>0, in which ΔT is the change of tumor volume after drug treatment and ΔC is the change of tumor volume in vehicle control group. Regression % was calculated by 100×ΔT/Tinitial if ΔT<0, in which ΔT is the change of tumor volume.

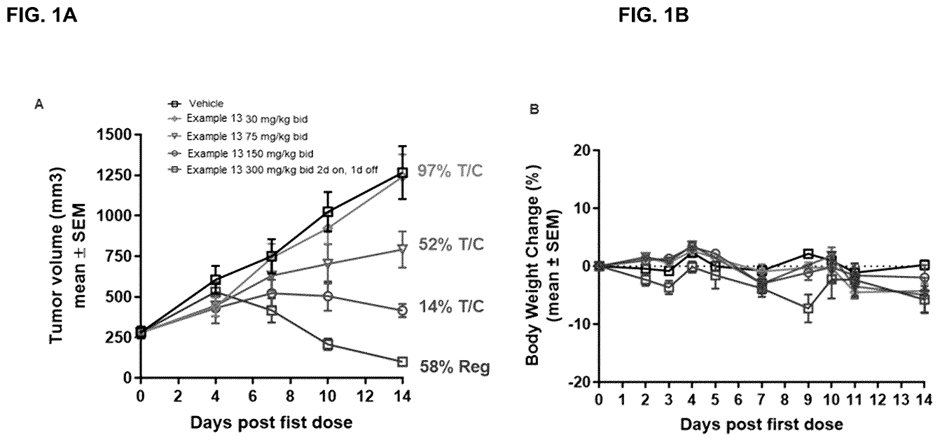

FIGS. 1A-1B show the anti-tumor activity and tolerability of Example 13 in WM793 BRAF^{V600E} tumor xenograft in nude mice. A) Treatment of tumor bearing mice with Example 13 at 30 mg/kg bid (orange line), 75 mg/kg bid (blue line), 150 mg/kg bid (red line) and 300 mg/kg bid 2 day on, 1 day off (green line) for 14 days leads to dose dependent antitumor activity, with regression achieved at the highest dose. B) Percent body weight change following first dose indicates Example 13 leads to minimal body weight loss and is well tolerated. One mouse at the highest dosing group was sacrificed on day 11 due to increased body weight loss.

### Anti-Tumor Activity and Tolerability in WM793 BRAF^{V600E} Tumor Xenograft in Nude Rats.

Female RNU rats (Charles River, 4-5 weeks of age) were acclimatized into animal facility and pretreated with 100 mg/kg cyclophosphamide IP at 10 ml/kg 24 hrs prior to tumor implant. Nude rats were subcutaneously implanted on the right side with WM793 BRAF^{V600E} tumors via fragments (20-40 mg) from a previously passaged tumor with sterile techniques under isoflurane anesthesia using a 10 gauge trocar (n=4/group). Body weights were monitored at least once a week and calculated as (BWcurrent−BWinitial)/(BWinitial)×100%. After 1-3 weeks when palpable tumors developed, tumor volumes were monitored at least once a week by caliper measurements. Tumor volumes (mm³) were calculated as (length×width×width)/2. Rats were randomized and selected for study based on tumor sizes (100-200 mm³) and appropriate health conditions. Compounds of Formula I, formulated in either 20% HPBCD at 5 mg/ml or 20% HPBCD at 2.5 mg/ml were dosed at 10 ml/kg per os. Animals were euthanized based on poor health conditions, tumor volumes exceeded 2500 mm³, or have reached the end of study. Regression % was calculated by 100×ΔT/Tinitial if ΔT<0, in which ΔT is the change of tumor volume.

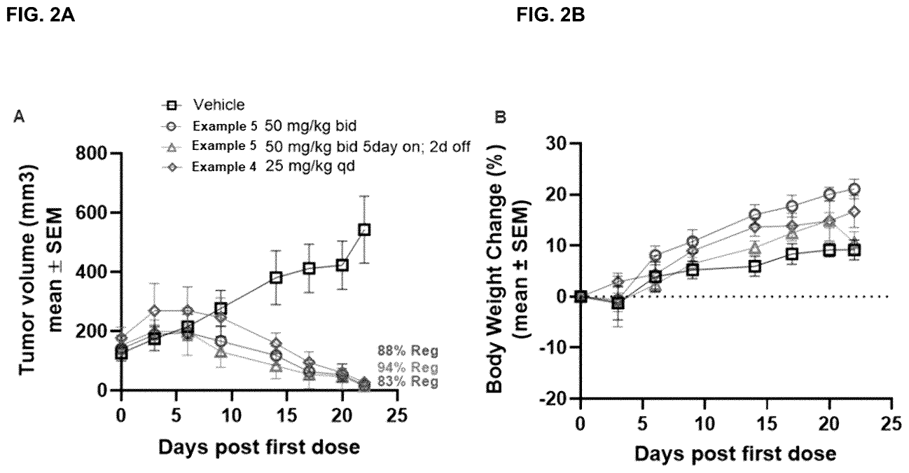

FIGS. 2A-2B show the anti-tumor activity and tolerability of Example 5 and Example 4 in WM793 BRAF^{V600E} tumor xenograft in nude rats. A) Treatment of tumor bearing rats with Example 5 at 50 mg/kg bid for 22 days (red line) leads to tumor regression, which is maintained when 2 day holiday break is provided (treatment with 50 mg/kg bid for 5 days with 2 day break-orange line). Similarly, treatment of tumor bearing rats with Example 4 for 22 days leads to regression (blue line). B) Percent body weight change following first dose indicates all compounds are well tolerated.

1. A compound of formula (I): 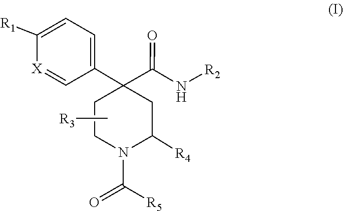
wherein:
X is selected from N and CR₆, wherein R₆ is selected from hydrogen and halo;
R₁ is selected from hydrogen and halo;
R₂ is selected from —X₁—R₂ₐ and R₂ₐ;
X₁ is selected from C₁-C₄alkylene and C₂-C₄haloalkylene;
R₂ₐ is selected from i) hydrogen, and ii) a ring substituted with 0 to 3 substituents R_{2b}, wherein the ring is selected from a) 3-6 membered saturated or partially unsaturated carbocyclic ring, b) 5-6 membered heteroaryl, c) phenyl, and d) 4 to 6 membered heterocyclyl (e.g. fully saturated heterocyclyl) containing 1 or 2 heteroatoms independently selected from oxygen and nitrogen;
each R_{2b} is independently selected from halo, hydroxyl, C₁-C₃alkyl, C₃-C₄cycloalkyl, C₁-C₃haloalkyl, C₁-C₃hydroxyalkyl, O—C₁-C₃alkyl, C₁-C₃alkylene-O—C₁-C₃alkyl, cyano, —CO₂R₁₁, —CO₂N(R₁₁)₂, —X₂—CO₂R₁₁ and —X₂—CO₂N(R₁₁)₂;
X₂ is selected from C₁-C₅alkylene and C₃-C₆cycloalkylene;
each R₁₁ is independently selected from hydrogen, C₁-C₅alkyl, C₃-C₅cycloalkyl, C₁-C₅haloalkyl and C₃-C₅cyclohaloalkyl, or two R₁ groups together with the nitrogen atom to which they are mutually attached join to form a 4 to 6 membered heterocyclic ring containing 1 heteroatom which is nitrogen;
R₃ is selected from hydrogen, C₁-C₃alkyl and C₁-C₃haloalkyl, and R₄ is hydrogen, or R₃ and R₄ together with the piperidinyl ring of formula (I) to which R₃ and R₄ are attached join to form a 7 or 8 membered bridged or fused heterocyclic ring;
R₅ is selected from: 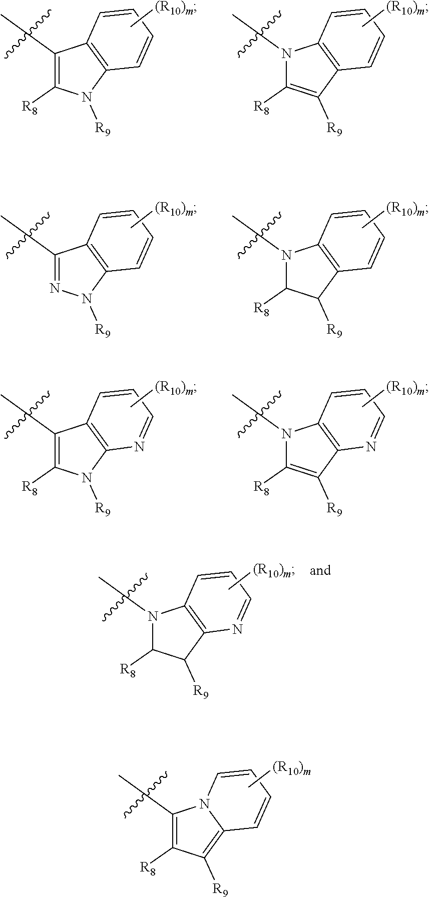
wherein:
R₈ is selected from hydrogen, halo, C₁-C₆alkyl, C₃-C₄cycloalkyl, C₁-C₆haloalkyl, C₁-C₆alkylene-O—C₁-C₄alkyl, C₁-C₆haloalkylene-O—C₁-C₄alkyl, C₁-C₆hydroxyalkyl, C₁-C₆haloalkylene-O—C₁-C₄haloalkyl, C₁-C₆alkylene-O—C₁-C₄haloalkyl, C(═O)H and cyano, and R₉ is selected from —X₃—R₉ₐ and R₉ₐ;
or R₈ and R₉ together with the carbon atoms to which R₈ and R₉ are attached form a ring substituted with 0 to 3 R_{9b} groups, wherein the ring is a) 5-6 membered saturated or partially unsaturated carbocyclic ring, or b) 5-6 membered heterocyclyl comprising 1 heteroatom which is O;
X₃ is selected from C₁-C₂alkylene and C₃-C₅cycloalkylene;
R₉ₐ is selected from i) hydrogen and ii) a ring substituted with 0 to 3 R_{9b} groups, wherein the ring is a) phenyl, b) 5-6 membered heteroaryl, c) C₃-C₇cycloalkyl, d) C₇-C₉spiroalkyl, e) 4 to 7 membered heterocyclyl comprising 1 or 2 heteroatoms which is/are each 0, or f) 7 to 9 membered spiroheterocyclyl comprising 1 or 2 heteroatoms which is/are each O;
each R_{9b} is independently selected from halo, hydroxy, C₁-C₄alkyl, C₁-C₄haloalkyl, O—C₁-C₄alkyl, O—C₁-C₄haloalkyl, C₁-C₄alkylene-O—C₁-C₄alkyl, C₁-C₄haloalkylene-O—C₁-C₄alkyl, C₁-C₄alkylene-O—C₁-C₄haloalkyl, C₁-C₄haloalkylene-O—C₁-C₄haloalkyl, C₃-C₇cycloalkyl substituted with 0 to 2 R_{9c} groups, C₃-C₇cyclohaloalkyl substituted with 0 to 2 R_{9c} groups, O—C₃-C₇cycloalkyl substituted with 0 to 2 R_{9c} groups, O—C₃-C₇cyclohaloalkyl substituted with 0 to 2 R_{9c} groups, 3-7 membered heterocyclyl comprising 1 heteroatom which is O substituted with 0 to 2 R_{9c} groups, O-3-7 membered heterocyclyl comprising 1 heteroatom which is O substituted with 0 to 2 R_{9c} groups, phenyl substituted with 0 to 2 R_{9c} groups, pyridinyl substituted with 0 to 2 Rec groups, O—C₁-C₃alkylene-C₃-C₇cycloalkyl substituted with 0 to 2 R_{9c} groups, O—C₁-C₃alkylene-C₃-C₇cyclohaloalkyl substituted with 0 to 2 R_{9c} groups and O—C₁-C₃alkylene-3-7 membered heterocyclyl comprising 1 heteroatom which is O substituted with 0 to 2 R_{9c} groups;
or wherein two R_{9b} groups in combination with the carbon atom to which they are mutually attached form a ring substituted with 0 to 2 R_{9c} groups, wherein the ring is i) C₃-C₆cycloalkyl, or ii) 4-6 membered heterocyclyl containing one heteroatom which is oxygen;
each R_{9c} is independently selected from halo (e.g. fluoro), CH₃ and OCH₃;
each R₁₀ is halo; and
m is an integer from 0 to 2;
or a pharmaceutically acceptable salt thereof.

2. The compound or pharmaceutically acceptable salt thereof of claim 1, wherein the compound is of formula (Ia): 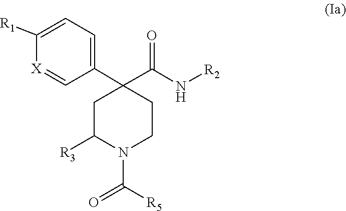
in which:
X is selected from N and CR₆; wherein R₆ is selected from hydrogen and halo;
R₁ is selected from hydrogen and halo;
R₂ is selected from —X₁—R₂ₐ and R₂ₐ;
X₁ is selected from C₁-C₂alkylene and C₂haloalkylene;
R₂ₐ is selected from i) hydrogen and ii) a ring substituted with 0 to 3 substituents R_{2b}; wherein the ring is selected from a) 3-6 membered saturated or partially unsaturated carbocyclic ring, b) 5-6 membered heteroaryl, c) phenyl, and d) 4 to 6 membered heterocyclyl (e.g. fully saturated heterocyclyl) containing 1 or 2 heteroatoms independently selected from oxygen and nitrogen;
each R_{2b} is independently selected from halo, hydroxyl, C₁-C₃alkyl, C₁-C₃haloalkyl, C₁-C₃hydroxyalkyl, O—C₁-C₃alkyl, C₁-C₃alkylene-O—C₁-C₃alkyl, cyano, —CO₂R₁₁, —CO₂N(R₁₁)₂, —X₂—CO₂R₁₁ and —X₂—CO₂N(R₁₁)₂;
X₂ is selected from C₁-C₅alkylene and C₃-C₆cycloalkylene;
each R₁₁ is independently selected from hydrogen, C₁-C₅alkyl and C₃-C₆cycloalkyl;
R₃ is selected from hydrogen and methyl;
R₅ is selected from: 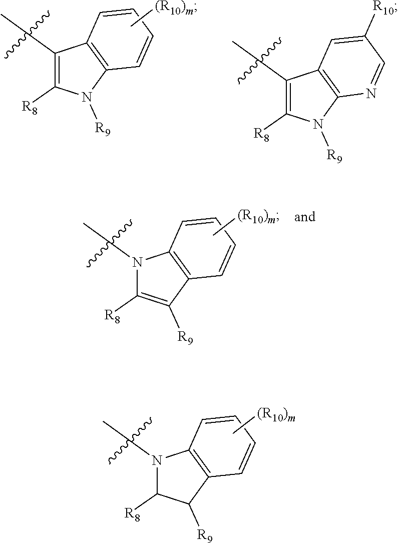
wherein:
R₈ is selected from hydrogen, methyl, ethyl, CHF₂, CH₂OCH₂CH₃, CH₂CH₂OCH₃, C(═O)H and cyano, and R₉ is selected from X₃—R₉ₐ and R₉ₐ;
or R₈ and R₉ together with the carbon atoms to which R₈ and R₉ are attached form a ring substituted with 0 to 1 R_{9b} groups, wherein the ring is a 5-6 membered heterocyclyl comprising 1 heteroatom which is O;
X₃ is C₁-C₂alkylene;
R₉ₐ is selected from i) hydrogen, ii) a ring substituted with 0 to 3 R_{9b} groups, wherein the ring is a) C₄-C₆cycloalkyl, b) 6 membered heterocyclyl comprising 1 heteroatom which is O or c) 7 to 9 membered spiroheterocyclyl comprising 1 or 2 heteroatoms which is/are each O;
each R_{9b} is independently selected from halo, C₁-C₃alkyl, O—C₁-C₃alkyl, O-4-6 membered heterocyclyl comprising 1 heteroatom which is O substituted with 0 to 2 R_{9c} groups, pyridinyl substituted with 0 to 2 R_{9c} groups and O—C₁-C₃alkylene-4-6 membered heterocyclyl comprising 1 heteroatom which is O substituted with 0 to 2 R_{9c} groups;
or wherein two R_{9b} groups in combination with the carbon atom to which they are mutually attached form a ring substituted with 0 to 2 R_{9c} groups, wherein the ring is i) C₃-C₆cycloalkyl, or ii) 4-6 membered heterocyclyl containing one heteroatom which is oxygen;
each R_{9c} is independently selected from halo (e.g. fluoro), CH₃ and OCH₃;
each R₁₀ is halo; and
m is an integer from 0 to 2;
or a pharmaceutically acceptable salt thereof.

3. The compound or pharmaceutically acceptable salt thereof according to claim 1, wherein X is CH or CF.

4. The compound or pharmaceutically acceptable salt thereof according to claim 3, wherein X is CH.

5. The compound or pharmaceutically acceptable salt thereof according to claim 1, wherein R₂ is R₂ₐ.

6. The compound or pharmaceutically acceptable salt thereof according to claim 5, wherein R₂ₐ is a ring substituted with 0 to 3 substituents R_{2b}; wherein the ring is selected from a) C₄-C₆ cycloalkyl, and b) 4 to 6 membered heterocyclyl (e.g. fully saturated heterocyclyl) containing 1 or 2 heteroatoms independently selected from oxygen and nitrogen.

7. The compound or pharmaceutically acceptable salt thereof according to claim 6, wherein R₂ₐ is a ring substituted with 1 to 3 substituents R_{2b}; wherein the ring is selected from a) C₅-C₆ cycloalkyl, and b) 4 to 6 membered heterocyclyl (e.g. fully saturated heterocyclyl) containing 1 or 2 heteroatoms independently selected from oxygen and nitrogen.

8. The compound or pharmaceutically acceptable salt thereof according to claim 1, wherein each R_{2b} is independently selected from halo, C₁-C₃alkyl and CO₂H.

9. The compound or pharmaceutically acceptable salt thereof according claim 1, wherein each R_{2b} is independently selected from fluoro, chloro, methyl and CO₂H.

10. The compound or pharmaceutically acceptable salt thereof according claim 1, wherein R₃ is H.

11. The compound or pharmaceutically acceptable salt thereof according to claim 1, wherein R₅ is selected from:

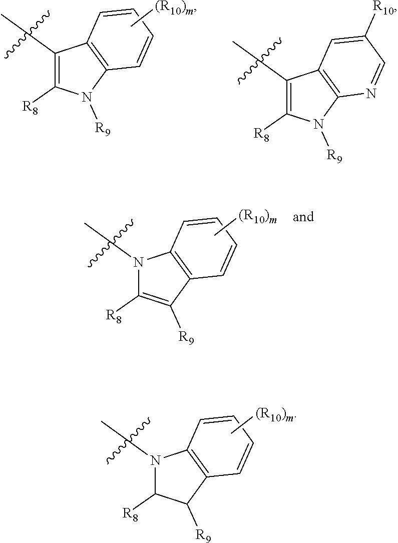

12. The compound or pharmaceutically acceptable salt thereof according to claim 11, wherein m is 1 or 2.

13. The compound or pharmaceutically acceptable salt thereof according to claim 1, wherein R₅ is selected from:

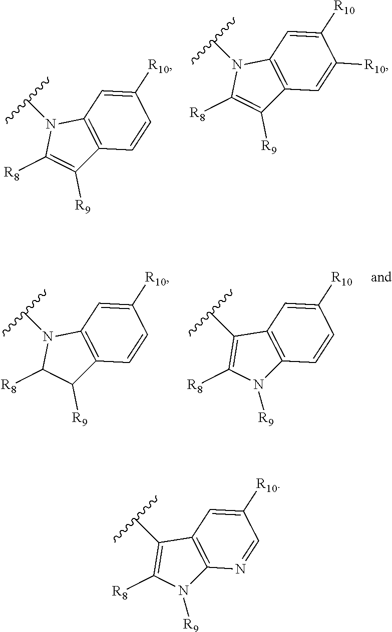

14. The compound or pharmaceutically acceptable salt thereof according to claim 1, wherein each R₁₀ is independently selected from fluoro, chloro and bromo.

15. The compound or pharmaceutically acceptable salt thereof according to claim 1, wherein R₂ₐ is C₅-C₆cycloalkyl substituted with 1 to 3 substituents R_{2b}.

16. The compound or pharmaceutically acceptable salt thereof according to claim 1, wherein R₂ₐ is C₆cycloalkyl substituted with 1 to 3 substituents R_{2b}.

17. The compound or pharmaceutically acceptable salt thereof according to claim 15, wherein one R_{2b} is CO₂H, and the other 0 to 2 R_{2b} groups are each independently selected from methyl, fluoro and chloro.

18. The compound or pharmaceutically acceptable salt thereof according to claim 1, wherein the compound is of formula (Ib): 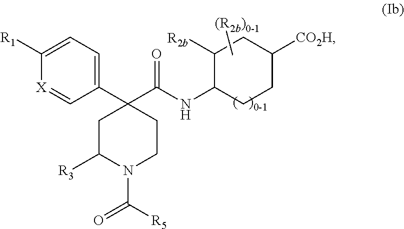
wherein X, R₁, R₃, R₅ and each R_{2b} are as defined in claim 1.

19. The compound or pharmaceutically acceptable salt thereof according to claim 18, wherein each R_{2b} is independently selected from methyl, fluoro and chloro.

20. The compound or pharmaceutically acceptable salt thereof according to claim 1, wherein R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b}.

21. The compound or pharmaceutically acceptable salt thereof according to claim 20, wherein R₂ₐ is 4 to 6 membered heterocyclyl (e.g. fully saturated heterocyclyl) containing 1 heteroatom which is nitrogen substituted with 0 to 2 substituents R_{2b}.

22. The compound or pharmaceutically acceptable salt thereof according to claim 20, wherein R₂ₐ is 4 to 6 membered heterocyclyl (e.g. fully saturated heterocyclyl) containing 1 heteroatom which is nitrogen substituted with 1 or 2 substituents R_{2b}, and wherein each R_{2b} substituent is independently selected from methyl and halo (e.g. fluoro).

23. The compound or pharmaceutically acceptable salt thereof according to claim 1, wherein R₈ is selected from hydrogen, methyl, ethyl, CHF₂, CH₂CH₂OCH₃, C(═O)H and cyano.

24. The compound or pharmaceutically acceptable salt thereof according to claim 1, wherein R₉ is X₃—R₉ₐ.

25. The compound or pharmaceutically acceptable salt thereof according to claim 24, wherein X₃ is CH₂.

26. The compound or pharmaceutically acceptable salt thereof according to claim 1, wherein R₉ₐ is a ring substituted with 0 to 2 R_{9b} groups, wherein the ring is a) C₅-C₆cycloalkyl or b) 6 membered heterocyclyl comprising 1 heteroatom which is O.

27. The compound or pharmaceutically acceptable salt thereof according to claim 26, wherein R₉ₐ is a ring substituted with 0 or 1 R_{9b} groups, wherein the ring is a) C₆cycloalkyl or b) 6 membered heterocyclyl comprising 1 heteroatom which is O.

28. The compound or pharmaceutically acceptable salt thereof according to claim 1, wherein each R_{9b} is independently selected from O—C₁-C₃alkyl and O-6 membered heterocyclyl comprising 1 heteroatom which is O.

29. The compound or pharmaceutically acceptable salt thereof according to claim 1, wherein R₈ and R₉ together with the carbon atoms to which R₈ and R₉ are attached form a ring substituted with 0 to 1 R_{9b} groups, wherein the ring is a 5-6 membered heterocyclyl comprising 1 heteroatom which is O.

30. The compound or pharmaceutically acceptable salt thereof according to claim 29, wherein R₈ and R₉ together with the carbon atoms to which R₈ and R₉ are attached form a ring substituted with an R_{9b} group, wherein the ring is a 6 membered heterocyclyl comprising 1 heteroatom which is O, and wherein R_{9b} is selected from O-4-6 membered heterocyclyl comprising 1 heteroatom which is O substituted with 0 to 2 (e.g. 0) R_{9c} groups, and phenyl substituted with 0 to 2 (e.g. 0) R_{9c} groups.

31. The compound or pharmaceutically acceptable salt thereof according to claim 29, of formula (Ic) or (Id): 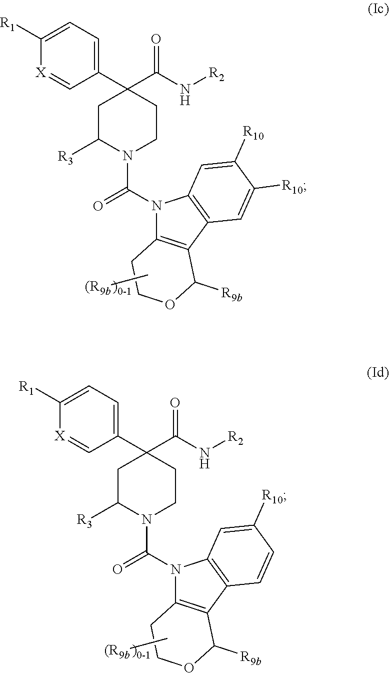
wherein X, R₁, R₂, R₃, R_{9b} and each R₁₀ independently are as defined in claim 29.

32. The compound or pharmaceutically acceptable salt thereof according to claim 31, of formula (Ic-1) or (Id-1): 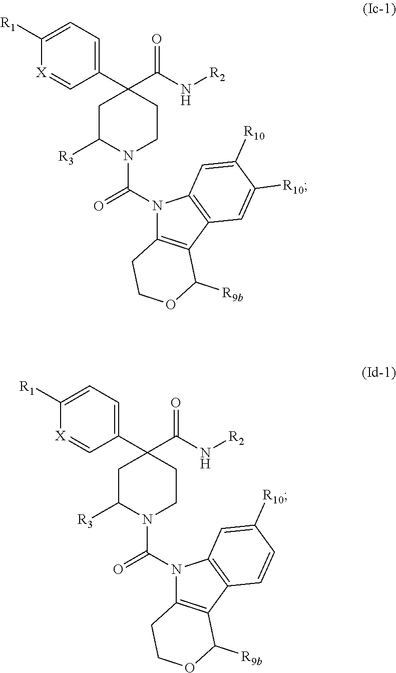
wherein X, R₁, R₂, R₃, R_{9b} and each R₁₀ independently are as defined in claim 31.

33. The compound or pharmaceutically acceptable salt thereof according to claim 32, of formula (Ic-2) or (Id-2): 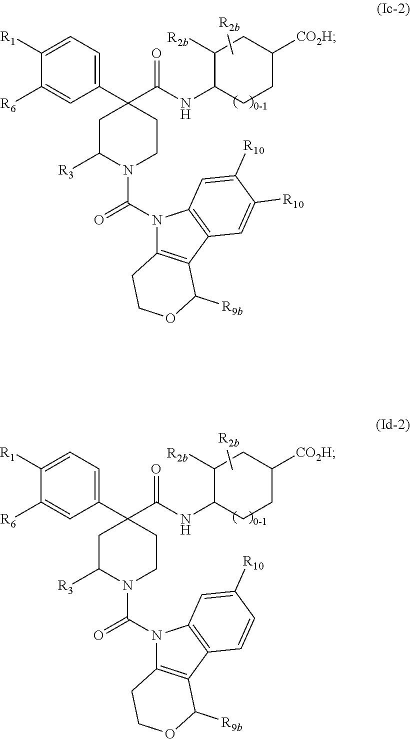
wherein R₁, each R_{2b} independently, R₃, R₆, R_{9b} and each R₁₀ independently are as defined in claim 32.

34. The compound or pharmaceutically acceptable salt thereof according to claim 32, wherein R_{9b} is selected from O-6 membered heterocyclyl comprising 1 heteroatom which is O, and phenyl.

35. The compound or pharmaceutically acceptable salt thereof according to claim 1, wherein the compound is of formula (II): 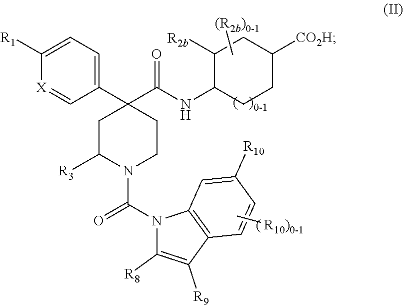
wherein X, R₁, R₃, each R_{2b} independently, and each R₁₀ independently are as defined in claim 1;
R₈ is selected from hydrogen, methyl, ethyl, CHF₂, CH₂CH₂OCH₃, C(═O)H and cyano;
R₉ is X₃—R₉ₐ;
X₃ is CH₂;
R₉ₐ is selected from i) hydrogen and ii) a ring substituted with 0 to 2 (e.g. 0 to 1) R_{9b} groups, wherein the ring is a) C₅-C₆cycloalkyl or b) 6 membered heterocyclyl comprising 1 heteroatom which is O; and each R_{9b} is as defined in claim 1 (e.g. each R_{9b} is independently selected from O—C₁-C₃alkyl and 6 membered heterocyclyl comprising 1 heteroatom which is O).

36. The compound or pharmaceutically acceptable salt thereof according to claim 1, wherein the compound is of formula (III): 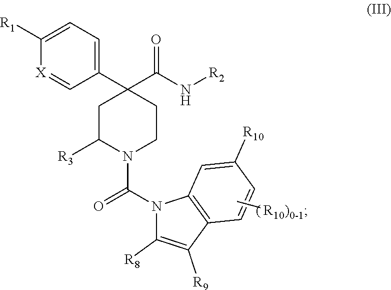
wherein X, R₁, R₃, and each R₁₀ independently are as defined in claim 1;
R₂ is R₂ₐ;
R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b};
each R_{2b} substituent is independently selected from methyl and halo (e.g. fluoro);
R₈ is selected from hydrogen, methyl, ethyl, CHF₂, CH₂CH₂OCH₃, C(═O)H and cyano;
R₉ is X₃—R₉ₐ;
X₃ is CH₂; and
R₉ₐ is selected from i) hydrogen and ii) a ring substituted with 0 to 2 (e.g. 0 to 1) R_{9b} groups, wherein the ring is a) C₅-C₆cycloalkyl or b) 6 membered heterocyclyl comprising 1 heteroatom which is O; and each R_{9b} is as defined in claim 1 (e.g. each R_{9b} is independently selected from O—C₁-C₃alkyl and 6 membered heterocyclyl comprising 1 heteroatom which is O).

37. The compound or pharmaceutically acceptable salt thereof according to claim 1, wherein the compound is of formula (IV): 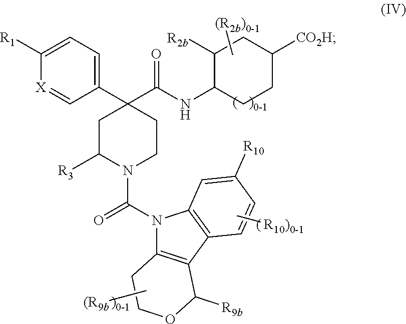
wherein X, R₁, R₃, each R_{2b} independently, each R_{9b} independently and each R₁₀ independently are as defined in claim 1.

38. The compound or pharmaceutically acceptable salt thereof according to claim 1, wherein the compound is of formula (V): 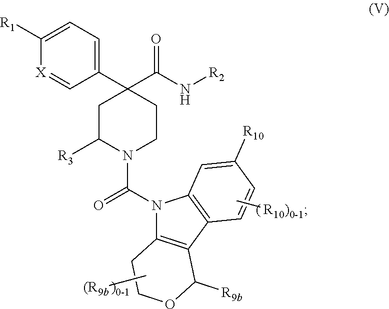
wherein X, R₁, R₃, each R_{9b} independently and each R₁₀ independently are as defined in claim 1;
R₂ is R₂ₐ;
R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b}; and
each R_{2b} substituent is independently selected from methyl and halo (e.g. fluoro).

39. The compound or pharmaceutically acceptable salt thereof according to claim 1, wherein the compound is of formula (VI): 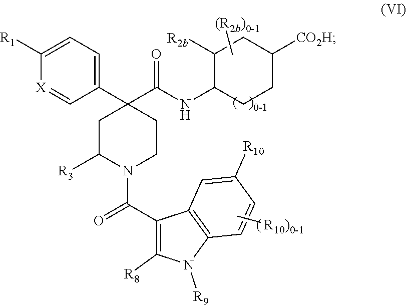
wherein:
X, R₁, R₃, each R_{2b} independently, and each R₁₀ independently are as defined in claim 1;
R₈ is selected from hydrogen, methyl, ethyl, CHF₂, CH₂CH₂OCH₃, C(═O)H and cyano;
R₉ is X₃—R₉ₐ;
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and each R_{9b} is as defined in claim 1 (e.g. each R_{9b} is independently selected from O—C₁-C₃alkyl and 6 membered heterocyclyl comprising 1 heteroatom which is O).

40. The compound or pharmaceutically acceptable salt thereof according to claim 1, wherein the compound is of formula (VII): 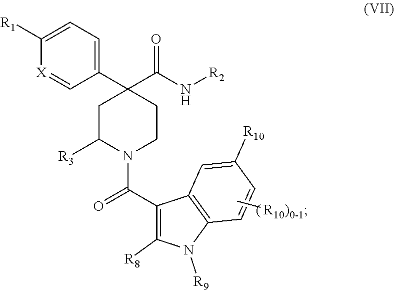
wherein:
X, R₁, R₃, and each R₁₀ independently are as defined in claim 1;
R₂ is R₂ₐ;
R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b};
each R_{2b} substituent is independently selected from methyl and halo (e.g. fluoro);
R₈ is selected from hydrogen, methyl, ethyl, CHF₂, CH₂CH₂OCH₃, C(═O)H and cyano;
R₉ is X₃—R₉ₐ;
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and each R_{9b} is as defined in claim 1 (e.g. each R_{9b} is independently selected from O—C₁-C₃alkyl and 6 membered heterocyclyl comprising 1 heteroatom which is O).

41. The compound or pharmaceutically acceptable salt thereof according to claim 1, wherein the compound is of formula (VIII): 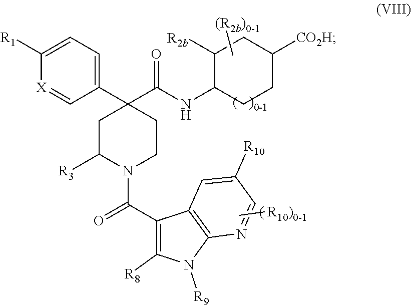
wherein:
X, R₁, R₃, each R_{2b} independently, and each R₁₀ independently are as defined in claim 1;
R₈ is selected from hydrogen, methyl, ethyl, CHF₂, CH₂CH₂OCH₃, C(═O)H and cyano;
R₉ is X₃—R₉ₐ;
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and each R_{9b} is as defined in claim 1 (e.g. each R_{9b} is independently selected from O—C₁-C₃alkyl and 6 membered heterocyclyl comprising 1 heteroatom which is O).

42. The compound or pharmaceutically acceptable salt thereof according to claim 1, wherein the compound is of formula (IX): 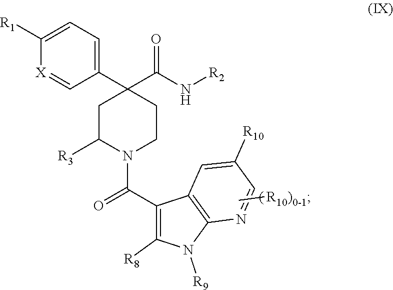
wherein:
X, R₁, R₃, and each R₁₀ independently are as defined in claim 1;
R₂ is R₂ₐ;
R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b};
each R_{2b} substituent is independently selected from methyl and halo (e.g. fluoro);
R₈ is selected from hydrogen, methyl, ethyl, CHF₂, CH₂CH₂OCH₃, C(═O)H and cyano;
R₉ is X₃—R₉ₐ;
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and each R_{9b} is as defined in claim 1 (e.g. each R_{9b} is independently selected from O—C₁-C₃alkyl and 6 membered heterocyclyl comprising 1 heteroatom which is O).

43. The compound or pharmaceutically acceptable salt thereof according to claim 1, wherein the compound is of formula (X): 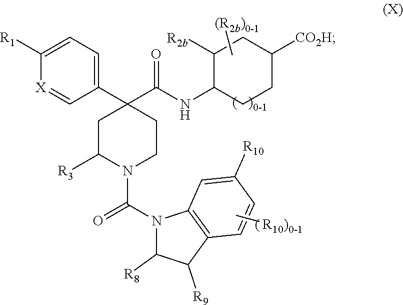
wherein:
X, R₁, R₃, each R_{2b} independently, and each R₁₀ independently are as defined in claim 1;
R₈ is selected from hydrogen, methyl, ethyl, CHF₂, CH₂CH₂OCH₃, C(═O)H and cyano;
R₉ is R₉ₐ or X₃—R₉ₐ (particularly R₉ₐ);
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and each R_{9b} is as defined in claim 1 (e.g. each R_{9b} is independently selected from O—C₁-C₃alkyl and 6 membered heterocyclyl comprising 1 heteroatom which is O).

44. The compound or pharmaceutically acceptable salt thereof according to claim 1, wherein the compound is of formula (XI): 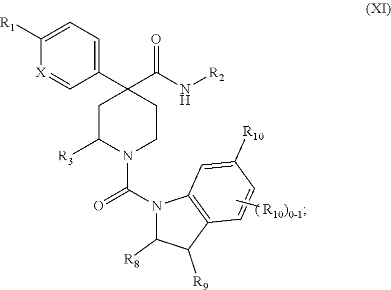
wherein:
X, R₁, R₃, and each R₁₀ independently are as defined in claim 1;
R₂ is R₂ₐ;
R₂ₐ is 4 to 6 membered heterocyclyl (e.g. fully saturated heterocyclyl) containing 1 or 2 heteroatoms independently selected from oxygen and nitrogen substituted with 0 to 3 substituents R_{2b};
each R_{2b} substituent is independently selected from methyl and halo (e.g. fluoro);
R₈ is selected from hydrogen, methyl, ethyl, CHF₂, CH₂CH₂OCH₃, C(═O)H and cyano;
R₉ is R₉ₐ or X₃—R₉ₐ (particularly R₉ₐ);
X₃ is CH₂; and
R₉ₐ is selected from i) hydrogen and ii) a ring substituted with 0 to 2 R_{9b} groups, wherein the ring is a) C₅-C₆cycloalkyl or b) 6 membered heterocyclyl comprising 1 heteroatom which is O; and each R_{9b} is as defined in claim 1 (e.g. each R_{9b} is independently selected from O—C₁-C₃alkyl and 6 membered heterocyclyl comprising 1 heteroatom which is O).

45. A compound selected from: 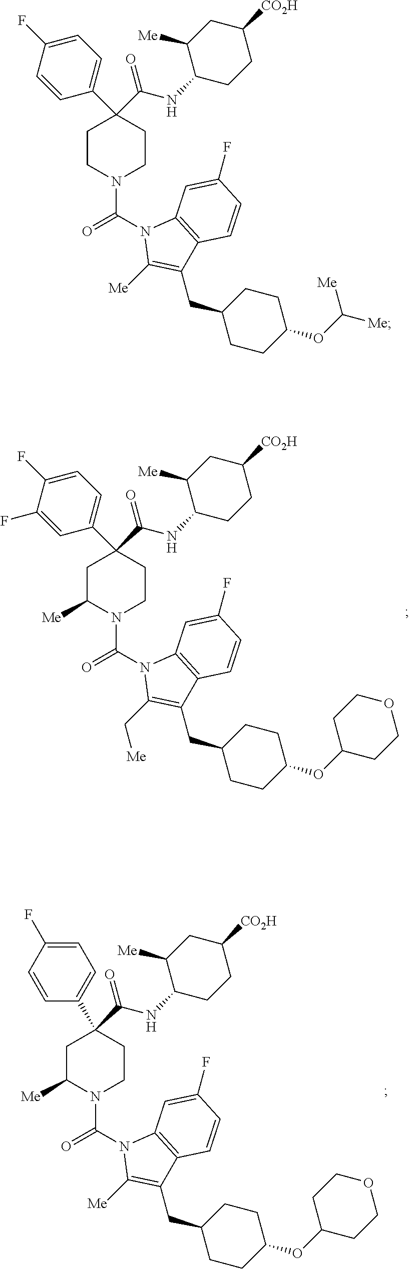 [SMILES: Cc1c(C[C@H]2CC[C@H](OC(C)C)CC2)c2ccc(F)cc2n1C(=O)N1CCC(C(=O)N[C@H]2CC[C@H](C(=O)O)C[C@@H]2C)(c2ccc(F)cc2)CC1 ; CCc1c(C[C@H]2CC[C@H](OC3CCOCC3)CC2)c2ccc(F)cc2n1C(=O)N1CC[C@@](C(=O)N[C@H]2CC[C@H](C(=O)O)C[C@@H]2C)(c2ccc(F)c(F)c2)C[C@@H]1C ; Cc1c(C[C@H]2CC[C@H](OC3CCOCC3)CC2)c2ccc(F)cc2n1C(=O)N1CC[C@@](C(=O)N[C@H]2CC[C@H](C(=O)O)C[C@@H]2C)(c2ccc(F)cc2)C[C@@H]1C] 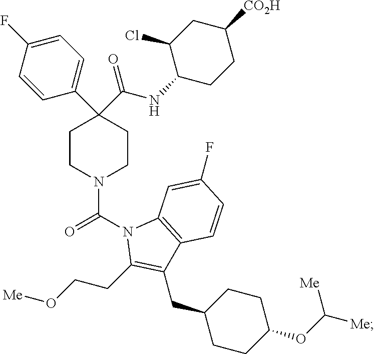 [SMILES: COCCc1c(C[C@H]2CC[C@H](OC(C)C)CC2)c2ccc(F)cc2n1C(=O)N1CCC(C(=O)N[C@H]2CC[C@H](C(=O)O)C[C@@H]2Cl)(c2ccc(F)cc2)CC1] 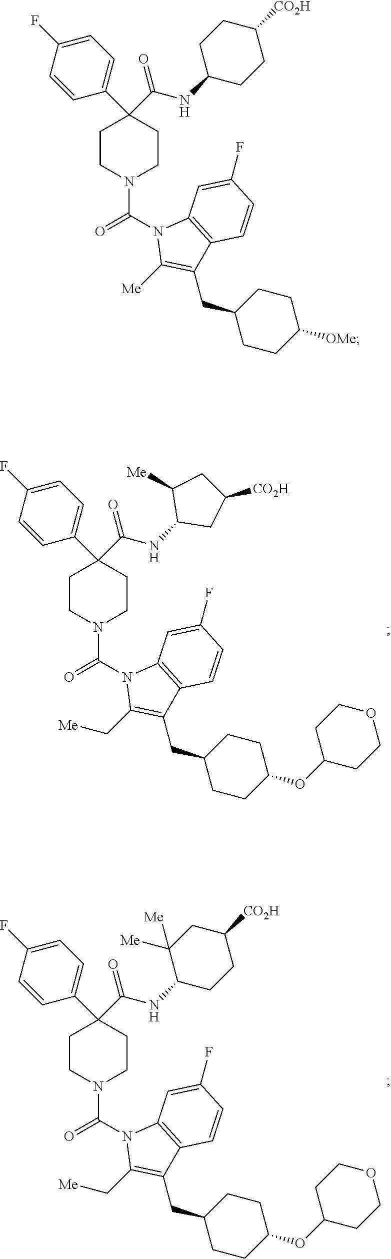 [SMILES: CO[C@H]1CC[C@H](Cc2c(C)n(C(=O)N3CCC(C(=O)N[C@H]4CC[C@H](C(=O)O)CC4)(c4ccc(F)cc4)CC3)c3cc(F)ccc23)CC1 ; CCc1c(C[C@H]2CC[C@H](OC3CCOCC3)CC2)c2ccc(F)cc2n1C(=O)N1CCC(C(=O)N[C@H]2C[C@H](C(=O)O)C[C@@H]2C)(c2ccc(F)cc2)CC1 ; CCc1c(C[C@H]2CC[C@H](OC3CCOCC3)CC2)c2ccc(F)cc2n1C(=O)N1CCC(C(=O)N[C@H]2CC[C@H](C(=O)O)CC2(C)C)(c2ccc(F)cc2)CC1] 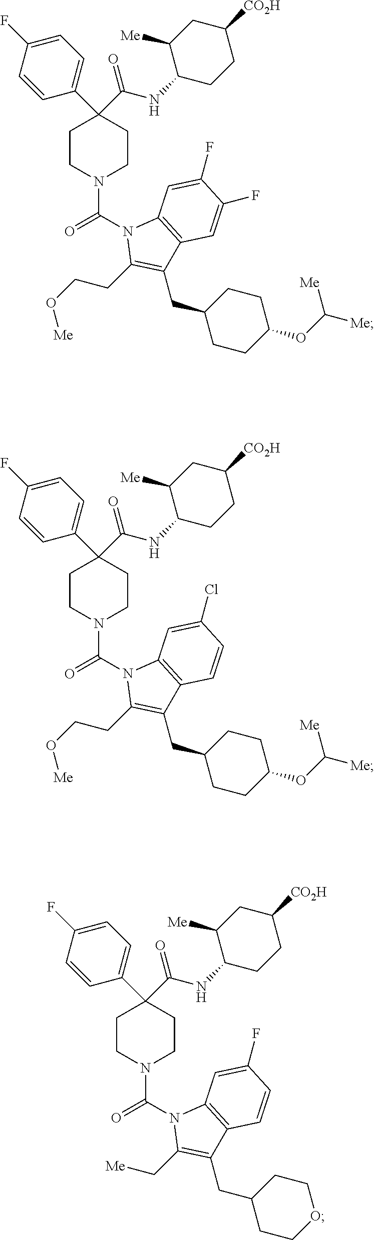 [SMILES: COCCc1c(C[C@H]2CC[C@H](OC(C)C)CC2)c2cc(F)c(F)cc2n1C(=O)N1CCC(C(=O)N[C@H]2CC[C@H](C(=O)O)C[C@@H]2C)(c2ccc(F)cc2)CC1 ; COCCc1c(C[C@H]2CC[C@H](OC(C)C)CC2)c2ccc(Cl)cc2n1C(=O)N1CCC(C(=O)N[C@H]2CC[C@H](C(=O)O)C[C@@H]2C)(c2ccc(F)cc2)CC1 ; CCc1c(CC2CCOCC2)c2ccc(F)cc2n1C(=O)N1CCC(C(=O)N[C@H]2CC[C@H](C(=O)O)C[C@@H]2C)(c2ccc(F)cc2)CC1] 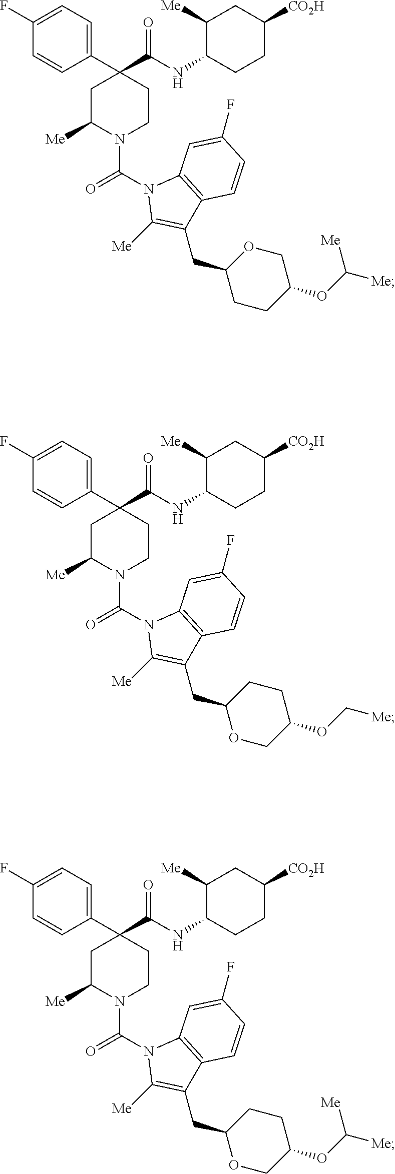 [SMILES: Cc1c(C[C@@H]2CC[C@@H](OC(C)C)CO2)c2ccc(F)cc2n1C(=O)N1CC[C@@](C(=O)N[C@H]2CC[C@H](C(=O)O)C[C@@H]2C)(c2ccc(F)cc2)C[C@@H]1C ; CCO[C@H]1CC[C@H](Cc2c(C)n(C(=O)N3CC[C@@](C(=O)N[C@H]4CC[C@H](C(=O)O)C[C@@H]4C)(c4ccc(F)cc4)C[C@@H]3C)c3cc(F)ccc23)OC1 ; Cc1c(C[C@H]2CC[C@H](OC(C)C)CO2)c2ccc(F)cc2n1C(=O)N1CC[C@@](C(=O)N[C@H]2CC[C@H](C(=O)O)C[C@@H]2C)(c2ccc(F)cc2)C[C@@H]1C] 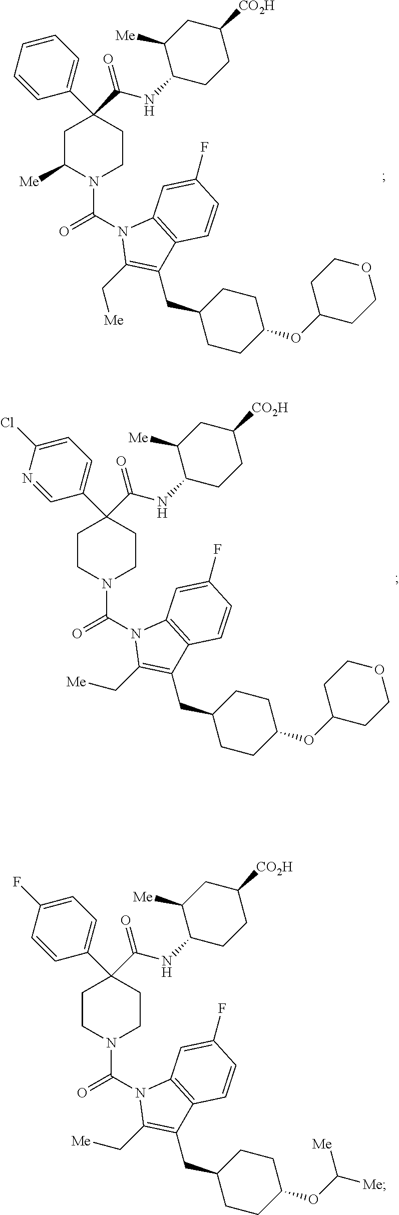 [SMILES: CCc1c(C[C@H]2CC[C@H](OC3CCOCC3)CC2)c2ccc(F)cc2n1C(=O)N1CC[C@@](C(=O)N[C@H]2CC[C@H](C(=O)O)C[C@@H]2C)(c2ccccc2)C[C@@H]1C ; CCc1c(C[C@H]2CC[C@H](OC3CCOCC3)CC2)c2ccc(F)cc2n1C(=O)N1CCC(C(=O)N[C@H]2CC[C@H](C(=O)O)C[C@@H]2C)(c2ccc(Cl)nc2)CC1 ; CCc1c(C[C@H]2CC[C@H](OC(C)C)CC2)c2ccc(F)cc2n1C(=O)N1CCC(C(=O)N[C@H]2CC[C@H](C(=O)O)C[C@@H]2C)(c2ccc(F)cc2)CC1] 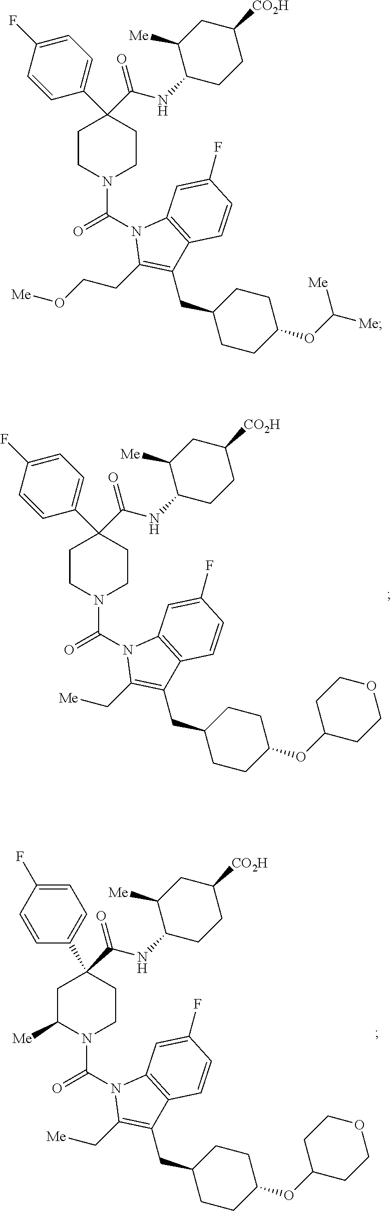 [SMILES: COCCc1c(C[C@H]2CC[C@H](OC(C)C)CC2)c2ccc(F)cc2n1C(=O)N1CCC(C(=O)N[C@H]2CC[C@H](C(=O)O)C[C@@H]2C)(c2ccc(F)cc2)CC1 ; CCc1c(C[C@H]2CC[C@H](OC3CCOCC3)CC2)c2ccc(F)cc2n1C(=O)N1CCC(C(=O)N[C@H]2CC[C@H](C(=O)O)C[C@@H]2C)(c2ccc(F)cc2)CC1 ; CCc1c(C[C@H]2CC[C@H](OC3CCOCC3)CC2)c2ccc(F)cc2n1C(=O)N1CC[C@@](C(=O)N[C@H]2CC[C@H](C(=O)O)C[C@@H]2C)(c2ccc(F)cc2)C[C@@H]1C] 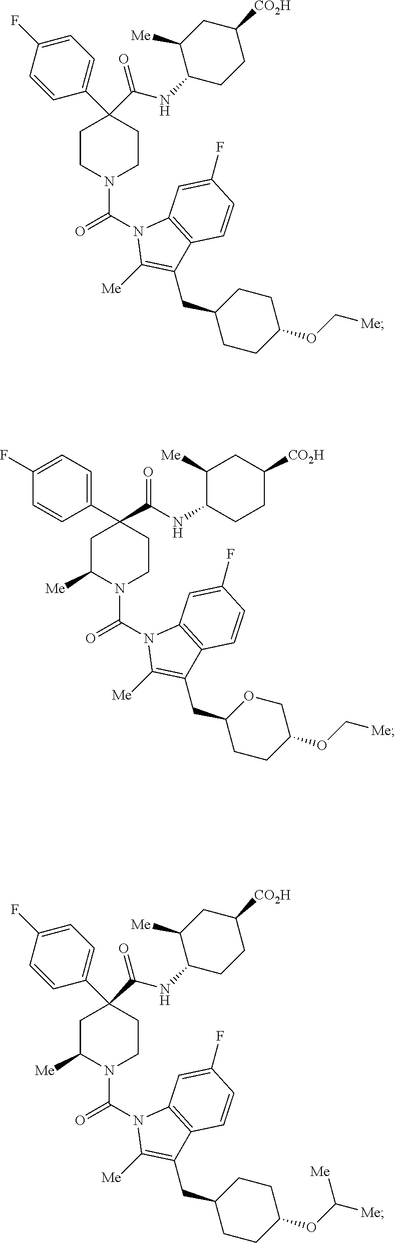 [SMILES: CCO[C@H]1CC[C@H](Cc2c(C)n(C(=O)N3CCC(C(=O)N[C@H]4CC[C@H](C(=O)O)C[C@@H]4C)(c4ccc(F)cc4)CC3)c3cc(F)ccc23)CC1 ; CCO[C@@H]1CC[C@@H](Cc2c(C)n(C(=O)N3CC[C@@](C(=O)N[C@H]4CC[C@H](C(=O)O)C[C@@H]4C)(c4ccc(F)cc4)C[C@@H]3C)c3cc(F)ccc23)OC1 ; Cc1c(C[C@H]2CC[C@H](OC(C)C)CC2)c2ccc(F)cc2n1C(=O)N1CC[C@@](C(=O)N[C@H]2CC[C@H](C(=O)O)C[C@@H]2C)(c2ccc(F)cc2)C[C@@H]1C] 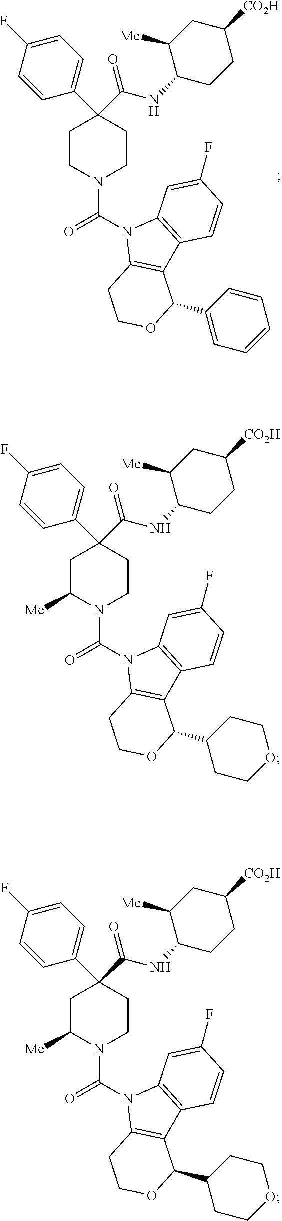 [SMILES: C[C@H]1C[C@@H](C(=O)O)CC[C@@H]1NC(=O)C1(c2ccc(F)cc2)CCN(C(=O)n2c3c(c4ccc(F)cc42)[C@H](c2ccccc2)OCC3)CC1 ; C[C@H]1C[C@@H](C(=O)O)CC[C@@H]1NC(=O)C1(c2ccc(F)cc2)CCN(C(=O)n2c3c(c4ccc(F)cc42)[C@H](C2CCOCC2)OCC3)[C@@H](C)C1 ; C[C@H]1C[C@@H](C(=O)O)CC[C@@H]1NC(=O)[C@@]1(c2ccc(F)cc2)CCN(C(=O)n2c3c(c4ccc(F)cc42)[C@@H](C2CCOCC2)OCC3)[C@@H](C)C1] 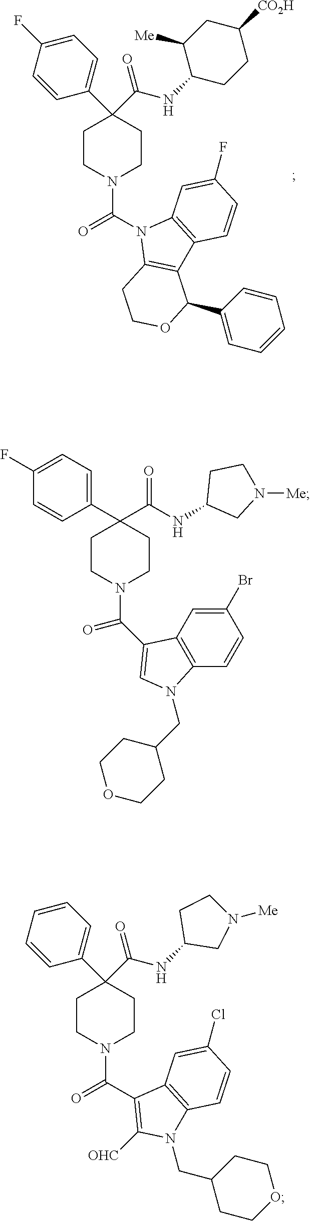 [SMILES: C[C@H]1C[C@@H](C(=O)O)CC[C@@H]1NC(=O)C1(c2ccc(F)cc2)CCN(C(=O)n2c3c(c4ccc(F)cc42)[C@@H](c2ccccc2)OCC3)CC1 ; CN1CC[C@@H](NC(=O)C2(c3ccc(F)cc3)CCN(C(=O)c3cn(CC4CCOCC4)c4ccc(Br)cc34)CC2)C1 ; CN1CC[C@@H](NC(=O)C2(c3ccccc3)CCN(C(=O)c3c(C=O)n(CC4CCOCC4)c4ccc(Cl)cc34)CC2)C1] 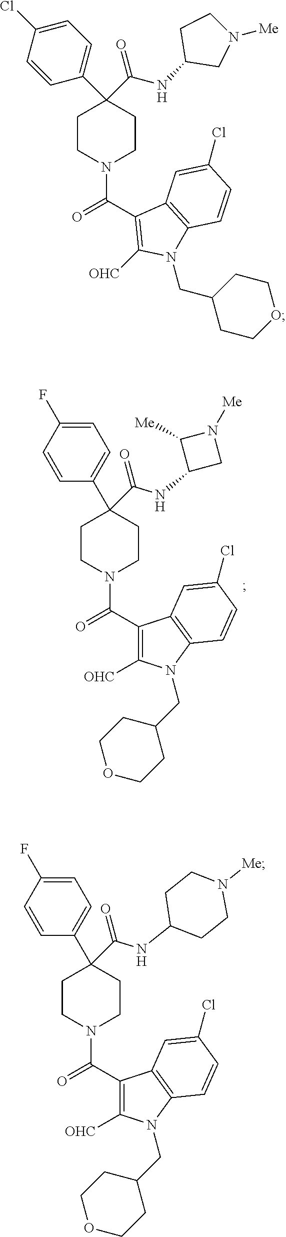 [SMILES: CN1CC[C@@H](NC(=O)C2(c3ccc(Cl)cc3)CCN(C(=O)c3c(C=O)n(CC4CCOCC4)c4ccc(Cl)cc34)CC2)C1 ; C[C@H]1[C@@H](NC(=O)C2(c3ccc(F)cc3)CCN(C(=O)c3c(C=O)n(CC4CCOCC4)c4ccc(Cl)cc34)CC2)CN1C ; CN1CCC(NC(=O)C2(c3ccc(F)cc3)CCN(C(=O)c3c(C=O)n(CC4CCOCC4)c4ccc(Cl)cc34)CC2)CC1] 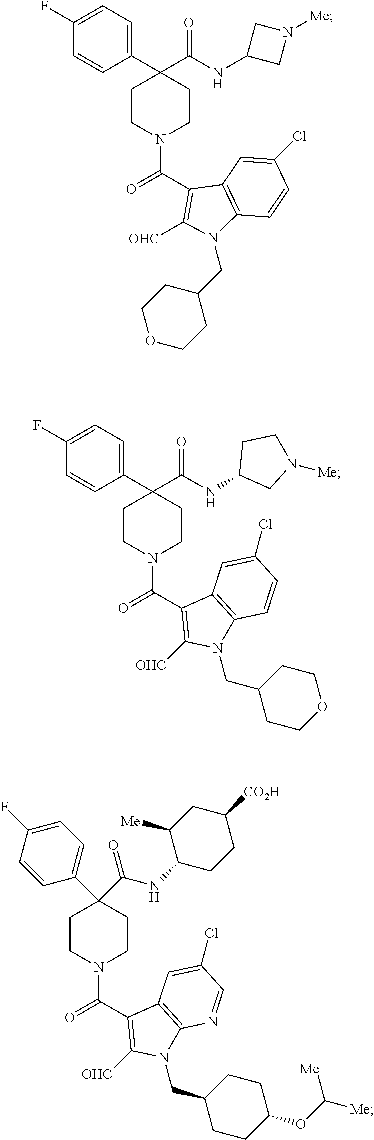 [SMILES: CN1CC(NC(=O)C2(c3ccc(F)cc3)CCN(C(=O)c3c(C=O)n(CC4CCOCC4)c4ccc(Cl)cc34)CC2)C1 ; CN1CC[C@@H](NC(=O)C2(c3ccc(F)cc3)CCN(C(=O)c3c(C=O)n(CC4CCOCC4)c4ccc(Cl)cc34)CC2)C1 ; CC(C)O[C@H]1CC[C@H](Cn2c(C=O)c(C(=O)N3CCC(C(=O)N[C@H]4CC[C@H](C(=O)O)C[C@@H]4C)(c4ccc(F)cc4)CC3)c3cc(Cl)cnc32)CC1] 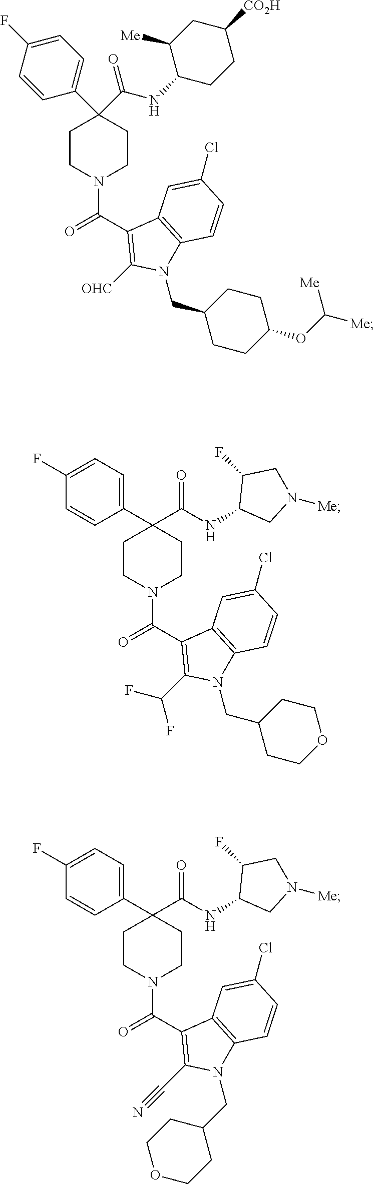 [SMILES: CC(C)O[C@H]1CC[C@H](Cn2c(C=O)c(C(=O)N3CCC(C(=O)N[C@H]4CC[C@H](C(=O)O)C[C@@H]4C)(c4ccc(F)cc4)CC3)c3cc(Cl)ccc32)CC1 ; CN1C[C@@H](F)[C@@H](NC(=O)C2(c3ccc(F)cc3)CCN(C(=O)c3c(C(F)F)n(CC4CCOCC4)c4ccc(Cl)cc34)CC2)C1 ; CN1C[C@@H](F)[C@@H](NC(=O)C2(c3ccc(F)cc3)CCN(C(=O)c3c(C#N)n(CC4CCOCC4)c4ccc(Cl)cc34)CC2)C1] 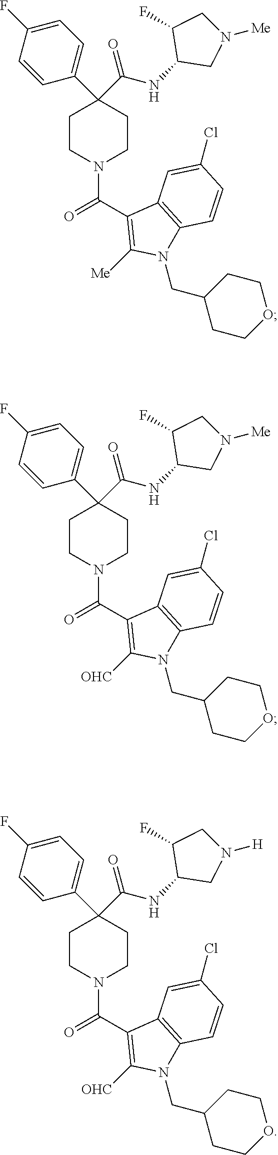 [SMILES: Cc1c(C(=O)N2CCC(C(=O)N[C@H]3CN(C)C[C@H]3F)(c3ccc(F)cc3)CC2)c2cc(Cl)ccc2n1CC1CCOCC1 ; CN1C[C@@H](F)[C@@H](NC(=O)C2(c3ccc(F)cc3)CCN(C(=O)c3c(C=O)n(CC4CCOCC4)c4ccc(Cl)cc34)CC2)C1 ; O=Cc1c(C(=O)N2CCC(C(=O)N[C@H]3CNC[C@H]3F)(c3ccc(F)cc3)CC2)c2cc(Cl)ccc2n1CC1CCOCC1] 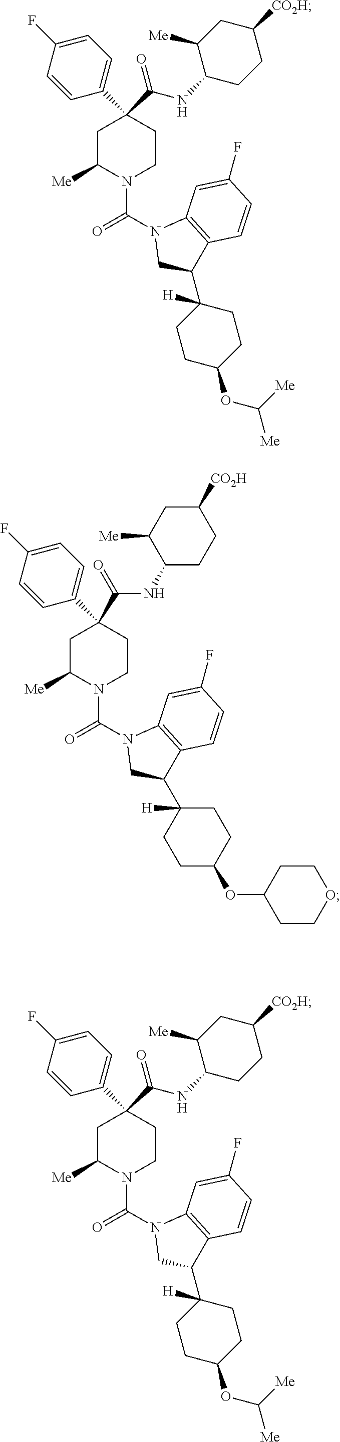 [SMILES: CC(C)O[C@H]1CC[C@H]([C@H]2CN(C(=O)N3CC[C@@](C(=O)N[C@H]4CC[C@H](C(=O)O)C[C@@H]4C)(c4ccc(F)cc4)C[C@@H]3C)c3cc(F)ccc32)CC1 ; C[C@H]1C[C@@H](C(=O)O)CC[C@@H]1NC(=O)[C@@]1(c2ccc(F)cc2)CCN(C(=O)N2C[C@H]([C@H]3CC[C@H](OC4CCOCC4)CC3)c3ccc(F)cc32)[C@@H](C)C1 ; CC(C)O[C@H]1CC[C@H]([C@@H]2CN(C(=O)N3CC[C@@](C(=O)N[C@H]4CC[C@H](C(=O)O)C[C@@H]4C)(c4ccc(F)cc4)C[C@@H]3C)c3cc(F)ccc32)CC1] 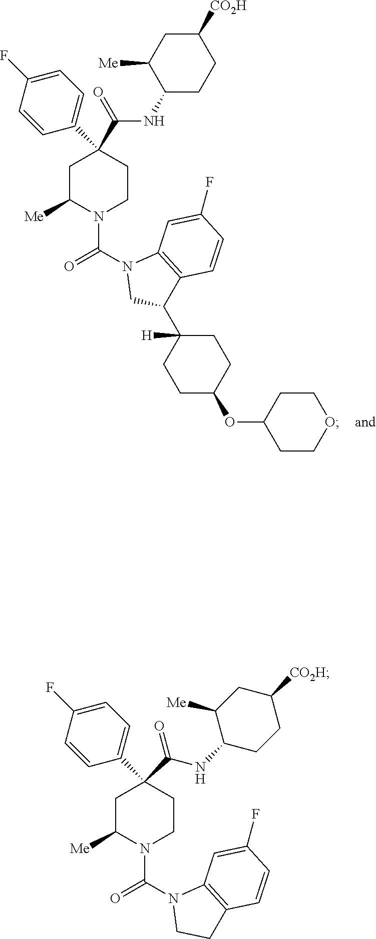 [SMILES: C[C@H]1C[C@@H](C(=O)O)CC[C@@H]1NC(=O)[C@@]1(c2ccc(F)cc2)CCN(C(=O)N2C[C@@H]([C@H]3CC[C@H](OC4CCOCC4)CC3)c3ccc(F)cc32)[C@@H](C)C1 ; C[C@H]1C[C@@H](C(=O)O)CC[C@@H]1NC(=O)[C@@]1(c2ccc(F)cc2)CCN(C(=O)N2CCc3ccc(F)cc32)[C@@H](C)C1]
or a pharmaceutically acceptable salt thereof.

46. A pharmaceutical composition comprising the compound or pharmaceutically acceptable salt thereof according to claim 1, and one or more pharmaceutically acceptable carriers.

47. A combination comprising the compound or pharmaceutically acceptable salt thereof according to claim 1, and one or more additional therapeutically active agents.

48. A method of modulating ERK activity in a subject, the method comprising administering to the subject a therapeutically effective amount of the compound or pharmaceutically acceptable salt thereof according to claim 1.

49. A method of treating a patient having a disease associated with aberrant activity of the MAP kinase pathway comprising administering to said patient a therapeutically effective amount of the compound or pharmaceutically acceptable salt thereof according to claim 1.

50. The method according to claim 49, wherein the disease associated with aberrant activity of the MAP kinase pathway is cancer.

51. The method according to claim 50, wherein the cancer is selected from melanoma, lung cancer, colorectal cancer (CRC), pancreatic cancer and thyroid cancer.

52. The method according to claim 50, wherein the cancer contains a BRAF and/or a RAS mutation.

53.-59. (canceled)
